(19) United States
(12) Patent Application Publication
(10) Pub. No.: US 2012/0065482 A1
(43) Pub. Date: Mar. 15, 2012

(54) DETERMINATION OF BLOOD PUMP SYSTEM PERFORMANCE AND SAMPLE DILUTION USING A PROPERTY OF FLUID BEING TRANSPORTED

(76) Inventors: Mark Ries Robinson, Albuquerque, NM (US); Mike Borrello, Carlsbad, CA (US); Richard P. Thompson, Dana Point, CA (US); Stephen Vanslyke, Carlsbad, CA (US); Shonn Hendee, Carlsbad, CA (US); Dan Welsh, Encinitas, CA (US); Steve Bernard, Andover, MN (US); John O'Mahony, Maple Grove, MN (US); Dave McMahon, Solana Beach, CA (US); Victor Gerald Grafe, Corrales, NM (US); Dave Tobler, Carlsbad, CA (US); William R. Patterson, Irvine, CA (US); Donald W. Landry, New York, NY (US); James H. Macemon, Poway, CA (US); Russell Abbink, Sandia Park, NM (US)

(21) Appl. No.: 13/193,602

(22) Filed: Jul. 29, 2011

Related U.S. Application Data
(63) Continuation-in-part of application No. 12/325,243, filed on Nov. 30, 2008.
Application No. 12/325,243 is a continuation-in-part of application No. 11/679,826, filed on Feb. 27, 2007.
Application No. 11/679,826 is a continuation-in-part of application No. PCT/US2006/060850, filed on Nov. 13, 2006.
Application No. PCT/US2006/060850 is a continuation-in-part of application No. 11/679,837, filed on Feb. 28, 2007.
Application No. 11/679,837 is a continuation-in-part of application No. PCT/US2006/060850, filed on Nov. 13, 2006.
Application No. PCT/US2006/060850 is a continuation-in-part of application No. 11/679,839, filed on Feb. 28, 2007.
Application No. 11/679,839 is a continuation-in-part of application No. PCT/US2006/060850, filed on Nov. 13, 2006.
Application No. PCT/US2006/060850 is a continuation-in-part of application No. 11/679,835, filed on Feb. 27, 2007.
Application No. 11/679,835 is a continuation-in-part of application No. 12/188,205, filed on Aug. 8, 2008, now abandoned.
Application No. 12/188,205 is a continuation-in-part of application No. 11/842,624, filed on Aug. 21, 2007, now abandoned.
Application No. 11/842,624 is a continuation-in-part of application No. 11/860,544, filed on Sep. 25, 2007, now abandoned.
Application No. 11/860,544 is a continuation-in-part of application No. 11/860,545, filed on Sep. 25, 2007, now abandoned.
Application No. 11/860,545 is a continuation-in-part of application No. 12/241,221, filed on Sep. 30, 2008.
Application No. 12/241,221 is a continuation-in-part of application No. 12/576,121, filed on Oct. 8, 2009.
Application No. 12/576,121 is a continuation-in-part of application No. 12/576,303, filed on Oct. 9, 2009.
Application No. 12/576,303 is a continuation-in-part of application No. 12/577,153, filed on Oct. 10, 2009, now abandoned.
Application No. 12/577,153 is a continuation of application No. 11/101,439, filed on Apr. 8, 2005, now abandoned.
Application No. 11/101,439 is a continuation-in-part of application No. 12/641,411, filed on Dec. 18, 2009.
Application No. 12/641,411 is a continuation-in-part of application No. 12/714,100, filed on Feb. 26, 2010.
Application No. 12/714,100 is a continuation-in-part of application No. 11/679,835, filed on Feb. 27, 2007.
Application No. 11/679,835 is a continuation-in-part of application No. 12/884,175, filed on Sep. 16, 2010.
Application No. 12/884,175 is a continuation-in-part of application No. PCT/US2009/037398, filed on Mar. 17, 2009.
Application No. PCT/US2009/037398 is a continuation-in-part of application No. PCT/US2009/037402, filed on Mar. 17, 2009.
Application No. PCT/US2009/037402 is a continuation-in-part of application No. 11/679,826, filed on Feb. 27, 2007.
Application No. 11/679,826 is a continuation-in-part of application No. PCT/US2006/060850, filed on Nov. 13, 2006.
(60) Provisional application No. 60/791,719, filed on Apr. 12, 2006.
(60) Provisional application No. 60/737,254, filed on Nov. 15, 2005.
(60) Provisional application No. 60/791,719, filed on Apr. 12, 2006.
(60) Provisional application No. 60/791,719, filed on Apr. 12, 2006.
(60) Provisional application No. 60/791,719, filed on Apr. 12, 2006.
(60) Provisional application No. 60/955,636, filed on Aug. 13, 2007.
(60) Provisional application No. 60/955,636, filed on Aug. 13, 2007.
(60) Provisional application No. 60/976,775, filed on Oct. 1, 2007.
(60) Provisional application No. 61/104,193, filed on Oct. 9, 2008.
(60) Provisional application No. 61/104,252, filed on Oct. 9, 2008.
(60) Provisional application No. 61/105,600, filed on Oct. 15, 2008.
(60) Provisional application No. 60/791,719, filed on Apr. 12, 2006.
(60) Provisional application No. 60/737,254, filed on Nov. 15, 2005.
(60) Provisional application No. 61/444,118, filed on Feb. 17, 2011.

(51) Int. Cl. A61B 5/145

(52) U.S. Cl. 600/309

(57) ABSTRACT

The present invention provides methods and apparatuses related to measurement of analytes, including measurements of analytes in samples withdrawn from a patient.

### CROSS-REFERENCE TO RELATED APPLICATIONS

This application claims priority as a continuation in part of the following U.S. application Ser. Nos. 11/679,826, filed Feb. 27, 2007, 11/679,837, filed Feb. 28, 2007, 11/679,839, filed Feb. 28, 2007, 11/860,544, filed Sep. 25, 2007, 11/860,545, filed Sep. 25, 2007, 12/241,221, filed Sep. 30, 2008, 12/576,303, filed Oct. 9, 2009, 12/577,153, filed Oct. 10, 2009, 12/641,411, filed Dec. 18, 2009, 12/714,100, filed Feb. 26, 2010, 12/884,175, filed Sep. 16, 2010, 11/679,835 filed Feb. 27, 2007, which claimed priority to U.S. provisional 60/791,719 filed Apr. 12, 2006, 11/842,624, filed Aug. 21, 2007, 11/101,439, filed Apr. 8, 2005, 12/188,205, filed Aug. 8, 2008, 12/108,250, filed Apr. 23, 2008, 12/576,121, filed Oct. 8, 2009, 10/850,646, filed May 21, 2004;

And claims priority to the following U.S. provisional applications: 60/791,719, filed Apr. 12, 2006, 60/737,254, filed Nov. 15, 2006, 61/105,600, filed Oct. 15, 2008, 61/104,252, filed Oct. 9, 2008, 61/104,193, filed Oct. 9, 2008, 60/955,636, filed Aug. 13, 2007, 60/913,582, filed Apr. 24, 2007, 60/991,373, filed Nov. 30, 2007, 61/044,004, filed Apr. 10, 2008, 60/976,775, filed Oct. 1, 2007, 61/444,118, filed Feb. 17, 2011;

And as a continuation in part of the following PCT applications: PCT/US2006/060850, filed Nov. 13, 2006, PCT/US2009/037398, filed Mar. 17, 2009, PCT/US2009/037402, filed Mar. 17, 2009. Each of the foregoing applications is incorporated herein by reference.

### FIELD OF THE INVENTION

This invention relates to the field of the measurement of blood analytes, and more specifically to the measurement of analytes such as glucose in blood that has been temporarily removed from a body.

### BACKGROUND OF THE INVENTION

More than 20 peer-reviewed publications have demonstrated that tight control of blood glucose significantly improves critical care patient outcomes. Tight glycemic control (TGC) has been shown to reduce surgical site infections by 60% in cardiothoracic surgery patients and reduce overall ICU mortality by 40% with significant reductions in ICU morbidity and length of stay. See, e.g., Furnary, Tony, Oral presentation at 2005 ADA annual, session titled “Management of the Hospitalized Hyperglycemic Patient;” Van den Berghe et al., NEJM 2001; 345:1359. Historically, caregivers have treated hyperglycemia (high blood glucose) only when glucose levels exceeded 220 mg/dl. Based upon recent clinical findings, however, experts now recommend IV insulin administration to control blood glucose to within the normoglycemic range (80-110 mg/dl). Adherence to such strict glucose control regimens requires near-continuous monitoring of blood glucose and frequent adjustment of insulin infusion to achieve normoglycemia while avoiding risk of hypoglycemia (low blood glucose). In response to the demonstrated clinical benefit, approximately 50% of US hospitals have adopted some form of tight glycemic control with an additional 23% expected to adopt protocols within the next 12 months. Furthermore, 36% of hospitals already using glycemic management protocols in their ICUs plan to expand the practice to other units and 40% of hospitals that have near-term plans to adopt TGC protocols in the ICU also plan to do so in other areas of the hospital.

Given the compelling evidence for improved clinical outcomes associated with tight glycemic control, hospitals are under pressure to implement TGC as the standard of practice for critical care and cardiac surgery patients. Clinicians and caregivers have developed TGC protocols that use IV insulin administration to maintain normal patient glucose levels. To be safe and effective, these protocols require frequent blood glucose monitoring. Currently, these protocols involve periodic removal of blood samples by nursing staff and testing on handheld meters or blood gas analyzers. Although hospitals are responding to the identified clinical need, adoption has been difficult with current technology due to two principal reasons.

Fear of hypoglycemia. The target glucose range of 80-110 mg/dl brings the patient near clinical hypoglycemia (blood glucose less than 50 mg/dl). Patients exposed to hypoglycemia for greater than 30 minutes have significant risk of neurological damage. IV insulin administration with only intermittent glucose monitoring (typically hourly by most TGC protocols) exposes patients to increased risk of hypoglycemia. In a recent letter to the editors of Intensive Care medicine, it was noted that 42% of patients treated with a TGC protocol in the UK experienced at least one episode of hypoglycemia. See, e.g., lain Mackenzie et al., “Tight glycaemic control: a survey of intensive care practice in large English Hospitals;” Intensive Care Med (2005) 31:1136. In addition, handheld meters require procedural steps that are often cited as a source of measurement error, further exacerbating the fear (and risk) of accidentally taking the blood glucose level too low. See, e.g., Bedside Glucose Testing systems, CAP today, April 2005, page 44.

Burdensome procedure. Most glycemic control protocols require frequent glucose monitoring and insulin adjustment at 30 minute to 2 hour intervals (typically hourly) to achieve normoglycemia. Caregivers recognize that glucose control would be improved with continuous or near-continuous monitoring. Unfortunately, existing glucose monitoring technology is incompatible with the need to obtain frequent measurements. Using current technology, each measurement requires removal of a blood sample, performance of the blood glucose test, evaluation of the result, determination of the correct therapeutic action, and finally adjustment to the insulin infusion rate. High measurement frequency requirements coupled with a labor-intensive and time-consuming test places significant strain on limited ICU nursing resources that already struggle to meet patient care needs.

Limitations of Finger-Stick Technology To implement TGC protocols using today's manual, finger-stick technologies requires many steps, is technique sensitive and has opportunities for user errors. Using these technologies require removal of a blood sample, placement of just the right amount of blood on a test strip, evaluation of the result, determination of the correct glucose or insulin dose using a complex algorithm, and finally adjustment to the insulin infusion rate. In a recent study published in the America College of Surgeons in 2006, Taylor et al. noted that while implementing a TGC protocol, errors were found in the implementation of the protocol in 47% of all patients. Half of the errors were considered major, such as missing two or more glucose measurements in a row and insulin dosing errors. See Taylor et al., Journal of American College of Surgeons, 202, 1 (2006), which is incorporated herein by reference. The current manual method of TGC requires multiple types of equipment and at least two hours of nursing time per patient per day to implement. Even with all of this equipment and time spent, the targeted glycemic range of 80-110 mg/dl is difficult to achieve and maintaining patients in this range is even more difficult.

Medication errors are a significant and growing problem that can result in tragic loss of life and significant cost increases to the health-care community. Recent studies have listed medical errors as the eighth leading cause of death, ahead of motor vehicle accidents, breast cancer or AIDS. The American Hospital Association estimates that medical errors account for between 44,000 and 98,000 U.S. deaths each year. From a financial perspective, research indicates that nationally, the annual cost of preventable adverse drug events in the U.S. is about $6 billion. Over 770,000 patients are injured because of medication errors every year. Medication errors occur in nearly 1 of every 5 doses given to patients in the typical hospital. Reported rates of adverse drug events (ADEs) range from 2.4 to 6.1 ADEs per 100 admissions or discharges, or 9.1 to 19 ADEs per 1000 patient days.

Medication errors often arise from errors in drug administration, which account for 38% of medication errors. Only 2% of drug administration errors are intercepted. Safety at the point of care is one of the greatest areas for potential improvement in the medication use process. 54% of potential ADEs are associated with IV medications. Studies have found that ADEs occur between 2.9 and 3.7 percent of hospitalizations. 61% of the serious and life-threatening errors are associated with IV medications. Insulin has been described as the most dangerous IV medicine, with special protocols and checks recommended to help prevent life-threatening errors. See “Reducing Variability in High Risk Intravenous Medication Use”, Center for Medication Safety and Clinical Improvement, 2005, Cardinal Health, which is incorporated herein by reference.

The first concepts of an artificial pancreas were conceived in the 1970's. Such systems offer the promise of complete automation—the patient's blood glucose would be completely and perfectly controlled with no human user intervention. See “Report of the Automated Control of Insulin Levels Committee”, Committee Report (DRA 5), Institute for Alternative Futures, p. 9, September, 2006, which is incorporated herein by reference. However, any error in the measurement, infusion determination, or infusion system can lead to catastrophic medication errors, and so such systems have seen little use.

Accordingly, there is a need for a semi-automated medication management system that reduces the chance of missed measurements, infusion calculation errors, or infusion control errors while still involving a human clinician in the final infusion decision.

Development of Continuous Glucose Monitors. There has been significant effort devoted to the development of in-vivo glucose sensors that continuously and automatically monitor an individual's glucose level. Such a device would enable individuals to more easily monitor their glucose light levels. Most of the efforts associated with continuous glucose monitoring have been focused on subcutaneous glucose measurements. In these systems, the measurement device is implanted in the tissue of the individual. The device then reads out a glucose concentration based upon the glucose concentration of the fluid in contact with the measurement device. Most of the systems implant the needle in the subcutaneous space and the fluid measured under measurement is interstitial fluid.

As used herein, a “contact glucose sensor” is any measurement device that makes physical contact with the fluid containing the glucose under measurement. Standard glucose meters are an example of a contact glucose sensor. In use a drop of blood is placed on a disposable strip for the determination of glucose. An example of a glucose sensor is an electrochemical sensor. An electrochemical sensor is a device configured to detect the presence and/or measure the level of analyte in a sample via electrochemical oxidation and reduction reactions on the sensor. These reactions are transduced to a electrical signal that can be correlated to an amount, concentration, or level of analyte in the sample. Another example of a glucose sensor is a microfluidic chip or micro post technology. These chips are a small device with micro-sized posts arranged in varying numbers on a rectangle array of specialized material which can measure chemical concentrations. The tips of the microposts can be coated with a biologically active layer capable of measuring concentrations of specific lipids, proteins, antibodies, toxins and sugars. Microposts have been made of Foturan, a photo defined glass. Another example of a glucose sensor is a fluorescent measurement technology. The system for measurement is composed of a fluorescence sensing device consisting of a light source, a detector, a fluorophore (fluorescence dye), a quencher and an optical polymer matrix. When excited by light of appropriate wavelength, the fluorophore emits light (fluoresces). The intensity of the light or extent of quenching is dependent on the concentration of the compounds in the media. Another example of a glucose sensor is an enzyme based monitoring system that includes a sensor assembly, and an outer membrane surrounding the sensor. Generally, enzyme based glucose monitoring systems use glucose oxidase to convert glucose and oxygen to a measurable end product. The amount of end product produced is proportional to the glucose concentration. Ion specific of electrodes are another example of a contact glucose sensor.

As used herein, a “glucose sensor” is a noncontact glucose sensor, a contact glucose sensor, or any other instrument or technique that can determine the glucose presence or concentration of a sample. As used herein, a “noncontact glucose sensor” is any measurement method that does not require physical contact with the fluid containing the glucose under measurement. Example noncontact glucose sensors include sensors based upon spectroscopy. Spectroscopy is a study of the composition or properties of matter by investigating light, sound, or particles that are emitted, absorbed or scattered by the matter under investigation. Spectroscopy can also be defined as the study of the interaction between light and matter. There are three main types of spectroscopy: absorption spectroscopy, emission spectroscopy, and scattering spectroscopy. Absorbance spectroscopy uses the range of the electromagnetic spectrum in which a substance absorbs. After calibration, the amount of absorption can be related to the concentration of various compounds through the Beer-Lambert law. Emission spectroscopy uses the range of the electromagnetic spectrum in which a substance radiates, The substance first absorbs energy and then I radiates this energy as light. This energy can be from a variety of sources including collision and chemical reactions. Scattering spectroscopy measure certain physical characteristics or properties by measuring the amount of light that a substance scatters at certain wavelengths, incidence angles and polarization angles. One of the most useful applications of light scattering spectroscopy is Raman spectroscopy but polarization spectroscopy has also been used for analyte measurements. There are many types of spectroscopy and the list below describes several types but should not be considered a definitive list. Atomic Absorption Spectroscopy is where energy absorbed by the sample is used to assess its characteristics. Sometimes absorbed energy causes light to be released from the sample, which may be measured by a technique such as fluorescence spectroscopy. Attenuated Total Reflectance Spectroscopy is used to sample liquids where the sample is penetrated by an energy beam one or more times and the reflected energy is analyzed. Attenuated total reflectance spectroscopy and the related technique called frustrated multiple internal reflection spectroscopy are used to analyze liquids. Electron Paramagnetic Spectroscopy is a microwave technique based on splitting electronic energy fields in a magnetic field. It is used to determine structures of samples containing unpaired electrons. Electron Spectroscopy includes several types of electron spectroscopy, all associated with measuring changes in electronic energy levels. Gamma-ray Spectroscopy uses Gamma radiation as the energy source in this type of spectroscopy, which includes activation analysis and Mossbauer spectroscopy. Infrared Spectroscopy uses the infrared absorption spectrum of a substance, sometimes called its molecular fingerprint. Although frequently used to identify materials, infrared spectroscopy also is used to quantify the number of absorbing molecules. Types of spectroscopy include the use of mid-infrared light, near-infrared light and uv/visible light. Fluorescence spectroscopy uses photons to excite a sample which will then emit lower energy photons. This type of spectroscopy has become popular in biochemical and medical applications. It can be used with confocal microscopy, fluorescence resonance energy transfer, and fluorescence lifetime imaging. Laser Spectroscopy can be used with many spectroscopic techniques to include absorption spectroscopy, fluorescence spectroscopy, Raman spectroscopy, and surface-enhanced Raman spectroscopy. Laser spectroscopy provides information about the interaction of coherent light with matter. Laser spectroscopy generally has high resolution and sensitivity. Mass Spectrometry uses a mass spectrometer source to produce ions. Information about a sample can be obtained by analyzing the dispersion of ions when they interact with the sample, generally using the mass-to-charge ratio. Multiplex or Frequency-Modulated Spectroscopy is a type of spectroscopy where each optical wavelength that is recorded is encoded with a frequency containing the original wavelength information. A wavelength analyzer can then reconstruct the original spectrum. Hadamard spectroscopy is another type of multiplex spectroscopy. Raman spectroscopy uses Raman scattering of light by molecules to provide information on a sample's chemical composition and molecular structure. X-ray Spectroscopy is a technique involving excitation of inner electrons of atoms, which may be seen as x-ray absorption. An x-ray fluorescence emission spectrum can be produced when an electron falls from a higher energy state into the vacancy created by the absorbed energy. Nuclear magnetic resonance spectroscopy analyzes certain atomic nuclei to determine different local environments of hydrogen, carbon and other atoms in a molecule of an organic compound. Grating or dispersive spectroscopy typically records individual groups of wavelengths. As can be seen by the number of methods, there are multiple methods and means for measuring glucose in a non-contact mode.

Note that the glucose sensors are referred to via a variety of nomenclature and terms throughout the medical literature. As examples, glucose sensors are referred to in the literature as ISF microdialysis sampling and online measurements, continuous alternate site measurements, ISF fluid measurements, tissue glucose measurements, ISF tissue glucose measurements, body fluid measurements, skin measurement, skin glucose measurements, subcutaneous glucose measurements, extracorporeal glucose sensors, in-vivo glucose sensors, and ex-vivo glucose sensors. Examples of such systems include those described in U.S. Pat. No. 6,990,366 Analyte Monitoring Device and Method of Use; U.S. Pat. No. 6,259,937 Implantable Substrate Sensor; U.S. Pat. No. 6,201,980 Implantable Medical Sensor System; U.S. Pat. No. 6,477,395 Implantable in Design Based Monitoring System Having Improved Longevity Due to in Proved Exterior Surfaces; U.S. Pat. No. 6,653,141 Polyhydroxyl-Substituted organic Molecule Sensing Method and Device; US patent application 20050095602 Microfluidic Integrated Microarrays For Biological Detection; each of the preceding incorporated by reference herein.

In the typical use of the above glucose sensors require calibration before and during use. The calibration process generally involves taking a conventional technology (e.g., fingerstick) measurement and correlating this measurement with the sensors current output or measurement. This type of calibration procedure helps to remove biases and other artifacts associated with the implantation of the sensor in the body. The process is done upon initiation of use and then again during the use of the device.

Testing of CGMS systems in the ICU setting. Since continuous glucose monitoring systems (CGMS) provide a continuous glucose measurement, it can be desirable to use these types of systems for implementation of tight glycemic control protocols. The use of a continuous glucose monitoring systems has been investigated by several clinicians. These investigations have generally taken two different forms. The first has been to use the continuous glucose monitors in the standard manner of placing them in the tissue such that they measure interstitial glucose. A second avenue of investigation has used the sensors in direct contact with blood via an extracorporeal blood loop. Summary information from existing publications is presented below.

“Experience with continuous glucose monitoring system a medical intensive care unit”, by Goldberg at al, Diabetes Technology and Therapeutics, Volume 6, Number 3, 2004. FIG. 1 shows the scatter plot of the 542 paired glucose measurements. For these measurements the r value was 0.88 overall with 63.4% of the measurement pairs fell within 20 mg/dl of one another while 87.8% fell within 40 mg/dl. Additionally the authors state that seven of the 41 sensors (17%) exhibited persistent malfunction prior to the study end point of 72 hours.

“The use of two continuous glucose sensors during and after surgery” by Vriesendorp et al., Diabetes Technology and Therapeutics, Volume 7, Number 2, 2005. In a summary conclusion the authors' state that the technical performance and accuracy of continuous glucose sensors need improvement before continuous glucose can sensors can be used to implement strict glycemic control protocols during and after surgery.

“Closed loop glucose control in critically ill patients using continuous glucose monitoring system in real-time”, by Chee et al, IEEE transactions on information technology in biomass and, volume 7, Number one, March 2003. The authors provide a summary comment that improvement of real-time sensor accuracy is needed. In fact the actual accuracy of the results generated showed that 64.6% of the sensor readings would be clinically accurate (zone b) while 28.8% would lead to in no treatment (zone b), as illustrated in FIG. 2. The authors state that the accuracy of subcutaneously measured glucose is dependent “on equilibration of glucose concentration to be reached before ISF, plasma and whole blood, taking into account a possible time delay. Skin perfusion on the site of the sensor insertion differs from patient to patient. Most patients admitted to the ICU have a degree of peripheral edema and glucose monitoring based on ISF readings under such conditions would be subjected to variation in ISF-plasma—whole blood equilibration. The problem is likely exacerbated by non-ambulatory patients with little dynamic circulation of ISF in the subcutaneous space.

Problems with Existing CGMS. The present invention can address various problems recognized in the use of CGMS. The performance of existing CGMS when placed in the tissue or an extracorporeal blood circuit is limited. The source of the performance limitation can be segmented into several discrete error sources. The first is associated with the actual performance of the sensor overtime, while the second error grouping is associated with the physiology assumptions needed for accurate measurements.

General performance limitations: in a simplistic sense electrochemical or enzyme based sensors use glucose oxidase to convert glucose and oxygen to gluconic acid and hydrogen peroxide. An electrochemical oxygen detector is then employed to measure the concentration of remaining oxygen after reaction of the glucose; thereby providing an inverse measure of the glucose concentration. A second enzyme, or catalyst, is optimally included with the glucose oxidase to catalyze the decomposition of the hydrogen peroxide to water, in order to prevent interference in measurements from the hydrogen peroxide. In operation the system of measuring glucose requires that glucose be the rate limiting reagent of the enzymatic reaction. When the glucose measurement system is used in conditions where the concentration of oxygen can be limited a condition of “oxygen deficiency” can occur in the area of the enzymatic portion of the system and results in an inaccurate determination of glucose concentration. Further, such an oxygen deficit contributed other performance related problems for the sensor assembly, including diminished sensor responsiveness and undesirable electrode sensitivity. Intermittent inaccuracies can occur when the amount of oxygen present at the enzymatic sensor varies and creates conditions where the amount of oxygen can be rate limiting. This is particularly problematic when seeking the use the sensor technology on patients with cardiopulmonary compromise. These patients are poorly perfused and may not have adequate oxygenation.

Performance over time: in many conditions an electrochemical sensor shows drift and reduced sensitivity over time. This alteration in performance is due to a multitude of issues which can include: coating of the sensor membrane by albumin and fibrin, reduction in enzyme efficiency, oxidation of the sensor and a variety of other issues that are not completely understood. As a result of these alterations in sensor performance the sensors must be recalibrated on a frequent basis. The calibration procedure typically requires the procurement of a blood measurement and a correlation of this measurement with the sensor performance. If a bias or difference is present the implanted sensor's output is modified so that there is agreement between the value reported by the sensor and the blood reference. This process requires a separate, external measurement technique and is quite cumbersome to implement.

Physiological assumptions: for the sensor to effectively represent blood glucose values a strong correlation between the glucose levels in blood and subcutaneous interstitial fluid must exist. If this relationship does not exist, a systematic error will be inherent in the sensor signal with potentially serious consequences. A number of publications have shown a close correlation between glucose levels in blood and subcutaneous interstitial fluid. However, most of these investigations were performed under steady-state conditions only, meaning slow changes in blood glucose (<1 mg/dl/min). This restriction on the rate of change is very relevant due to the compartmentalization that exists between the blood and interstitial fluid. Although there is free exchange of glucose between plasma and interstitial fluid, a change in blood glucose will not be immediately accompanied by an immediate change of the interstitial fluid glucose under dynamic conditions. There is a so-called physiological lag time. The physiological lag time is influenced by many parameters, including the overall perfusion of the tissue. In conditions where tissue perfusion is poor and the rate of glucose change is significant the physiological lag can become very significant. In these conditions the resulting difference between interstitial glucose and blood glucose can become quite large. As noted above the overall cardiovascular or perfusion status of the patient can have significant influence on the relationship between ISF glucose and whole blood glucose. Since patients in the intensive care unit or operating room typically have some type of cardiovascular compromise the needed agreement between ISF glucose and whole blood is not present.

Additional understanding with respect to the calibration of continuous glucose monitors can be obtained from the following references. U.S. Pat. No. 7,029,444, Real-Time Self Adjusting Calibration Algorithm. The patent defines a method of calibrating glucose monitor data that utilizes to reference glucose values from a reference source that has a temporal relationship with the glucose monitor data. The method enables calibrating the calibration characteristics using the reference glucose values and the corresponding glucose monitor data. US patent application 2005/0143636 System and Method for Sensor Recalibration. The patent application described a methodology for sensor recalibration utilizing an array of data which includes historical as well as recent data, such as, blood glucose readings and sensor electrode readings. The state in the application, the accuracy of the sensing system is generally limited by the drift characteristics of the sensing element over time and the amount of environmental noise introduced into the output of the sensing element. To accommodate the inherent drift in the sensing element in the noise inherent in the system environment the sensing system is periodically calibrated or recalibrated.

Additional understanding with respect to sensor drift can be obtained from the following references. Article by Gough et al. in Two-Dimensional Enzyme Electrode Sensor for Glucose, Vol. 57, Analytical Chemistry pp 2351 et seq (1985). U.S. Pat. No. 6,477,395 Implantable Enzyme-based Monitoring System Having Improved Longevity Due to Improved Exterior Surfaces. The patent describes an implantable enzyme based monitoring system having an outer membrane that resists blood coagulation and protein binding. In the background of the invention, columns 1 and 2 the authors describe in detail the limitations and problems associated with enzyme-based glucose monitoring systems.

The operation of many of the embodiments disclosed herein involves the use of a maintenance fluid. A maintenance fluid is a fluid used in the system for any purpose. Fluids can include saline, lactated ringers, mannitol, amicar, isolyte, heta starch, blood, plasma, serum, platelets, or any other fluid that is infused into the patient. In addition to fluids that are infused into the patient, maintenance fluids can include fluids specifically used for calibrating the device or for cleaning the system, for other diagnostic purposes, and/or can include fluids that perform a combination of such functions.

Glucose sensors, both contact and noncontact, have different capabilities with respect to making accurate measurements in moving blood. For example, most strip based measurement technologies require an enzymatic reaction with blood and therefore have an operation incompatible with flowing blood. Other sensors can operate in a mode of establishing a constant output in the presence of flowing blood. Noncontact optical or spectroscopic sensors are especially applicable to conditions where the blood is flowing by the fact that they do not require an enzymatic reaction. For the blood access system described herein, one objective is to develop a system that does not result in blood clotting. Generally speaking blood that is stagnant is more prone to clotting than blood that is moving. Therefore the use of measurement systems that do not require stationery blood is beneficial. This benefit is especially relevant if the blood is to be re-infused into the patient.

In an instrument that operates in the intensive care unit on critically ill patients, infection risk is an important consideration. A closed system is typically desired as the system has no mechanism for external entry into the flow path after initial set-up and during operation. The system can function without any opening or closing or the system. Any operation that “opens” the system is a potential site of infection. Closed system transfer is defined as the movement of sterile products from one container to another in which the container's closure system and transfer devices remain intact throughout the entire transfer process, compromised only by the penetration of a sterile, pyrogen-free needle or cannula through a designated closure or port to effect transfer, withdrawal, or delivery. A closed system transfer device can be effective but risk of infection is generally higher due to the mechanical closures typically used.

In the development of a glucose measurement system for frequent measurements in the intensive care unit, the ability to operate in a sterile or closed manner is extremely important. In the care of critically ill patients the desire to avoid the development of systemic or localized infections is considered extremely important. Therefore, any system that can operate in a completely closed manner without access to the peripheral environment is desired. For example, blood glucose measurement systems that require the removal of blood from the patient for glucose determination result in greater infection risk due to the fact that the system is exposed to a potentially non-sterile environment for each measurement. There are many techniques to minimize this risk of infection but the ideal approach is simply a system that is completely closed and sterilized. With respect to infection risk, a noncontact spectroscopic glucose measurement is almost ideal as the measurement is made with light which is able to evaluate the sample without any increase in infection risk.

Sampling from a central venous catheter. The effective implementation of tight glycemic control protocols generally requires the frequent measurement of glucose. This measurement process typically requires the procurement of a blood sample that is representative of the patient's physiological status. Samples can be obtained from a variety of means, including without limitation peripheral IV's, arterial blood lines, midline catheters peripherally inserted central catheters, and central venous catheters. Central venous catheters can be a preferred means of access due to the frequency of use in the ICU and the ability to make blood withdrawals on a regular basis. Most central venous catheters are multi-lumen catheters with the number of lumens being selected based upon patient needs. Catheters are referred to as monoluminal, biluminal or triluminal, dependent on the actual number of tubes or lumens (1, 2 and 3 respectively). Some catheters have 4 or 5 lumens, depending on the reason for their use. The termination of the lumen in the body occurs at different locations. The termination point is typically referred to as a port. In the case of a multi-lumen catheter the port at the end of the catheter is defined as the distal port, with intervening ports referred to as medial ports and the port closest to the insertion into the body referred to as the proximal port. The catheter is usually held in place by a suture or staple and an occlusive dressing. Regular flushing with saline or a heparin-containing solution is performed to keep the line patent and prevent infection.

Central venous blood samples can be obtained through a variety of catheter types including a central venous catheter. Central venous catheters are utilized for many purposes to include drug infusion as well as blood sampling. When central venous catheters are utilized for procurement of a blood draw, nursing standards are very specific with respect to the procedure to be used. These standards require that all IV infusions be stopped and recommend a one minute wait time before drawing blood from the catheter. The rationale for both the stoppage and waiting period is to allow IV fluids and medications to be carried away from the catheter location such that the blood sample is not contaminated by the fluids being infused (the “infusate”). The mixing of IV fluids or medications in the blood sample is generally referred to as cross-contamination. Cross-contamination is the general process by which fluids being infused into the patient become present in the blood sample and can contaminate resulting measurements. FIG. 1 is a schematic illustration of the terms involved. A central vein 101 has disposed within it a multi-lumen catheter 102, and normal blood flow from left to right in the figure at a rate denoted FR. The catheter 102 has a first port 103 from which it is desired that a sample be withdrawn at a withdrawal rate denoted WR. The catheter 102 has a second port 104 through which an infusate is infused into the vessel at a rate denoted IR.

Although central venous catheters can be placed in a variety of locations, the typical placement is to have the tip 3-4 cm above the entrance to the right atrium. This places the tip in the center of the superior vena cava and the proximal opening about 6 cm back from the tip. The proximal port will typically be in the vein where the device was introduced; i.e. the brachial cephalic or internal jugular vein. The flow characteristics surrounding the ports of the central venous catheter can have direct influence on the possibility of cross-contamination. The superior vena cava is the main vein for the drainage of the superior aspect of the body. It is about 7 cm in length and is formed by the confluence of the brachiocephalic veins. It has no valves and ends in the right atrium. It is approximately 20 mm in diameter. The inferior vena cava has similar flow characteristics but the flow rates are strongly dependent upon exercise involving the lower extremity. Flow in the central vena cava is variable and is affected by the cardiac cycle and respiration. FIG. 2 is an illustration of a typical tracing of the flow rates as a function of the cardiac cycle. In normal physiology, peak flow is during systole and is 30-45 cm/sec. At the beginning of the cardiac cycle, the flow rate is zero or slightly negative. There is a brief period of retrograde flow as the right ventricle contracts and it takes a finite amount of time for the valve to shut. Furthermore the valve tends to push out into the right atrium as the ventricle contracts.

Difficulties in tight glycemic control when using a central venous catheter. For blood glucose measurement systems that utilize a central venous access catheter for procurement of a blood sample for subsequent analysis or place a catheter in the superior or inferior vena cava, the potential impact of cross-contamination involving a glucose containing fluid can be quite dramatic. For example, if the patient is being infused with a 5% dextrose solution (5000 mg/dl), and 1% cross-contamination occurs, the measured glucose value can be in error by 50 mg/dl. Given that the typical target range for tight glycemic control is between 80 and 120 mg/dl, a potential over-estimation by 50 mg/dl can have serious consequences. As an example, the patient might be given additional insulin due to the inaccurately high glucose measurement result. The actual overall systemic glucose would be consequently decreased while the measured glucose might remain high due to the presence of glucose via cross-contamination. Cross-contamination with non-glucose containing fluids also can affect the measurement, but are typically less significant since they generally result in a decreased glucose measurement. The impact is simply volumetric so at a glucose value of 100 mg/dl a 10% dilution can result in a glucose measurement of 90 mg/dl, and such slightly low glucose readings are less likely to have such dramatic undesirable treatment errors.

Accordingly, there is a need for methods and apparatuses that allow accurate glucose measurements from catheters, especially central venous catheters, in the presence of infusion of substances including glucose.

Arterial Catheter method Since 2001, a number of intensive care units have adopted tight glycemic control protocols for the maintenance of glucose at close to physiological levels. The process of maintaining tight glycemic control requires frequent blood glucose measurements. The blood utilized for these measurements is typically obtained by procurement of a sample from a fingerstick, arterial line, or central venous catheter. Fingerstick measurements are generally considered undesirable due to the pain associated with the fingerstick process and the nuisance associated with procurement of a quality sample. Sample procurement from central venous catheters can also present problems since current clinical protocols recommend the stoppage of all fluid infusions prior to the procurement of a sample. Consequently, the use of arterial catheters has become more common. Arterial catheters are typically placed for hemodynamic monitoring of the patient and provide real-time continuous blood pressure measurements. These catheters are maintained for a period of time and used for both hemodynamic monitoring and blood sample procurement. Arterial catheters are not typically used for drug or intravenous feedings so issues associated with cross-contamination are minimized.

The process of procuring an arterial blood sample for measurement typically involves the following steps. The slow saline infusion used to keep the artery open is stopped and some type of valve mechanism such as a stopcock is opened to allow fluid connectivity to the mechanism for blood draw. The process of opening the stopcock and concurrently closing off fluid connectivity to the pressure transducer will cause a stoppage of patient pressure monitoring as the transducer no longer has direct fluid access to the patient. The sample procurement process is initiated. The initial volume drawn through the stopcock is saline followed by a transition period of blood and saline and subsequently pure blood. Generally, at the point where there is no or very little saline in the blood sample at the stopcock (or a knowable saline concentration), the measurement sample is obtained. The blood and saline sample obtained previously can be discarded or infused back into the patient.

In many intensive care units, a significant portion of blood samples obtained from arterial catheters are procured using blood sparing systems. In this process a leading sample containing both saline and blood is withdrawn from the patient and stored in a reservoir that lies beyond the sample acquisition port. A sample of blood that is free of saline contamination can then be procured at the sample port for measurement. Example embodiments of such blood sparing techniques include the Edward's VAMP system, shown in FIG. 1, and the Abbott SafeSet system. The Edward's VAMP in-service poster is incorporated by reference. Following procurement of an undiluted sample for measurement, the remaining blood/saline mixture can be re-infused into the patient. FIG. 1 is a schematic depiction of Edward's VAMP Plus System, an example blood sparing device. In the example device, a blood access system attached to arterial line, blood withdrawn and re-infused. A pressure monitoring transducer is remote from patient (60 inches). The tubing used between patient and pressure transducer is very stiff so compliance is minimized. A saline wash of transducer is provided after a clean sample is drawn into the syringe.

Air bubbles represent a significant problem for hemodynamic monitoring systems as they change the overall performance of the system. Air bubbles can become trapped in the monitoring system during filling, blood sampling, or added later by manual flushing or continuous flush devices. The presence of an air bubble adds undesirable compliance to the system and tends to decrease the resonant frequency and increase the damping coefficient. The resonant frequency typically falls faster than the damping increases, resulting in a very undesirable condition. FIG. 2 illustrates the effect of adding microliter air bubbles of various sizes to a transducer-tubing system. As more and more air is added to the system, the decrease in resonant frequency produces larger and larger errors in the systolic pressure, even though damping is increasing at the same time. Eventually, so much air could be added that the system produces only damped sine waves. Air bubbles diminish, not enhance, the performance of blood pressure monitoring systems. The preceding information was obtained from the Association for the Advancement of Medical Instrumentation, technical information report titled “Evaluation of clinical systems for invasive blood pressure monitoring”.

In clinical use, a pressure monitoring system should be able to detect changes quickly. This is known as its “frequency response”. The addition of damping to a monitoring system will tend to decrease its responsiveness to changes in the frequency of the pressure waveform but prevents unwanted resonances. This is especially so if changes are occurring rapidly such as occur at high heart rates or with a hyperdynamic heart. During these conditions it is essential that the system have a high “natural” or “untamed” frequency response. The optimal pressure monitoring system should have a high frequency such that over damped or under damped waveforms are unlikely regardless of the degree of damping present. The relationship of frequency and camping coefficient have been explored and defined by Reed Gardner. This relationship is well described in “Direct Blood Pressure Measurements—Dynamic Response Requirements” anesthesiology pages 227-236, 1981, incorporated herein by reference. FIG. 3 shows the resulting relationship between damping and natural frequency.

Due to the existing performance requirements and the fact that air bubbles dramatically alter the performance of a typical hemodynamic monitoring system, it is clinical practice to have the clinician evaluate the system carefully for the presence of any air bubbles. As stated by Michael Cheatham in “Hemodynamic Monitoring: Dynamic Response Artifacts” (available from www.surgicalcriticalcare.net), perhaps the single most important step in optimizing dynamic response is ensuring that all transducers, tubing, stopcock, and injection ports are free of air bubbles. Air, by virtue of being more compressible than fluid, tends to act as a shock absorber within a pressure monitoring system leading to a over damped waveform with its attendant underestimation of systolic blood pressure and over estimation of diastolic blood pressure. The identification of air bubbles is typically done by visual inspection of the system as well as by a dynamic response test. In practice this dynamic response test is achieved by doing a fast-flush test. A fast flesh or square wave test is performed by opening the valve of the continuous flush device such that flow through the catheter tubing is actually increased to approximately 30 ml/hr versus the typical 1-3 ml/hr. This generates an acute rise in pressure within the system such that a square wave is generated on the bedside monitor. With closure of the valve, a sinusoidal pressure wave of a given frequency and progressively decreasing implicated is generated. A system with appropriate dynamic response characteristics will return to the baseline pressure waveform within one or two oscillations, as illustrated in FIG. 4. If the fast-flush technique produces dynamic response characteristics that are inadequate, the clinician should troubleshoot the system to remove air bubbles, minimize tubing junctions, etc., until acceptable dynamic response is achieved.

In almost any automated blood glucose monitoring system, the device must procure or withdraw a sample of blood from the body. This process may require a few milliliters of blood or only a few micro liters. Regardless of the amount, the process exposes the associated fluid column to pressure gradients, potentially different pressures and fluid flows. Therefore, the process of procuring a blood sample has the potential to create bubbles within the fluid column. The fluid column is not intended to be restrictive but to apply to any of the fluid associated with the automated sample measurement system. Solubility is the property of a solid, liquid or gas called solute to dissolve in a liquid solvent to form a homogeneous solution. The solubility of a substance strongly depends on the used solvent as well as on temperature and pressure. In the application of automated blood measurements, the liquid solvent is blood, saline or any intravenous solution. The solute is air, oxygen or any gas in the liquid solvent. Changers in solubility due to temperature or pressure may result in bubble formation. As a solution warms it will typically outgas due to a decrease in solubility with temperature. Changes in pressure can also result in bubbles. The solubility of gas in a liquid increases with increasing pressure. Henry's Law states that: the solubility of a gas in a liquid is directly proportional to the pressure of that gas above the surface of the solution. If the pressure is increased, the gas molecules are forced into the solution since this will best relieve the pressure that has been applied.

Bubbles may be formed due to cavitation. Cavitation is the formation of bubbles in a flowing liquid in a region where the pressure of the liquid falls below its vapor pressure. Cavitation can occur due to pumping at the low pressure or suction side of the pump. Cavitation can occur via multiple methods but the most common are vaporization, air ingestion (not always considered cavitation, but has similar symptoms), and flow turbulence

In a typical process of procuring a blood sample, a negative or reduced pressure is created so that the blood flows out of the body. This reduction in pressure creates an opportunity for bubble creation. Additionally, temperature differences between the human body, the ambient air, and any IV solutions also create the opportunity for bubble creation. Almost any form of pumping device creates some small degree of cavitation. Therefore, the process of attaching or combining a hemodynamic monitoring system with an automated blood measurement system creates the opportunity for bubble formation which in turn can result in poor performance of the hemodynamic monitoring system.

Hemodynamic pressure monitoring is unavailable during the procurement of the blood sample by either the syringe method or by use of a blood sparing system. If the standard stopcock is replaced with a 4-way stopcock it would allow the transducer and the blood sampling system to be in fluid connectivity with the patient. In such a situation the withdrawal process creates a pressure gradient that will limit the accuracy of the existing hemodynamic monitoring system.

The development of an automated blood glucose measurement system for use in the intensive care unit is highly desired due to reductions in labor, increased measurement frequency, and an improved ability to limit potentially dangerous conditions of hypoglycemia. The ability to attach such a system to an arterial access site is desired as catheter patency for blood sample procurement is typically better at an arterial access location than at a venous access site. As placement of an arterial catheter is considered a moderately invasive procedure, it is undesirable to require placement of two such catheters, one used for pressure monitoring and another for blood access. Thus, in clinical practice it is desirable to use one arterial access site for both hemodynamic monitoring as well as a blood access site for automated glucose measurement. Such sharing of a single site can result in hemodynamic monitoring disruption during the blood procurement process. For example, if the automated blood measurement system acquires a sample every 15 minutes, it will likely interfere with the hemodynamic pressure monitoring system so as to cause an alarm or produce inaccurate pressure measurements. The management of such an alarm typically requires nurse intervention, defeating some of the advantages sought with an automated blood measurement system. In addition to nuisance alarms, the real-time hemodynamic monitoring may be disrupted during the automated measurement process. In those patients that are hemodynamically unstable, such a disruption may be an unacceptable consequence of automated blood glucose monitoring.

Diabetes mellitus is an endocrine metabolic disorder resulting from a lack of insulin that affects over 170 million people worldwide. Improved glucose sensing would enable improved glycemic control, thereby delaying the onset of serious medical complications associated with diabetes. An indispensable tool for both diabetic and critically ill patients is a reliable blood glucose measurement method. Most diabetic patients currently use self-monitoring via finger pricking and test strips to check their blood glucose level and adjust their insulin dosage to maintain normal blood glucose concentrations. Although such self-monitoring of blood glucose has been an indispensable tool for diabetes therapy, it is fraught with difficulties. Frequent finger pricking is painful, costly, and inconvenient for the patient. As a result of this invasiveness, many diabetics frequently skip self-monitoring tests. Further, tight control of blood glucose is difficult to achieve without frequent glucose measurements. Intermittent measurements can be influenced by other changes in the patient's physical state and testing conditions. Glucose fluctuations during the day, and particularly during the night, are often missed using self-monitoring techniques.

One desirable system for the management of glycemia is a continuous in-vivo glucose monitoring method that could be coupled with an automated insulin pump for active closed-loop control of glucose level. In-vivo glucose sensing devices being developed comprise both implanted and noninvasive sensors. Invasive devices can be implanted intravascularly in the blood stream or interstitially under the skin, since the concentration of glucose within the interstitial fluid correlates with the glucose concentration in the blood. Alternate invasive technologies to measure blood glucose remove blood from the body for interrogation and analysis. This blood might be discarded or infused back into the body. Typically, if blood is infused, saline is also used which adds more fluid to the body. Noninvasive glucose sensors measure glucose concentrations in vivo without direct physical contact between the sensor and the biological fluid. Such noninvasive sensors are patient friendly and can eliminate biocompatibility problems. Most in-vivo glucose sensors are based on electrochemical or colorimetric/photometric detection techniques.

Colorimetric and photometric approaches can be used to monitor glucose levels directly. For example, vibrational spectroscopic approaches can use the unique vibration transitions within the glucose molecule. Vibrational spectroscopies include Raman spectroscopy and absorption spectroscopy in the mid- and near-infrared spectral regions. Raman spectroscopy can measure fundamental vibrational bands, but sensing applications have been hampered by the presence of a strong background fluorescence signal and low signal-to-noise ratio due to an inherently weak Raman signal. Glucose is a relatively simple monosaccharide molecule with strong and distinctive absorption features in the mid-infrared (MIR) region. Unfortunately, water and other non-glucose metabolites, such as proteins, amino acids, urea, fatty acids, and triglycerides also strongly absorb in the MIR.

Therefore, emphasis has shifted to the detection of molecular absorptions in the near-infrared (NIR) spectral region corresponding to combinations and overtones of fundamental glucose molecular vibrations. The strong OH and CH stretch bands in the 2900 to 3600 cm⁻¹ MIR region can generate overtone and combination bands in the 700 to 1700 nm NIR region. Additional glucose-specific combinations of CH stretch and ring deformation bands occur at wavelengths greater than 2000 nm. Although the glucose absorptions in the NIR are unique, they are weaker and broader than the fundamental bands and also overlap with bands from other tissue components, such as water, fat, and hemoglobin. Therefore, multivariate chemical analysis methods can be used to extract glucose-specific spectral information.

Noninvasive optical sensors can use optical radiation to probe regions of tissue, such as the finger, tongue, or ear, and extract glucose concentration from a measured spectrum. Noninvasive NIR sensors use the “optical window” in the near infrared in which the absorbance by human biological tissue is lower compared to the visible or ultraviolet regions. However, these noninvasive NIR sensors can have measurement difficulties due to the weak glucose absorption peaks, relatively low glucose concentrations in human tissue, multiple interferences with non-glucose metabolites, variations in tissue hydration, blood flow, environmental temperature, and light scattering.

Fiber optic probes can be used for minimally invasive optical sensors. See Utzinger and Richards-Kortum, J. Biomedical Optics 8(1), 121 (2003), which is incorporated herein by reference. Fiber optic probes provide a flexible optical interface between a light source, spectrometric detector, and the tissue being interrogated so that the light source and detector can be located remote from the patient. A dual-fiber arrangement can be used for separate illumination and collection. The collection fiber optic can transport the remitted light from the interrogated tissue to the spectrometer.

An individual optical fiber typically comprises a core, a cladding, and a protective jacket. Fibers can be packed into bundles to provide a larger optical active area. Coupling optics can adapt the f-number of the light source to the numerical aperture of the fiber to optimize irradiance into the fiber. The ends of a fiber can be cleaved or polished for optimal coupling. Further, the exit surface can be beveled to deflect the light output or input. Probe geometries can comprise side-looking probes that use obliquely polished ends to deflect the output of the fiber in respect to the fiber axis, probes with diffuser tips to provide homogeneous illumination of large areas in canals and on surfaces, and refocusing probes that refocus the illumination or collection beam path to decrease or increase the sample volume illuminated.

Probe assemblies have also been used for indwelling light scattering spectroscopy for biomedical applications. See U.S. Pat. No. 6,366,726 to Wach et al., which is incorporated herein by reference. In particular, Raman spectroscopy can provide a means for chemical identification. With Raman spectroscopy, incident laser light is transmitted over an optical fiber to the sample medium and the Raman-scattered is returned via the same or another fiber to a spectrometer for analysis. The Raman-scattered light is color shifted from the incident illumination beam by a specific amount related to molecular band vibrations. Further, the intensity of the shifted return light correlates with the chemical concentration. However, in-vivo Raman spectroscopy using flat face, parallel illumination and collection fiber probes has been hampered by the inefficiency of scattered light collection. Wach describes several approaches to direct and manipulate illumination and receptivity zones to improve Raman-scattered light collection efficiency. These approaches include varying the numerical apertures of the illumination and collection fibers, use of confocal optics, bending the tips of the fibers to increase the overlapping region, shaping the fibers' end faces to create a refractive surface to manipulate the illumination and collection zones, and manipulating the light with light-shaping structures within the confines of the fiber assembly's internal structure. Therefore, the probe can be designed to have selective sensitivity to the Raman scattering signal by delivering light at one angle and collecting light at the appropriate angle to maximize the response. However, sensing applications based on Raman spectroscopy have been hampered by the silica-Raman effect and fiber fluorescence and the inherently low weak Raman signal.

Therefore, a need remains for an in-vivo continuous glucose monitoring method that uses an indwelling fiber optic probe to measure glucose concentration or presence in the near-infrared spectral region.

In-Vivo Glucose Sensors This invention relates to the measurement of blood analytes, and more specifically to the measurement of glucose in blood that has been temporarily removed from the body. Over the past 10 years there has been significant effort devoted to the development of in-vivo glucose sensors that continuously and automatically monitor an individual's glucose level. Such a device enables individuals to more easily monitor their glucose levels. Most of the efforts associated with continuous glucose monitoring have been focused on subcutaneous glucose measurements. In these systems, the measurement device is implanted into the tissue of the individual. The device then reads out a glucose concentration based upon the glucose concentration of the fluid in contact with the measurement device. Most of such systems implant a needle in the subcutaneous space and measure interstitial fluid.

As used herein, a contact glucose sensor is any measurement device that makes physical contact with a fluid containing the glucose to be measured. An example of a contact glucose sensor is an electrochemical sensor. A noncontact glucose sensor is any measurement method that does not require physical contact with the fluid containing the glucose under measurement. Example noncontact glucose sensors include sensors based upon spectroscopy, meaning sensors based on the interaction between light and matter. For the purposes of this application “glucose sensor” includes both contact sensors and noncontact sensors.

Almost all types of glucose sensors are subject to drift over time. Therefore the ability to periodically calibrate these sensors is often desired and necessary. Within the context of automated blood glucose measurements for use in the intensive care unit, a simple and easy to use calibration procedure is desired. Such a calibration procedure should not require nurse intervention and should maintain the overall sterility of the device. Calibration techniques that infuse excessive amounts of glucose into a patient can be undesirable (since maintenance of tight glycemic control is important in many medical settings, including OR and ICU settings).

In the case where the sensor drifts over time, a bias and slope correction can require subsequent validation. The use of bias and slope adjustments to improve calibration or prediction statistics for multivariate models is appropriate provided that the calibration is fully revalidated whenever bias and slope is adjusted. Bias and slope adjustments are another form of calibration transfer and use of bias and slope adjustments can be handled in the same fashion as any other calibration transfer. Prediction errors requiring continued bias and slope corrections indicate drift in reference method or changes in the character of the samples, sample handling, sample presentation, instrument response function, or wavelength stability. If a calibration model fails during the QC monitoring step, the performance of the instrument can be evaluated using the appropriate ASTM instrument performance test [E1944-98 (reapproved 2007), incorporated herein by reference], and any instrument problem that is identified can be corrected. If control samples are used, checks can be performed on the reference method to ensure that reference values are correct. If instrument maintenance is performed, calibration transfer or instrument standardization procedures, or both, can be followed to reestablish the calibration. The preceding information is cited from ASTM International E 1655-05, “Standard Practices for Infrared Multivariate Quantitative Analysis,” Copyright © ASTM International, 100 Barr Harbor Drive, PO Box C700, West Conshohocken, Pa. 19428-2959, United States, 2007, incorporated herein by reference.

In general terms, the ability to calibrate a system and provide subsequent validation is a desired attribute of a blood analyte system. When evaluating an analyte sensor that has multiple analytes, a multitude of calibration samples can be needed to create confidence in the calibration and validation procedure.

In creating a blood access system for measurement of blood analytes, the process generally involves removing the blood from the patient to a measurement site. The measurement is then made by a variety of methods and the blood is either discarded or re-infused into the patient. Access to the patient is typically through a catheter including, as examples, peripheral venous lines, PIC lines, arterial lines and central venous lines. In many cases, the access line between the patient and the pumping system is typically filled with a fluid, such as saline. It is common practice to infuse a small amount of saline between blood draws or measurements to help maintain the patency of the access site. This is referred to herein as a “keep vein open” or “KVO” rate. At the initiation of a draw the fluid-filled line reverses flow and blood is pulled toward the measurement site. The junction between the blood and the fluid is referred to as the “blood-fluid junction”; mixing of the fluid with the blood near the junction creates a “transition zone”. As the blood is drawn from the patient through the tubing, the blood/fluid interface exhibits a parabolic flow profile and is characterized by a broadened transition zone of blood mixed with fluid. Additional dilution can occur due to tubing discontinuities. The transition zone between undiluted blood and fluid increases in extent as the draw continues. Since analyte measurement systems are often sensitive to dilution effects, measurement accuracy can be enhanced by providing a sample for measurement that has a known or controlled dilution, for example a constantly diluted sample, a minimally diluted sample, or an undiluted sample, can facilitate accurate measurements. Hereafter the reference to an “undiluted” sample simply refers not only to a blood sample that has not been diluted but also to any sample that is suitable for accurate determination of blood analytes due to a known or controlled dilution characteristic. Accordingly, an “undiluted” sample can have dilution but of a quality that can be controlled, sensed, or managed. To obtain a blood sample representative of the blood in the patient, the blood access system can pull the diluted blood in the transition zone beyond the measurement site. Thus, the total amount of blood drawn is greater than the volume of the tubing between the measurement site and the patient. This dilution issue is known in the medical community and is generally addressed by drawing a discard sample or by filling an extra reservoir with diluted blood. As an example, the Edward's VAMP system includes such a reservoir.

In some systems, it can be desirable to also follow the sample with fluid so as to minimize the amount of blood that is removed from the patient. In this case, a second transition zone is created behind the undiluted sample.

In a system with defined and predictable operating characteristics, the withdrawal volume needed for procurement of an undiluted sample can be established and fixed. In most real-world blood access systems too many variables change over time and the system must have the capability of determining the presence of an undiluted sample. Some of the variables that change over time and between patients include:

Length and/or volume of the access catheter: central venous catheters generally have more volume and a longer length than peripheral catheters;

Extension tubing: the clinical staff might add extension tubing to the blood access system;

Blood viscosity changes due to differences in blood composition;

Blood hematocrit differences that influence the pressure needed to move the fluid and mixing characteristics at the blood-saline junction;

Pump tubing differences, including differences in internal volume and or pumping efficiency;

Pump efficiency changes over time.

Due to these and other variables that can change over time, the system must be able to determine the presence of an undiluted sample and then initiate the analyte measurement process.

Peristaltic pumps are commonly used in medical applications because they enable bidirectional pumping and can also prevent flow when the pump motor is not moving. However peristaltic pumps can be prone to pump volume differences between tubing sets and within a tubing set over time. In a peristaltic pump the volume accuracy is dependent on the volume captured between two or more occluding points, the pump rollers. The captured volume between the rollers is then propagated through the pump creating flow. For the pump to be accurate this captured volume must be constant. When a peristaltic pump withdraws fluid from a line there is a vacuum generated in the inlet of the pump. This vacuum can cause the tubing to collapse, and the captured volume between the occluding rollers will be less than in non-collapsed tubing. This can be compensated to an extent by monitoring the pressure at the inlet of the pump, and by adjusting the pump speed to withdraw the correct total volume. However, over time the tubing can fatigue so that it collapses more easily and the capture volume drifts down. As a result, the accuracy of the pump decreases over time. When withdrawing fluid from a line, the amount of fatigue varies from tubing set to tubing set and the change in fatigue varies increasingly over time (see, e.g., FIG. 1).

The determination of volume can be made with a flow meter. A number of ultrasonic flow meters are available commercially. By knowing the flow rate and the time period the amount of volume pumped can be determined. Volume determination helps to compensate for pump efficiency changes but does not completely compensate for blood changes. Additionally, such flow meters are expensive relative to overall system cost objectives.

For a blood access system designed to measure blood anatytes, the system should be able to determine when the fluid withdrawn is suitable for measurement. Due to the possibility of changing parameters associated with the blood being withdrawn, the physical volume of the blood access system and the efficiency of the pump system, the use of a fixed draw volume or draw time is inadequate. It can be desirable to minimize the total amount of blood withdrawn due to fluid infusion needs, the desire to remove from the patient as little blood as possible, and the desire to expose the tubing set to a minimum amount of blood over time.

Proper determination of an analyte for a biological system requires procurement or acquisition of a sample that is representative of the biological system prior to analyte determination. For example, measurement of blood analyte values and other blood parameters (such as blood counts, coagulation parameters, and oxygenation status) in patients usually requires that a blood sample be drawn from the patient for analysis. Caregivers frequently draw blood samples for analysis from arterial or venous access lines that are also used to infuse fluids to the patient. This generally requires that a volume of blood and fluid be pre-drawn from the access line to clear the line of the infusion fluid between the sample port and the tip of the catheter in the patient's vessel so that the desired measurement is performed on sample of blood and not on infusion fluid that may be still in the line. After the pre-draw is complete, the pure blood sample is drawn for analysis. When the pre-draw is not performed or is of insufficient volume to completely clear the line of the non-blood fluid, the blood sample that is procured for analysis can contain an unknown amount of the infusion fluid. The result is a sample that provides an erroneous result, either due to simple dilution (in the case where the infusion fluid is simple saline) or due to a false change in the analyte or parameter of interest due to the contamination of the sample by the constituents of the infusion fluid. Errors of this type that are associated with sample procurement prior to analyte or parameter determination are known in the clinical community as pre-analytical errors, and are among the most common errors encountered in measurements of blood chemistry and other biological fluid samples. Such errors can result in the need to repeat tests, causing delays in making medical decisions or administering treatment. In some cases, such errors can lead to erroneous medical decisions, leading to serious and sometimes even fatal medical consequences for the patient.

In addition to dilution or contamination of a blood sample by infusion fluid due to insufficient volume of pre-sample, there are several other situations that can compromise the quality of the biological sample. Examples include:

Acquisition of a blood sample simultaneously with administration through an adjacent vascular access line of a therapeutic agent or fluid. This can cause acquisition of a non-representative sample if the blood sample were drawn before the fluid were evenly distributed and equilibrated throughout the systemic blood volume. Acquisition of a sample during administration of a fluid or agent can be contaminated with the co-infused substance.

Administration of large volume physiological therapy, such as blood transfusion or blood volume expanders. As before, a blood sample drawn during such therapy can be an unstable or nonrepresentative sample.

It can be desirable to determine the quality of the sample prior to making the determination of the analyte or parameter of interest of the biological sample, thereby preventing the reporting of analytical values that have pre-analytical error due to improper or inadequate sample procurement or acquisition.

Intensive Insulin Therapy Critically ill patients that require intensive care for more than five days have a 20% risk of death and substantial morbidity. Hyperglycemia associated with insulin resistance is common in critically ill patients, even those who do not suffer from diabetes. A recent paper published in November 2003 in the NEJM by Greet Van den Burghe et al hypothesized that hyperglycemia or relative insulin deficiency during critical illness may directly or indirectly confer a predisposition to complications such as severe infections, polyneuropathy, multiple-organ failure, and death. In nondiabetic patients with protracted critical illnesses, high serum levels of insulin-like growth factor-binding protein 1, which reflect an impaired response of hepatocytes to insulin, increase the risk of death. They performed a prospective, randomized, controlled trial at one center to determine whether normalization of blood glucose with intensive insulin therapy reduces mortality and morbidity among the critically ill patients.

Van Den Berghe et al were able to show dramatic improvements in patient's outcomes when patients had their blood glucose controlled tightly between 80 and 110 mg per deciliter during their ICU stay.

The trial performed was a prospective, randomized, controlled study involving adults admitted to the surgical intensive care unit who were receiving mechanical ventilation. On admission, patients were randomly assigned to receive intensive insulin therapy (maintenance of blood glucose at a level between 80 and 110 mg per deciliter [4.4 and 6.1 mmol per liter]) or conventional treatment (infusion of insulin only if the blood glucose level exceeded 215 mg per deciliter [11.9 mmol per liter] and maintenance of glucose at a level between 180 and 200 mg per deciliter [10.0 and 11.1 mmol per liter]).

At 12 months, with a total of 1,548 patients enrolled, intensive insulin therapy reduced mortality during intensive care from 8.0 percent with conventional treatment to 4.6 percent (P<0.04, with adjustment for sequential analyses). The benefit of intensive insulin therapy was attributable to its effect on mortality among patients who remained in the intensive care unit for more than five days (20.2 percent with conventional treatment, as compared with 10.6 percent with intensive insulin therapy, P=0.005). The greatest reduction in mortality involved deaths due to multiple-organ failure with a proven septic focus. Intensive insulin therapy also reduced overall in-hospital mortality by 34 percent, bloodstream infections by 46 percent, acute renal failure requiring dialysis or hemofiltration by 41 percent, the median number of red-cell transfusions by 50 percent, and critical-illness polyneuropathy by 44 percent. Also patients receiving intensive therapy were less likely to require prolonged mechanical ventilation and intensive care.

Intensive insulin therapy to maintain blood glucose at or below 110 mg per deciliter was shown to reduce morbidity and mortality among critically ill patients in the surgical intensive care unit. These results are even more exciting when overlaid with Oye et al. (Chest 99:685, 1991) findings that 8% of patients consumed 50% of cumulative ICU resources (measured by TISS points) (Therapeutic Intervention Scoring System). Garland et al. (AJRCCM 157:A302, 1998) had similar findings; 5% with the longest ICU lengths of stay consumed 20-48% of various ICU resources.

In the intensive treatment group, an insulin infusion was started if the blood glucose level exceeded 110 mg/dl, adjustment of insulin does was based upon whole-blood glucose measurements in arterial blood at 1 to 4 hour intervals with the use of a blood glucose analyzer. The dose of insulin was adjusted based upon a predetermined algorithm by a team if ICU nurses assisted by a study physician. These manual methods were extremely labor intensive and are not feasible for therapy adoption. In the conventional treatment group a continuous infusion of insulin was started if the blood glucose level exceeded 215 mg/dl and the infusion was adjusted to maintain a level between 180 and 200 mg/dl. On admission all patients were continuously with intravenous glucose (200 to 300 grams per 24 hrs). The next day total parenteral, combined parenteral and enteral feeding was instituted.

Diabetes companies are currently focused on implementing closed loop control for ambulatory diabetic patients where they have encountered a myriad of problems associated with blood glucose sensor accuracy and glucose level control due to the large fluctuations in patient metabolism and eating patterns, changes in sensor sensitivity due to the elapse of time and differences in patients, safety detection systems etc. Much research work is currently being focused to commercially produce an accurate long term implanted blood glucose sensor. It has been found that ensuring blood glucose sensor accuracy and having a fast responsive time are mutually exclusive for an implantable blood glucose sensor. Some glucose sensor manufacturers have focused on subcutaneous implanted sensors to avoid the pitfalls of sensor degradation due to fouling and clotting but these devices, while avoiding the need for blood contact, suffer from longer time constants and transport delays that make closed loop control very difficult. Non-invasive optical methods using near-infrared spectroscopy suffer from the affects of tissue variation and some manufacturers require the use of individual patient calibration making their use less desirable. Other sensors extract glucose through the skin by iontophoresis and measures the extracted sample electrochemically, using the glucose oxidase reaction. Direct contact with blood has been avoided due to clotting and fouling issues.

Thevenot in 1982 (Diabetes Care, Vol. 5 No. 3:184-189) recognized in his article that an implanted sensor would have to survive long-duration implantation in chemically harsh environment of the body. That the sensitivity would have 2 to 5% of the actual glucose level with a range of 10 to 200 mg/dl with little or no change due to long term drift or temperature dependence. Oberhardt in 1982 (Diabetes Care, Vol. 5 No. 3:213-217) recommended that the response of the sensor be 30 sec or less and that the sampling rate be 10 sec averaged over a 1 minute interval. No glucose has yet been proven to meet these requirements.

Many of the design constraints imposed by the ambulatory market are not valid for inpatient hospital ICU use and thus afford a new look at the design requirements. ICU patients are not ambulatory diabetic patients and are fed both parenterally and entrally. This avoids the large swings in levels of blood glucose seen in diabetic patients due to calorie intake at meal times and makes for a more even and predictable control system. Avoiding these large perturbations to the control system makes it easier to maintain glucose control. Implanted glucose sensors would be expected to work accurately for at least one year. This imposes a very large burden upon the sensor design which is currently one the biggest limitation in developing a viable implanted system. If the calibration of such a sensor were to fail it could have deleterious consequences for the patients. Schemes have been proposed to cross check the readings between the implanted sensor and standard finger stick sensors to overcome some of these limitations. Such a limitation does not exist if the sensor is only required for 3 to 5 days of use and independent periodic calibration can be instituted off line ensuring the accuracy of the sensor.

There is a significant need for an easy to use accurate glucose control therapy that can be instituted safely and effectively in the inpatient hospital setting in post surgical ICU patients. Such a therapy will reduce the incidence of mortality, sepsis and renal failure and can have dramatic costs savings for both hospitals and health care providers while improving patient quality of life and outcomes.

### SUMMARY OF THE INVENTION

The present invention comprises methods and apparatuses that can provide measurement of glucose and other analytes with a variety of sensors without many of the performance-degrading problems of conventional approaches. An apparatus according to the present invention comprises a blood access system, adapted to remove blood from a body and infuse at least a portion of the removed blood back into the body. Such an apparatus also comprises an analyte sensor, mounted with the blood access system such that the analyte sensor measures the analyte in the blood that has been removed from the body by the blood access system. A method according to the present invention comprises removing blood from a body, using an analyte sensor to measure an analyte in the removed blood, and infusing at least a portion of the removed blood back into the body. The use of a non-contact sensor with a closed system creates a system with minimal infection risk.

A method according to the present invention can comprise measuring the value of an analyte such as glucose at a first time; determining a second time from a patient condition, an environmental condition, or a combination thereof; then measuring the value of the analyte at the second time. The invention can be used with automated measurement systems, allowing the system to determine measurement times and automatically make measurements at the determined times, reducing operator interaction and operator error. The present invention also comprises methods and apparatuses for medication management based upon active authorization of medication infusion by a clinician that can provide for effective management of an analyte in a patient's blood, reducing the opportunities for human error common with current manual systems while still placing final control of the medication management with the human clinician.

The present invention comprises methods and apparatuses that can provide accurate measurement of glucose or other analytes from a multilumen catheter in the presence of infusion of substances, including glucose. Alternatively, the present invention provides an indwelling fiber optic probe that can be used to make blood glucose measurements through a central venous catheter. The probe can also be used to measure other metabolites, such as blood gases, lactate, hemoglobin and urea. The present invention comprises methods and apparatuses that can provide measurement of glucose and other analytes with a variety of sensors in connection with hemodynamic monitoring.

The invention relates to an automated calibration procedure for analyte sensors such as glucose sensors. The system can provide a calibration point at zero analyte concentration as well as a second calibration point at a known analyte concentration or other pre-determined points. The present invention enables a multitude of options in both calibration and validation to ensure effective operation of the system.

Example embodiments of the present invention provide methods and apparatuses that enable the detection of bubbles so that hemodynamic performance can be assured following an automated blood analyte measurement. An example apparatus according to the present invention comprises a blood access system, adapted to remove blood from a body and infuse at least a portion of the blood back into the body. The infusion of at least a portion of the blood back in to the body can be done in a manner to assure that no bubbles of clinical significance are injected into the patient. Additionally an example embodiment can assess for the presence of bubbles in the fluid column that can affect hemodynamic monitoring performance. If a condition exists where hemodynamic monitoring performance cannot be assured, an example embodiment can provide appropriate warning or corrective actions.

The present invention relates to a blood analyte measurement system for the procurement of blood samples for measurement of blood properties such as analyte concentration or analyte presence. A blood access system can be coupled with a measurement system such as an electrochemical sensor, and can also be used with other measurement modalities.

The use of an optical measurement in the blood access system enables the determination of a fluid sample appropriate for measurement on a real time basis. This information can be used to control the blood access system and related measurement processes. The optical measurement system can take a variety of forms, including light emitting diodes and detectors, spectrometers, and interferometers. Wavelength regions of relevance can span from the ultraviolet to the far infrared. The visible, near infrared and mid infrared spectral regions can be of particular interest.

The invention disclosed is not dependent upon the measurement method used and is applicable to indwelling electrochemical sensors, enzymatic sensors, sensors that work when in contact with blood, such as those made by Dexcom and Abbott, standard sensors that work on a sample of blood and other optical sensing methods that use serum, plasma, or ultra filtrate. Additionally, the method can work on any fluid-sample junction. Examples of such possible junctions include saline-serum, saline-plasma, and saline-ultra filtrate, and saline-supernatant from a centrifuged sample.

Advantages and novel features will become apparent to those skilled in the art upon examination of the following description or can be learned by practice of the invention. The advantages of the invention can be realized and attained by means of the methods, instrumentation architectures, and combinations specifically described in the disclosure and in the appended claims.

### BRIEF DESCRIPTION OF THE DRAWINGS

FIG. 1 is a scatter plot of 542 paired glucose measurements from “Experience with continuous glucose monitoring system a medical intensive care unit”, by Goldberg at al, Diabetes Technology and Therapeutics, Volume 6, Number 3, 2004.

FIG. 2 is an illustration of error grid analysis of glucose readings.

FIG. 3 is a schematic illustration of an example embodiment of the present invention comprising a blood access system using a blood flow loop.

FIG. 4 is a schematic illustration of a blood loop system with a peristaltic pump.

FIG. 5 is a schematic illustration of a blood access system implemented based upon a pull-push mechanism with a second circuit provided to prevent fluid overload.

FIG. 6 is a schematic illustration of a blood access system based upon a pull-push mechanism with a second circuit provided to prevent fluid overload.

FIG. 7 is a schematic illustration of a blood access system based upon a pull-push mechanism.

FIG. 8 is a schematic illustration of a blood access system implemented based upon a pull-push mechanism with a second circuit provided to prevent fluid overload.

FIG. 9 is a schematic illustration of an example embodiment that allows a blood sample for measurement to be isolated at a point near the patient and then transported to the instrument for measurement.

FIG. 10 is an illustration of the control of the blood volume and the integration of the total amount of glucose measured.

FIG. 11 is a schematic illustration of an example embodiment that allows a blood sample for measurement to be isolated at a point near the patient and then transported to the instrument for measurement through the use of leading and the following air gaps.

FIG. 12 is a schematic illustration of an example embodiment of the present invention.

FIG. 13 is a schematic illustration of an example embodiment of the present invention.

FIG. 14 is a schematic illustration of an example embodiment of the present invention.

FIG. 15 is a schematic illustration of an example embodiment of the present invention.

FIG. 16 is a plot showing the relationship between pressure, tubing diameter and blood fraction.

FIG. 17 is a plot showing the relationship between pressure, tubing diameter and blood fraction.

FIG. 18 is a schematic illustration of an example embodiment of the present invention.

FIG. 19 is a schematic illustration of an example embodiment of the present invention.

FIG. 20 is a schematic illustration of an example embodiment of the present invention.

FIG. 21 is a schematic illustration of the operation of an example embodiment of the present invention.

FIG. 22 is a schematic illustration of the operation of an example embodiment of the present invention.

FIG. 23 is a schematic illustration of an example embodiment of the present invention.

FIG. 24 is a schematic illustration of an example embodiment of the present invention.

FIG. 25 is a schematic illustration of the present invention in use with a patient.

FIG. 26 is a schematic illustration of the present invention in use with a patient.

FIG. 27(a,b,c) is a schematic illustration of the operation of an example embodiment of the present invention.

FIG. 28 is a Netter physiological response diagram illustrating interactions governing glucose consumption and production.

FIG. 29 is a block diagram of interactions governing glucose consumption and production.

FIG. 30 is a presentation of equations governing the Chase et al. model as well as the input parameters.

FIG. 31 is a state diagram of the Chase model showing inputs and relationships of the model.

FIG. 32 is a schematic illustration of an example of using a physiological model such as the Chase model as an estimator of glucose concentration and the use of such an estimate to determine a next measurement time.

FIG. 33 is a graphical representation of automated determination of a next measurement time.

FIG. 34 is a schematic illustration of an example embodiment of the present invention.

FIG. 35 is a schematic illustration of an example embodiment of the present invention in operation with an automated blood removal system

FIG. 36 is a schematic illustration of a semi-automated glucose management system comprising separate glucose measurement, infusion recommendation, and infusion control systems.

FIG. 37 is a schematic illustration of a semi-automated glucose management system comprising integrated glucose measurement, infusion recommendation, and infusion control systems.

FIG. 38 is a schematic illustration of a semi-automated glucose management system comprising integrated glucose measurement, infusion recommendation, and infusion control systems.

FIG. 39 shows a schematic illustration of a glucose monitoring device comprising an indwelling fiber optic probe.

FIGS. 40A-40F. show schematic illustrations of example optical configurations for the indwelling fiber optic probe.

FIGS. 41A and 41B show fiber optic probes comprising a catheter containing a plurality of illumination and collection fibers.

FIGS. 42A-42C show three types of fiber optic probe constructions.

FIGS. 43A and 43B show a fiber optic probe for collecting a reference saline background measurement.

FIGS. 44A and 44B show fiber optic probe configurations for an auxiliary fiber optic measurement.

FIG. 45 is an example of a blood sparing device.

FIG. 46 is an graphical representation of Gardner's criteria, often referred to as Gardner's wedge.

FIG. 47 is an example of a standard arterial catheter pressure monitoring configuration.

FIG. 48 is an example of an automated blood analyte system attached to an arterial pressure monitoring system.

FIG. 49 is an example configuration which enables creation of a surrogate pressure trace.

FIG. 50 is an example of an actual pressure trace and a surrogate signal trace.

FIG. 51 is an example of an actual pressure trace and a surrogate signal trace.

FIG. 52 is an example of an automated blood analyte monitoring circuit.

FIG. 53 is an example of a blood access system that enables concurrent pressure monitoring.

FIG. 54 is an example of a blood access system where the sensor is located near the patient.

FIG. 55 is a block diagram showing the key components of the model estimation process.

FIG. 56 is a model of the blood access system.

FIG. 57 is an example demonstration of the equations used to provide concurrent pressure monitoring during the withdrawal sequence.

FIG. 58 is an example display of an automated blood analyte system.

FIG. 59 is a diagram showing the system used to create an artificial patient with a variable pressure, variable volume chamber.

FIG. 60 shows the test configuration used for accessing pressure differences.

FIG. 61 shows a test waveform.

FIG. 62 shows results Bode plot of several test configurations.

FIG. 63 shows a waveform test result from several test configurations.

FIG. 64 shows the hemodynamic monitoring errors introduced by a measurement cycle.

FIG. 65 shows the various flow types used in a measurement cycle.

FIG. 66 shows the periods during which hemodynamic monitoring information has a potential error.

FIG. 67 is a summary table of the errors generated during testing as a function of flow type.

FIG. 68 is an illustration of the waveform results from a representative flow type.

FIG. 69 is an illustration of the waveform results from a representative flow type.

FIG. 70 is an illustration of the waveform results from a representative flow type.

FIG. 71 is an illustration of the waveform results from a representative flow type.

FIG. 72 is an illustration of the waveform results from a representative flow type.

FIG. 73 is an illustration of the waveform results from a representative flow type.

FIG. 74 is an illustration of the waveform results from a representative flow type.

FIG. 75 is an illustration of the key components of a dual access system using a sheath and catheter.

FIG. 76 is a illustration of a dual access system using a sheath and catheter as it relates to a patient artery.

FIG. 77 is a block diagram showing the key components of a preferred embodiment.

FIG. 78 is a block diagram showing the key components of a preferred embodiment.

FIG. 79 is a block diagram showing the key components of a preferred embodiment.

FIG. 80 is a block diagram showing the key components of a preferred embodiment.

FIG. 81 is a block diagram showing the key components of a preferred embodiment.

FIG. 82 is a block diagram showing the key components of a preferred embodiment.

FIG. 83 is a block diagram showing the key components of a preferred embodiment.

FIG. 84 is a block diagram showing the key components of a preferred embodiment.

FIG. 85 is an illustration of an example embodiment of a blood access and measurement system suitable for use with the present invention. Fig.

FIG. 86 is an illustration of an example embodiment of a blood access and measurement system suitable for use with the present invention. Fig.

FIG. 87 is an illustration of an example embodiment where the sensor is located near the patient. Fig.

FIG. 88 is an illustration of an example embodiment allowing multilevel calibration. Fig.

FIG. 89 is an illustration of an example embodiment which enables mixing of glucose into blood obtained from the patient. Fig.

FIG. 90 is an illustration of an example embodiment which enables mixing of glucose into blood obtained from the patient. Fig.

FIG. 91 is an illustration of an example embodiment of a blood access and measurement system suitable for use with the present invention.

FIG. 92 is an illustration of an example implementation of a multi-level sensor calibration system.

FIG. 93 is an illustration of an example embodiment which enables mixing of glucose into blood obtained from the patient. Fig.

FIG. 94 is an illustration of an example embodiment which enables mixing of glucose into blood obtained from the patient. Fig.

FIG. 95 is an illustration of an example embodiment where the sensor is located near patient and where the tube junction between the blood pump and saline pump is located distal the sensor.

FIG. 96 is an illustration of an example of how a relative addition to a sample of unknown glucose concentration can be used to calibrate a system.

FIG. 97 is an illustration of an example of methods of additions.

FIG. 98 is an illustration of an example of methods of additions.

FIG. 99 is an illustration of an example of methods of additions.

FIG. 100 is an illustration of an example of methods of additions.

FIG. 101 illustrates the treatment of a patient with an ultrafiltration system (an exemplary extracorporeal blood circuit) using a controller to monitor and control the glucose concentration of a patient.

FIG. 102a illustrates the operation and fluid path of the extracorporeal blood circuit shown in FIG. 101 with one way valves for facilitating glucose sensor calibration.

FIG. 102b illustrates the operation and fluid path of the extracorporeal blood circuit shown in FIG. 101 with a three port two-way valve for facilitating glucose sensor calibration.

FIG. 103 is a diagram of the control glucose sensor embedded within the fiber bundle of the filter.

FIGS. 104a to 104d are a series of diagrams shown in plan (104a and 104c) and in cross-section (104b and 104d) to depict the operation of a three port three-way stopcock.

FIGS. 105a to 105c are a series of diagrams depicting the operation of the rotary solenoid.

FIG. 106 is a component diagram of the controller (including controller CPU (central processing unit), monitoring CPU and motor CPU), and of the sensor inputs and actuator outputs that interact with the controller.

FIG. 107 is a schematic diagram of the glucose controller.

FIG. 108 is an illustration of the system response to the partial occlusion of the withdrawal vein in a patient.

FIG. 109 is an illustration of the system response to the complete occlusion and temporary collapse of the withdrawal vein in a patient.

FIG. 110 is a diagram of the filter used on the control glucose sensor for comparison with the reference glucose sensor.

FIG. 111 is a schematic depiction of Edward's VAMP Plus System, an example blood sparing device.

FIG. 112 is an illustration of the effect of adding microliter air bubbles of various sizes to a transducer tubing system.

FIG. 113 is an illustration of Gardner's wedge showing the relationship between damping and frequency.

FIG. 114 is an illustration of an example arterial waveform tracing obtained from a monitoring system following a fast flush technique.

FIG. 115 is a schematic depiction of an arterial catheter pressure monitoring configuration.

FIG. 116 is a schematic depiction of an arterial catheter pressure monitoring configuration with an automated analyte measurement system.

FIG. 117 is a schematic depiction of a bubble and a fluid column.

FIG. 118 is a schematic depiction of the influence of bubbles on a measured arterial waveform.

FIG. 119 is a schematic depiction of the difference between measured waveforms.

FIG. 120 is a diagram showing a system used to create an artificial patient with a variable pressure, variable volume chamber.

FIG. 121 is a schematic depiction of a test configuration for accessing pressure differences.

FIG. 122 is an illustration of waveform recordings from both a reference transducer and a test transducer with no bubble present.

FIG. 123 is an illustration of waveform recordings from both a reference transducer and a test transducer following multiple automated measurements.

FIG. 124 is an illustration of an air bubble in a stopcock.

FIG. 125 is an illustration of the spectral power density for waveform recordings pre-measurement and post-measurement.

FIG. 126 is a flowchart depicting an example comparison sequence that can be used in clinical practice.

FIG. 127 is a schematic depiction of an example embodiment of an automated blood analyte measurement system.

FIG. 128 is a schematic depiction of an example embodiment of an automated blood analyte measurement system.

FIG. 132 is a schematic depiction of an example embodiment of the present invention having a syringe push-pull operation.

FIG. 133 is a schematic depiction of an example embodiment of the present invention having a syringe push-pull operation with an added calibration bag.

FIG. 134 is a schematic depiction of an example embodiment of the present invention having a push-pull operation.

FIG. 135 is a schematic depiction of an example embodiment of the present invention with a sensor close to a reservoir.

FIG. 136 is a schematic depiction of an example embodiment of the present invention with a sensor close to a patient.

FIG. 137 is a schematic depiction of an example embodiment of the present invention with a calibration bypass circuit.

FIG. 138 is a schematic depiction of an example embodiment of the present invention with a waste pathway.

FIG. 139 is a schematic depiction of an example embodiment of the present invention with a calibration pathway circuit and a waste pathway circuit.

FIG. 140 is a schematic depiction of an example embodiment of the present invention with a sensor with manual access.

FIG. 141 is a schematic depiction of an example embodiment of the present invention with two syringes.

FIG. 142 is a schematic depiction of an example embodiment of the present invention with two reservoirs and a peristaltic pump.

FIG. 143 is a schematic depiction of an example embodiment of the present invention with a peristaltic pump and reservoir.

FIG. 144 is a schematic depiction of an example embodiment of the present invention with a flow divider bypass circuit.

FIG. 145 is a schematic depiction of an example embodiment of a flow divider.

FIG. 146 is a schematic depiction of an example embodiment of the present invention including a sensor bypass loop.

FIG. 147 is a schematic depiction of an example embodiment of the present invention illustrating a general system configuration.

FIG. 148 is a schematic depiction of an example embodiment of the present invention illustrating a general system configuration.

FIG. 149 shows several reaction equations and the resulting products that lead to sensor suppression.

FIG. 150 shows a blood access circuit with two potential fluid sources and enabling the use of a low concentration maintenance fluid.

FIG. 151 shows a blood access circuit with two potential fluid sources and enabling the use of a low concentration maintenance fluid.

FIG. 152 shows a blood access circuit with two potential fluid sources and enabling the use of a low concentration maintenance fluid.

FIG. 153 is a plot of peristaltic pump withdrawal volume under various operating conditions.

FIG. 154 is a schematic illustration of an example blood access system.

FIG. 155 is an illustration of blood flow into a saline-filled flowcell.

FIG. 156 is a flow diagram of an example optical termination operation.

FIG. 157 is a plot of a linear predictor (Bhat) for blood concentration in a blood saline mixture (0 to 100% blood).

FIG. 158 comprises plots illustrating glucose accuracy comparison between YSI and measurement using an optical termination method.

FIG. 159 is a schematic illustration of an example system that incorporates a parameter sensor to evaluate sample quality during acquisition or measurement of a biological sample.

FIG. 160 is a plot of an example of a parameter monitored continuously during sample acquisition with normal parameter variance.

FIG. 161 is a plot of an example of a parameter with a time trend during sample acquisition with normal parameter variance.

FIG. 162 is a schematic illustration of an example measurement system suitable for use with the present invention.

FIG. 163 is a flow diagram of a measurement cycle according to an example embodiment of the present invention.

FIG. 164 is a schematic illustration of measurement cycle metrics according to an example embodiment of the present invention.

FIG. 165 comprises plots of a sample parameter in a typical sample and in a sample with high variance.

FIG. 166 comprises plots of a sample parameter in a typical sample and in a sample with a trending in the value.

FIG. 167 is a plot of a parameter response exhibiting excessive noise without trending.

FIG. 168 is a schematic illustration of terms relevant to the present invention.

FIG. 169 is an illustration of a typical tracing of the flow rates as a function of the cardiac cycle.

FIG. 170 is a schematic illustration of the laboratory system.

FIG. 171 is a schematic depiction of three blood flow velocity profiles investigated in an experiment related to the present invention.

FIG. 172 is a schematic illustration of sample contamination in an experiment related to the present invention.

FIG. 173 is a schematic illustration of the placement of the catheter and the orientation of the proximal port in an experiment related to the present invention.

FIG. 174 is an illustration of a test circuit and test procedure related to the present invention.

FIG. 175 is an illustration of a test circuit used in an experiment related to the present invention.

FIG. 176 is an illustration of glucose level as a function of time in an experiment related to the present invention.

FIG. 177 is a summary of parameters related to cross contamination.

FIG. 178 is an illustration of glucose level as a function of time in an experiment related to the present invention.

FIG. 179-186 are illustrations of experimental conditions and results.

FIG. 187 is an illustration of relationships between pressure and mechanical ventilation.

FIG. 188 is a schematic illustration of a blood access circuit used for demonstration of measurement instability due to cross-contamination.

FIG. 189 is an illustration of the overall stability of the measurement during the withdrawal period when the system is simply pulling blood from the beaker.

FIG. 190 is an illustration of the stability of the measurement when injecting a 60 microliter bolus but where the blood bolus has the same glucose concentration as the blood being withdrawn from the beaker.

FIG. 191 is an illustration of the stability of the measurement when injecting a 60 microliter bolus but where the blood bolus has a 2560 mg/dl glucose concentration.

FIG. 192 is an illustration of the stability of the measurement when injecting a 60 microliter bolus but where the blood bolus has a 1240 mg/dl glucose concentration.

FIG. 193 is a schematic illustration of a blood access used in connection with the present invention.

FIG. 194 is an illustration of pressure tracing obtained during eight automated sample withdrawal, measurement, re-infusion and cleaning cycles.

FIG. 195 is an illustration of intravascular pressure changes due to ventilation.

FIG. 196 is a schematic illustration of a compliance isolation method according to the present invention.

FIG. 197 is an illustration of the simulated pressure and flow responses during a withdrawal where the compliance isolation method is used.

FIG. 198 is a schematic illustration of a flow feedback method, using a flow sensor in the blood line to sense fluid flow which can be compared to a desired flow.

FIG. 199 is an illustration of the operation of the flow feedback control method during a withdrawal.

FIG. 200 is a schematic block diagram of a cascade, pressure-flow control method according to the present invention.

FIG. 201 is an illustration of the operation of the cascade, pressure-flow control method.

FIG. 202 is a schematic block diagram of a pressure control method according to the present invention.

FIG. 203 is an illustration of the operation of the pressure control method.

FIG. 204 is an illustration of catheter flow with no active control.

FIG. 205 is an illustration of catheter flow with clamping or isolation compliance control.

FIG. 206 is an illustration of catheter flow with pressure control.

### DETAILED DESCRIPTION OF THE INVENTION

The present invention comprises methods and apparatuses that can provide measurement of glucose and other analytes with a variety of sensors without many of the performance-degrading problems of conventional approaches. An apparatus according to the present invention comprises a blood access system, adapted to remove blood from a body and infuse at least a portion of the removed blood back into the body. Such an apparatus also comprises an analyte sensor, mounted with the blood access system such that the analyte sensor measures the analyte in the blood that has been removed from the body by the blood access system. A method according to the present invention comprises removing blood from a body, using an analyte sensor to measure an analyte in the removed blood, and infusing at least a portion of the removed blood back into the body.

The performance of the analyte sensor in the present invention can be dramatically improved compared with conventional applications by minimizing various issues that contribute to degraded sensor performance over time and by providing for cleaning and calibrating the measurement sensor over time. The physiological lag problems associated with conventional tissue measurements can also be reduced with the present invention by making a direct measurement in blood or by ensuring that there is appropriate agreement between the ISF glucose level and that in whole blood.

Some embodiments of the present invention provide for effective cleaning of the sensor. If effectively cleaned at the end of each measurement, the amount of sensor fouling and/or drift can be minimized. Saline or another physiologically compatible solution can be used to clean the sensing element.

A typical glucose sensor used relies on a glucose-dependent reaction to measure the amount of glucose present. The reaction typically uses both oxygen and glucose as reactants. If either oxygen or glucose is not present, the reaction can not proceed; some embodiments of the present invention provide for total removal of one or the other to allow a zero point calibration condition. Saline or another physiological compatible solution that does not contain glucose could be used to effectively create a zero point calibration condition.

There can be limitations associated with a zero point calibration so that one may desire to use a calibration point with a glucose value above zero and preferably within the physiological range. Some embodiments of the present invention provide for such a calibration by exposing the sensor to a glucose containing solution with a known glucose concentration. This can effectively recalibrate the sensor and improve its accuracy. The ability to make frequent recalibrations enables a simplistic approach to maintaining overall sensor accuracy.

In many medical laboratory measurement products a two point calibration is used. Some embodiments of the present invention provide two types of calibrations to provide a two point calibration capability. A two point calibration can allow both bias and slope to be effectively determined and mitigated.

In practice the degree or amount of physiological lag observed between ISF glucose levels in whole blood glucose levels creates a significant error source. Some embodiments of the present invention reduce this source of error by placing the sensor in direct contact with blood.

Recognizing the several error sources, the present invention provides an accurate continuous or semicontinuous blood glucose measurement system for use in applications such as the intensive care unit. Some embodiments of the present invention place blood in contact with a sensing mechanism for a defined measurement period and then clean the sensor. Following cleaning of the sensor, a calibration point or points can be established. The present invention contemplates a variety of blood access circuits that can enable the sensor to be cleaned on a periodic basis and can allow for recalibration; illustrative examples are described below. In addition to providing a mechanism for improved sensor performance, the disclosed blood access systems can also provide methods for occlusion management, minimization of blood loss and minimization of saline used for circuit cleaning.

The example embodiments generally show a blood access system with the ability to control fluid flows at a location removed from the blood access console and near the patient. The ability to control fluid flows at this remote location does not necessitate the use of a mechanical valve or other similar apparatus that similarly directs or control flow at a point near the patient. Additionally it does not require nurse or other human intervention. For multiple reasons, including safety and reliability, it is desirable not to have a mechanical device, wires, or electrical power near the patient. As shown in many example embodiments, this capacity is enabled through the use of a pumping mechanism that provides for both fluid stoppage and movement. Additional capabilities are provided by bidirectional operation of the pumps, and by operation at variable speeds including complete stoppage of fluid flow. As used in the disclosure, operation may be the use of the pump as a flow control device to prevent flow. As shown in the example embodiments these capabilities can be provided through peristaltic pumps and syringe pumps. It is recognized by one of ordinary skill in the art that these capabilities can also be provided by other fluid handling devices, including as examples linear “finger” pumps, valveless rotating and reciprocating piston metering pumps, piston pumps, lifting pumps, diaphragm pumps, and centrifugal pumps. “Plunger” pumps to include syringe pumps as well as those that can clean a long thin flexible piece of tubing are considered. These types of plunger pumps have the advantage of removing or transporting the fluid without the need for a following fluid volume. For example, no follow volume is required when using a syringe pump.

The example embodiments generally show a sensor in contact with a blood access system. The sensor can be immersed or otherwise continuously exposed to fluid in the system. It can also comprise a noncontact sensor that interacts with fluid in the system. It can also comprise a sensor remote from the blood access system, where the sensor element in the example comprises a port or other sampling mechanism that allows a suitable sample of fluid from the system to be extracted and presented to the remote sensor. This type of sampling can be used with existing technology glucose meters and reagent strips.

Example Embodiment Comprising a Sensor and a Fluid Management System.

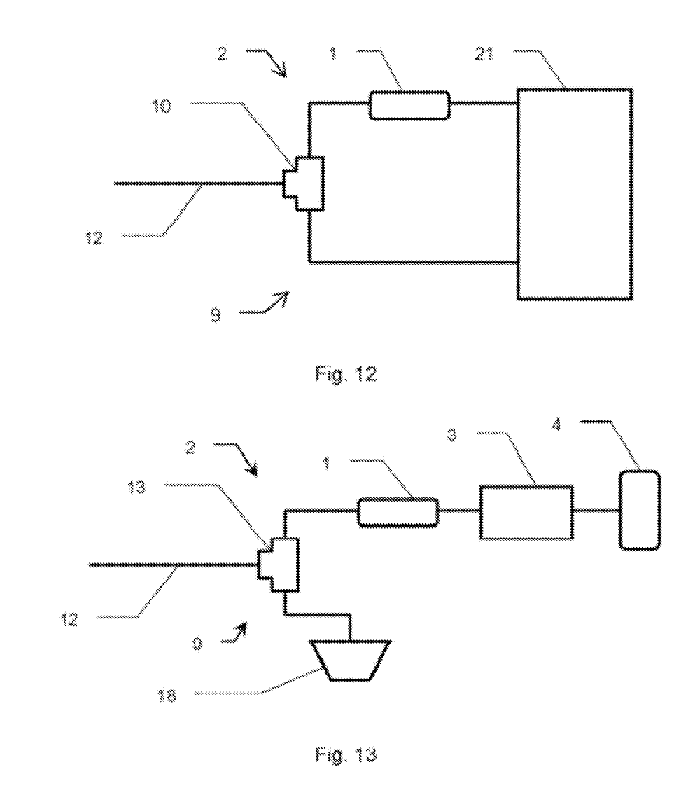

FIG. 12 is a schematic illustration of an example embodiment of the present invention comprising a sensor and a fluid management system. The system comprises a catheter (or similar blood access device) (12) in fluid communication with the vascular system of a patient. A tubing extension (if required) extends from the catheter (12) to a junction (10). A first side of the junction (10) connects with fluid transport apparatus (2) such as tubing (for reference purposes called the “left side” of the blood system); a second side of the junction (10) connects with fluid transport apparatus (9) such as tubing (for reference purposes called the “right side” of the blood system). A sensor (1) mounts with the left side (2) of the blood loop. A fluid management system (21) is in fluid communication with the left side (2) and right side (9) of the blood system. In operation, the fluid management system (21) acts to draw blood from the patient through the catheter 12 and into the left side (2) of the blood system to the sensor 1. The sensor 1 determines a blood property of interest, for example the concentration of glucose in the blood. The fluid management system (21) can push the blood back to the patient through the left side (2) of the blood system, or can further draw the measured blood into the right side (9) of the blood system, and through junction (10) to catheter (12) and back into the patient.

The fluid management system (21) can control the fluid volume flow and fluid pressure in the left (2) and right (9) sides of the blood system to control whether fluid is being withdrawn from the patient, infused into the patient, or neither. The fluid management system (21) can also comprise a source of a suitable fluid such as saline, and manage fluid flow in the system such that saline is circulated through the left (2) and right (9) sides to flush or clean the system. The fluid management system can further comprise an outlet to a waste container or channel, and manage fluid flow such that used saline, blood/saline mix, or blood that is not desired to be returned to the patient (depending on the requirements of the application) is delivered to the waste container or channel.

Example Embodiment Comprising a Blood Loop System with a Syringe Pump.

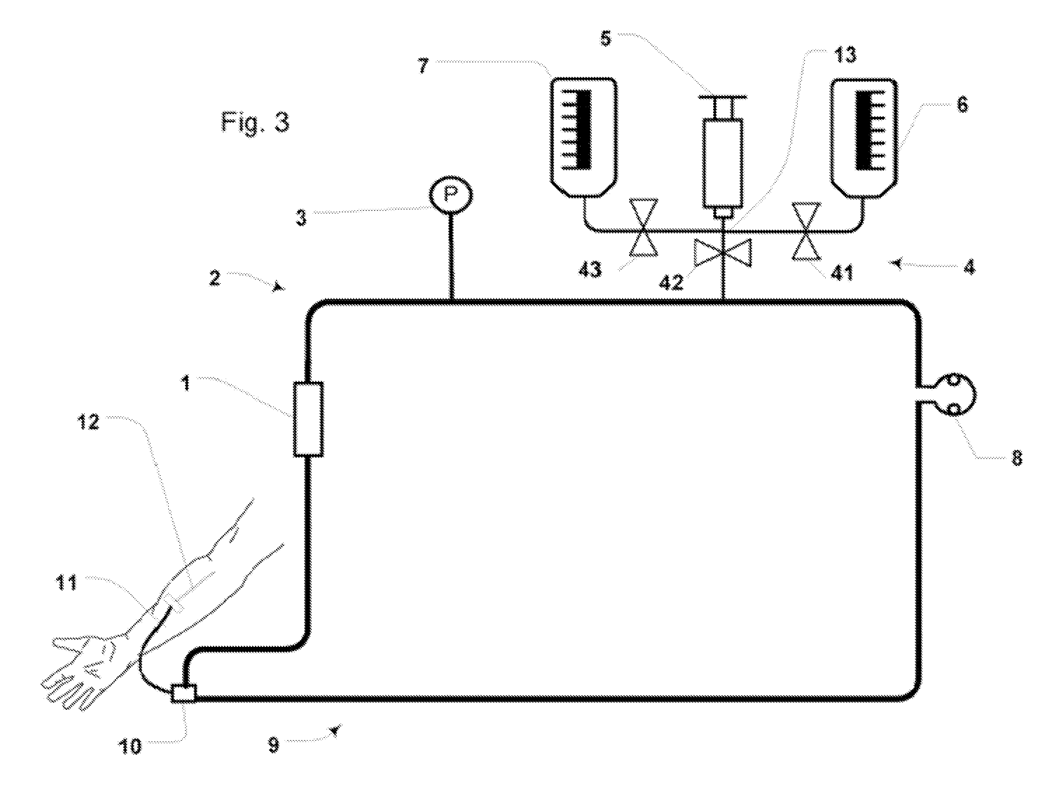

FIG. 3 is a schematic illustration of an example embodiment of the present invention comprising a blood access system using a blood flow loop. The system comprises a catheter (or similar blood access device) (12) in fluid communication with the vascular system of a patient. A tubing extension (11) (if required) extends from the catheter (12) to a junction (10). A first side of the junction (10) connects with fluid transport apparatus (2) such as tubing (for reference purposes called the “left side” of the blood loop); a second side of the junction (10) connects with fluid transport apparatus (9) such as tubing (for reference purposes called the “right side” of the blood loop). A sensor measurement cell (1) and a pressure measurement device (3) mount with the left side (2) of the blood loop. A peristaltic pump (8) mounts between the left side (2) and the right side (9) of the blood loop. A pinch valve (42) (“pinch valve” is used for convenience throughout the description to refer to a pinch valve or any suitable flow control mechanism) mounts between the left side (2) of the blood loop and a junction (13), controlling fluid communication therebetween. A pinch valve (43) mounts between the junction (13) and a waste channel (7) (such as a bag), controlling fluid communication therebetween. A pinch valve 41 mounts between the junction (13) and a source of wash fluid (6) (such as a bag of saline), controlling fluid flow therebetween. A syringe pump (5) mounts in fluid communication with the junction (13). The system can be operated as described below. The description assumes a primed state of the system wherein saline or another appropriate fluid is used to initially fill some or all channels of fluid communication. Those skilled in the art will appreciate that other start conditions are possible. Note that “left side” and “right side” are for convenience of reference only, and are not intended to limit the placement or disposition of the blood loops to specific left-right relationship.

Blood sample and measurement process. A first sample draw with the example embodiment of FIG. 3 can be accomplished with the following steps:

1. Syringe pump (5) initiates a draw along the left side (2) of the blood loop.
2. The blood interacts with the sensor measurement cell (1). The volume of the catheter (12) and extension tubing (11) can be determined from the syringe pump (5) operating parameters and the time until blood is detected by the sensor measurement cell (1) and used for future reference.
3. Sensor measurements can be made as the blood moves through the measurement cell (1).
4. As blood nears junction (13) the system can be stopped and the saline that was drawn into the syringe pump (5) placed in waste bag (7) by the appropriate use of pinch valves (43, 42, 41).
5. Blood drawn via the left side can continue via the withdrawal of syringe (5).
6. Withdrawal of blood by the syringe, either fully or partially, is stopped. Sensor sampling of the measurement cell can be continued or stopped.
7. Initially saline and then blood is re-infused into the subject via combination of peristaltic pump (8) and syringe (5). The two pump mechanisms operate at the same rate such that blood is moved along the right side (9) of the circuit only. Note, blood does not substantially progress up the left side (2) of the circuit but is re-infused past junction (10) and into the patient.
8. One or more weight scales (not shown) can be used to measure the waste and saline solution together or independently. Such weight scales can allow real time compensation between the pumps, e.g., to ensure that the rates match, or to ensure that a desired rate difference or bias is maintained. For instance it can be desirable that a certain volume of saline be infused into the patient during a recirculation cycle. In such an application, the combined weight of the waste and saline bag should decrease by the weight of the desired volume of saline. If the weight or weights do not correspond to the expected weight or weights, then one or both pumps can be adjusted. If a net zero balance is required then the combined weight at the start of recirculation mode and at the end of recirculation mode should be the same; again, one or both pumps can be adjusted to reach the desired weight or weights.

Subsequent Blood Sampling. For subsequent samples, the blood residing in the catheter (12) and extension tubing (11) has already been tested and can be considered a “used” sample. The example embodiment of FIG. 3 can prevent this sample from contaminating the next measurement, by operation as follows.

1. Syringe pump (5) and peristaltic pump (8) initiate the blood draw by drawing blood up through the right side of the blood loop.
2. The withdrawal continues until all of the used blood has passed junction (10). The volume determination made during the initial draw can enable the accurate determination of the location of the used blood sample.
3. Once the used sample has passed the junction (10), the peristaltic pump (8) can be turned off and blood withdrawn via the left side (2) of the circuit. Sensor measurement of the blood can be made during this withdrawal.
4. The withdrawal process can continue for a predetermined amount of time. Following completion of the sensor sampling (or overlapped in time), the blood can be re-infused into the patient. The blood is re-infused into subject via combination of peristaltic pump (8) and syringe pump (5). The two pumps operate at the same rate such that blood is moved along the right side (9) of the circuit only. Note, blood does not progress up the left side (2) of the circuit but is re-infused past junction (10) and into the patient. There is no requirement that the withdrawal and infusion rates be the same for this blood loop system.

Cleaning of system and saline calibration procurement. A cleaning and calibration step can clean the system of any residual protein or blood build-up, and can characterize the system; e.g., the performance of a measurement system can be characterized by making a saline calibration reference measurement, and that characterization used in error reporting, instrument self-tests, and to enhance the accuracy of blood measurements. The cleaning process can be initiated at the end of a standard blood sampling cycle, at the end of each cycle, or at the end of each set of a predetermined number of cycles, at the end of a predetermined time, when some performance characterization indicates that cleaning is required, or some combination thereof. A cleaning cycle can be provided with the example embodiment of FIG. 3 with a method such as the following.

1. The start condition for initiation of the cleaning cycle has the syringe substantially depressed following infusion of blood into the patient.
2. Pinch valve (42) closed and pinch valve (41) opened and syringe (5) withdraws saline from the wash bag (6).
3. Following the withdrawal, pinch valve (42) is opened and (41) and (43) are closed.
4. Syringe pump (5) pushes saline toward patient at first rate while peristaltic pump (8) operates at a second rate equal to one half of the first rate. This rate relationship means that saline is infused into the two arms for the loop at equal rates and the blood present in the system is re-infused into the patient.
5. Following completion of the saline infusion, both arms of the loop system (2, 9) as well as the tubing (11) and catheter (12) are filled with saline.
6. Pinch valve (42) is closed and peristaltic pump (8) is turned on in a vibrate mode or pulsatile flow mode to completely clean the loop.
7. Pinch value (42) is opened. Syringe begins pull at a third rate and peristaltic pump pulls saline at fourth rate equal to one half of the third rate. This process effectively fills the entire loop with blood while concurrently placing the saline used for cleaning into the syringe (5). Sensor measurements can occur after the blood/saline junction has passed the measurement cell.
8. Pinch valve (43) opened and pinch valve (42) closed and saline is infused into waste bag (7).
9. Pinch valve (43) closed, (42) opened and blood pulled from patient and back to measurement mode.

Characteristics of the example embodiment. The example embodiment of FIG. 3 allows sensor measurements of blood to be made on a very frequent basis in a semi-continuous fashion. There is little or no blood loss except during the cleaning cycle. Saline is infused into the patient only during cleaning, and very little saline is infused into the patient. The gas dynamics of the system can be fully equilibrated, allowing the example embodiment to be used with arterial blood. There are no blood/saline junction complications except during cleaning. The system contains a pressure monitor that can provide arterial, central venous, or pulmonary artery catheter pressure measurements after compensation for the pull and push of the blood access system. The system can compensate for different size catheters through the volume pulled via the syringe pump. The system can determine occlusions or partial occlusions with the blood sensor or the pressure sensor. Due to the flexibility in operation and the direction of flow, the system can determine if the occlusion or partial occlusion is in the left side of the circuit, the right side of the circuit or in the tubing between the patient and the T-junction. If the occlusion is in the right or left sides, the system can enter a cleaning cycle with agitation and remove the clot build-up. If a microembolus is detected the system can initiate a mode of operation such that the problematic blood is taken directly to waste. The system can then enter into a mode such that it becomes saline filled but does not initiate additional blood withdrawals. In the case of microemboli detection, the system has effectively managed the potentially dangerous situation and the nurse can be notified to examine the system for emboli formation centers such as poorly fitting catheter junctions.

Example Embodiment Comprising a Blood Loop System with a Peristaltic Pump.

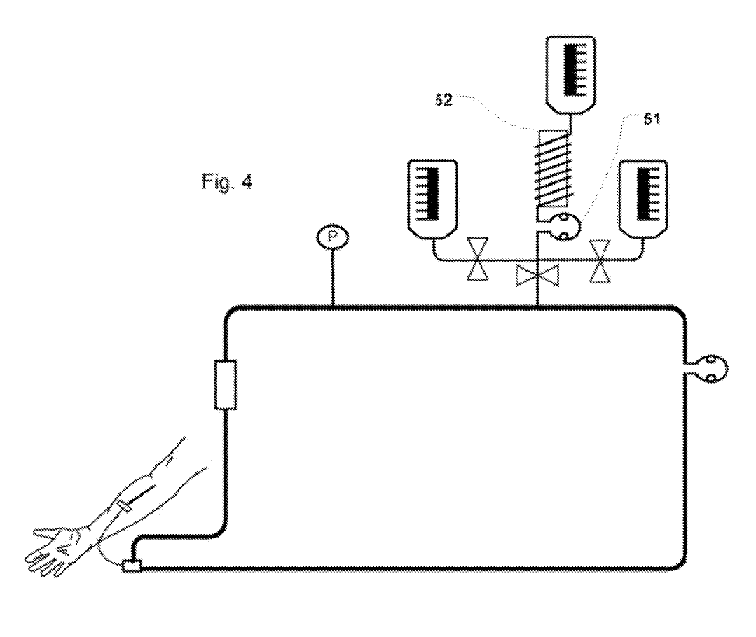

FIG. 4 is a schematic illustration of a blood loop system with a peristaltic pump. The system of FIG. 4 is similar to that of FIG. 3, with the syringe pump of FIG. 3 replaced by a peristaltic pump (51) and a tubing reservoir (52). The reservoir as used in this application is defined as any device that allows for the storage of fluid. Examples included are a piece of tubing, a coil of tubing, a bag, a flexible pillow, a syringe, a bellows device, or any device that can be expanded through pressure, a fluid column, etc. The operation of the system is essentially unchanged except for variations that reflect the change from a syringe pump to a peristaltic or other type of pump. The blood loss and saline consumption requirements of the system are of course different due to the blood saline interface present in the operation of the second peristaltic pump. Unlike the syringe pump of FIG. 3, the example embodiment of FIG. 4 must maintain a sterile compartment and minimize the contact between air and blood for many applications. A saline fluid column can fill the tubing, and effectively moves up and down as fluid is with drawn by the peristaltic pump.

Push Pull System.

FIG. 13 is a schematic illustration of a blood access system according to the present invention. The system comprises a catheter (or similar blood access device) (12) in fluid communication with the vascular system of a patient. A tubing extension (if required) extends from the catheter (12) to a junction (13). A first side of the junction (13) connects with fluid transport apparatus (2) such as tubing (for reference purposes called the “left side” of the blood system); a second side of the junction (13) connects with fluid transport apparatus (9) such as tubing (for reference purposes called the “right side” of the blood system). A sensor (1) is in fluid communication with the left side (2) of the system. A pump (3) is in fluid communication with the left side (2) of the system (shown in the figure as distal from the patient relative to the sensor (1); the relative positions can be reversed). A source (4) of suitable fluid such as saline is in fluid communication with the left side (2) of the system. A waste container (18) or connection to a waste channel is in fluid communication with the right side (9) of the system. In operation, the pump (3) operates to draw blood from the patient through the catheter (12) and junction (13) into the left side (2) of the system. The sensor (1) determines a desired property of the blood, e.g., the glucose concentration in the blood. The pump (3) operates to draw saline from the container (4) and push the blood back into the patient through junction (13) and catheter (12). After a sufficient quantity of blood has been reinfused (e.g., by volume, or by acceptable blood/saline mixing threshold), then the pump (3) operates to push remaining blood, blood/saline mix, or saline into the right side (9) of the system and into the waste container (18) or channel. The transport of fluid from the left side (2) to the right side (9) of the system can be used to clear undesirable fluids (e.g., blood/saline mixtures that are not suitable for reinfusion or measurement) and to flush the system to help in future measurement accuracy. Valves, pumps, or additional flow control devices can be used to control whether fluid from the left side (2) is infused into the patient or transported to the right side (9) of the system; and to prevent fluid from the right side (9) of the system from contaminating blood being withdrawn into the left side (2) of the system for measurement.

Push Pull System with Two Peristaltic Pumps.

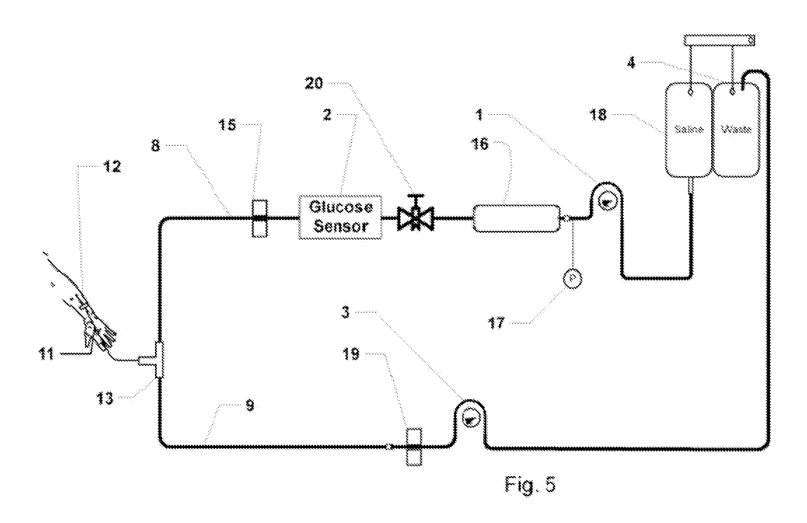

FIG. 5 is a schematic illustration of a blood access system implemented based upon a pull-push mechanism with a second circuit provided to prevent fluid overload of the patient. The system comprises a catheter (or similar blood access device) (12) in fluid communication with the vascular system of a patient. A tubing extension (11) (if required) extends from the catheter (12) to a junction (13). A first side of the junction (13) connects with fluid transport apparatus (8) such as tubing (for reference purposes called the “left side” of the blood loop); a second side of the junction (13) connects with fluid transport apparatus (9) such as tubing (for reference purposes called the “right side” of the blood loop). An air detector (15) that can serve as a leak detector, a pressure measurement device (17), a glucose sensor (2), and a needle-less blood access port (20) mount with the left side of the blood loop. A tubing reservoir (16) mounts with the left side of the blood loop, and is in fluid communication with a blood pump (1). Blood pump (1) is in fluid communication with a reservoir (18) of fluid such as saline. A blood leak detector (19) serves as a safety that can serve as a leak detector mounts with the right side of the blood loop. A second blood pump (3) mounts with the right side of the blood loop, and is in fluid communication with a receptacle or channel for waste, depicted in the figure as a bag (4). Elements of the system and their operation are further described below.

Blood Sample and Measurement Process—First Sample Draw.

1. Pump (1) initiates a draw of blood from the catheter (12).
2. The blood interacts with the sensor measurement cell (2). The volume of the catheter (12) and tubing (11) can be determined and used for future reference and for the determination of blood-saline mixing.
3. Sensor measurements can be made as the blood moves through the measurement cell.
4. Pump (1) changes direction and sensor measurements continue.
5. Pump (1) reinfuses blood into the patient. As the mixed blood-saline junction passes the junction (13), it becomes progressively more dilute.
6. Following re-infusion of the majority of the blood, peristaltic pump (3) is turned on and the saline with a small amount of residual blood is taken to the waste bag (4).
7. The system can be washed with saline after each measurement if desired.
8. Additionally the system can go into an agitation mode that fully washes the system
9. Finally the system can enter into a keep vein open mode (KVO). In this mode a small amount of saline is continuously or periodically infused to keep the blood access point open.

Blood sample and measurement process—Subsequent Blood Sampling. For subsequent samples, the tubing between the patient and the pump (1) is filled with saline and it can be desirable that this saline not become mixed with the blood. This can be achieved with operation as follows:

1. Pump (1) initiates the blood draw by drawing blood up through junction (13).
2. The withdrawal continues as blood passes through the sensor measurement cell (2). The blood after passing the measurement cell can be effectively stored in the tubing reservoir (5).
3. Sensor measurements can be made during this withdrawal period.
4. Following completion of the blood withdrawal, the blood can be re-infused into the patient by reversing the direction of pump (1).
5. Sensor measurements can also be made during the re-infusion period.
6. As the mixed blood-saline passes through the junction (13), it becomes progressively more dilute.
7. Following re-infusion of the majority of the blood, peristaltic pump (3) is turned on at a rate that matches the rate of pump (1). The small amount of residual blood mixed with the saline is taken to the waste bag (4).
8. This process results in a washing of the system with saline.
9. Additional system cleaning is possible through an agitation mode. In this mode the fluid is moved forward and back such that turbulence in the flow occurs.
10. Between blood samplings, the system can be placed in a keep vein open mode (KVO). In this mode a small amount of saline can be infused to keep the blood access point open.

Characteristics of Push Pull with Peristaltic Pumps. The example embodiment of FIG. 5 can operate with minimal blood loss since the majority of the blood removed can be returned to the patient. The diversion of saline into a waste channel can prevent the infusion of significant amounts of saline into the patient. The pump can be used to compensate for different sizes of catheters. The system can detect partial or complete occlusion with either the analyte sensor or use of pressure sensor (17) or additional pressure sensors not shown. An occlusion can be cleared through a variety of means. For example if the vein is collapsing and the system needs to re-infuse saline either the blood pump or the flush pump can be used to effectively refill the vein. If there is evidence of occlusion in the measurement cell area, the both the blood pump and flush pumps can be activated such that significant fluid can be flushed through the system for effective cleaning. In addition to high flow rates the bidirectional pump capabilities of the pumps can be used to remove occlusions. If a microembolus is detected the system can initiate a mode of operation such that the problematic blood is taken directly to waste. The system can then enter into a mode such that it becomes saline filled but does not initiate additional blood withdrawals. In the case of microemboli detection, the system has effectively managed the potentially dangerous situation and the nurse can be notified to examine the system for emboli formation centers such as poorly fitting catheter junctions.

Push Pull System with Syringe Pump.

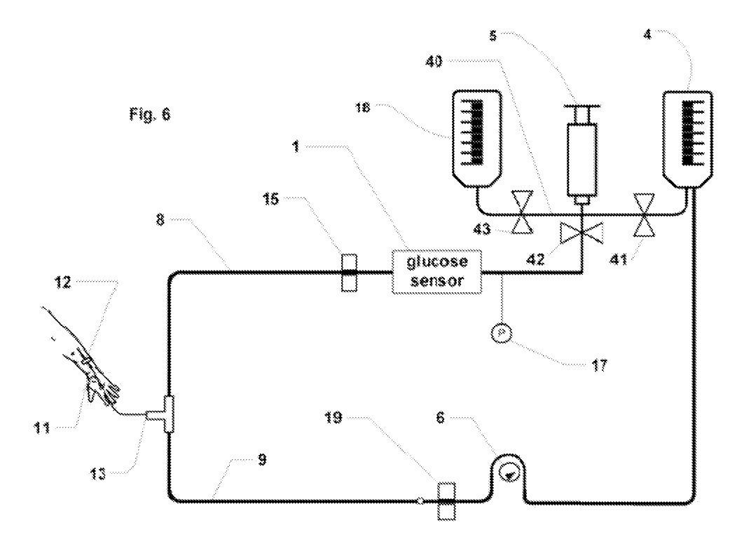

FIG. 6 is a schematic illustration of a blood access system based upon a pull-push mechanism with a second circuit provided to prevent fluid overload of the patient. The system comprises a catheter (or similar blood access device) (12) in fluid communication with the vascular system of a patient. A tubing extension (11) (if required) extends from the catheter (12) to a junction (13). A first side of the junction (13) connects with fluid transport apparatus (8) such as tubing (for reference purposes called the “left side” of the blood loop); a second side of the junction (13) connects with fluid transport apparatus (9) such as tubing (for reference purposes called the “right side” of the blood loop). An air detector (15) that can serve as a leak detector, a pressure measurement device (17), and a glucose sensor (1) mount with the left side of the blood loop. A pinch valve (42) mounts between the left side (2) of the blood loop and a junction (40), controlling fluid communication therebetween. A pinch valve (41) mounts between the junction (40) and a waste channel (4) (such as a bag), controlling fluid communication therebetween. A pinch valve (43) mounts between the junction (40) and a source of wash fluid (18) (such as a bag of saline), controlling fluid flow there between. A syringe pump (5) mounts in fluid communication with the junction (40). A blood leak detector (19) that can serve as a leak detector mounts with the right side of the blood loop. A second blood pump (6) mounts with the right side of the blood loop, and is in fluid communication with a receptacle or channel for waste, depicted in the figure as a bag (4). Elements of the system and their operation are further described below.

Blood Sample and Measurement Process—First Sample Draw.

1. Syringe pump (5) initiates a draw.
2. The blood interacts with the sensor measurement cell (1). The volume of the catheter (12) and tubing (11) can be determined and used for future reference and for the determination of blood-saline mixing.
3. Sensor measurements can be made as the blood moves through the measurement cell.
4. The syringe pump changes direction and sensor measurements can continue.
5. Blood is re-infused into the patient. As the mixed blood-saline junction passes the junction (13), it becomes progressively more dilute.
6. Following re-infusion of a portion (e.g., the majority) of the blood, peristaltic pump (6) is turned on and the saline with a small amount of residual blood is taken to the waste bag.
7. The system can be washed with saline after each measurement if desired.
8. Additionally the system can go into an agitation mode that fully washes the system.
9. Finally the system can enter a keep vein open mode (KVO). In this mode a small amount of saline is infused to keep the blood access point open.

Blood sample and measurement process—Subsequent Blood Sampling. For subsequent samples, the tubing between the patient and the syringe is filled saline and it can be desirable that this saline not become mixed with the blood. The pinch valves enable the saline to be pushed to waste and the amount of saline/blood mixing to be minimized. This can be achieved with operation as described below.

1. Syringe pump (5) initiates the blood draw by drawing blood up through junction (13).
2. The withdrawal continues until blood saline juncture reaches the base of the syringe. At this point in the sequence, pinch valve (42) is closed and valve (41) is opened, and the syringe pump direction reversed. This process enables the resident saline to be placed into the waste bag.
3. Valve (42) is opened, valve (41) closed and the syringe is now withdrawn so that only blood or blood with very little saline contamination is pulled into the syringe.
4. Sensor measurements can be made during this withdrawal period.
5. Following completion of the blood withdrawal, the blood is re-infused into the patient by reversing the direction of the syringe pump. As the mixed blood-saline passed through the junction (13), it becomes progressively more dilute.
6. Following re-infusion of the majority of the blood, peristaltic pump (6) is activated with the concurrent infusion from the syringe pump and the saline with a small amount of residual blood it taken to the waste bag.
7. This process results in a washing of the system with saline.
8. Additional system cleaning is possible through an agitation mode. In this mode the fluid is moved forward and back such that turbulence in the flow occurs.
9. Between blood samplings, the system can be placed in a keep vein open mode (KVO). In this mode a small amount of saline is infused to keep the blood access point open.

Characteristics of Push Pull with Syringe Pump. The system can operate with little blood loss since the majority of blood is re-infused into the patient. The diversion of saline to waste can result in very little saline infused into the patient. Saline mixing occurs only during blood infusion. The pressure monitor can provide arterial, central venous, or pulmonary artery catheter pressure measurements after compensation for the pull and push of the blood access system. The system can compensate for different size catheters through the volume pulled via the syringe pump.

The system can detect partial or complete occlusion with either the analyte sensor or the pressure sensor. An occlusion can be cleared through a variety of means. For example if the vein is collapsing and the system needs to re-infuse saline either the syringe pump or the flush pump can be used to effectively refill the vein. If there is evidence of occlusion in the measurement cell area, both the syringe pump and flush pumps can be activated such that significant fluid can be flushed through the system for effective cleaning. In addition to high flow rates the bidirectional pump capabilities of the pumps can be used to remove occlusions.

The syringe pump mechanism can also have a source of heparin or other anticoagulant attached through an additional port (not shown). The anticoagulant solution can then be drawn into the syringe and infused into the patient or pulled through the flush side of the system. The ability to rinse the system with such a solution can be advantageous when any type of occlusion is detected.

If a microembolus is detected the system can initiate a mode of operation such that the problematic blood is taken directly to waste. The system can then enter into a mode such that it becomes saline filled but does not initiate additional blood withdrawals. In the case of microemboli detection, the system has effectively managed the potentially dangerous situation and the nurse can be notified to examine the system for emboli formation centers such as poorly fitting catheter junctions.

Push Pull System with Syringe & Peristaltic Pump.

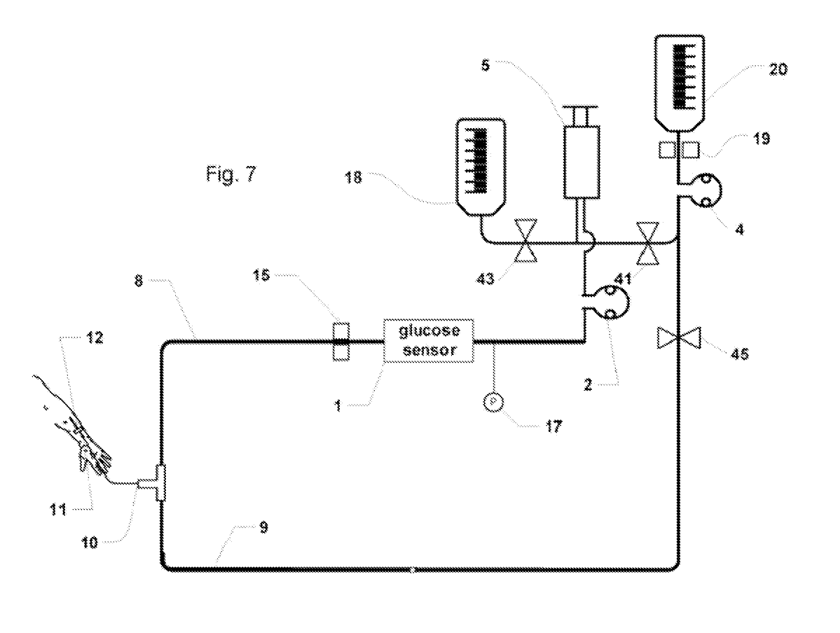

FIG. 7 is a schematic illustration of another example push pull system. The system comprises a catheter (or similar blood access device) (12) in fluid communication with the vascular system of a patient. A tubing extension (11) (if required) extends from the catheter (12) to a junction (10). A first side of the junction (10) connects with fluid transport apparatus (8) such as tubing (for reference purposes called the “left side” of the blood loop); a second side of the junction (10) connects with fluid transport apparatus (9) such as tubing (for reference purposes called the “right side” of the blood loop). An air detector (15) that can serve as a leak detector, a pressure measurement device (17), and a glucose sensor (1) mount with the left side of the blood loop. A blood pump (2) mounts with the left side of the blood loop such that it controls flow between a passive reservoir (5) and the left side of the blood loop. A pinch valve (45) mounts with the right side of the blood loop, controlling flow between the right side of the blood loop and a second pump (4). The second pump (4) is also in fluid communication with a waste channel such as a bag (20), with a leak detector (19) mounted between the pump (4) and the bag (20). A pinch valve (41) mounts between the pump (4) and a port of the passive reservoir (5), which port is also in fluid communication with a pinch valve (43) between the port and a source of saline such as a bag (18). Elements of the system and their operation are further described below.

Blood Sample and Measurement Process—Sampling Process.

1. The passive reservoir is not filled and valve (41) is open.
2. Peristaltic pump (4) & pump (2) initiate the blood draw. The saline in the line moves into the saline bag.
3. As the blood approaches the syringe, pump (4) stops and valve (41) closes. The blood now moves into the passive reservoir.
4. Sensor sampling of the blood occurs in sensor (1).
5. Pump (2) reverses direction and the blood is infused into the patient.
6. The reservoir goes to minimum volume, at which point valve (43) opens and saline washes the reservoir and is used to push the blood back to the patient.
7. As the mixed blood-saline passes through the junction (13), it becomes progressively more dilute.
8. Following re-infusion of the majority of the blood or all of the blood, peristaltic pump (4) is turned on at the same rate as pump (2) and valves (45) and (43) are open. The combination of pumps creates a wash circuit that cleans the system.
9. Further washing of the syringe reservoir can occur by opening valves (43, 41) with pump (4) active.
10. Keep vein open infusions can occur by having pump (2) active with valve (43) open.

Characteristics of the Push Pull System with Syringe and Peristaltic Pump. Blood is always moving either into or out of the access system. Circuit cleaning can be independent of syringe cleaning. Blood loss is zero or minimal since the majority of blood is re-infused in to the patient. Very little saline is infused due to diversion of saline into waste and the fact that the mixing period is only during infusion. Saline mixing during blood infusion only. The system contains a pressure monitor that can provide arterial, central venous, or pulmonary artery catheter pressure measurements after compensation for the pull and push of the blood access system. The system can compensate for different size catheters through the volume pulled via the syringe pump.

The system can detect partial or complete occlusion with either the analyte sensor or the pressure sensor. An occlusion can be cleared through a variety of means. For example if the vein is collapsing and the system needs to re-infuse saline via either syringe pump. If there is evidence of occlusion in the measurement cell area, the both syringe pumps can be activated such that significant fluid can be flushed through the system for effective cleaning. In addition to high flow rates the bidirectional pump capabilities of the pumps can be used to remove occlusions. The flexibility of the described system with the various pinch valves allows one to identify the occlusion location and establish a proactive cleaning program to minimize further occlusion.

The syringe pump mechanism can also have a source of heparin or other anticoagulant attached through an additional port (not shown). The anticoagulant solution can then be drawn into the syringe and infused into the patient or pulled through the flush side of the system. The ability to rinse the system with such a solution could be advantageous when any type of occlusion is detected.

Push Pull System.

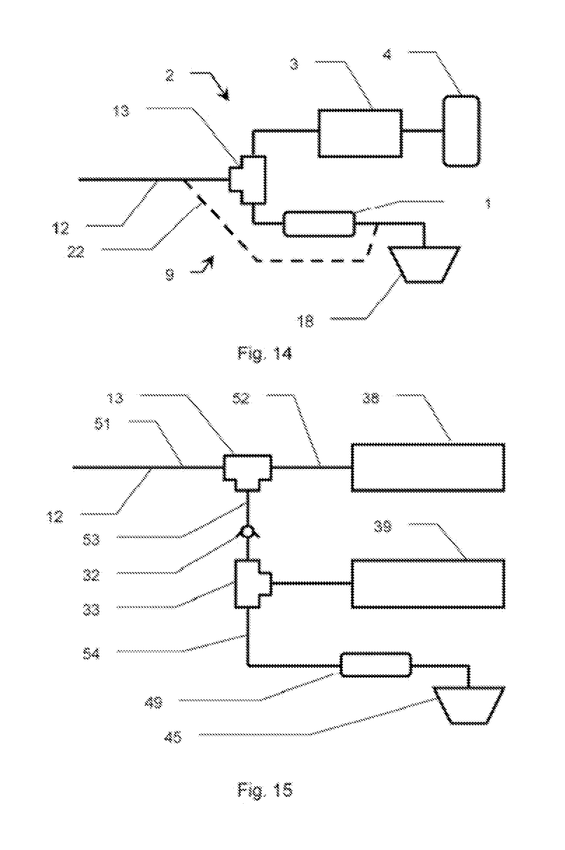

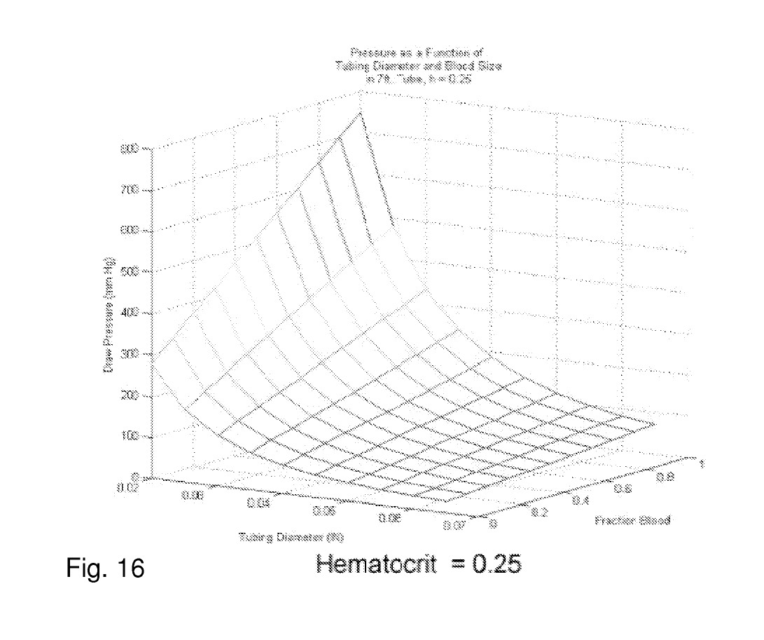

FIG. 14 is a schematic illustration of a blood access system according to the present invention. The system comprises a catheter (or similar blood access device) (12) in fluid communication with the vascular system of a patient. A tubing extension (if required) extends from the catheter (12) to a junction (13). A first side of the junction (13) connects with fluid transport apparatus (2) such as tubing (for reference purposes called the “left side” of the blood system); a second side of the junction (13) connects with fluid transport apparatus (9) such as tubing (for reference purposes called the “right side” of the blood system). A pump (3) is in fluid communication with the left side (2) of the system. A source (4) of suitable fluid such as saline is in fluid communication with the left side (2) of the system. A sensor (1) is in fluid communication with the right side (9) of the system. A waste container (18) or connection to a waste channel is in fluid communication with the right side (9) of the system. An optional fluid transport apparatus 22 is in fluid communication with the right side (9) of the system between the sensor (1) and the waste container (18) or channel, and with the patient (e.g., via the catheter (12)).

In operation, the pump (3) operates to draw blood from the patient through the catheter (12) and junction (13) into the left side (2) of the system. Once a sufficient volume of blood has been drawn into the left side (2), the pump operates to push the blood from the left side (2) to the right side (9), wherein the sensor (1) determines a desired blood property (e.g., the concentration of glucose in the blood). The pump (3) can draw saline from the bag (4) to push the blood through the system. Blood from the sensor (1) can be pushed to the waste container (18) or channel, or can optionally be returned to the patient via the optional return path (22). The transport of fluid through from the left side (2) to the right side (9) of the system can be used to clear undesirable fluids (e.g., blood/saline mixtures that are not suitable for reinfusion or measurement) and to flush the system to help in future measurement accuracy. Valves, pumps, or additional flow control devices can be used to control whether fluid is drawn from patient into the left side (2) or transported to the right side (9) of the system; and to prevent blood/saline mix and saline from the left side (9) of the system from being infused into the patient.

Push Pull with Additional Path.

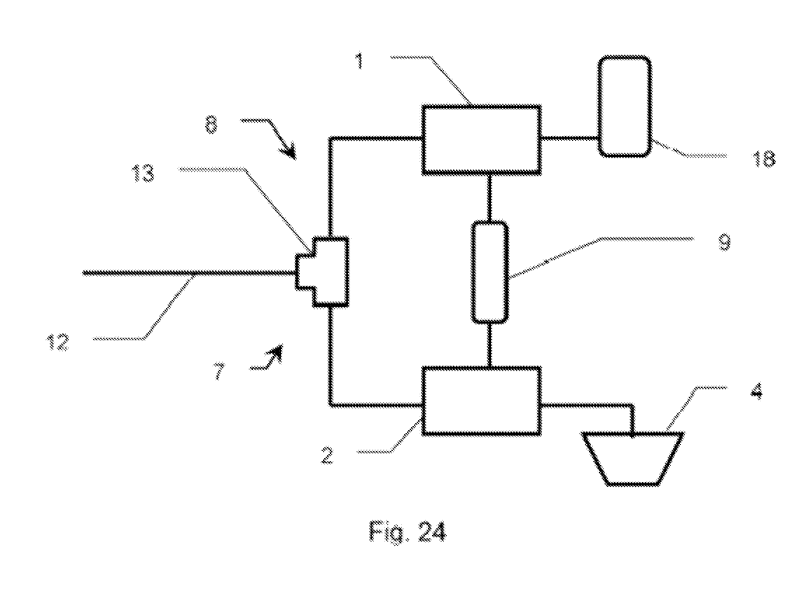

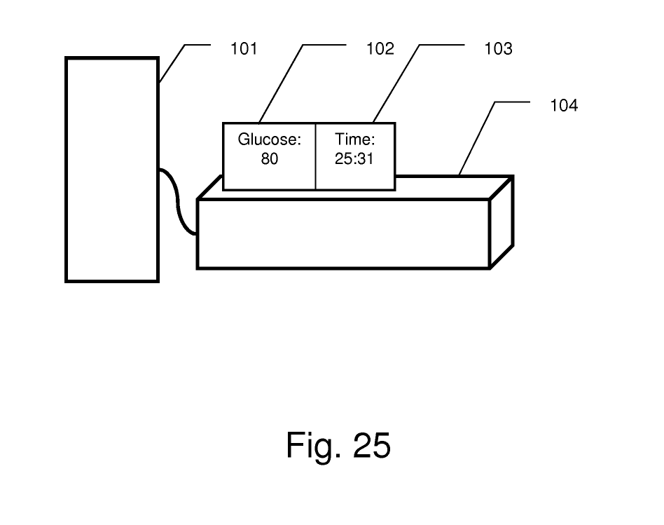

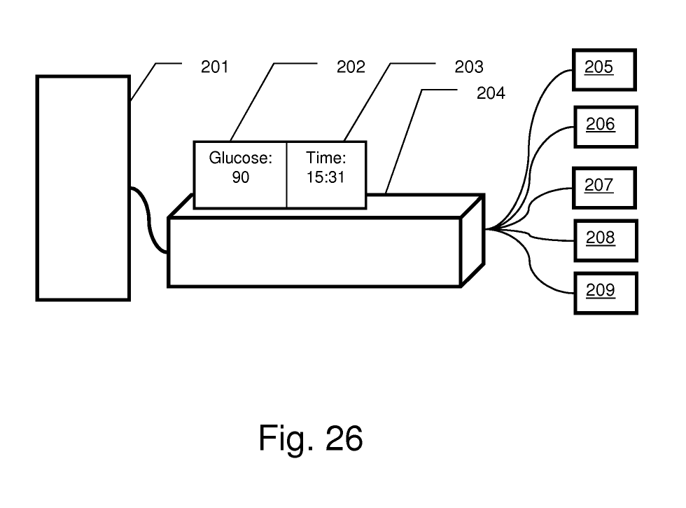

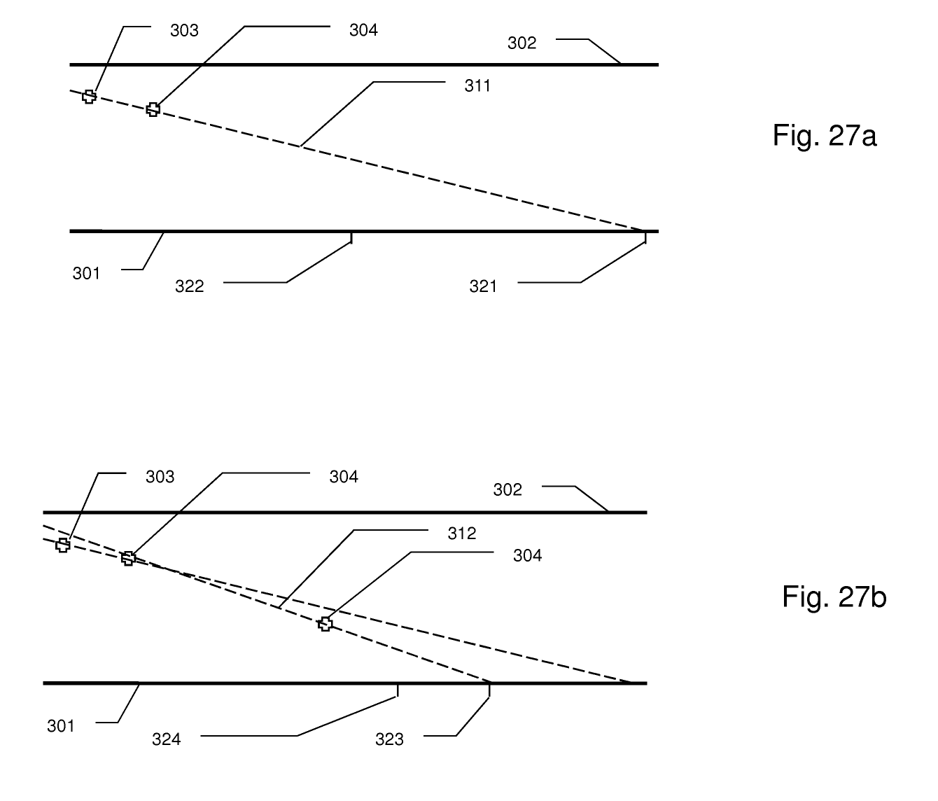

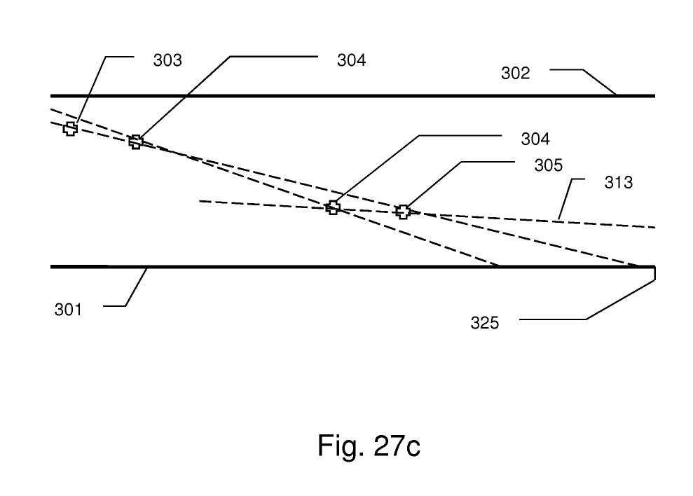

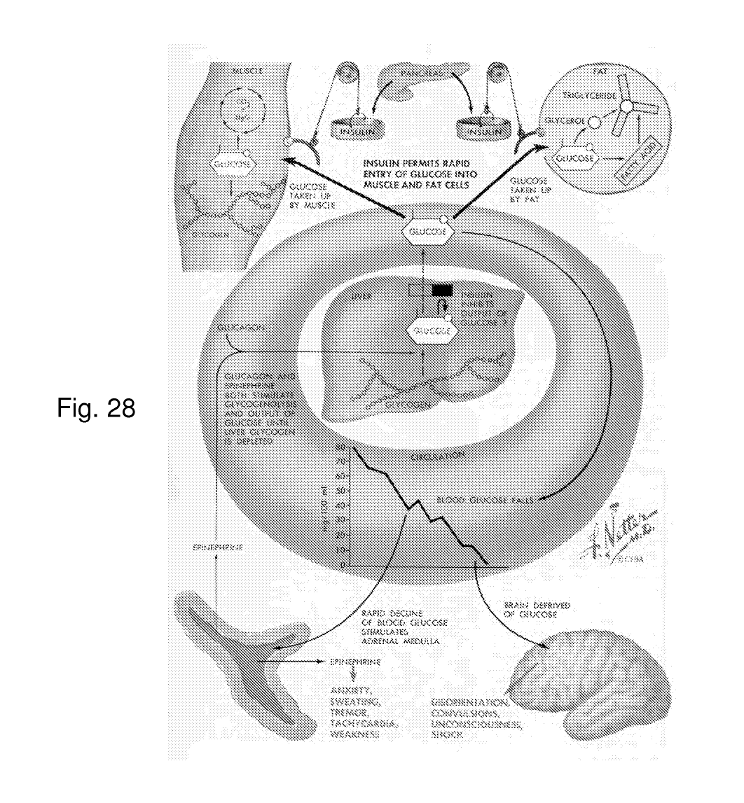

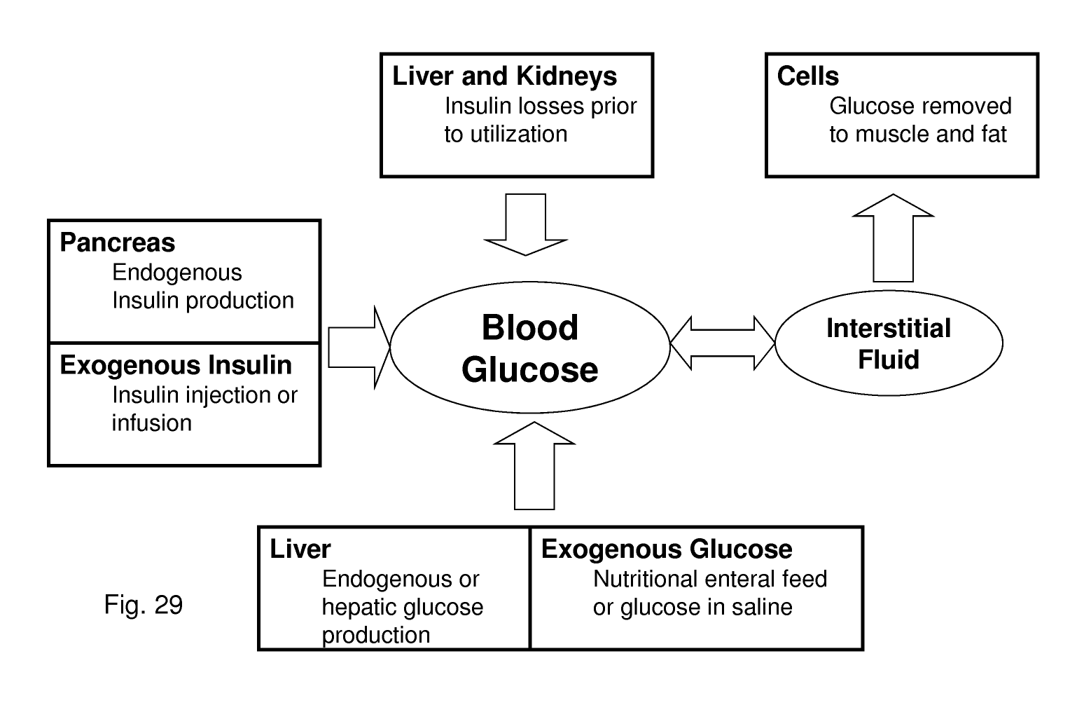

FIG. 24 is a schematic illustration of an example embodiment. The system comprises a catheter (or similar blood access device) (12) in fluid communication with the vascular system of a patient, and in fluid communication with a junction (13). A first side of the junction (13) connects with fluid transport apparatus (8) such as tubing (for reference purposes called the “left side” of the system). The left side of the system further comprises a source of maintenance fluid (18) and a connection to one side of a flow through glucose sensor system (9). A first fluid control system (1) controls fluid flow within the left side of the system. A second side of the junction (13) connects with fluid transport apparatus (7) such as tubing (for reference purposes called the “right side” of the system). The right side of the system further comprises a channel or receptacle for waste (4), and a connection to a second side of the flow through glucose sensor system (9). A second fluid control system (2) controls fluid flow within the left side of the system. In operation, the first and second fluid control systems are operated to draw blood from the patient to the junction (13), and then into either the left or right side of the system. The fluid control systems can then be operated to flow at least a portion of the blood to the glucose measurement system (9), where the glucose concentration of the blood (or other analyte property, if another analyte sensor is employed) can be determined. The fluid control systems can then be operated to flow the blood, including at least a portion of the blood measured by the glucose measurement system, into either the left or right side of the system and then back to the patient. As desired, the fluid control systems can be operated to flow maintenance fluid from the maintenance fluid source (18) through the glucose measurement system (9) to the waste channel (4) to facilitate cleaning or calibration of the system. The fluid control systems can also be operated to flow maintenance fluid through the left and right sides to facilitate cleaning of the tubing or other fluid transport mechanisms. The fluid control systems can also be operated to flow maintenance fluid into the patient, for example at a low rate to maintain open access to the circulatory system of the patient.

Push Pull with Additional Path.

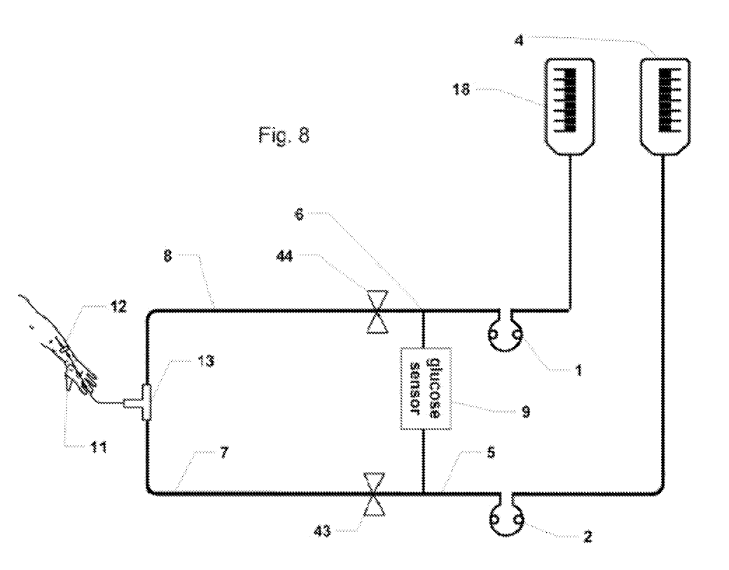

FIG. 8 is a schematic illustration of an example embodiment. The system comprises a catheter (or similar blood access device) (12) in fluid communication with the vascular system of a patient. A tubing extension (11) (if required) extends from the catheter (12) to a junction (13). A first side of the junction (13) connects with fluid transport apparatus (8) such as tubing (for reference purposes called the “left side” of the blood loop); a second side of the junction (13) connects with fluid transport apparatus (7) such as tubing (for reference purposes called the “right side” of the blood loop). A pinch valve (44) controls flow between the left side (8) of the blood loop and an intermediate fluid section (6). A pump (1) mounts between the intermediate fluid section (6) and a source of saline such as a bag (18). A pinch valve (43) controls flow between the right side (7) of the blood loop and an intermediate fluid section (5). A pump (2) mounts between the intermediate fluid section (5) and a waste channel such as a bag (4). A glucose sensor (9) mounts between the two intermediate fluid sections (6, 5). Elements and their operation are further described below.

Blood Sample and Measurement Process.

1. Blood is removed from the patient via the blood pump (1) while pinch valve (44) is open and pinch valve (43) is closed.
2. At the end of the draw blood is diverted into the tubing path containing the measurement cell (9) by activation of pump (2) with the concurrent closure of pinch valve (43).
3. A volume of blood appropriate for the measurement can be pulled into (or past as needed) glucose sensor (9) and into tubing (5). The rate at which the blood is pulled into tubing (5) can be performed such that the draw time is minimized.
4. At this juncture the re-infusion process can be initiated. Pump (2) initiates a re-infusion of the blood at a rate consistent with the measurement of the blood sample. In general terms this rate is slow as the blood simply needs to flow at a rate that results in a substantially constant sensor sampling. Concurrently, pump (1) initiates a re-infusion of the blood.
5. As has been described previously, the amount of saline infused into the patient can be controlled via the use of the flush line (7).
6. The system can then be completely cleaned via the use of the two pumps (1, 2) as well as pinch valves (43, 44).

Characteristics of Push Pull with Additional Path. This example embodiment can perform measurement and infusion concurrently. In the previously-described push-pull system the withdrawal, measurement, and re-infusion generally occur in a sequential manner. In the system of FIG. 8 the measurement process can be done in parallel with the infusion. The reduction in overall cycle time can be approximately 30%.

In addition to the reduction in total cycle time, the system has the ability to provide independent cleaning paths. By closing or opening the pinch valves in combination with the two pumps, the system can create bi-directional flows and clean the sensor measurement cell independent of the rest of the circuit. Such independent cleaning paths are especially useful when managing either complete or partial occlusions.

The push pull with additional path system as illustrated in FIG. 8 is an example embodiment of one possible configuration. The pump mechanism can be moved to the portion of tubing between the junction leading to the glucose sensor and the patient. Many other pump and flow control devices can be used to create the operational objectives defined above. Additionally, the system can be realized with only one pump.

The push pull with additional path system as illustrated in FIG. 8 also has the advantage of being able to deliver a sample to the glucose sensor without it being preceded by saline. As the blood is withdrawn up the left side of the circuit the saline/blood transition area can be moved beyond the location where blood sensor (9) connects with tubing (6). At this point the blood that is moved into sensor (9) could have a very small or no leading saline boundary. The lack of such a leading saline boundary can facilitate the use of the system with existing blood glucose meters. Typically, these meters make the assumption that all fluid in contact with the disposal strip is blood, not a mixture of blood and saline.

Sample Isolation at the Arm with Subsequent Discard.

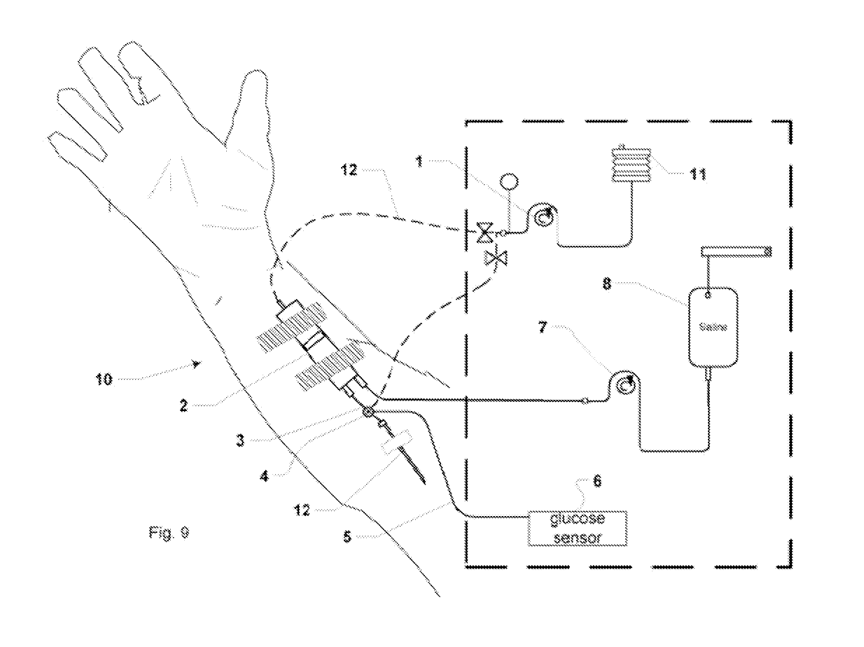

FIG. 9 is a schematic illustration of an example embodiment that allows a blood sample for measurement to be isolated at a point near the patient and then transported to the instrument for measurement. The system shown does not require electronic systems attached to the patient. A hydraulically actuated syringe (10) is provided, with a pump (1) and saline reservoir (11) and tubing (12) provided to control actuation of the syringe (10). A catheter (12) is in fluid communication with the vascular system of a patient. The syringe (10) can mount such that it draws blood from the patient via the catheter (12). A valve (4) controls flow between the catheter and a transport mechanism (5) in fluid communication with a glucose measurement device (6). The syringe (10) is also in fluid communication with a pump (7) and an associated fluid reservoir such as a bag of saline (8). The system can be described as one that is remotely activated by hydraulic action. Elements of the system and their operation are further described below.

Blood Sample and Measurement Process.

1. The blood is withdrawn from the patient using hydraulically activated syringe (1). The syringe is controlled by pump (1).
2. The removal of some blood into syringe (2) creates an undiluted and clean blood sample in catheter (3).
3. Valve (4) is activated into an open position such that a small sample of blood is diverted into tubing pathway (5). The blood is subsequently transported to measurement cell (6) for measurement. The blood transport into glucose sensor (6) can be via air, saline or other appropriate substances.
4. The blood in syringe (2) is re-infused by activation of pump (1). Following re-infusion of the blood the system can be cleaned with saline by activation of pump (7).
5. The blood located in the measurement cell is measured and subsequently discarded to waste (not shown).

The system can be operated in several different modes. The delivery of a small sample to the measurement site can be easily accomplished by the use of air gaps to isolate the sample from other fluids that can otherwise tend to dilute the sample. In this measurement method the volume of the sample does not need to be tightly controlled and the measurement system measures the glucose (mg/dl) in the sensor cell.

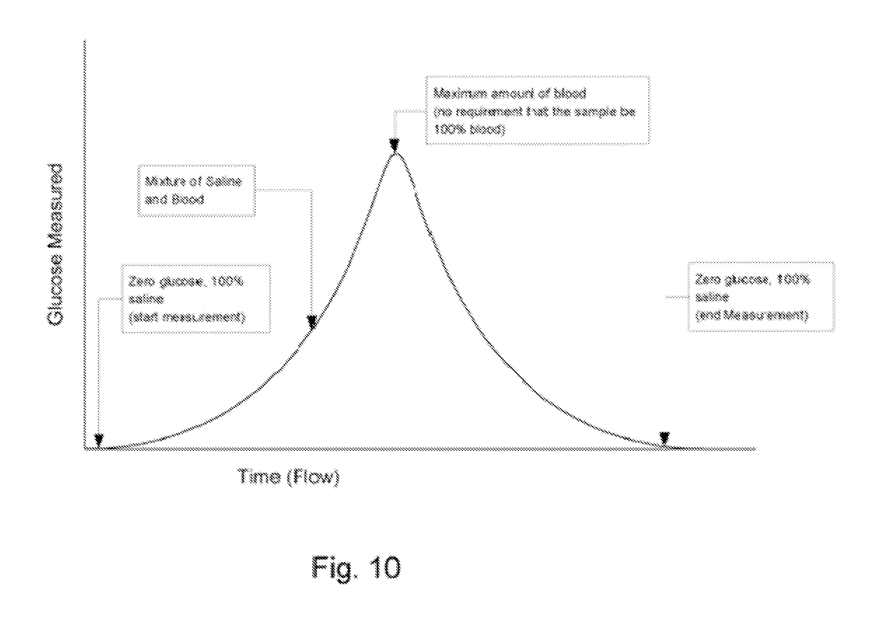

An alternative approach involves either reproducible control of the volume of blood or determination of the volume of blood and integration of the total amount of glucose measured, as illustrated in FIG. 10. The blood sample can then undergo significant mixing with the transport fluids since there is no requirement that an undiluted sample be delivered to the sensor cell. The system can effectively determine the total amount of glucose measured. The total amount of glucose could be determined by a simple integration for the area under the curve. With both the total amount of glucose known and the volume of blood processed, an accurate determination of the blood glucose can be made.

Characteristics of Sample Isolation at the Arm with Subsequent Discard. The total amount of blood removed during the sampling process is minimized by this system. Additionally the amount of saline infused is also minimized.

The pressure needed to withdrawal the blood sample can be monitored for partial or complete occlusion. If such a situation is observed the flush pump can be used to either clean the catheter or to clean the circuit over to the measurement cell. In addition the activation of the flush pump in conjunction with the hydraulic syringe can be used to create rapid flows, turbulent flows and to isolate particular components of the circuit for cleaning.

Sample Isolation System.

FIG. 15 is a schematic illustration of a blood access system according to the present invention. The system comprises a catheter (or similar blood access device) (12) in fluid communication with the vascular system of a patient. A tubing extension (51) (if required) extends from the catheter (12) to a junction (13). A first side of the junction (13) connects with fluid transport apparatus (52) such as tubing; a second side of the junction (13) connects with fluid transport apparatus (53) such as tubing. A sample system (38) is in fluid communication with fluid transport apparatus (52). A one-way fluid control device (32) (e.g., a check valve) receives connects so as to receive fluid from fluid transport apparatus (53) and deliver to a junction (33). A first side of the junction (33) is in fluid communication with a drive system (39); a second side of the junction is in fluid communication with fluid transport apparatus (54) such as tubing. A sensor (49) is connected so as to receive fluid from fluid transport apparatus (54). A waste container or channel (45) is connected so as to receive fluid from the sensor (49). (53), (32) and (33) can be separate components or be integrated as a single component to minimize dead space volume between the functions of each component.

In operation, the sample system (38) draws blood from the patient into fluid transport apparatus (51) and (52). After a sufficient volume of blood has been drawn into (51) and (A2), the sample system (38) pushed blood from (52) through one-way device (32) to junction (33). Drive system (39) pushes a “plug” into junction (33), where a plug can comprise a quantity of a substance relatively immiscible with blood and suitable for transport through tubing or other components in transport apparatus (54) and suitable for transport through sensor (49) without contamination of the sensor (49). Examples of suitable plug materials include air, inert gases, polyethylene glycol (PEG), or other similar materials. An alternative type of plug can comprise fixing or clotting the blood at the leading and trailing edges. Specifically, glutaraldehyde is a substance that causes the hemoglobin in the red blood cell to become gelatinous. The net result is a gelatinous plug that can be used effectively to separate the blood used for measurement from the surrounding fluid. After the initial plug is pushed into junction (33), sample system (38) pushes additional fluid into (52), forcing blood from (53) past junction (33) forcing the initial plug in front of the blood into transport apparatus (54). Sample system can push blood into (52), or can push another suitable fluid such as saline into (52), or can reduce the volume of (52), or any other method that moves the blood in (B) into junction (33) and transport apparatus (54). Once a sufficient quantity of blood is present in transport apparatus (54), drive system (39) can push a second or trailing plug into junction (33). Transport system (39) can then push the plug-blood-plug packet through transport apparatus (54) so that the blood can be measured by sensor (49). The blood can be immediately pushed to waste (45), or pushed to waste by the transport of a subsequent sample. Since the blood in transport apparatus (54) is surrounded by relatively immiscible plugs, and since the drive system (39) can push the plug-blood-plug packet using techniques optimized for transport (e.g., pressurized air or other gas, or mechanical compression of transport apparatus (54)), the blood can be transported more quickly, and over greater distances, than if the patient's blood or saline were used as the motive medium.

Sample Isolation Though Use of Air Gaps.

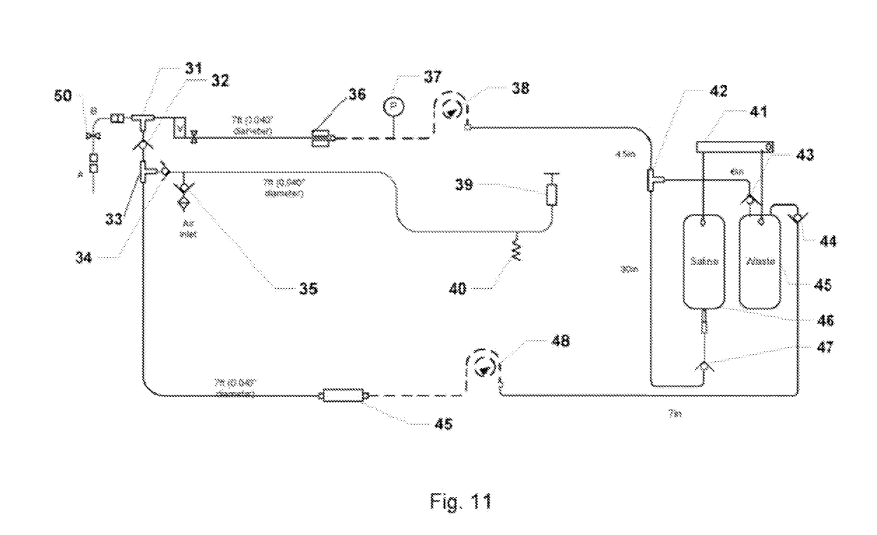

FIG. 11 is a schematic illustration of an example embodiment that allows a blood sample for measurement to be isolated at a point near the patient and then transported to the instrument for measurement through the use of leading and the following air gaps. The system is able to effectively introduce air gaps through a series of one-way valves while concurrently preventing air from being infused into the patient. The system is adapted to connect with the circulation system of a patient through blood access device (50). A recirculating junction (31) has a first port in fluid communication with a patient, with a second port in fluid communication with a one-way (or check) valve (32). The valve (32) allows flow only away from the recirculating junction (31) toward a port of a second junction (33). A second port of the second junction (33) is in fluid communication with a one-way valve (34), which allows flow only towards the second junction (33). The one-way valve (34) is in fluid communication with another one-way valve (35) and with an air pump (39). The communication between the air pump (39) and the one-way valve (35) can be protected with a pressure relief valve (40). The one-way valve (35) accepts air from an external source. A third port of the second junction (33) is in fluid communication with a glucose sensor (49), which in turn is in fluid communication with a pump (48), and then to a one-way valve (44) that allows flow from the pump to a waste channel such as a waste bag (45). Another port of recirculating junction (31) is in fluid communication with a pump (38). The path from the recirculating junction (31) to the pump (38) can also interface with a pressure sensor (37) and an air detector (36). The pump (38) is in fluid communication with a junction (42). Another port of junction (42) is in fluid communication with a one-way valve (43) that allows fluid flow from the pump (38) to a waste channel such as waste bag (45). Another port of junction (42) is in fluid communication with a one-way valve (47) that allows fluid flow from a saline source such as saline bag (46) to the pump (38). Manual pinch clamps and access ports can be provided at various locations to allow disconnection and access, e.g., to allow disconnection from the patient.

Blood Sample and Measurement Process.

1. Blood is withdrawn from the patient utilizing the blood pump until a clean or uncontaminated sample has been pulled pass the recirculation junction.
2. Additional blood is withdrawn from the patient by activation of the pump labeled recirculation pump. Blood is pulled to the air junction.
3. An air plug is created by pulling back on the air pump (39). The one-way valve at the air intake allows air into the tubing set for the formation of a small air gap.
4. The air gap is infused through valve (34) to create a leading air gap in junction (33) which is located at the leading edge of the uncontaminated blood sample.
5. The recirculation pump (48) then withdraws blood from the patient until an appropriate volume of uncontaminated blood has been procured.
6. The air pump (39) is again operated in the mode to create a second air gap that will be used as a trailing air segment.
7. The second air plug is infused through valve (34) to create a following air gap.
8. The blood residing in the line leading to the blood pump is infused into the patient.
9. The blood sample with leading and trailing air gaps is now transported over to the glucose sensor (45). Once in contact with the glucose sensor, an accurate glucose measurement can be made.
10. Following completion of the measurement sample is discarded to waste (45).
11. The circuit is now completely filled with saline and additional cleaning the circuit can be performed.

Characteristics of sample isolation by leading and trailing air gaps. There are a number of advantages associated with this isolation system, specifically the total amount of blood removed from the patient can be significantly less due to the fact that the blood sample is isolated at a point very close to the patient. The isolation of the blood sample and transportation of that small amount of blood to the measurement has advantages relative to a system that transports a large amount of blood to the measurement site. The fact that a small amount of total blood is withdrawn results in decreased overall measurement time or dwell time. The decreased amount of blood removed enables the system to operate at lower overall withdrawal rates and with lower pressures. Additionally, the isolation the blood sample has the advantage at the isolated sample can be measured for a prolonged period of time, can be altered in ways that are incompatible with reinfusion into the patient. Due to pressure monitoring on the blood withdrawal and the possible inclusion of a second pressure sensor on the recirculation side of the circuit (not shown), the circuit design has extremely good occlusion management capabilities. The isolation of the blood sample and inability to re-infuse the sample due to the use of one-way valves, can create the opportunity to use non-sterile measurement methodologies.

Hematocrit Influence on Withdrawal Pressures.

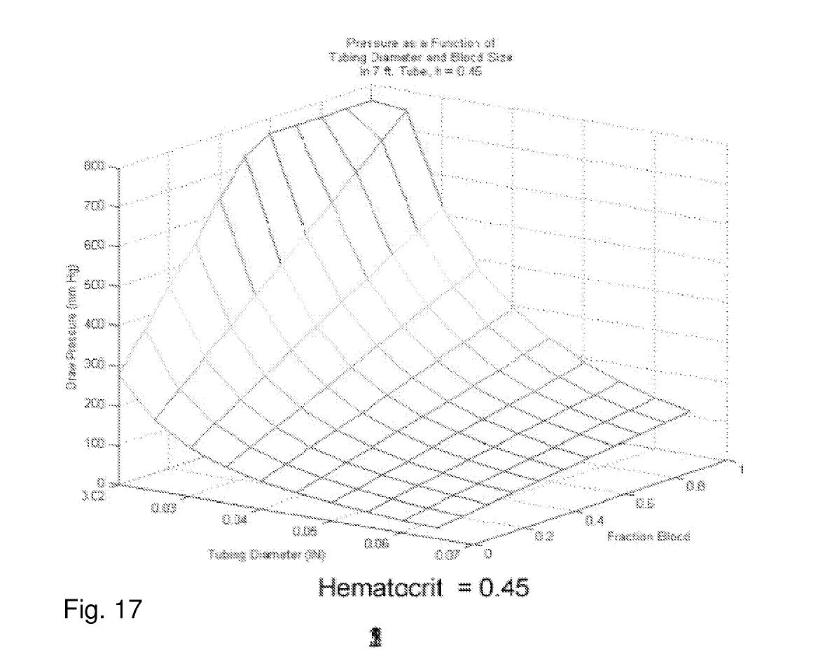

FIG. 16 is an illustration of a relationship between withdrawal pressure, tubing diameter and blood fraction at a fixed hematocrit. As used here blood fraction is the percent volume occupied by blood assuming a 7 foot length of tubing. FIG. 16 depicts this relationship assuming a hematocrit of 25%. FIG. 17 is the same information but assuming a hematocrit of 45%. Examination of these graphs shows significant pressure increases associated with increasing hematocrit, decreasing tube size and increasing blood fraction. In general terms, it can be desirable to use smaller tubing as the amount of blood required is less and the length of the blood saline junction is less. These generally desirable attributes are offset by the fact that smaller tubing requires higher pump pressures. Comparison of FIG. 16 with FIG. 17 also shows that there is strong sensitivity to the fraction of blood and the tubing diameter. With a glucose measurement methodology that requires only a small sample of blood, it can be desirable to use a smaller blood fraction which results in lower overall circuit pressures.

Hematocrit Influence on Blood Saline Junction.

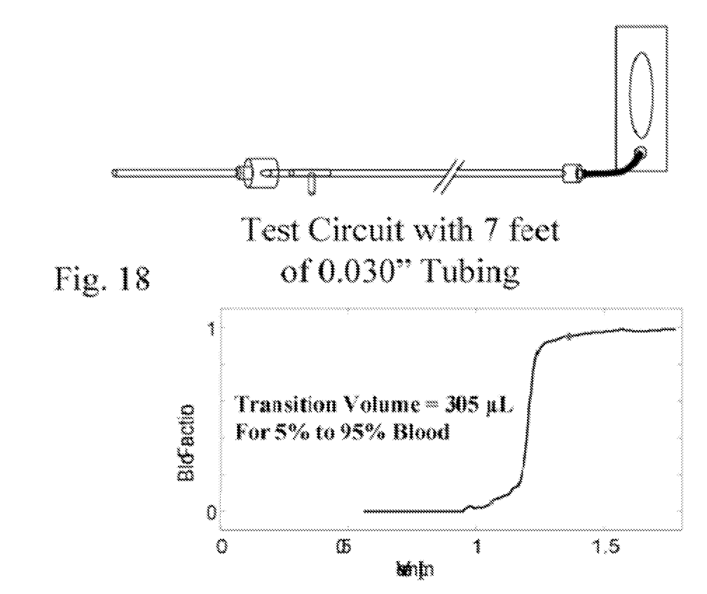

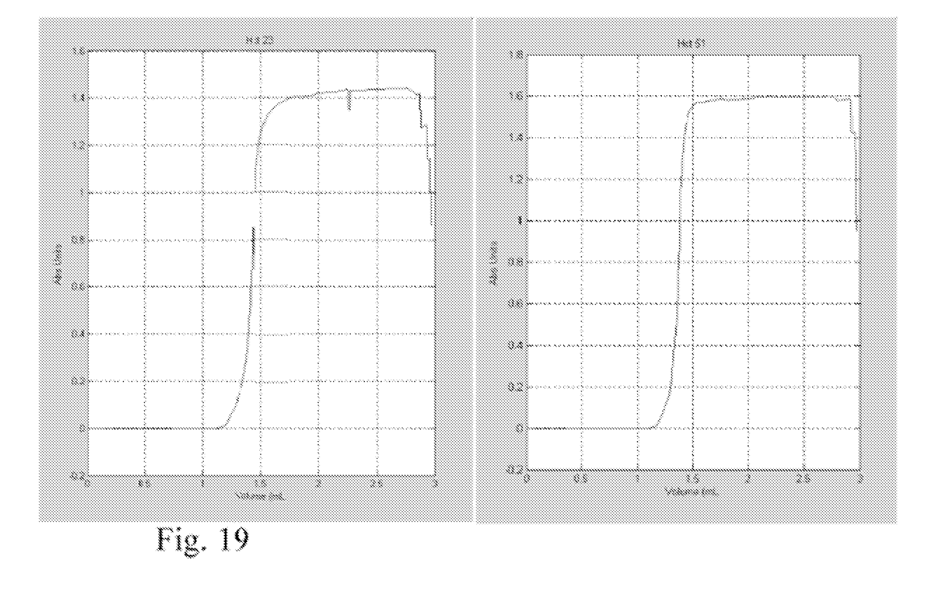

FIG. 18 shows a test system used to determine the amount of blood saline mixing that occurs during transport of the blood through the tubing, including the luer fittings, junctions, and the subsequent filling of the optical cuvette. In testing, the system is initially filled with saline and blood is withdrawn into the tubing set. An optical measurement is performed throughout the withdrawal cycle. As the transition from saline to blood occurs the optical density indicated by the optical measurement of the sample changes. A transition volume representing the volume needed to progress from 5% absorbance to 95% absorbance can be calculated from the recorded data. FIG. 19 shows the results from the above test apparatus for two hematocrit levels, 23% and 51%. As can be seen from FIG. 19, the transition volume is greater for the lower hematocrit blood. The dependence of the transition volume on hematocrit level can be used as an operating parameter for improved blood circuit operation.

Use of Blood/Saline Transition for Measurement Predictions

As shown in FIG. 19, the transition from saline to blood is a systematic and a repeatable transition. By using the fact that the transition is repeatable for a given hematocrit, the measurement process can be initiated at the start of this transition zone. In the case of 23% hematocrit, the measurement process could be initiated falling withdrawal of 1.5 ml. The measurement process could then account for the fact that there is a known dilution profile as a function of withdrawal amount. For, example the system can make measurements at discrete intervals and project to the correct undiluted glucose concentration.

Modified Operation of Push Pull System with Two Peristaltic Pumps.

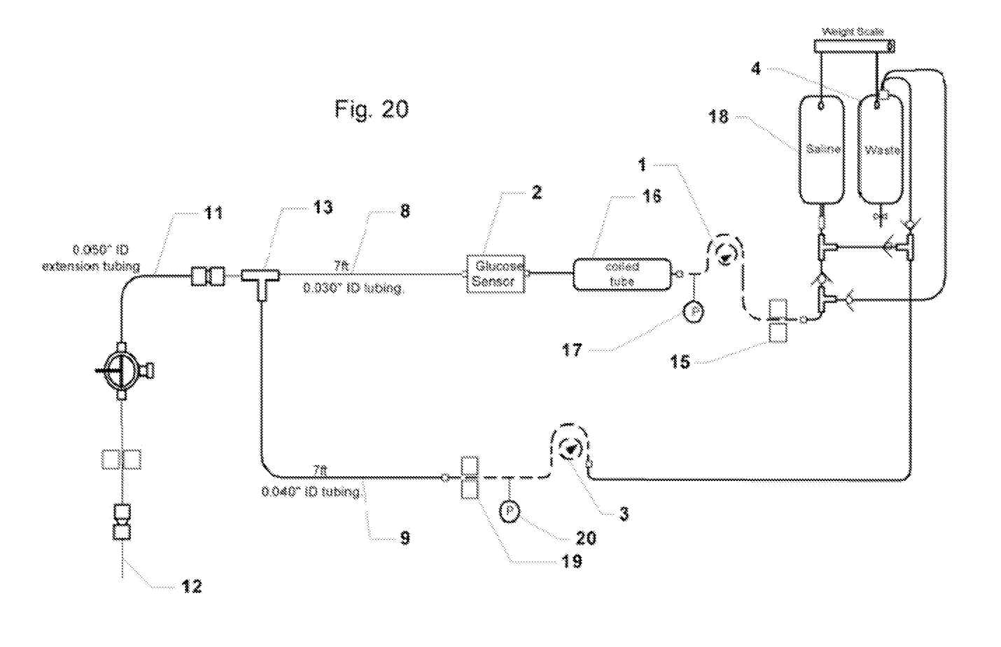

FIG. 20 is a schematic illustration of a blood access system based upon a push-pull mechanism with a second circuit provided to prevent fluid overload in the patient. The circuit is similar to that depicted in FIG. 5 but is operated in manner that optimizes several operational parameters. The system comprises a catheter (or similar blood access device) (12) in fluid communication with the vascular system of a patient. A tubing extension (11) (if required) extends from the catheter (12) to a junction (13). A first side of the junction (13) connects with fluid transport apparatus (8) such as tubing (for reference purposes called the “left side” of the blood loop); a second side of the junction (13) connects with fluid transport apparatus (9) such as tubing (for reference purposes called the “right side” of the blood loop). An air detector (15) that can serve as a leak detector, a pressure measurement device (17), and a glucose sensor (2) mounted on the left side of the blood loop. A tubing reservoir (16) mounts with the left side of the blood loop, and is in fluid communication with a blood pump (1). Blood pump (1) is in fluid communication with a reservoir (18) of fluid such as saline. A second air detector (19) that can serve as a leak detector mounts with the right side of the blood loop. A second blood pump (3) mounts with the right side of the blood loop, and is in fluid communication with a receptacle or channel for waste, depicted in the figure as a bag (4). A second pressure sensor (20) can mount with the right side of the blood loop. An additional element shown in FIG. 20 is the specific identification of an extension set. The extension set is a small length of tubing used between the standard catheter and the blood access circuit. This extension set adds additional dead volume and other junctions that can be problematic from cleaning perspective. Elements of the system and their operation are further described below.

Modified operations. As shown in the preceding plots, high hematocrit blood requires a large pressure gradient but the increased viscosity of the blood results in smaller transition volumes. Lower hematocrit blood is the opposite, requiring lower pressures and larger transition volumes. In simple terms, the device can be operated to withdraw only enough blood such that an undiluted sample can be tested by the glucose sensor. Due to the lower transition volumes associated with higher hematocrit blood the amount of blood drawn can be appreciably smaller than the volume needed with lower hematocrit blood. For operation on a human subject the following general criteria can be desirable:

1) Minimize the total amount of blood withdrawn, this lowers overall exposure of blood to non-human surfaces.
2) Minimize the maximum pressure needed for withdrawal, this reduces the power requirements and pump sizes needed to move the blood.
3) Utilize the smallest tubing diameter possible, this reduces the blood volume and reduces mixing at the blood/saline interface.
4) Clean out the tubing between the blood vessel and the junction as soon as possible, this can help reduce the likelihood of clotting at this location.

Blood Sample and Measurement Process—Subsequent Blood Pump.

The example circuit shown in FIG. 20 can be operated in the manner that balances the four potentially competing objectives set forth above. The system can achieve improved performance by taking advantage of the small amount of undiluted blood sample actually required for sensor operation. Notice that, while a blood sample must be transported through the left side, the left side does not need to be completely filled with blood. Saline (or another suitable fluid or material) can be used to push a blood sample to the sensor. An example sequence of steps are set forth below:

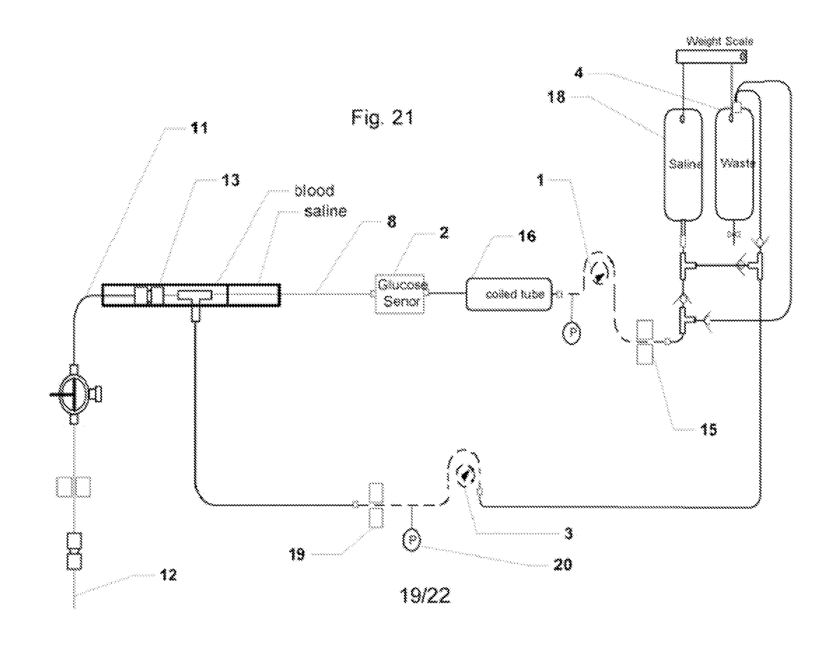

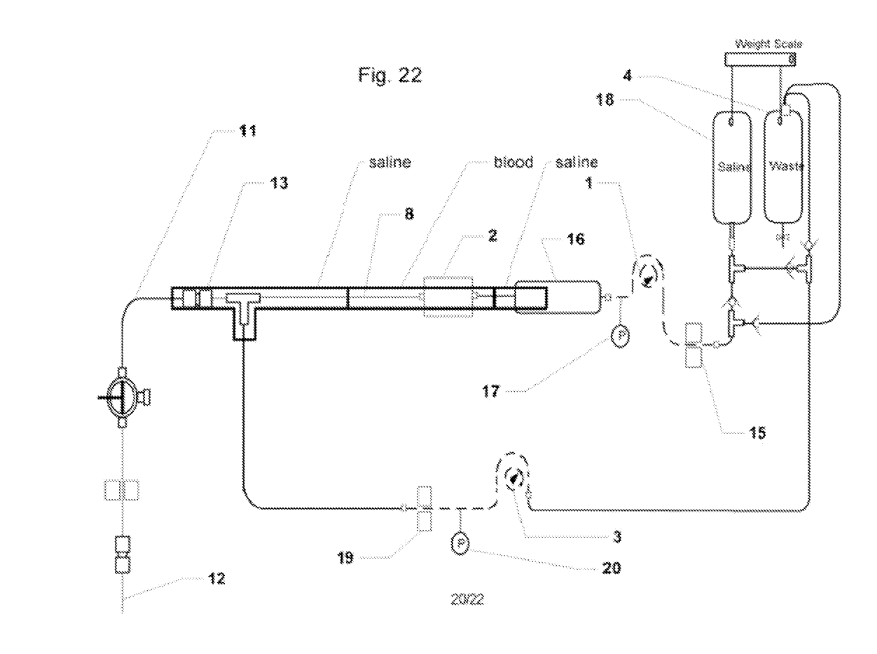

1. Pump (1) initiates a blood draw by drawing blood through junction (13).
2. The withdrawal continues until enough blood has been withdrawn past the junction of junction (13) and the right side (9) of the loop such than an undiluted and appropriately sized blood segment can be delivered to the glucose sensor, as illustrated schematically in FIG. 21. As mentioned above the amount of blood needed can be hematocrit dependent. Therefore, the amount of blood withdrawn past the junction (13) can be controlled based on measured hematocrit: smaller blood segments with higher hematocrit and larger blood segments with lower hematocrit. Following the withdrawal of an appropriate blood segment, the blood pump (1) continues to operate but the flush pump (3) is also turned on, as illustrated schematically in FIG. 22. The flush pump (3) can be operated at a rate equivalent to or greater than the blood pump (1). If operated at a rate greater then the blood pump (1), the flow rate imbalance forces saline (or other suitable fluid or material) into the right side (8), transporting the blood sample segment to the sensor, and also back into the extension tubing (11), cleaning the junction (13) and the extension tubing (11). As an example, the flush pump can initially be actuated at very high rate to rapidly clean the tubing connected to the patient and then decreased to primarily facilitate transport of the blood segment to the sensor measurement site.
3. As blood passes through the sensor measurement cell (2), it is stored in the tubing reservoir (16).
4. Sensor measurements can be made during this withdrawal period.
5. The blood can be moved back and forth over the sensor for an increased measurement performance (in some sensor embodiments) without the requirement for greater blood volumes.
6. Following completion of the blood measurement, the blood can be re-infused into the patient by reversing the direction of pump (1).
7. Sensor measurements can also be made during the re-infusion period.
8. As the mixed blood-saline passes through the junction (13), it becomes progressively more dilute.
9. Following re-infusion of the majority of the blood, flush pump (3) is turned on at a rate equal to or less than the rate of pump (1). If less than the rate of pump (1) then there is a small amount of saline re-infused into the patient. If operated at the same rate then there is substantially no net infusion into the patient. A small amount of residual blood mixed with the saline is taken to the waste bag (4).
10. This process results in a washing of the system with saline.
11. Additional system cleaning is possible through an agitation mode. In this mode the fluid is moved forward and back such that turbulence in the flow occurs. During this process both pumps can be used.
12. As a final step, the tubing between the junction and the patient, including the extension set (11), can be further cleaned by the infusion of saline by both the flush pump and the blood pump. The use of both pumps in combination increases the overall for flow through this tubing area and helps to create turbulent flow that aids in cleaning
13. Between blood samplings, the system can be placed in a keep vein open mode (KVO). In this mode a small amount of saline can be infused to keep the blood access point open.

Characteristics of Modified Push Pull Example Embodiment. The example embodiment of FIG. 20 has similar characteristics as those of the example embodiment depicted in FIG. 5, and has the additional advantage of using a smaller overall blood withdrawal amount. The example embodiment of FIG. 20 can also rapidly clean the tubing section between the junction and the patient, and operate with reduced overall pressures. Additionally, the circuit can be operated in a manner where the hematocrit of the patient's blood is used to optimize circuit performance by modifying the pump control. The use of hematocrit as a control variable can further reduce the amount of blood withdrawn and the maximum pressures required.

The use of the flush line in a bidirectional mode has several distinct advantages. During the final washing the rate of flow to the extension set at reasonable pressures can be greater than those obtained by using only the blood pump. In addition to improved washing, the flush line can be used to “park” a diluted leading segment. Specifically, the initial draw can be performed by the flush pump (3) such that the blood saline junction is moved into the right side of the circuit. After the blood/saline junction has passed and an undiluted sample has progressed to the T-junction, the left side of the circuit can be activated via the blood pump and a blood segment with a better defined saline/blood boundary transported to the measurement sensor. As leuer fittings between the extension set and the standard catheter are a major source of blood/saline mixing the ability to “park” this mixed segment can be advantageous.

Central Venous Operation. The ability to “park” the blood segment can be especially important when using the system on a central venous catheter (CVC). All figures in this disclosure show the use of the system on peripheral venous catheters, which typically have volumes of less than 500 μL. In the case of a central venous catheter, the volumes in the catheter can become quite large, around 1 ml, since that they can extend for up to 3 feet in the patient. This increased volume and length of tubing increases the amount of dead volume that must be withdrawn and increases the mixing at with the blood/saline boundary. Given the larger volumes preceding the undiluted blood segment, it can be desirable to “park” the blood from the CVC near the access location instead of transporting it through 7 feet of tubing to the measurement sensor. In operation, it has been found advantageous to use larger diameter tubing in the right side of the circuit and smaller diameter tubing in the left side. The use of larger diameter tubing enables a more rapid draw from the CVC line, while smaller tubing used to connect the glucose sensor has been found to minimize the total volume of blood removed from the patient.

Push Pull System with Two Peristaltic Pumps and Modified Sensor Location.

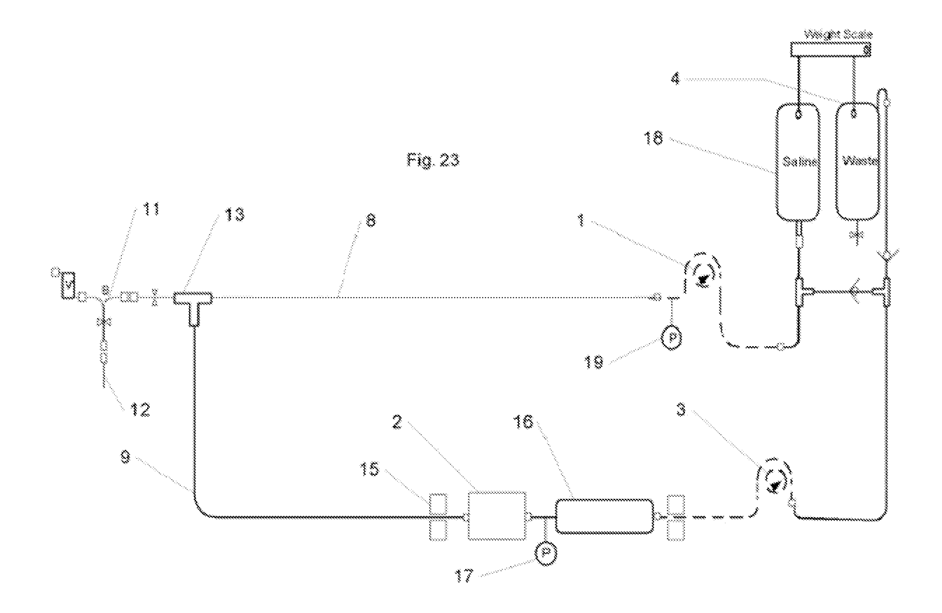

FIG. 23 is a schematic illustration of an example blood access system implemented based upon a pull-push mechanism. The example circuit is similar to that depicted in FIG. 20 but the glucose sensor is in a different location. The system comprises a catheter (or similar blood access device) (12) in fluid communication with the vascular system of a patient. A tubing extension (11) (if required) extends from the catheter (12) to a junction (13). A first side of the junction (13) connects with fluid transport apparatus (8) such as tubing (for reference purposes called the “left side” of the blood loop); a second side of the junction (13) connects with fluid transport apparatus (9) such as tubing (for reference purposes called the “right side” of the blood loop). An air detector (15) that can serve as a leak detector, a pressure measurement device (17), and a glucose sensor (2) mount on the right side of the blood loop. A tubing reservoir 16 mounts with the right side of the blood loop, and is in fluid communication with a blood pump (3), which is in fluid communication with a receptacle or channel for waste, depicted in the figure as a bag (4). A blood pump (1) mounts with the left side (8) of the system, and is in fluid communication with a reservoir (18) of fluid such as saline. A blood detector (19) serves as a leak detector mounts on the left side of the blood loop. An extension tubing set (11) can (and in many applications, will be required to) mount between the blood access device (12) and the junction (13). An extension set is generally a small length of tubing used to between a standard catheter and the blood access circuit. This extension set adds additional dead volume to the system, and adds other junctions that can complicate cleaning. Elements of the system and their operation are further described below.

Blood sample and measurement process—Subsequent Blood Sampling. In operation the circuit shown in FIG. 23 operates in a manner very similar to the “park” method described above. A blood sample can be drawn into the right side (9) and transported to the glucose measurement site, or a portion of the blood can be drawn and parked into the left side (8) first (as discussed more fully above). The following example operational sequence can be suitable; other sequences can also be used. For an initial sample, the tubing between the patient and the pump (1) can be filled with saline as a start condition. Subsequent measurements can be achieved with operation as follows:

1. Pump (1) initiates the blood draw by drawing blood up through junction (13).
2. The withdrawal continues as blood passes through the junction (13) until an undiluted segment of blood is present at the junction (13)
3. Pump (1) stops and pump (3) draws the undiluted segment toward the glucose sensor (2).
4. Following removal of an appropriate blood segment, pump (1) can be activated in a manner that cleans the tubing from the junction (13) to the patient and concurrently helps to push the undiluted segment to the glucose sensor (2).
5. Following completion of the glucose measurement, pump (3) can be activated such that majority of blood is re-infused into the patient.
6. At the point the majority of blood has been returned to the patient, pump (1) can be activated and the direction of pump (3) reversed such that the circuit is effectively cleaned. The small amount of residual blood mixed with the saline is taken to the waste bag (4).
7. Between blood samplings, the system can be placed in a keep vein open mode (KVO). In this mode a small amount of saline can be infused to keep the blood access point open.

Advantages of pressure measurement. The systems as shown throughout this disclosure can use two pressure measurement devices which may or may not be specifically identified in each figure. These devices can be utilized to identify occlusions in the circuit during withdrawal and infusion as well as the location of the occlusion. Additionally, the pressure sensors can be used to effectively estimate the hematocrit of the blood. The pressure transducer on the flush line effectively measures pressures close to the patient, while the pressure measurement device on the blood access line measures the pressure at the blood pump. The pressure gradient is a function of volume and hematocrit. The volume pumped is known, and thus the pressure gradient can be used to estimate the hematocrit of the blood being withdrawn.

FIG. 20 shows the use of two peristaltic pumps. In use peristaltic pumps create a pressure wave when the tubing is no longer compressed by the roller mechanism. The characteristics of this pressure wave when transmitted through blood or saline are defined. When the air or an air bubble is present in the system the overall compliance of the system is dramatically altered and the characteristics of this pressure wave are altered. By using one or both of the pressure measurement devices as a pressure wave characterization system, the device can detect the presence of air emboli in the circuit.

The particular sizes and equipment discussed above are cited merely to illustrate particular embodiments of the invention. It is contemplated that the use of the invention can involve components having different sizes and characteristics. It is intended that the scope of the invention be defined by the claims appended hereto.

Embodiments that automated testing intervals The present invention comprises methods and apparatuses that can provide measurement of glucose with variable intervals between measurements, allowing more efficient measurement with greater patient safety. A method according to the present invention can comprise measuring the value of an analyte such as glucose at a first time; determining a second time from a patient condition, an environmental condition, or a combination thereof; then measuring the value of the analyte at the second time. In one embodiment, the second-time can be determined from a comparison of the analyte value at the first time with a threshold. The interval between the first time and the second time can be related to the difference between the analyte value at the first time and the threshold; e.g., the closer to the threshold, the closer the two measurement times.

In another embodiment, the second time can be determined from a prediction of the value of the analyte. For example, the patient's conditions or environmental conditions, or both, can be used to predict a time at which the analyte level will reach a threshold, and the second time determined to be a predicted time, taking into consideration the physiological model; information related to infusion of nutrients, insulin, glucose, or other substances; a linear extrapolation of previous measurements; a nonlinear curve fitting of three or more previous measurements; and certain changes in patient or environmental conditions.

In some embodiments of the present invention, a second measurement can be made when a physiologic model of the patient, considering patient conditions, environmental conditions, or a combination, predicts a glucose level that has reached a threshold value. Both high and low thresholds can be established, with symmetric or asymmetric safety margins if desired.

Some embodiments of the present invention can use an optical measurement of analyte in whole blood. Some embodiments of the present invention can use measurements of analyte in portions of blood samples after removal of substantially all the red blood cells in the portion.

Such apparatuses can comprises a fluid access system, adapted to withdraw a sample of a bodily fluid such as blood from a patient; an analyte measurement system, adapted to measure the value of an analyte such as glucose concentration from the blood sample; and a controller, adapted to cause the fluidics system to withdraw a fluid sample for measurement at times determined by patient conditions, environmental conditions, or a combination thereof.

All variations can be used with automated measurement systems, allowing the system determine measurement times and automatically make measurements at the determined times, reducing operator interaction and operator error.

The determination of the next measurement time can rely on any of, or a combination of, factors such as the following.

Glucose level: as the patient begins to approach the blood glucose target limits the rate of sampling can increase such the time outside this target range is minimized.
Rate of glucose change: if the patient's blood glucose is changing rapidly the glucose may quickly exceed a target limit.
Insulin dosing history: the insulin dosing history will influence the expected rate of change and the level of blood glucose.
Caloric intake history: the caloric intake history will influence the expected change and magnitude of the blood glucose.
Medications: medications can influence the body's regulation of blood glucose and response to insulin.
Insulin sensitivity: insulin sensitivity is a general measure of the body's response to insulin dosing.
Target glucose range: the lower and tighter the range the more difficult it can be to maintain the patient's blood glucose level within this target range.
Duration of time in the intensive care unit: upon admission to the intensive care unit most patients will have a high glucose level with an initial therapy goal of getting the patient in the target range.
Model based parameters, estimated states and state predictions: The response of the glucose level to the factors noted above can be mathematically modeled to estimate model parameters and states. Such models include a) a model based on the interactions illustrated in the Netter diagram, (b) an AIDA model, (c) a Chase model, (d) a Bergman model, (e) a compartment model with differential equations, (f) an insulin pharmacokinetics and distribution model, (g) a glucose pharmacokinetics and distribution model, (h) a meal model, (i) a glucose/insulin pharmacodynamic model, and (j) an insulin secretion and kinetics model, or (k) a combination of two or more of the preceding.

The next sampling time can be determined as an interval from the previous sampling time.

### Example Embodiment

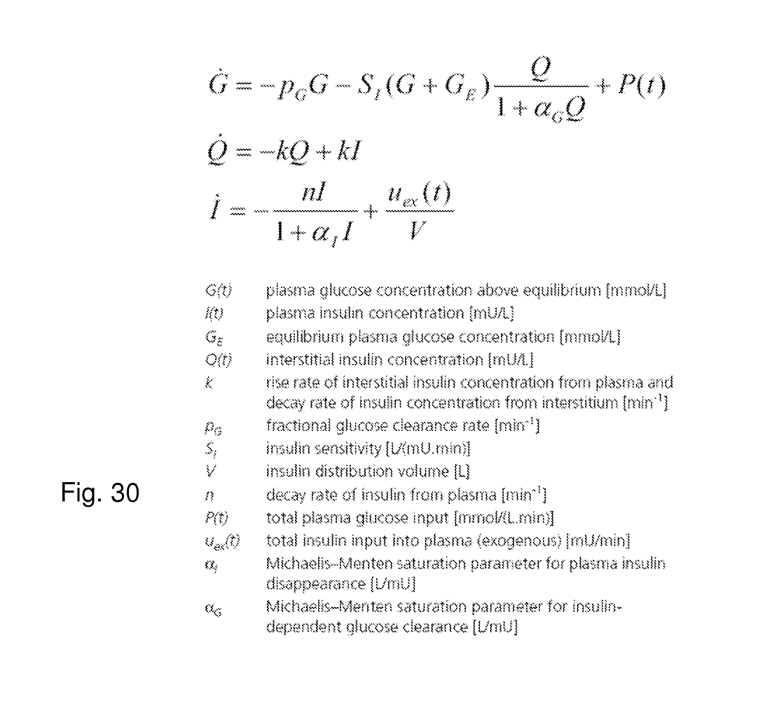

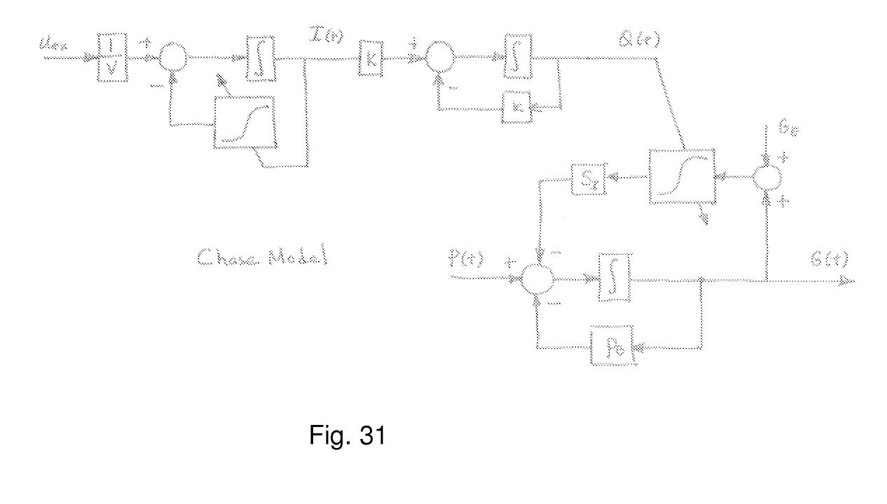

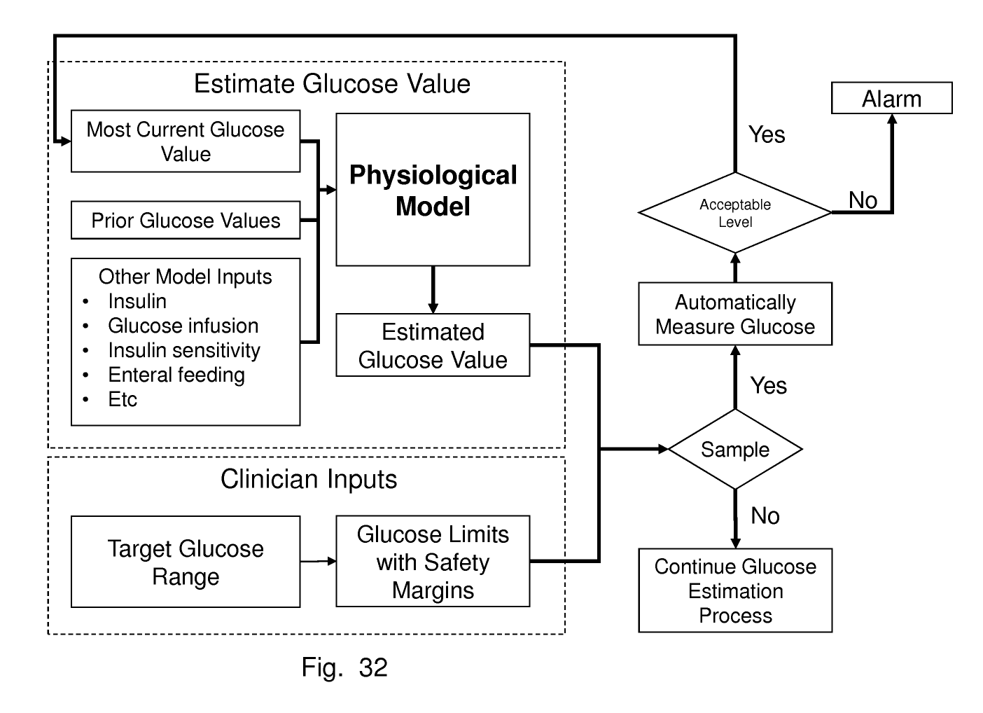

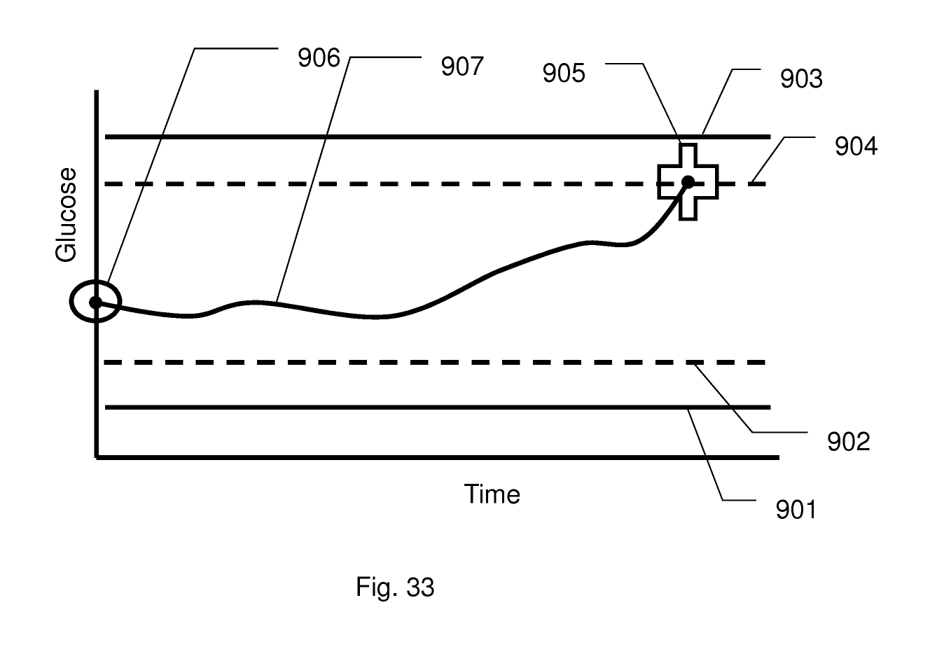

FIG. 30 presents the equations governing the Chase et al. model as well as the input parameters. Chase et al. use a model loosely based on Bergman's minimal model with additional non-linear terms and a grouped term for insulin sensitivity. The model effectively incorporates the effect of previously infused insulin with an accounting for the effective life of insulin in the system. The patient's endogenous glucose clearance and insulin sensitivity are represented in the model. The model also used Michaelis-Menton functions to model saturation kinetics associated with insulin disappearance and insulin-dependent glucose clearance. The P(t) term can also be based upon glucose appearance from enteral nutrition via feeding tubes or by direct glucose administration. FIG. 31 is a state diagram of the Chase model showing the key inputs and relationships of the model.

### Example Embodiment

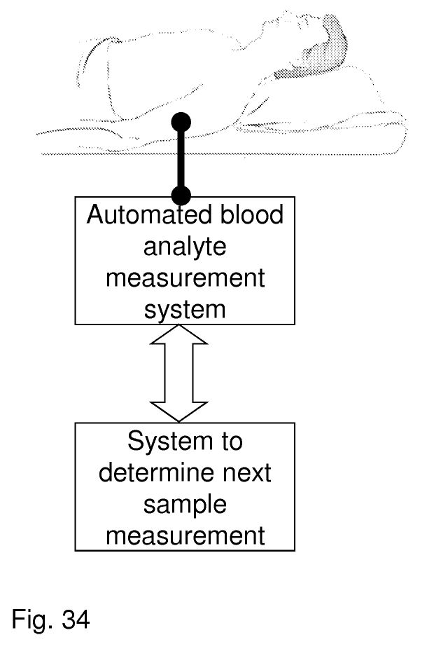

FIG. 34 shows a generic embodiment of the system. The operational implementation of the system requires interaction with the patient for the procurement of a blood measurement. This measurement value is then communicated via a variety of possible means to the system that determines the time for the next measurement.

### Example Embodiment

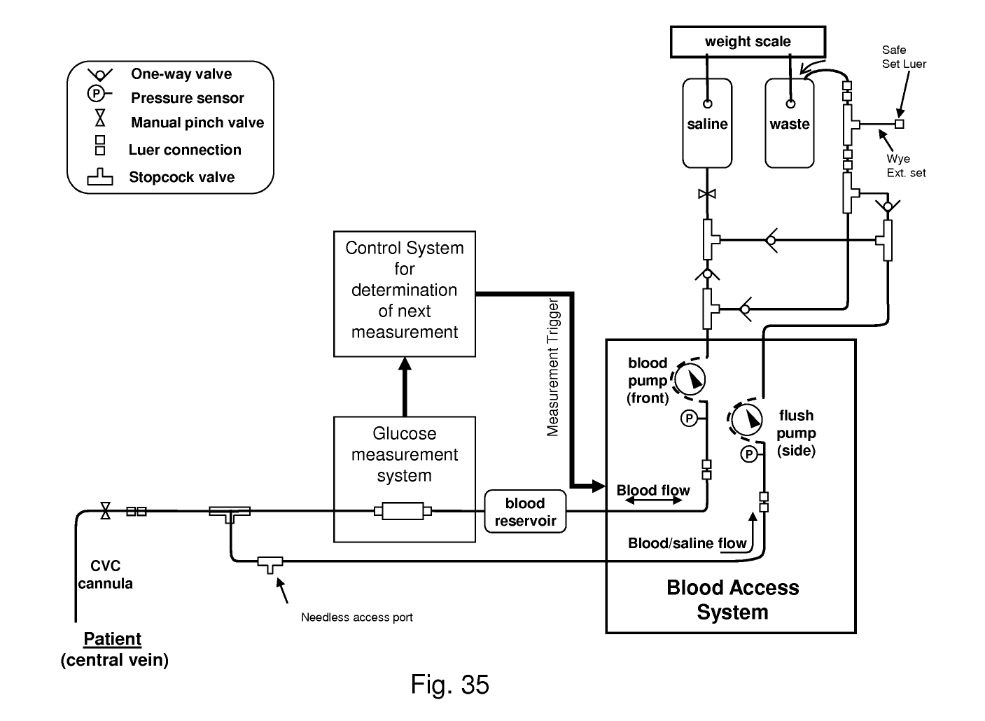

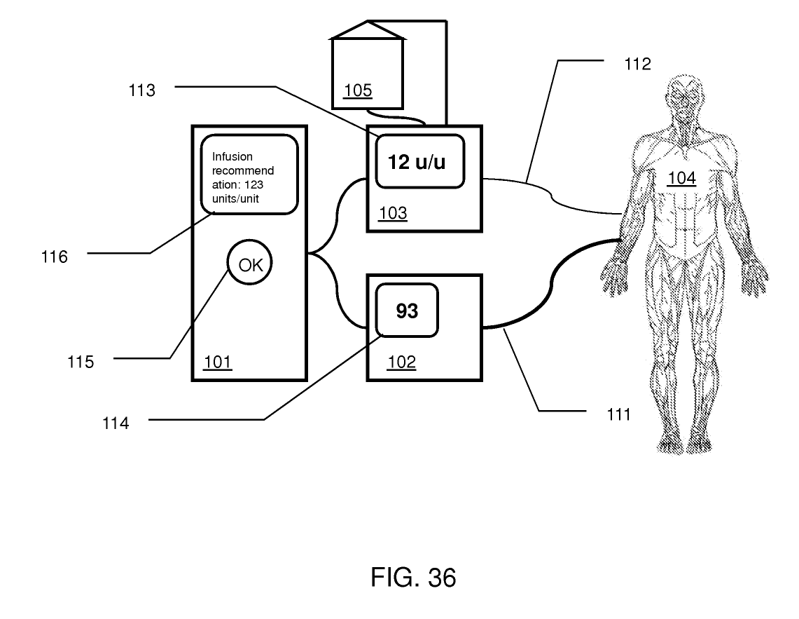

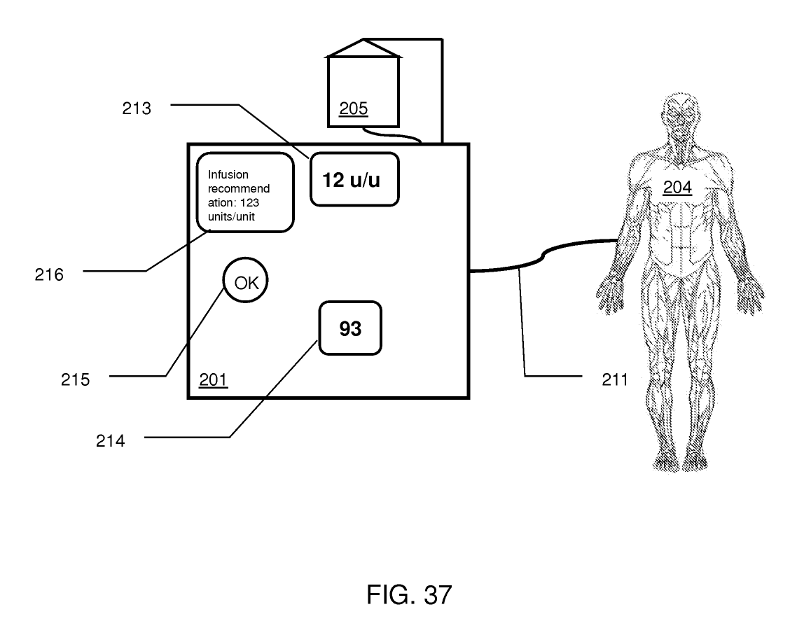

FIG. 35 shows an example system in operation on an automated blood removal system. In operation the module labeled “control system for determination of next measurement” initiates the procurement of a glucose measurement. The blood access system initiates blood sample procurement. The blood is presented to the glucose measurement system and a glucose value obtained. The glucose value or related information is communicated to the control system and the time for the next sample determined. The exact methods used for sample procurement can include a manual sample, noninvasive sample, indwelling measurements, or invasive measurement methods. The glucose measurement methods can include existing enzymatic or electrochemical techniques as well as optical measurement methods.

The particular sizes and equipment discussed above are cited merely to illustrate particular embodiments of the invention. It is contemplated that the use of the invention can involve components having different sizes and characteristics.

Embodiments of Semi-Automated Glucose Management System An embodiment of the present invention is a semi-automated glucose management system, comprising a glucose measurement system, adapted to measure the glucose level in a patient's blood, or an indicator thereof; an infusion recommendation system, adapted to recommend infusion parameters based on information comprising the measured blood glucose level; an infusion control system, adapted to infuse glucose or insulin into the patient, and means for a clinician to authorize an infusion of glucose or insulin into the patent by the infusion control system based on a recommendation of infusion parameters by the infusion recommendation system. The glucose measurement system, infusion recommendation system, and infusion control system can be integrated in a single unit. The glucose management system can further comprise means for automated record keeping for blood glucose level measurements, glucose and insulin infusion parameters, identity of the authorizing clinician, and the timing of blood glucose level measurements and infusion parameters.

The present invention can comprise apparatuses useful for automatically determining analyte values such as blood glucose levels. Such apparatuses can comprises a fluid access system, adapted to withdraw a sample of a bodily fluid such as blood from a patient; an analyte measurement system, adapted to measure the value of an analyte such as glucose level from the blood sample; and a controller, adapted to cause the fluidics system to withdraw a fluid sample for measurement at times determined by patient conditions, environmental conditions, or a combination thereof.

The information of the infusion recommendation system can further comprise previous values of the patient's blood glucose level, the patient's previous response to previous glucose or insulin infusion, or the patient's glucose treatment characteristics.

The infusion recommendation system can further be used as a glucose measurement recommendation system. It can comprise an imbedded algorithm to recommend the infusion parameters. The clinician can vary the infusion of glucose or insulin from the recommendation of the infusion recommendation system only if a certain clinician authorization level is provided.

The glucose management system can also provide for automated record keeping. For example, an electronic or paper log can be created, with information such as glucose measurements, infusion parameters, infusion recommendations, identity of the authorizing clinician, and times of various events. The authorization system comprises means for the clinician to communicate remotely with the infusion recommendation system or the infusion control system.

The infusion control system can comprise an IV infusion pump.

Embodiments to manage cross-contamination in blood samples drawn from a multi-lumen catheter A computational fluid dynamics investigation was performed to more fully understand the potential for cross-contamination. The investigation used reasonable variations of several variables to examine the potential for cross-contamination. Variables examined and varied within reasonable limits were normal physiology flow rates in both the inferior and superior vena cava, typical intravenous infusion rates of 5% dextrose solutions, typical catheter port separation distances, and blood withdrawal rates. The investigation concluded that under the conditions investigated there was no reasonable potential for cross-contamination to occur. An identified limitation of the investigation was the relationship of the port to the wall of the vessel. Specifically, if the catheter is resting in the bottom of the vessel and the port is located on the bottom of the catheter, the flow characteristics surrounding the port would be quite different than in the center of the vessel as modeled in the computational fluid dynamics investigation.

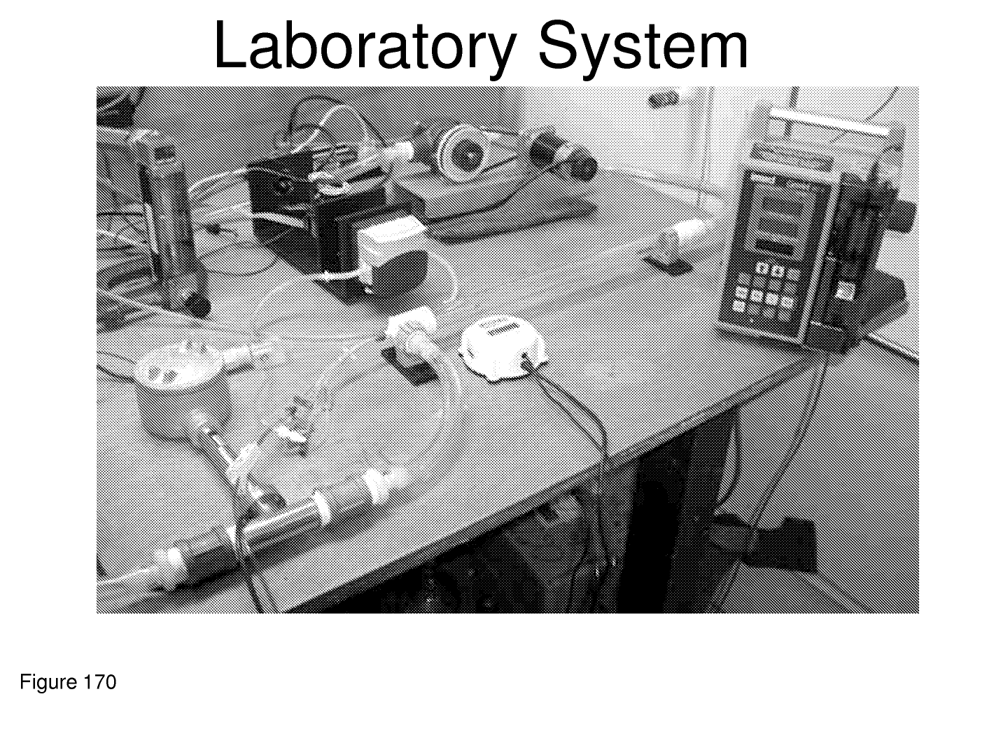

A laboratory experiment was performed to investigate variation in flow rates and variations in catheter orientation. FIG. 170 is a schematic illustration of the laboratory system. The laboratory system comprised a pump 301 capable of simulating velocity profiles in the vena cava, a Gemini infusion pump 302, a peristaltic withdrawal pump 303, an insertion type flow meter 304, a TDS conductivity meter 305, and the test section 306. The test section was constructed to simulate the superior vena cava (SVC), and is transparent acrylic with an internal diameter of 19.1 mm. The simulated blood flow travels through the flow meter 304, and enters the test section 306 through a 90 degree elbow 307, inducing turbulence in the fluid as it enters. The catheter 308 is inserted into the end of the elbow 307 and continues down inside the simulated SVC. The flow travels horizontally through the test section 306. The blood substitute is pumped from a source reservoir 309, and dumped into a sink reservoir 310 after it passes through the system. The infusion pump 302 injects either dye or potassium chloride solution into any desired catheter port, and the withdrawal pump 303 pulls fluid from any desired catheter port, through the TDS meter 305, and into the sink reservoir 310.

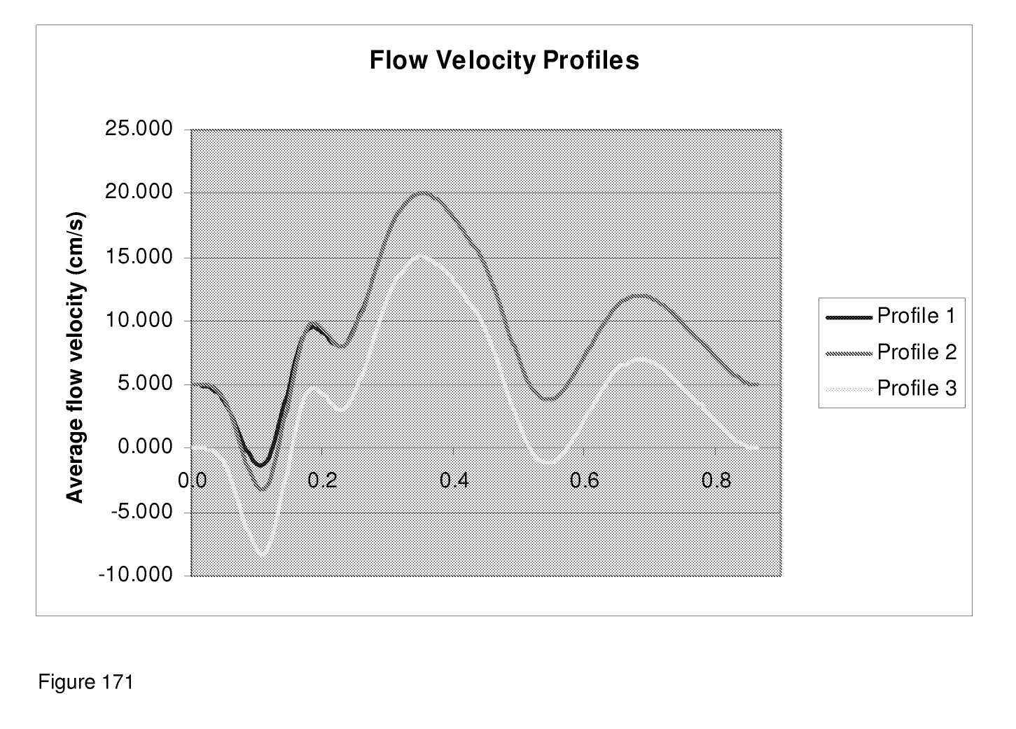

FIG. 171 is a schematic depiction of three blood flow velocity profiles investigated in the experiment. Profile 1 approximated a typical velocity profile in the SVC of a healthy adult. Profile 2 is similar to Profile 1, but with an exaggerated reverse flow region. Profile 3 was designed to encourage cross-contamination, and is similar to Profile 2 but with the velocity offset by −5 cm/sec throughout.

System verification. The conductivity meter was tested in both the installed and uninstalled conditions. While installed, it underreported the conductivity of the solutions sampled by a factor of 0.692. Because all of the conductivity measurements taken during testing were with the sensor installed in the system, the final cross-contamination values should not be affected. If desired, the true conductivity values can be obtained by multiplying all of the ppm readings by a factor of 1.45. All of the conductivity values presented in this description are the uncorrected numbers.

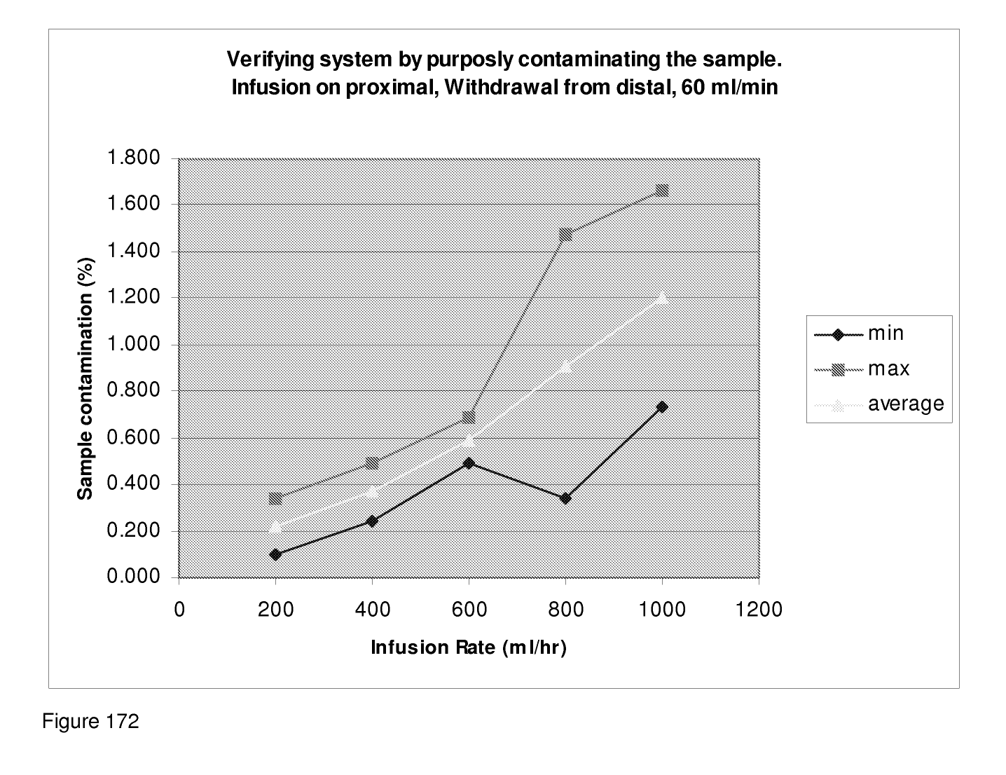

To verify that the system worked as intended, infusion and withdrawal ports were switched to purposely cause cross-contamination. A 3% potassium chloride solution was infused on the proximal port 321, and the sample was withdrawn from the distalport 322 at a rate of 60 ml/hr. Flow velocity profile 1 was used. The infusion rate was increased in steps of 200, 400, 600, 800, and 999 ml/hr. The results are presented in Table 1 and FIG. 172.

TABLE 1

| Infusion | | Venous | Infusion | Sample concentration | | | | | |
|---|---|---|---|---|---|---|---|---|---|
| rate | Temp. | concentration | concentration | (PPM) | | | Cross-contamination (%) | | |
| (ml/hr) | (deg C.) | (PPM) | (PPM) | Min | max | average | min | max | average |
| 200 | 27.1 | 430 | 20800 | 450 | 500 | 475 | 0.098 | 0.344 | 0.221 |
| 400 | 26.3 | 420 | 20800 | 470 | 520 | 495 | 0.245 | 0.491 | 0.368 |
| 600 | 26.3 | 420 | 20800 | 520 | 560 | 540 | 0.491 | 0.687 | 0.589 |
| 800 | 26.3 | 420 | 20800 | 490 | 720 | 605 | 0.343 | 1.472 | 0.908 |
| 999 | 22.8 | 410 | 20800 | 560 | 750 | 655 | 0.736 | 1.667 | 1.202 |

The verification data shows that sample contamination increases as infusion rate increases, verifying that the laboratory system works as expected when contamination is known to be present. The expanding range of minimum and maximum values might be due to turbulence caused by higher infusion rates. The percentage of cross-contamination was calculated using the following function:

$$Cross \text{-} contamination \% = \frac{( {conc}_{sample} - {conc}_{blood} )}{( {conc}_{infusion} - {conc}_{blood} )} \cdot 100$$

This function can also be used to calculate the minimum detectable level of cross-contamination. Inserting the measured concentrations of the simulated blood and infused fluid, and with the minimum detectable sample concentration rise of 10 ppm

$$Detectable cross \text{-} contamination = \frac{( 430 - 420 )}{( 20800 - 420 )} \cdot 100 = 0.049 \%$$

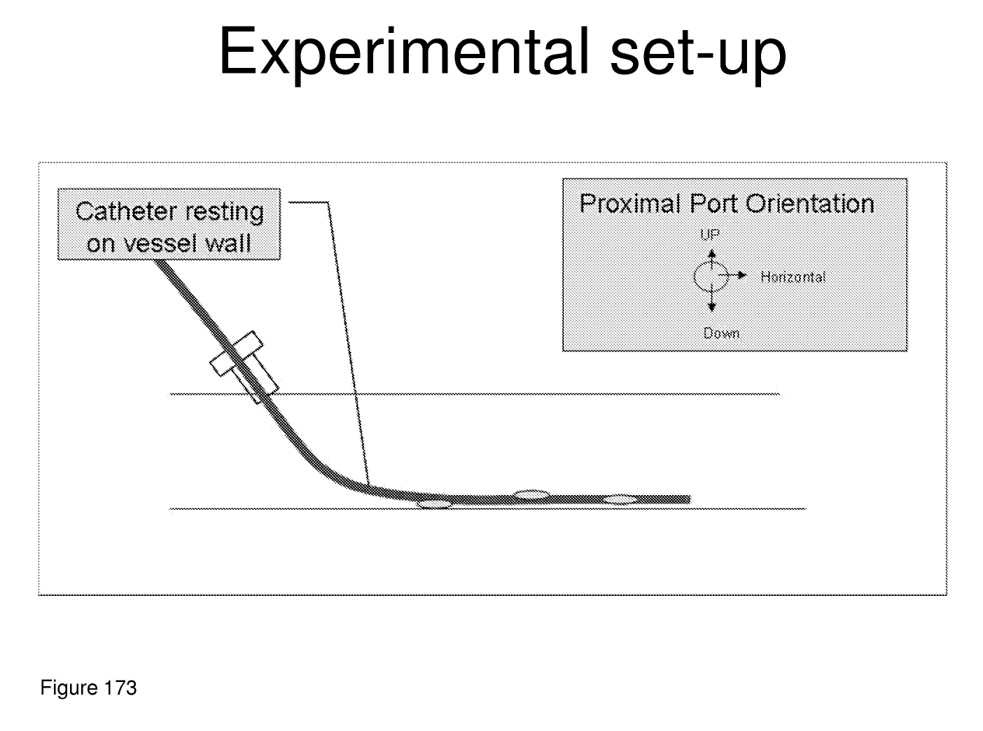

Experimental Design. Several sets of experiments were conducted to measure the cross-contamination during operation. The parameters were chosen in an attempt to increase cross-contamination as testing progressed. Infusion rates from 200 ml/hr to 999 ml/hr were tested. The concentration of the infused fluid was increased to 4% (uncorrected measurement of 27200 ppm) in order to increase the sensitivity in measured contamination levels. In addition, a test was performed with an Intralipid 20% solution consisting of about 10% potassium chloride (uncorrected measurement of 61000 ppm). FIG. 173 is a schematic illustration of the placement of the catheter and the orientation of the proximal port. The infusion rate was held constant at 500 ml/hour. Flow Profile 2 was used for all experiments.

Table 2 presents the results of the experiments with an infusion fluid of KCL and water.

TABLE 2

| Proximal Port | Average | Withdrawal | |
|---|---|---|---|
| Orientation | Velocity | Rate | % Cross-contamination |
| Down | 10 | 100 | 0.000 |
| Down | 4 | 100 | 0.000 |
| Up | 10 | 100 | 0.000 |
| Up | 4 | 100 | −0.015 |
| Horizontal | 10 | 100 | 0.015 |
| Horizontal | 4 | 100 | 0.000 |
| Down | 10 | 20 | 0.000 |
| Down | 4 | 20 | −0.015 |
| Up | 10 | 20 | 0.000 |
| Up | 4 | 20 | 0.015 |
| Horizontal | 10 | 20 | 0.015 |
| Horizontal | 4 | 20 | 0.000 |

Table 3 presents the results of the experiments with an infusion fluid of KCL and 20% Intralipid

TABLE 3

| Proximal Port | Average | Withdrawal | |
|---|---|---|---|
| Orientation | Velocity | Rate | % Cross-contamination |
| Down | 10 | 100 | 0.000 |
| Down | 4 | 100 | 0.000 |
| Up | 10 | 100 | 0.000 |
| Up | 4 | 100 | 0.000 |
| Horizontal | 10 | 100 | 0.000 |
| Horizontal | 4 | 100 | 0.000 |
| Down | 10 | 20 | 0.000 |
| Down | 4 | 20 | 0.000 |
| Up | 10 | 20 | 0.000 |
| Up | 4 | 20 | 0.000 |
| Horizontal | 10 | 20 | 0.000 |
| Horizontal | 4 | 20 | 0.000 |

There was no detectable cross-contamination during any of the tests. Calculating the minimum detectable contamination with the 4% (uncorrected measurement of 27200 ppm) solution, and assuming a detectable rise in 10 ppm, gives:

$$Detectable cross \text{-} contamination = \frac{( 420 - 410 )}{( 27200 - 410 )} \cdot 100 = 0.037 \%$$

And the minimum detectable contamination with the 10% (uncorrected measurement of 61000 ppm) solution gives:

$$Detectable cross \text{-} contamination = \frac{( 390 - 380 )}{( 61000 - 380 )} \cdot 100 = 0.017 \%$$

Therefore, the level of cross-contamination is below 0.037% in the KCl-water tests, and below 0.017% in the KCl-Intralipid testing. The laboratory testing demonstrated that the potential for cross-contamination is very low during typical use, and in experiments depicting cases worse than the typical operating conditions, cross-contamination was less than the detectable level of 0.017%.

Animal testing. A cross-contamination study on a mechanically ventilated pig was conducted to complete the investigation into cross-contamination. The protocol for investigation was (1) Place the catheter and confirm location by fluoroscopy; (2) Evaluate flow characteristics by injecting contrast agent; (3) Evaluate for cross-contamination; (4) Move catheter to next location. The testing procedure was

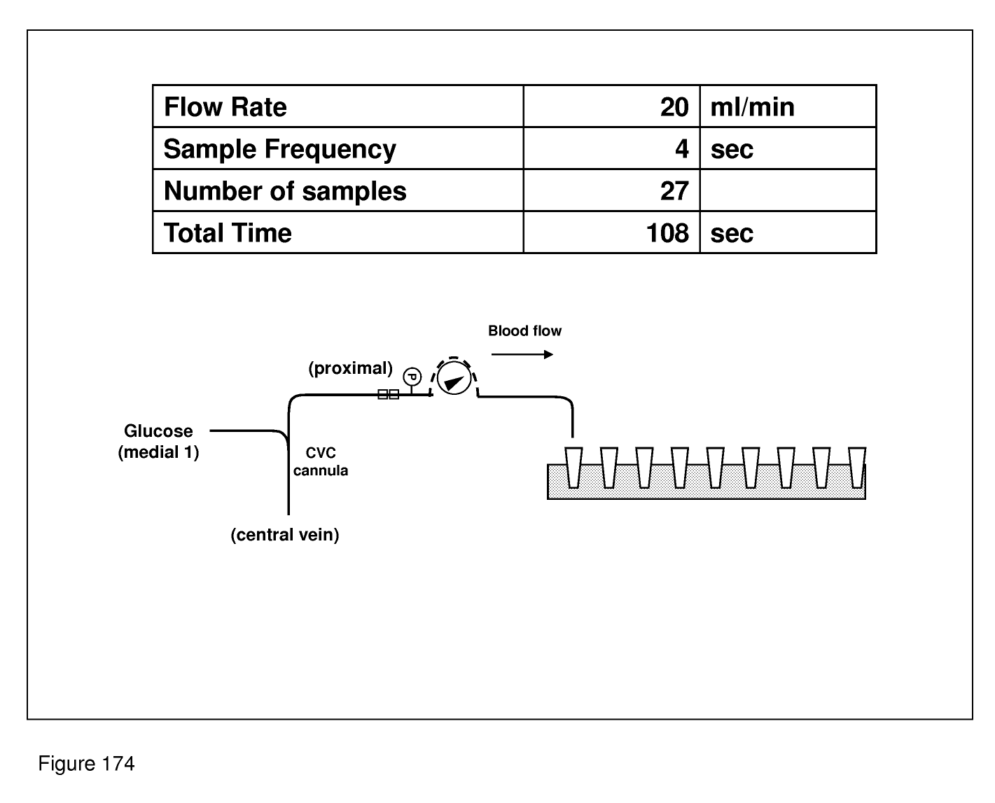

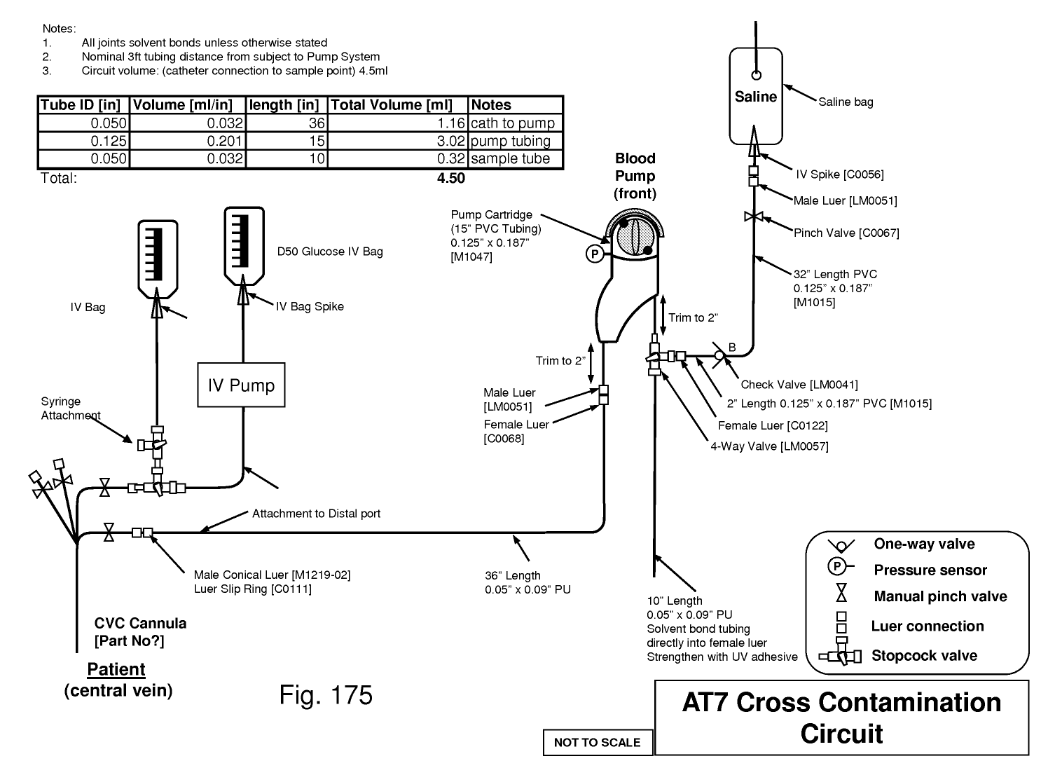

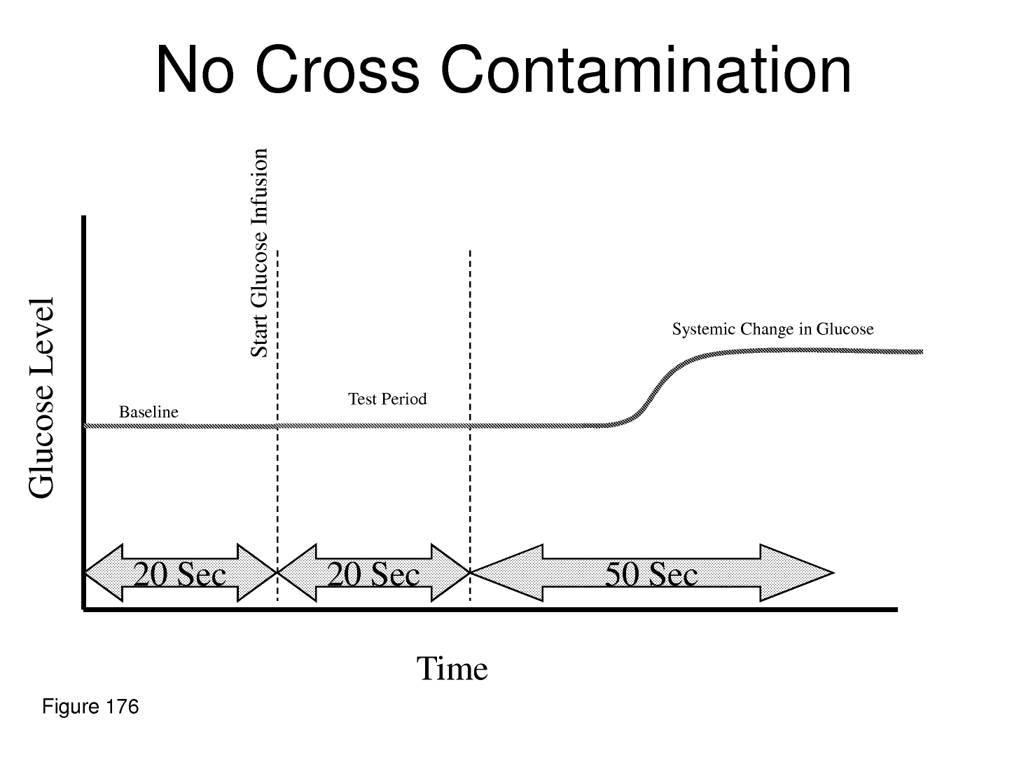

1. Initiate a sampling period where blood samples are acquired from the catheter every 4 seconds, as in FIG. 174. The actual circuit used for the test is shown in FIG. 175.
2. The initial phase establishes a baseline glucose level, as shown in FIG. 176.
3. Initiate an infusion of 50% glucose at a rate of 1000 ml/hr for a duration of 20 seconds, as shown at the start of infusion in FIG. 176.
4. Continue acquiring samples for the duration of the infusion and for a period of 50 seconds after infusion stopped.
5. Measure the glucose levels in the samples obtained.

Evaluation of Results. In a condition without cross-contamination, the initial glucose levels and those during glucose infusion will be approximately equivalent until the infused glucose has circulated in the vascular system. The amount of infused glucose will result in approximately a 50 mg/dl systemic change assuming a total blood volume of 5 liters. This end of study glucose level will be referred to as the ending glucose level. FIG. 176 is an illustration of an idealized response when no cross-contamination is present.

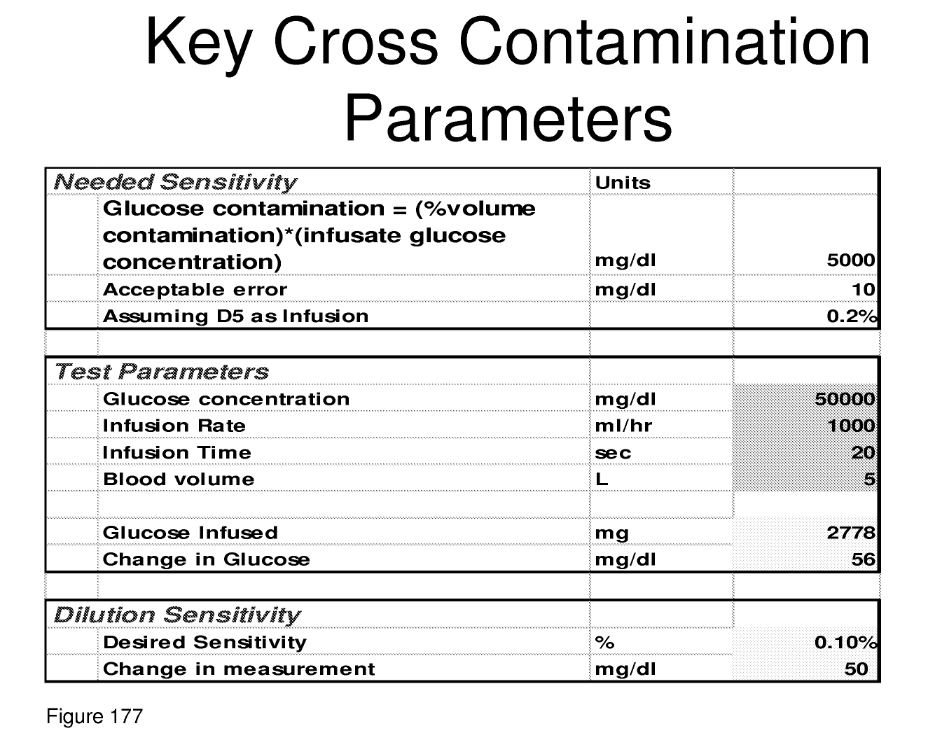

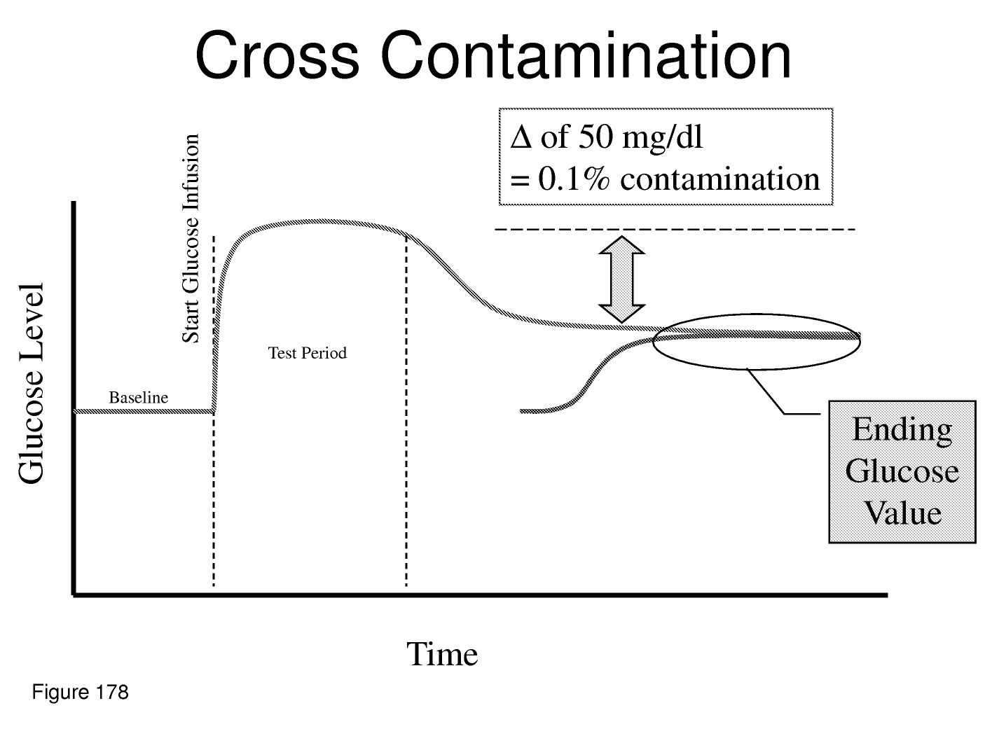

If cross-contamination occurs as a result of the infused glucose then the measured glucose will increase concurrently with the start of the glucose infusion. The use of a 50% glucose solution results in a significant glucose change even when the percentage of cross-contamination is less than 1%. FIG. 177 provides a reasonable outline of the key study parameters. If the acceptable error of cross-contamination is defined as 10 mg/dl and the solution being infused is 5% glucose, then the maximum acceptable percentage of contamination is 0.2%. If cross-contamination does occur during the glucose infusion stage, the amount of change can be easily detected. By using a 50% glucose solution (50,000 mg/dl) a 0.1% cross-contamination will result in a 50 mg/dl change relative the end of study glucose level. As shown in FIG. 178, cross-contamination results in a rapid rise during infusion with a decrease to the end of study glucose level. The maximum measured glucose level is then compared to the end of study glucose level (indicative of the final systemic glucose level) and a simple subtraction performed. A 50 mg/dl increase is indicative of approximately 0.1% cross-contamination while 100 mg/dl is indicative of 0.2% cross-contamination. In the clinical setting where 5% glucose solutions are commonly used 0.2% cross-contamination would result in glucose over prediction of 10 mg/dl.

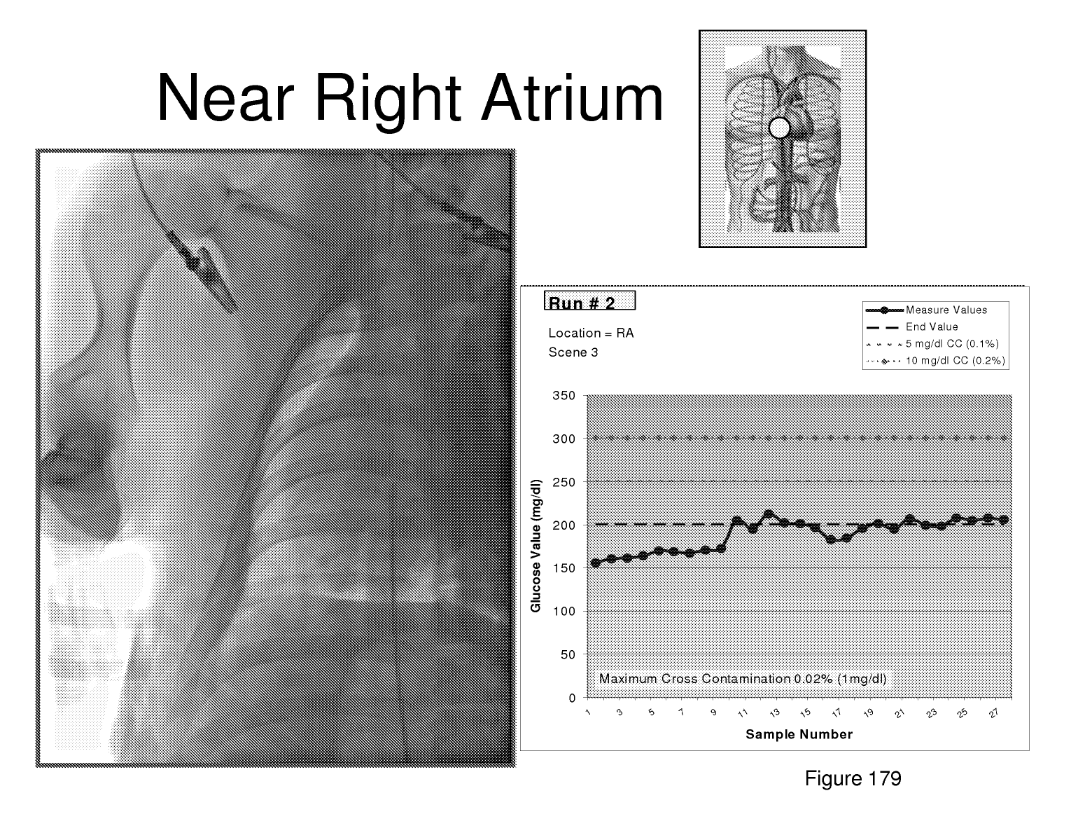

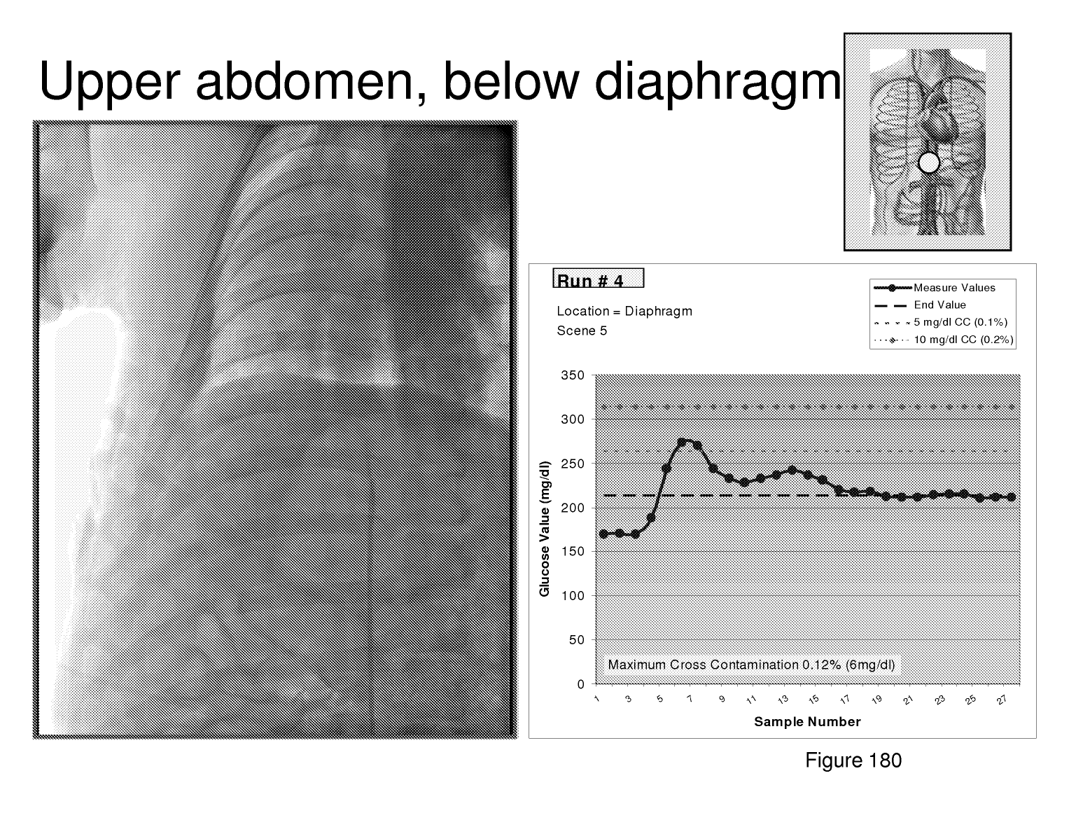

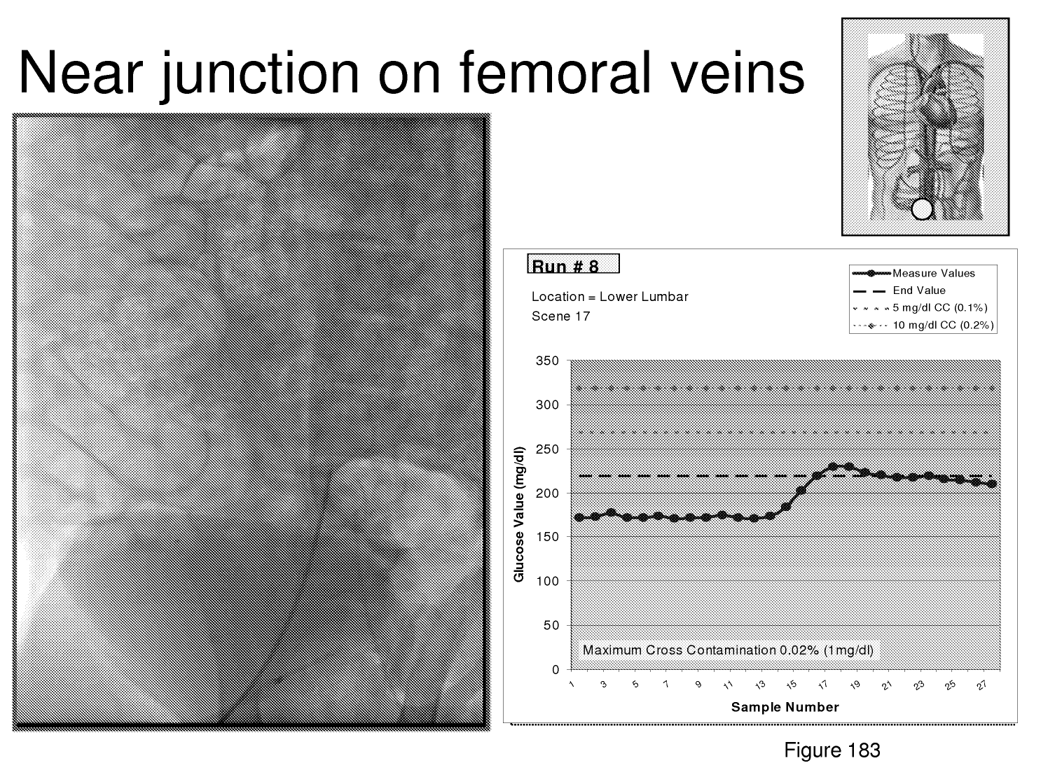

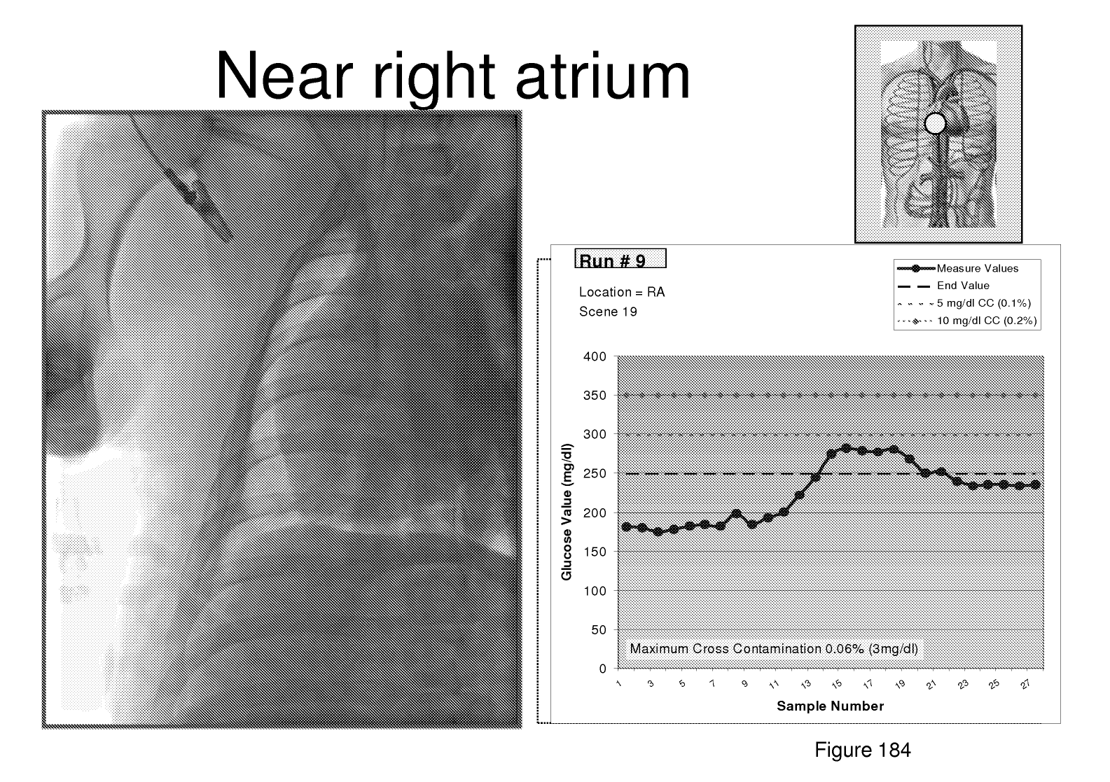

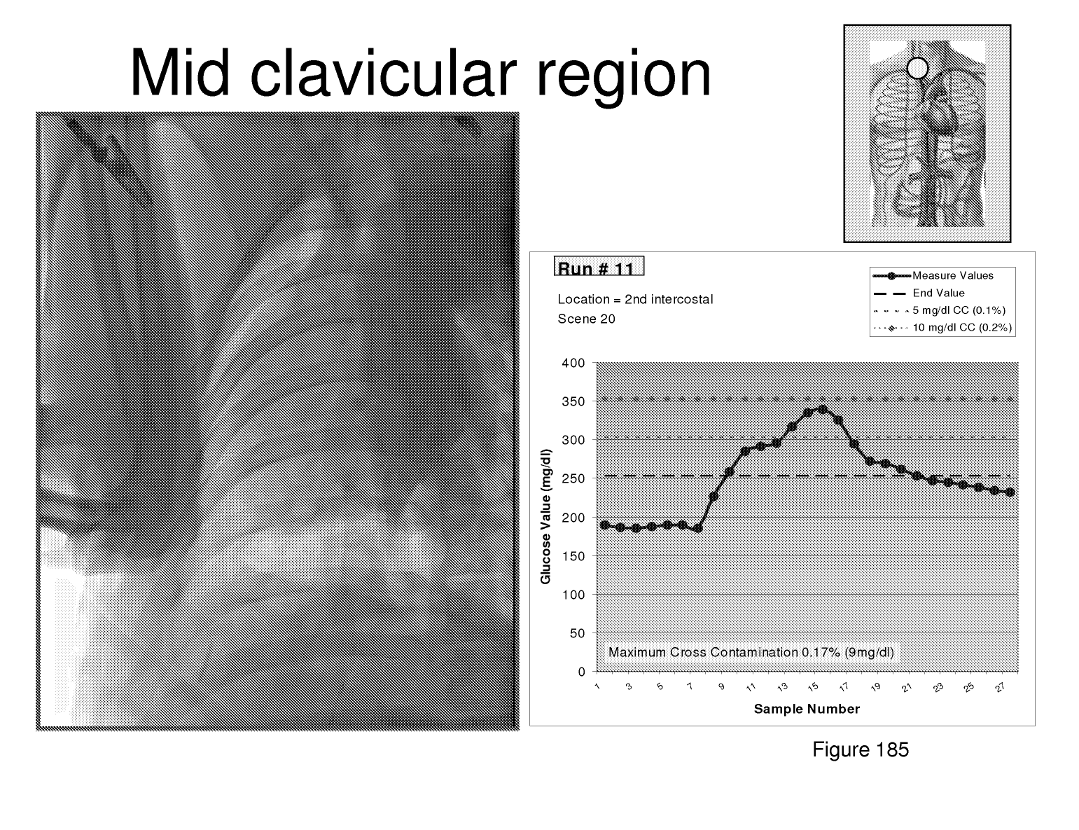

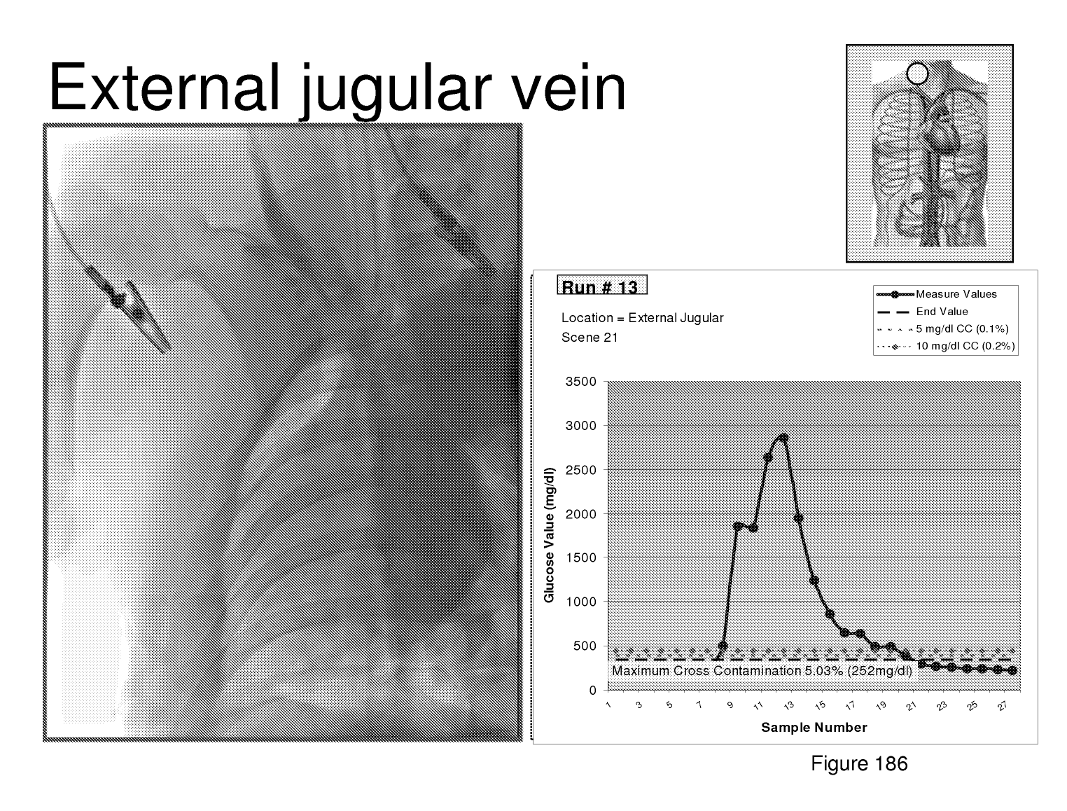

FIG. 179-187 are illustrations of experimental results, summarized in Table 4. In each figure, the radiographic image on the left side indicates catheter location. The vascular diagram shows the catheter location relative to the overall vasculature system. The graph shows test results. The x-axis is the sample number procured over the approximately 2 minutes of testing. The y-axis is the measured glucose concentration. The lowest horizontal line is the end of study glucose value which corresponds to the systemic increase in glucose concentration due to the glucose infusion. The next line is 50 mg/dl higher and corresponds to 0.1% contamination. The next line is 100 mg/dl higher then the end of study line and corresponds to 0.2% contamination. The glucose measurements from the study are plotted on the same axis.

TABLE 4

| | Figure | | |
|---|---|---|---|
| Catheter Location | Number | Ventilation | % Cross-contamination |
| Near right atrium | 179 | Yes | 0.02% |
| Upper abdomen | 180 | Yes | 0.12% |
| Mid abdomen | 181 | Yes | 0.26% |
| Mid Abdomen | 182 | NO | 0.06% |
| Junction of femoral veins | 183 | Yes | 0.02% |
| Right atrium | 184 | Yes | 0.06% |
| Mid clavicular | 185 | Yes | 0.17% |
| External jugular | 186 | Yes | 5.3% |

Four of the seven locations resulted in cross-contamination greater than 0.1%. This contrasts with the results anticipated and obtained from the computational fluid dynamics study and the laboratory investigation.

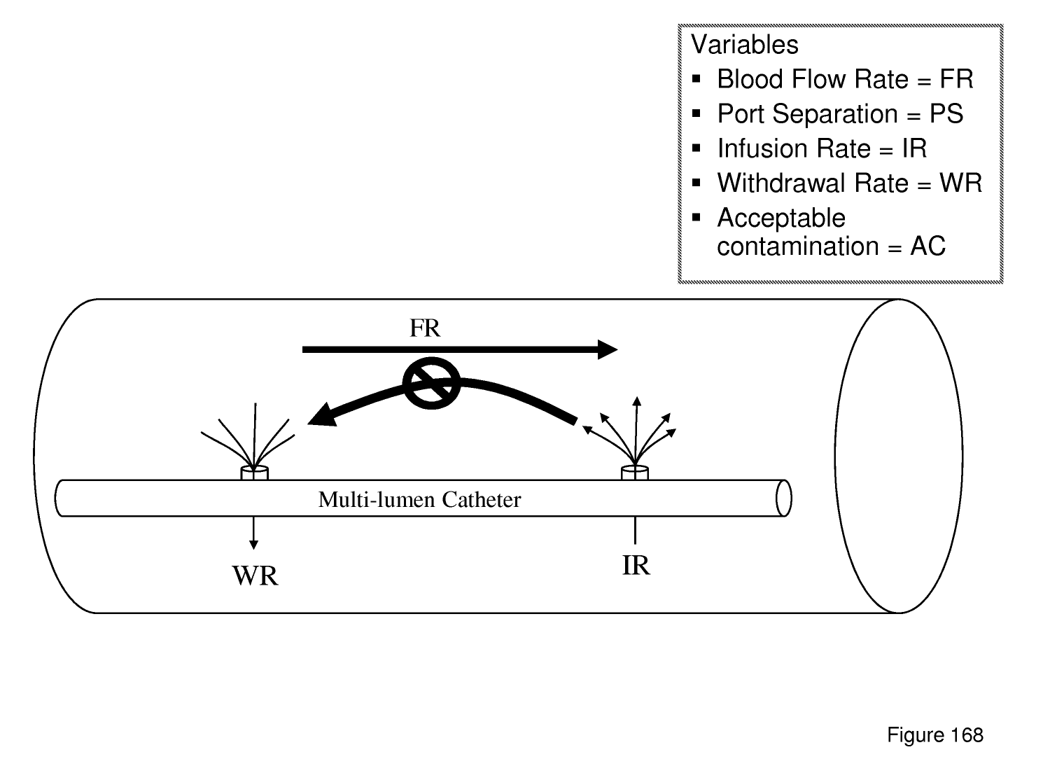

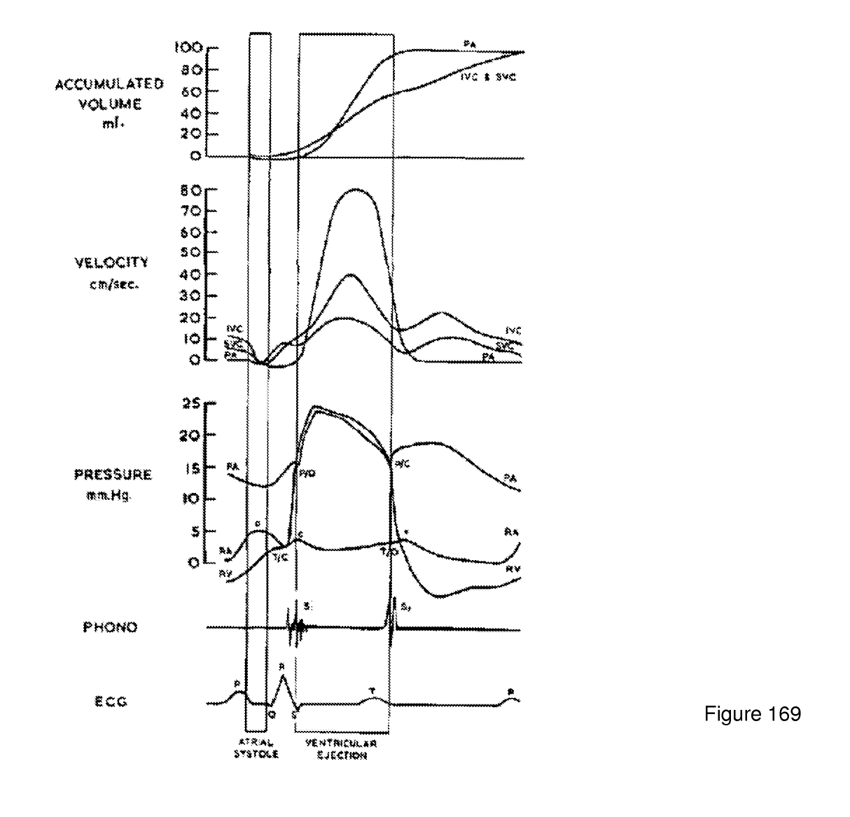

Mechanism for cross-contamination. As discussed in conjunction with FIG. 168, conditions of stagnant flow or reversed flow from the distal end of the catheter to the proximal end can result in cross-contamination. Any medical state, physiological condition or medical treatment of the subject that results in retrograde flow in large venous vessels creates an opportunity for cross-contamination. A number of medical conditions or treatments can cause such a retrograde flow; two common causes of retrograde flow in the vena cava are mechanical ventilation and abnormal cardiac function.

The normal venous pulse (Jugular venous pulse, JVP) reflects phasic pressure changes in the right atrium and consists of three positive waves and two negative troughs. In considering this pulse it is useful to refer to the events of the cardiac cycle. The positive presystolic “a” wave is produced by right atrial contraction and is the dominant wave in the JVP particularly during inspiration. During atrial relaxation, the venous pulse descends from the summit of the “a” way. Depending on the PR interval, this descent may continue until a plateau (“z” point) is reached just prior to right ventricular systole. More often the descent is interrupted by a second positive venous wave, “c” wave, which is produced by a bulging of the tricuspid valve into the right atrium during right ventricular isovolumic systole and by the impact of the crowded artery adjacent to the jugular vein. Following the summit of the “c” wave, the JVP contour declines, forming the normal negative systolic wave, the “x” wave. The “x” descent is due to a combination of atrial relaxation, the downward displacement of the tricuspid valve during right ventricular systole, and the ejection of blood from both the ventricles.

In the case of abnormal cardiac function, at least three mechanisms are known to cause a retrograde flow: tricuspid valve regurgitation, increased flow resistance out of the right atrium, and atrial fibrillation. In the case of tricuspid regurgitation, the right ventricle contracts but the tricuspid valve does not prevent retrograde flow into the right atrium and subsequently the thoracic veins. Possible conditions of retrograde flow can be associated with larger than normal “a” waves. Giant “a” waves are present with each beat, the right atrium is contracting against an increased resistance. This may result from obstruction at the tricuspid valve (tricuspid stenosis or atresia), right atrial myxoma or conditions associated with increased resistance to right ventricular filling. Abnormally large “a” waves can occur in patients with pulmonary stenosis or pulmonary hypertension in whom both the atrial and right ventricular septa are intact. Abnormally large and typically narrow “a” waves, often referred to as Cannon “a” waves, occur when the right atrium contracts while the tricuspid valve is closed during right ventricular systole. Cannon waves can occur either regularly or irregularly and are most common in the presence of arrhythmias. Atrial fibrillation is a condition known to cause the irregular occurrence of cannon “a” waves.

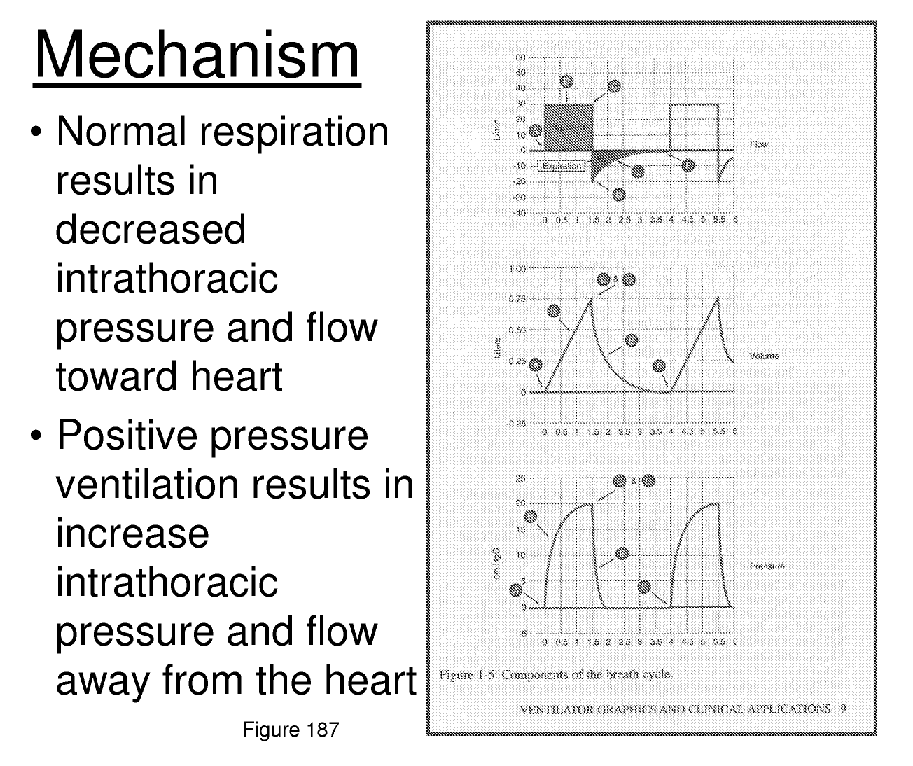

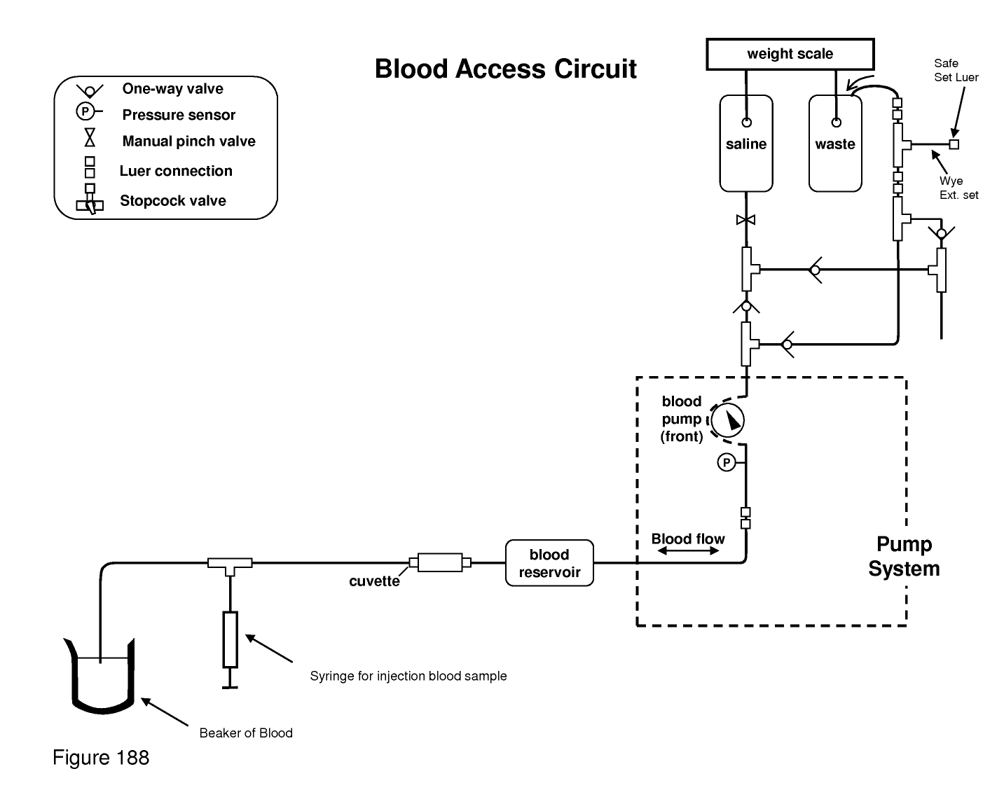

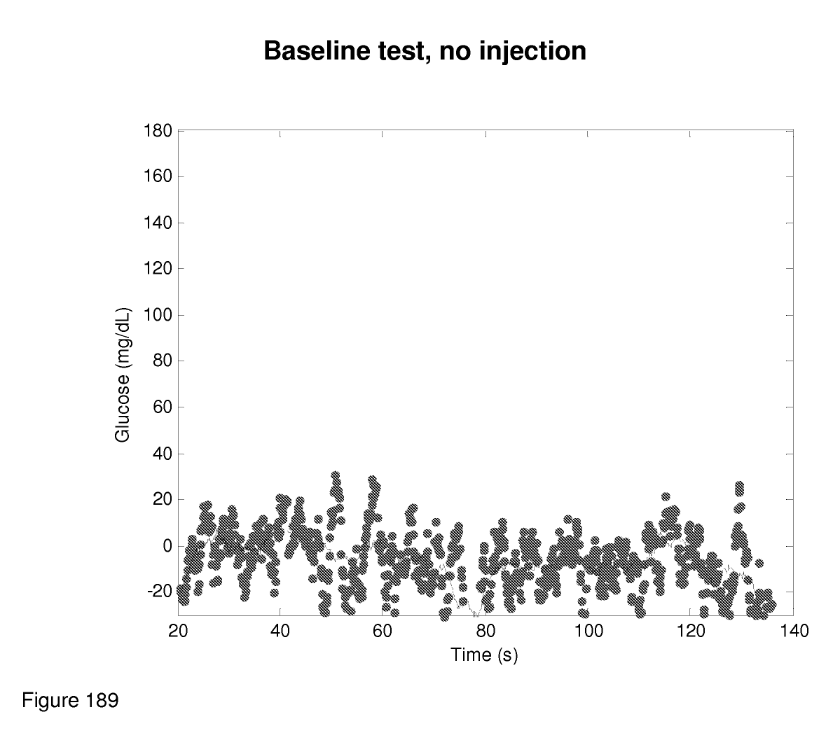

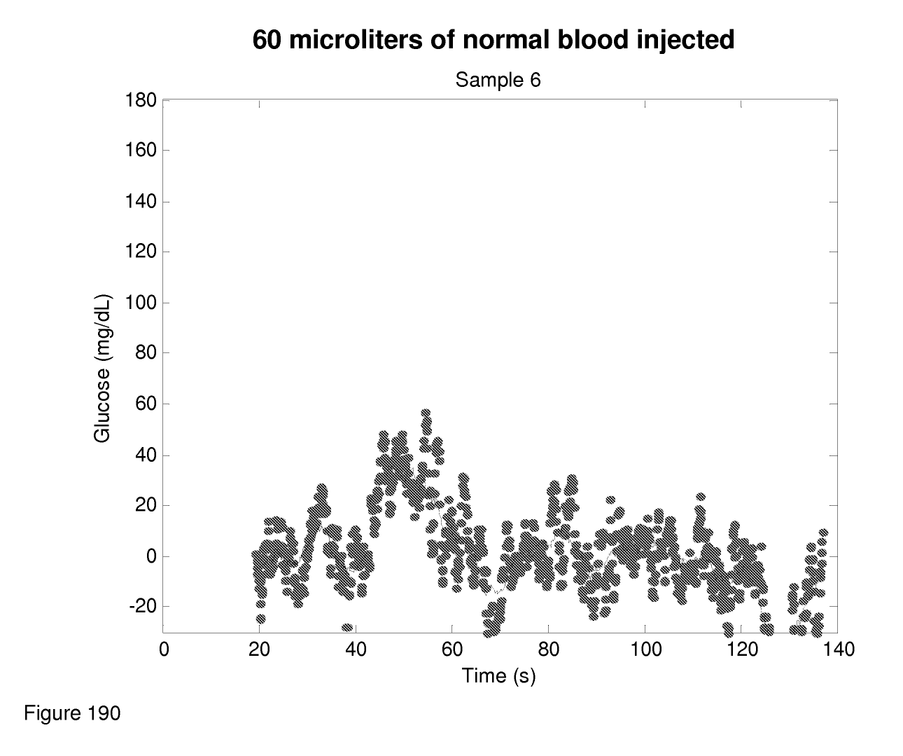

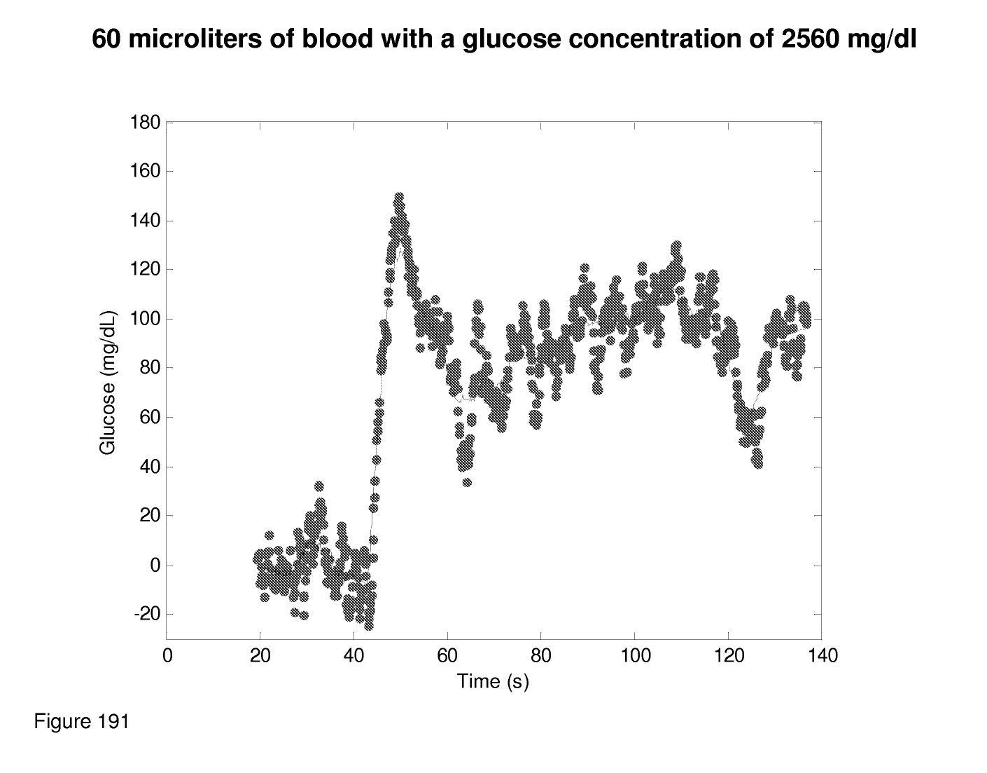

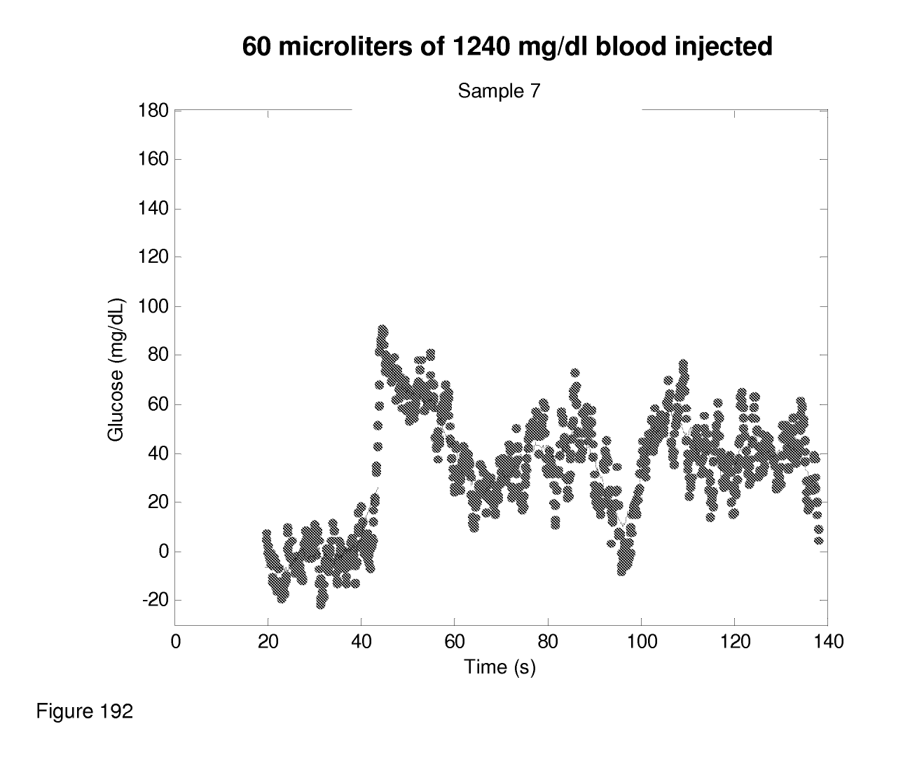

Another known source of stagnant or retrograde flow is mechanical ventilation. During normal breathing the diaphragm is lowered creating a negative pressure in the thoracic cavity. This negative pressure creates the gradient for air movement and for the filling of the lungs with each new breath. The negative pressure in the thoracic cavity also helps blood return to the heart. In the case of positive pressure ventilation, the pressure gradients are reversed. As shown in FIG. 187, the process of inflating the lung results in increased thoracic pressures. The impact of positive pressure ventilation on right heart filling pressures and volume has been documented in the literature. See, e.g., Principles and Practice of Mechanical Ventilation, by Martin J. Tobin, McGraw-Hill, copyright 2006, incorporated herein by reference. Additionally other peer-reviewed publications review the interactions between positive pressure ventilation and heart function. See, e.g., “Heart-lung interactions: applications in the critically ill” by H. E. Fessler, European Respiratory Journal, 1997; 10: 226-237, and “Cardiovascular Issues in Respiratory Care” by Michael R. Pinsky, Chest 2005: 128: 592-597; each of which is incorporated herein by reference. The impact on blood flow in the large veins leading to the heart was investigated in the 1960s but has received very little documentation or re-examination since then. Key papers covering blood flow in the large thoracic vessels are as follows and are incorporated herein by reference: Chevalier P A, Weber K C, Engle J C, et al. Direct measurement of right and left heart outputs in ValSalva-like maneuver in dogs. Proc Soc Exper Biol Med 1972; 139:1429-1437: Guntheroth W C, Gould R, Butler J, et al. Pulsatile flow in pulmonary artery, capillary and vein in the dog. Cardiovascular Res 1974; 8:330-337: Guntheroth W G, Morgan B C, Mullins G L. Effect of respiration on venous return and stroke volume in cardiac tamponade. Mechanism of pulsus paradoxus. Circ Res 1967; 20:381-390; Holt J P. The effect of positive and negative intrathoracic pressure on cardiac output and venous return in the dog. Am J Physiol 1944; 142:594-603; Morgan B C, Abel F L, Mullins G L, et al. Flow patterns in cavae, pulmonary artery, pulmonary vein and aorta in intact dogs. Am J Physiol 1966; 210; 903-909; Morgan B C, Martin W E, Hornbein T F, et al. Hemodynamic effects of intermittent positive pressure respiration. Anesthesiology 1960; 27:584-590. Upon review of the above literature, there are a number of unobvious characteristics of the large veins that enable mechanical ventilation induced retrograde flow. First, the superior and inferior vena cava do not have valves that prevent reverse flow. In the smaller veins of the body there are one way valves that allow flow toward the heart but not retrograde flow. The lack of valves in the vena cava creates an opportunity where blood can flow toward the heart or away from heart solely based upon pressure. Additionally this compliant effectively runs across three different atmospherically related but different segments. The segments for examination are the abdominal cavity, the thoracic cavity and the ambient/jugular cavity. Large asymmetric pressure changes in any of these segments can induce flow within the vena cava.

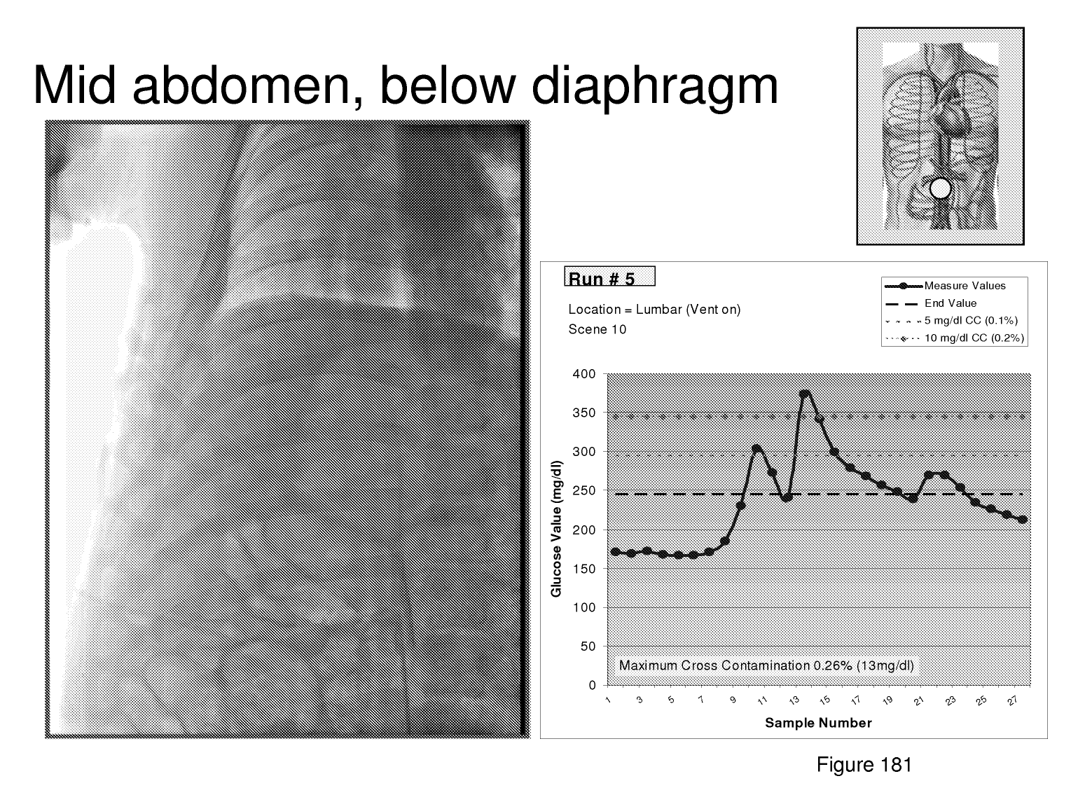

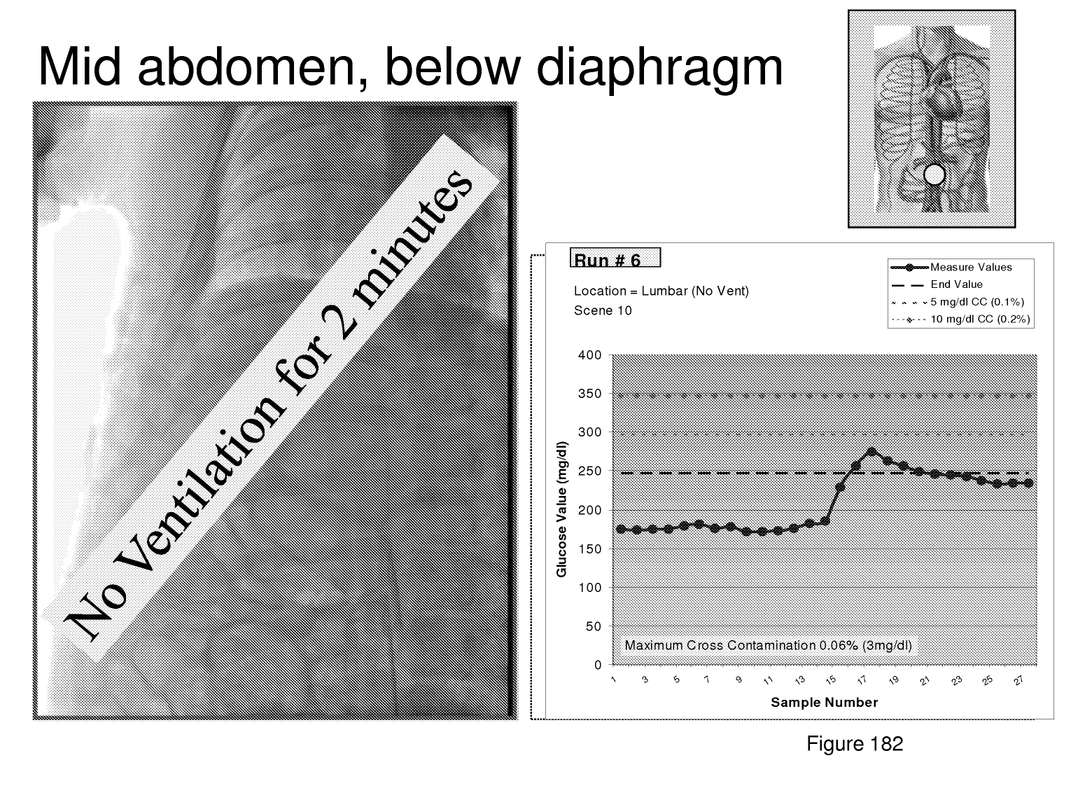

In the study animal conducted, reverse flow occurred during the periods of positive pressure ventilation. To help confirm that mechanical ventilation is the major source of retrograde flow and subsequent contamination, one location was examined with and without ventilation. For the catheter location in the mid abdomen, two tests were conducted. The first was conducted with mechanical ventilation on and the second test with no ventilation. The rate of ventilation was 10 breaths per minute. As can be seen by comparing FIG. 181 and FIG. 182, the degree of cross-contamination is very significant when the animal was ventilated while there is little or no evidence of contamination when the ventilation was stopped for the duration of the study. Careful examination of FIG. 181 also shows a variation of cross-contamination that has a frequency that is well correlated with the ventilation frequency. Since the pressure gradients vary over the ventilation cycle, the amount of cross-contamination can vary as a function of these changes.

Detection of cross-contamination. Reliable detection of conditions that are likely to lead to cross-contamination can be beneficial, since glucose measurements made during such conditions can be adjusted or discarded as possibly inaccurate. Pressure changes can be used to detect conditions likely to lead to cross-contamination. The measured glucose values can themselves be used to detect when one or more measured values are likely to have been compromised by cross-contamination. The physics describing the potential for cross-contamination indicate that the amount of cross-contamination can be sensitive to the withdrawal rate. Cross-contamination can be detected by comparing two different analyte values. Cross-contamination can be assessed by making two measurements where the difference between the measurements is the operation of the infusion pumps.

Reducing the influence of cross-contamination. It can be convenient for a measurement system to automatically adjust its operation to reduce the influence of cross-contamination. As one example, if the measured response shows variations in glucose values that are consistent with the ventilation frequency then the resulting data stream can be processed to remove the values likely to be influenced by cross-contamination. In a simple example, the lowest 10% of values in a sequence of measurement values can be averaged and this number be reported as the measured glucose value. More sophisticated process methods such as digital filtering or Fourier transformation can also be used.

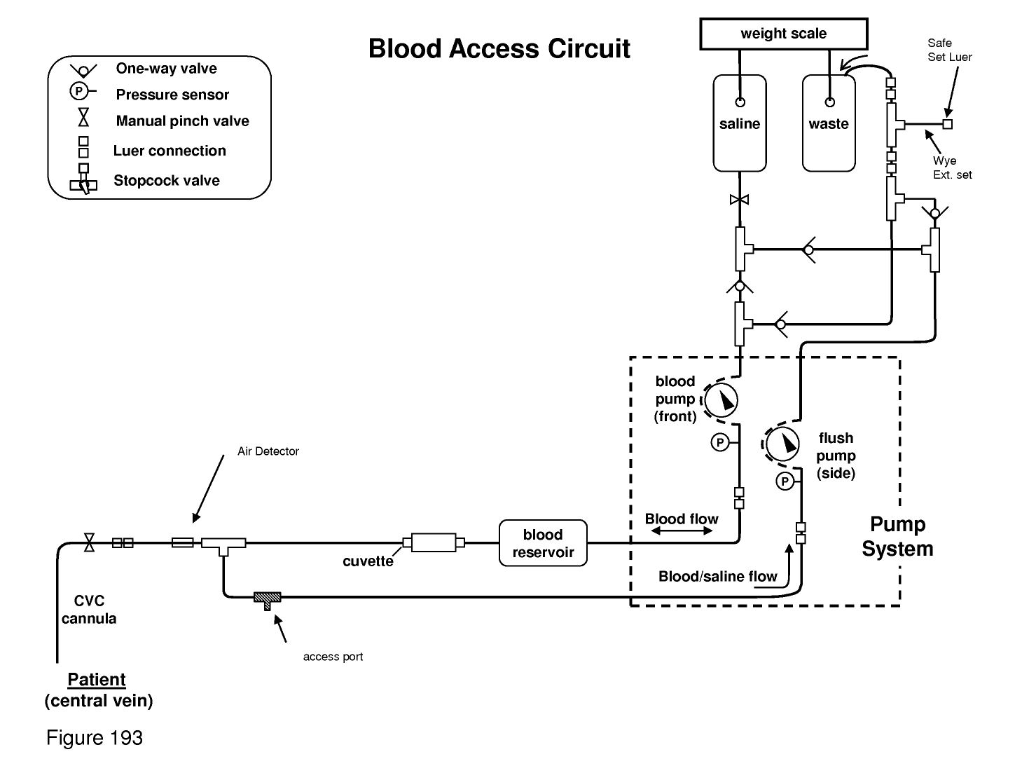

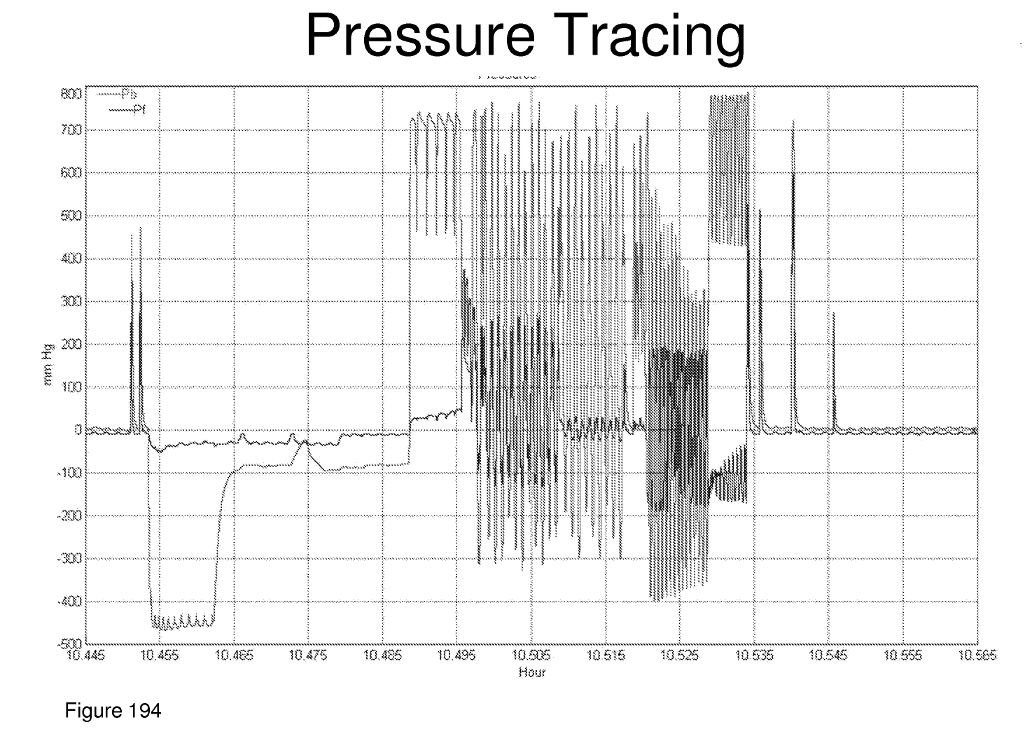

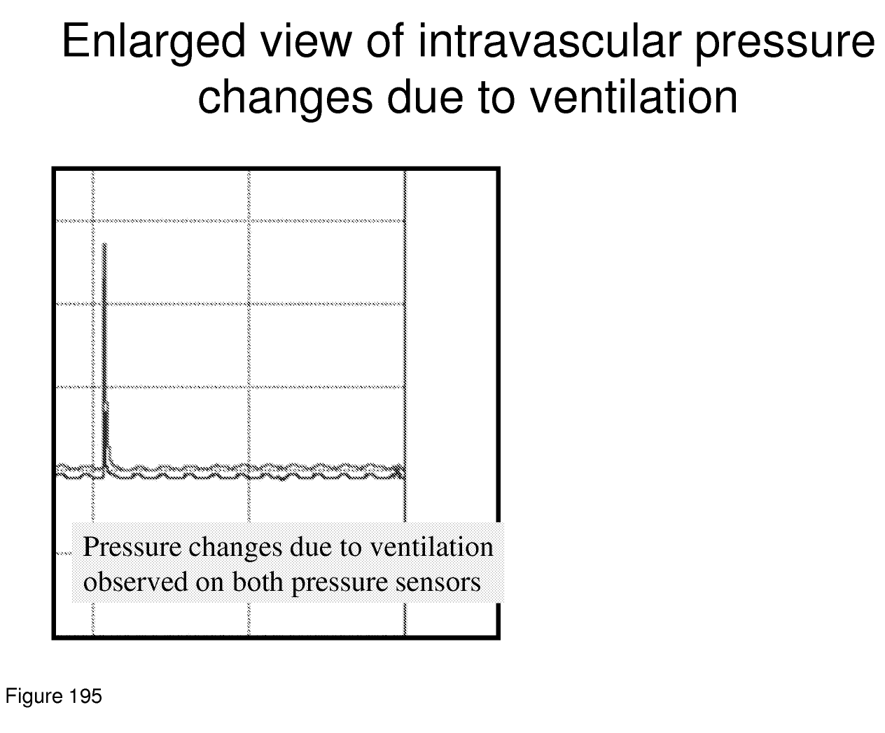

Under conditions where the patient is not ventilated or the influence of ventilation is moderately small, the withdrawal rate can be a more important factor. In the animal testing conducted with catheter locations near the right atrium or in the pelvis, there was no appreciable cross contamination but the withdrawal rate was only 20 ml/min. A nurse can easily generate withdrawal rates in excess of 60 ml/min. The potential for cross-contamination is influenced by the flow rate of blood at the site of the central venous catheter, the rate of infusion, the rate of withdrawal, the glucose concentration of the infused fluid, catheter port orientation and the distance between the point of infusion and withdrawal. The rate of withdrawal is an important parameter in determining cross-contamination: control of this parameter can reduce the likelihood of cross-contamination. In the hospital environment the rate of withdrawal can vary appreciably due to the type of syringe used, the force applied by the nurse or clinical care provider, and a variety of other uncontrolled variables. Under a variety of conditions, the withdrawal rate of the blood access system can specified and controlled such that the amount of cross-contamination does not affect the clinical efficacy of the device. Based upon medical data, the typical flow in a non-ventilated patient in the superior vena cava will average between 10 and 20 cm per second with a peak at 35 cm per second in the direction towards the heart. This will overwhelm both the infusion velocity and the withdrawal velocity of the infused drugs except for periods of about 200 ms during which the flow is retrograde at about one to 2 cm per second for about 150 ms. The retrograde flow will cause the infused fluid from the medial port to move in a retrograde manner over a distance of about 0.3 cm. The typical distance between ports on most central venous catheters is about 1 cm. The use of withdrawal rates that do not create enough suction to pull the glucose infusion across the port separation distance should be used when procuring blood samples for glucose measurement. Cross contamination can be prevented during blood withdrawal by interrupting the withdrawal of the sample during the inflation of the lung or at any point where cross-contamination is sufficiently likely. As noted previously, the large venous vessels in the thoracic cavity do not have valves, therefore flow is determined by pressure gradients. For the purposes of determining the presence of reverse flow, measurement of intravascular pressures or pressure changes can be beneficial. In the blood access circuit shown in FIG. 193, the two pressure transducers located on the pump console have the capability of measuring intravascular pressure. FIG. 194 shows the pressure tracing obtained during eight automated sample withdrawal, measurement, re-infusion and cleaning cycles. FIG. 195 illustrates the influence of ventilation during those periods of constant infusion typically referred to as KVO (“keep vein open”). During periods when one or more of the pumps are active the quality or information content of the intravascular pressure can be diminished by the influence of the withdrawal pumps. Due to this diminished signal it can be desirable to use a signal from the ventilator, or measured based on the ventilator, as the true signal of ventilator status. While this provides an assessment of ventilator status, it might not be an exact indicator of intravascular pressure due to a number of lags or pressure delays present in the body. For example, in the case of central venous catheter located in the abdomen, there can be an appreciable delay between the initiation of positive pressure ventilation and a corresponding pressure change at the catheter. Assuming that the catheter does not move appreciably, this delay can be quantified by examining the difference between the pressure response as measured from the ventilator and the corresponding pressure response measured in the vessel. This lag can be well-characterized during periods when the intravascular pressure signal is not corrupted by the withdrawal pumps. Such a period exists during KVO infusions. Multiple methodologies can be used to determine intravascular pressure and/or the correlation between intravascular pressure and the stage of ventilation. The following example embodiments include an example method for measuring the ventilator stage, concurrently measuring intravascular pressure and defining the associated lag.

In practice, it can be desirable to minimize the total time needed to withdraw the blood and eliminate any unwanted flow characteristics at the catheter tip due to the overall compliance of the circuit. These desired requirements can be achieved with responsive and active control of fluid flows, pressures, or a combination thereof. Four methods of interrupting flow for the purpose of anti-cross contamination controls have been identified: 1) a compliance isolation method, 2) a flow feedback method 3) a cascade pressure-flow feedback control method and 4) a pressure feedback method. For completeness of the description of operation, the block diagrams of these example circuits include direct measurement of the ventilator stage and include the determination of lag between the ventilator stage and the intravascular pressure change, although as described herein variations are possible.

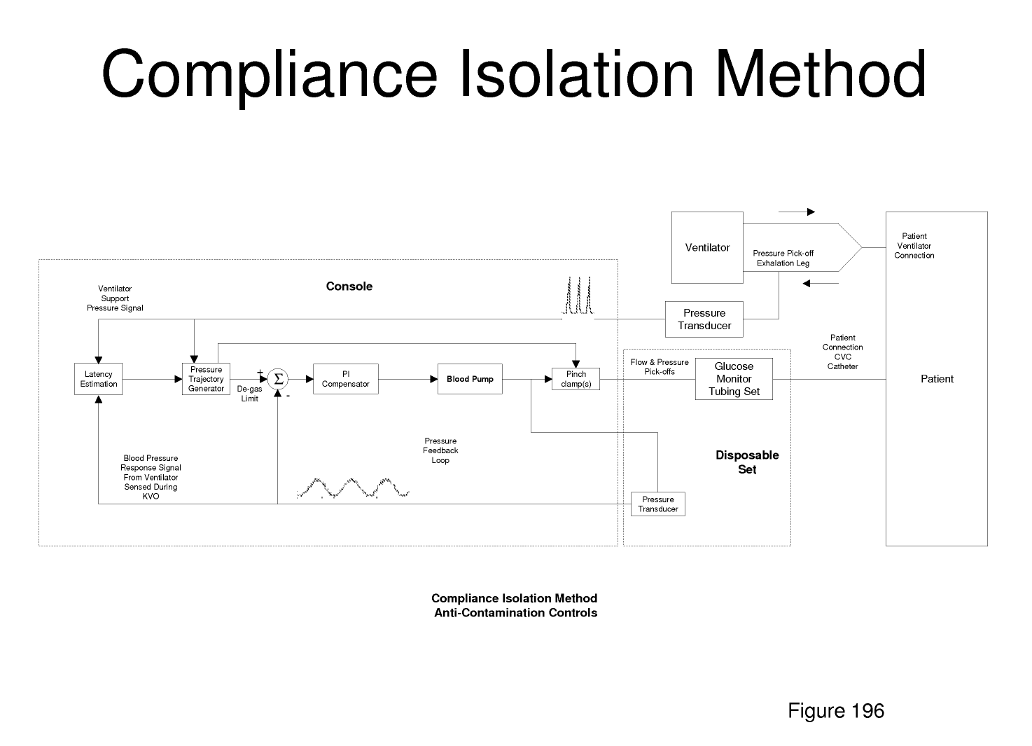

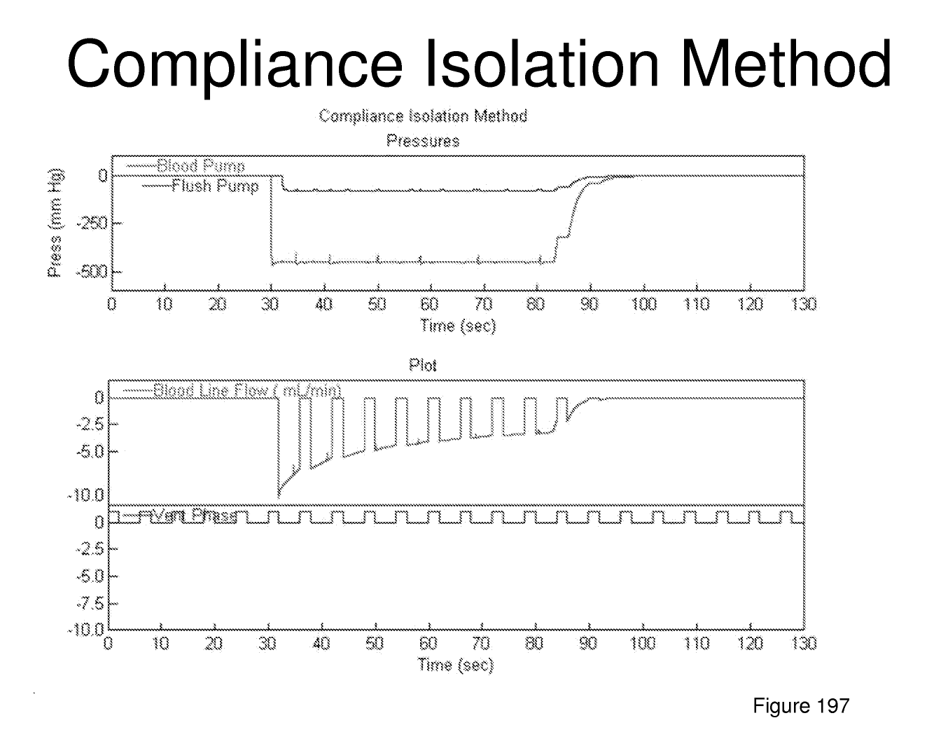

Compliance Isolation: The compliance isolation method provides anti-contamination control by using pinch valves that close fluid connections between the pump loop tubing and the sensor set at the blood optionally and optionally the flush pump, interrupting flow during the interval of lung inflation. This method works with the example pressure based withdrawal technique shown and prevents or minimizes flow reversal during the intervals of interruption by isolating the soft compliance of the pump loops from the stiffer portions of the sensor set. The pinch valves are activated immediately upon the signal of lung inflation and the pumps are allowed to continue operating at the pressure target with zero flow. Any alarms that would normally sense occlusions during the withdrawal can be deactivated during this interval. FIG. 196 shows a block diagram of the compliance isolation method. The pressure feedback loop comprises the sensor set blood line pressure transducer that provides a true measure of blood line pressure. This pressure is compared to the desired blood line pressure and the difference is used to control the blood pump through a control compensator that is structured and tuned to minimize this difference in transient and steady state conditions. With the pinch clamp open, the blood pump affects the flow and pressure in the tubing set. With the pinch clamp closed, the blood pump no longer affects either flow or pressure in the tubing set. Pressure between the blood pump and pinch valve are controlled to the desired pressure, but pressure and flow downstream of the pinch valve both drop to zero. FIG. 197 shows the simulated pressure and flow responses during a withdrawal where the compliance isolation method is used.

As shown in FIG. 196, the desired pressure target command shaping and timing can be determined according to a pressure reference trajectory generator that determines the latency between the ventilator pressure signal and ventilator induced pressure changes on the blood pressure measurement. These latencies can be determined during KVO operation and used to delay the command to stop flow with the pinch valves accordingly.

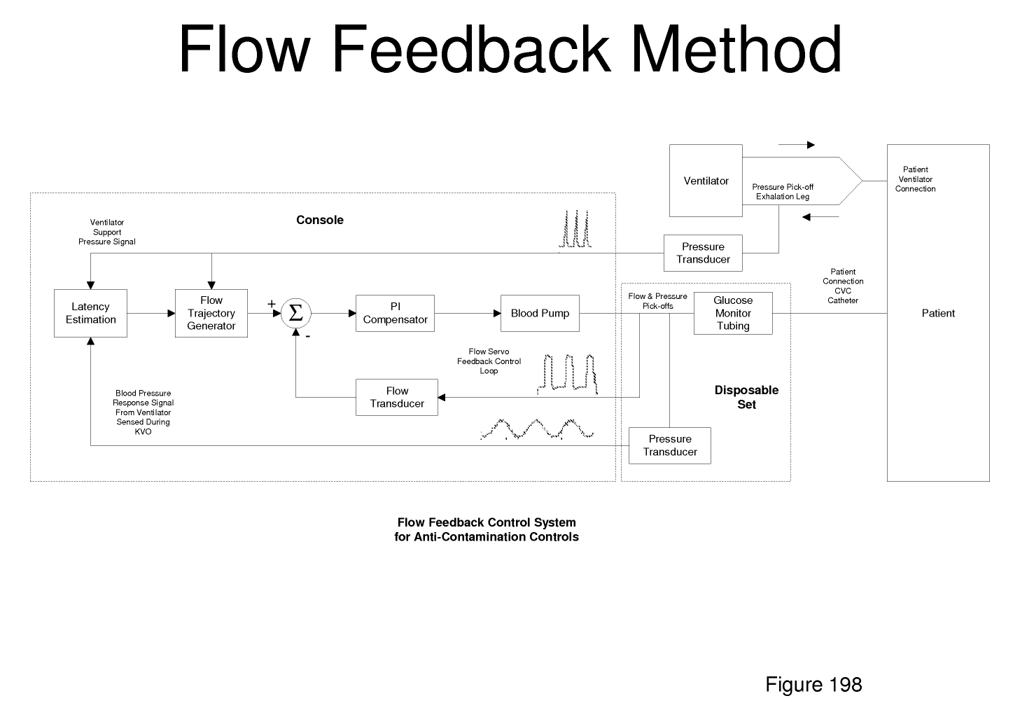

Flow Feedback Control: FIG. 198 illustrates a flow feedback method, using a flow sensor in the blood line to sense fluid flow which can be compared to a desired flow. The difference is fed to a controller which, when correctly tuned, commands the pump and minimizes the flow difference both during transient and steady states of flow. Thus the true flow will follow the desired flow. The flow feedback loop is operational all the time during the draw however the desired flow (command) is adjusted according to the state of lung inflation. During the state where the lung is not inflated, the desired flow is set to a constant flow target, and the withdrawal proceeds. When lung inflation is sensed, the desired flow is commanded to zero (or near zero) interrupting the withdrawal. The flow feedback loop stiffens the effective flow impedance of the sensor set. This results in a faster time constant in the flow response as compared to the sensor set without flow feedback where changes in flow are limited by the intrinsic compliance and resistance of the sensor set. Without flow feedback, the natural response of the sensor set causes flow withdrawal to continue even after the pump is stopped. With flow feedback the pump actually reverses direction to counteract this natural response and achieve zero flow in a more rapid manner.

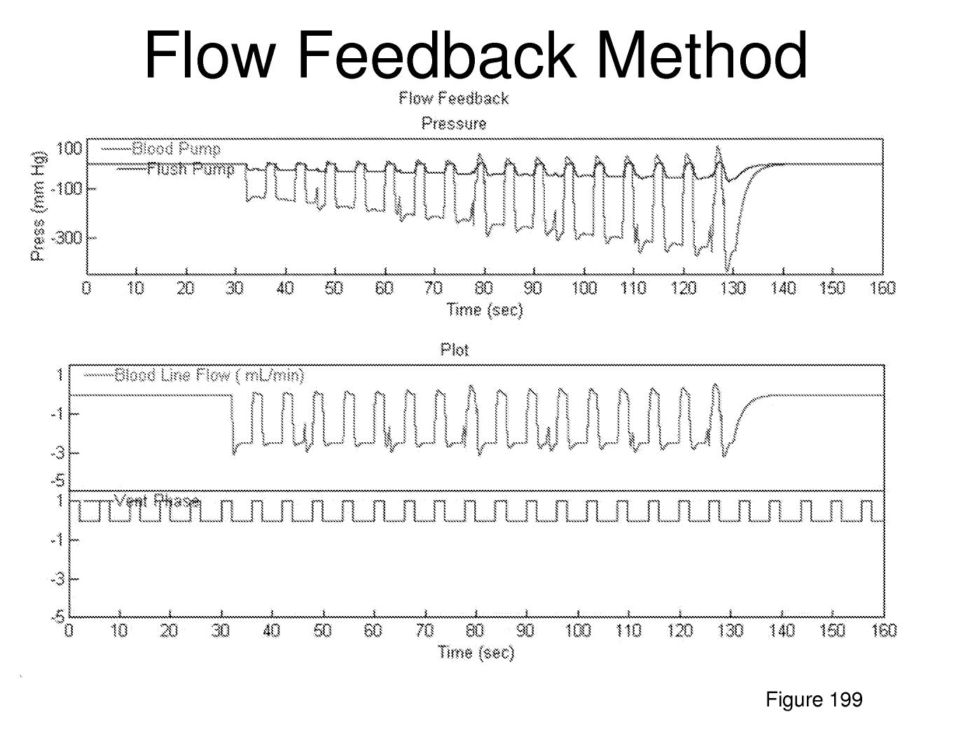

For this method to work properly, the desired flow target must be set at a value that does not cause pressure to exceed the pressure limit beyond which degassing of the fluids might be expected to occur. Pressure can increase as additional blood is drawn into the blood line so the flow target must be set so that pressure is maintained within the limit at the end of the draw. The desired flow target command shaping and timing are determined according to a flow reference trajectory generator that determines the latency between the ventilator pressure signal and ventilator induced pressure changes on the blood pressure measurement. These latencies are determined during KVO and used to delay the command to stop flow accordingly. FIG. 199 illustrates a simulated operation of the flow feedback control method during a withdrawal.

Cascaded Flow-Pressure Feedback Control: The cascade control method enhances the benefits of 1) maximum draw rate at a target negative upstream pressure which limits the de-gas rate of fluids during intervals where cross contamination is not expected, and 2) rapid deceleration of fluid flow rate to zero (or near zero) during intervals where cross contamination is expected. These benefits are achieved by using an inner, flow feedback control loop, and an outer, pressure feedback control loop. These inner and outer loops comprise the control cascade.

The inner flow feedback loop is operational all the time during the draw as well as draw interruptions, and the outer pressure feedback loop is only active between the flow interruptions. The inner flow feedback loop effectively stiffens the flow impedance of the sensor set. This results in a faster time constant in the flow response as compared to the sensor set without flow feedback where changes in flow rate are limited by the intrinsic compliance and resistance of the sensor set.

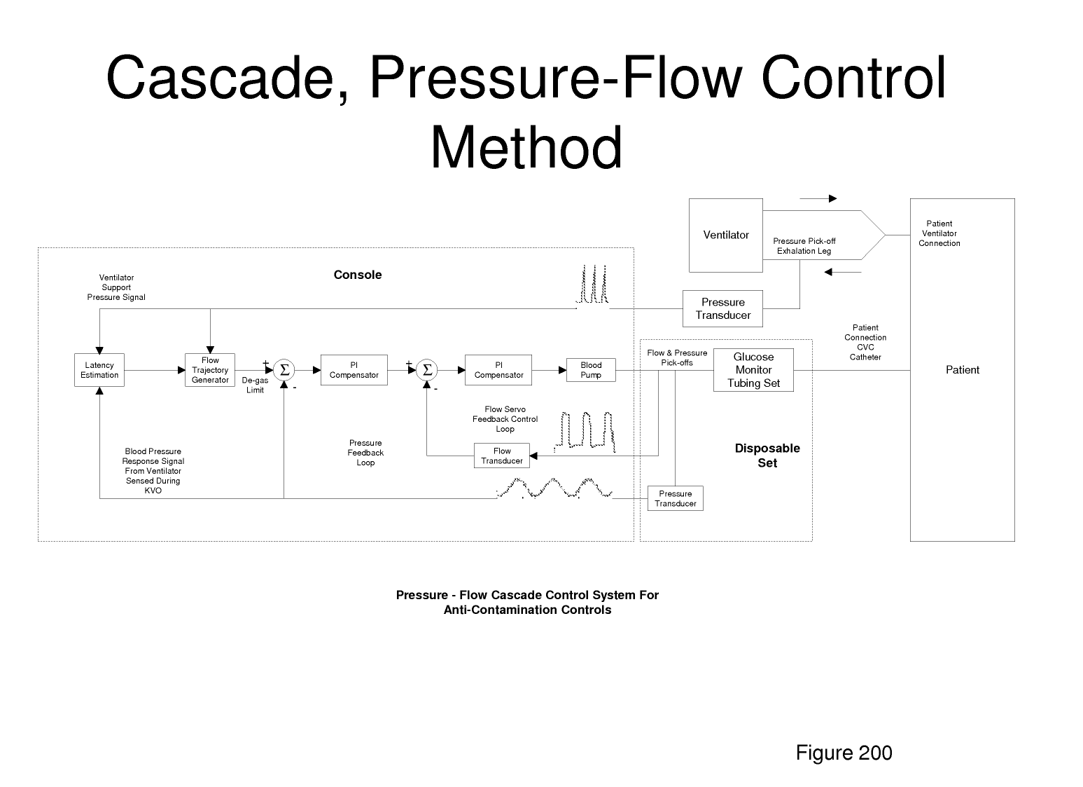

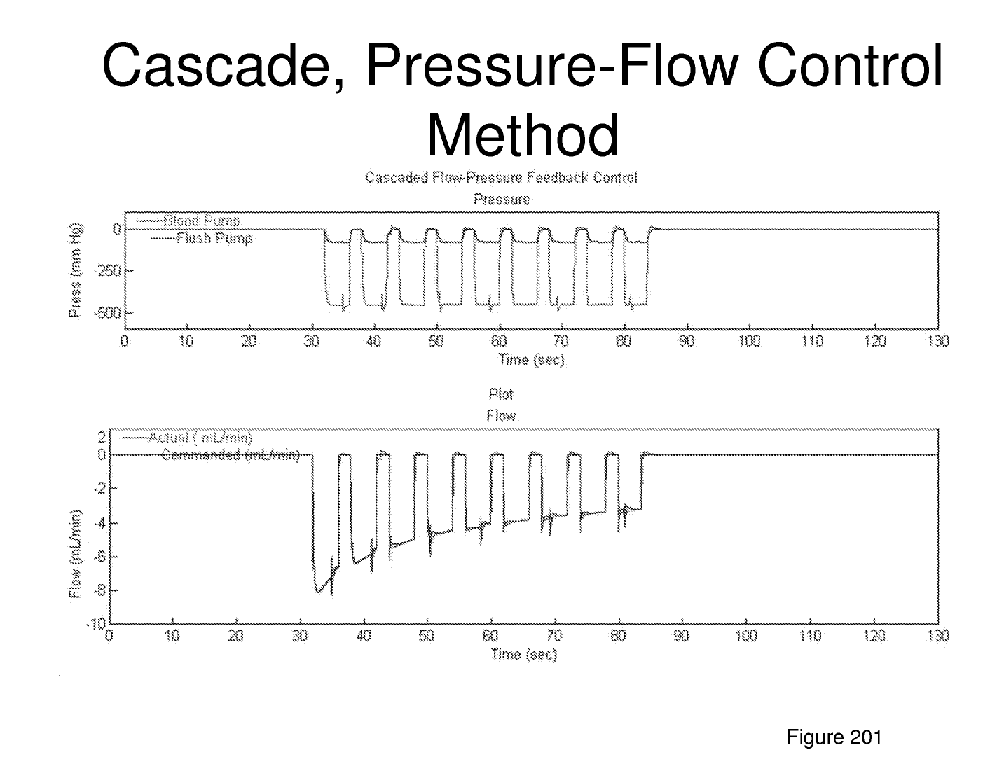

The outer pressure feedback loop provides the command signal to the inner flow feedback loop during the interval of lung deflation, where cross contamination is not expected to occur. The pressure loop targets a high negative pressure during that interval to maximize the draw rate however within a pressure constraint that prevents or minimizes degassing of the blood and maintenance fluid. During lung inflation the pressure controller is reset and held inactive with a command of zero flow to the inner flow loop. FIG. 200 illustrates, by block diagram, the cascade control method. FIG. 201 illustrates simulated operation of the cascade control method.

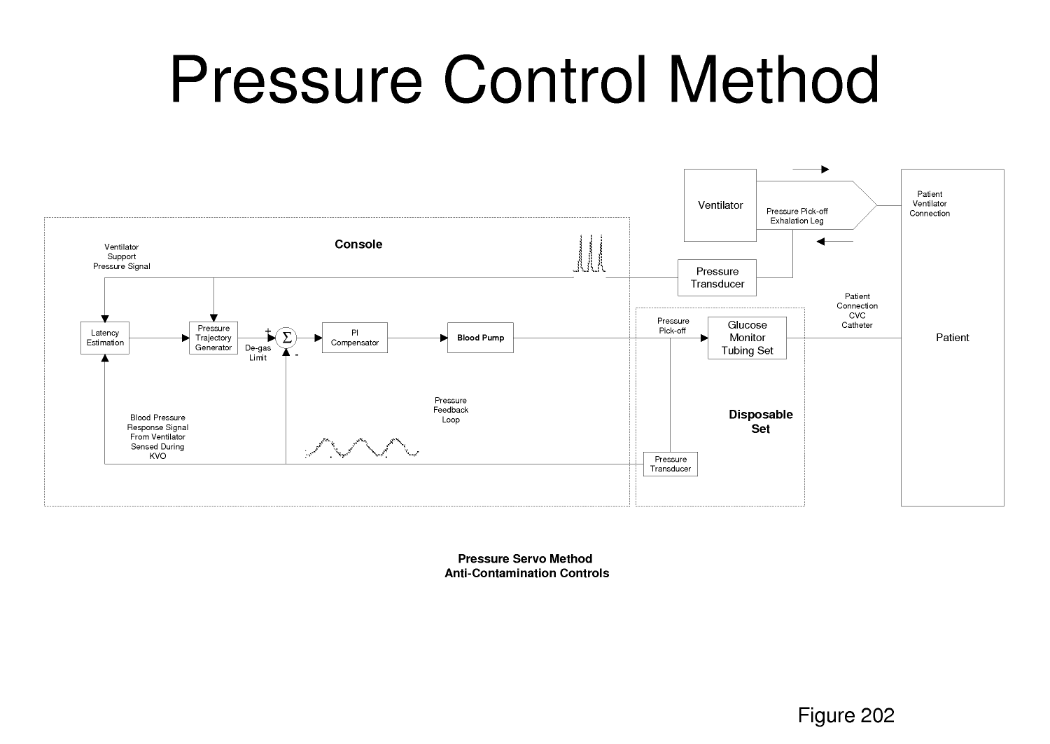

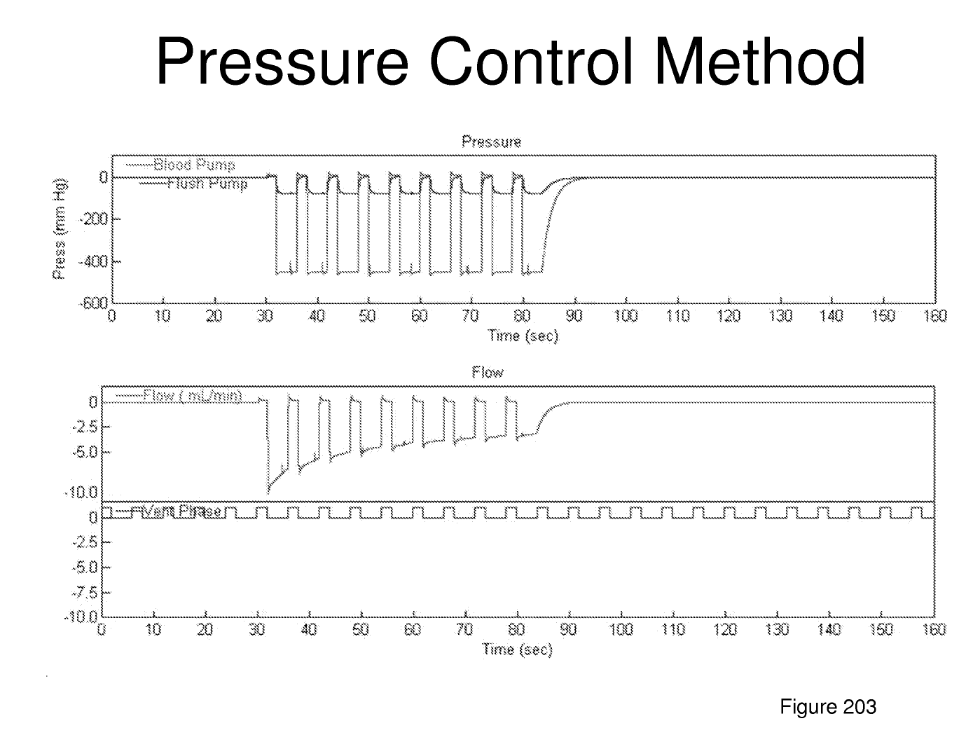

Pressure Feedback Control: The pressure feedback control method utilizes the same pressure feedback control servo used during the draw intervals for the intervals that interrupt withdrawal by substituting a slightly positive pressure target during these intervals. This results in an immediate reversal of the pump just after the draw which prevents reversal of flow during the interrupts and maintains a slight positive flow from the canula. FIG. 174 is a schematic block diagram of this approach where the pressure trajectory generator decides between the positive or negative pressure target based on the phase of ventilation. As described in the other methods, the pressure fluctuations observed from the blood pressure transducer are used to determine latency, if any, between pressure changes in the blood and those measured from the ventilator during KVO to delay action. FIG. 202 shows an example of the pressure feedback control method in simulation. FIG. 203 shows a simulator response using the pressure feedback control method.

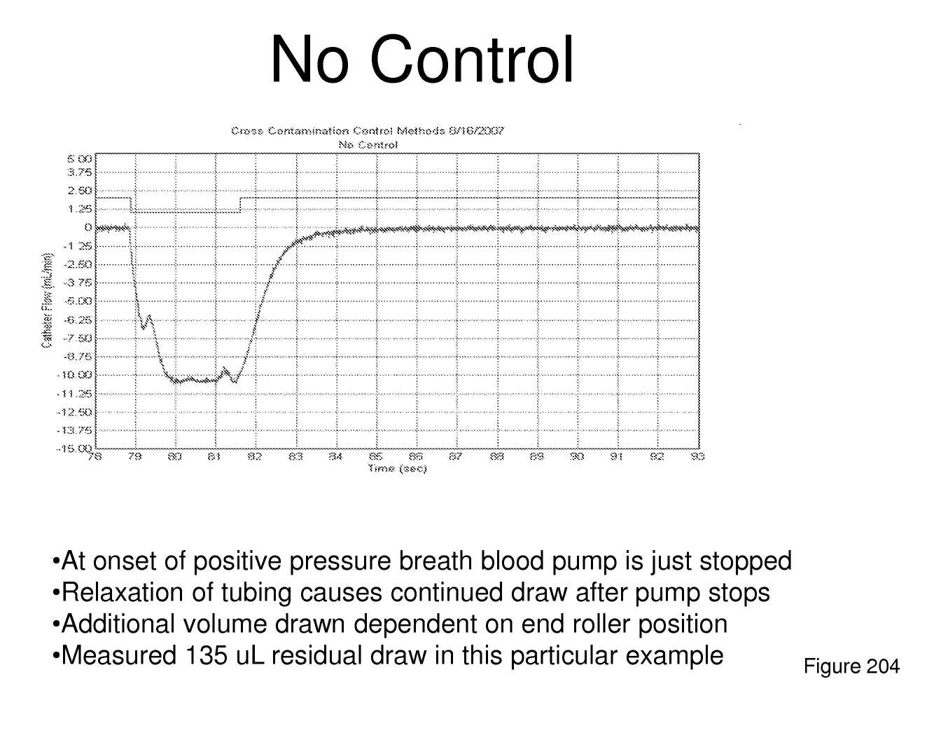

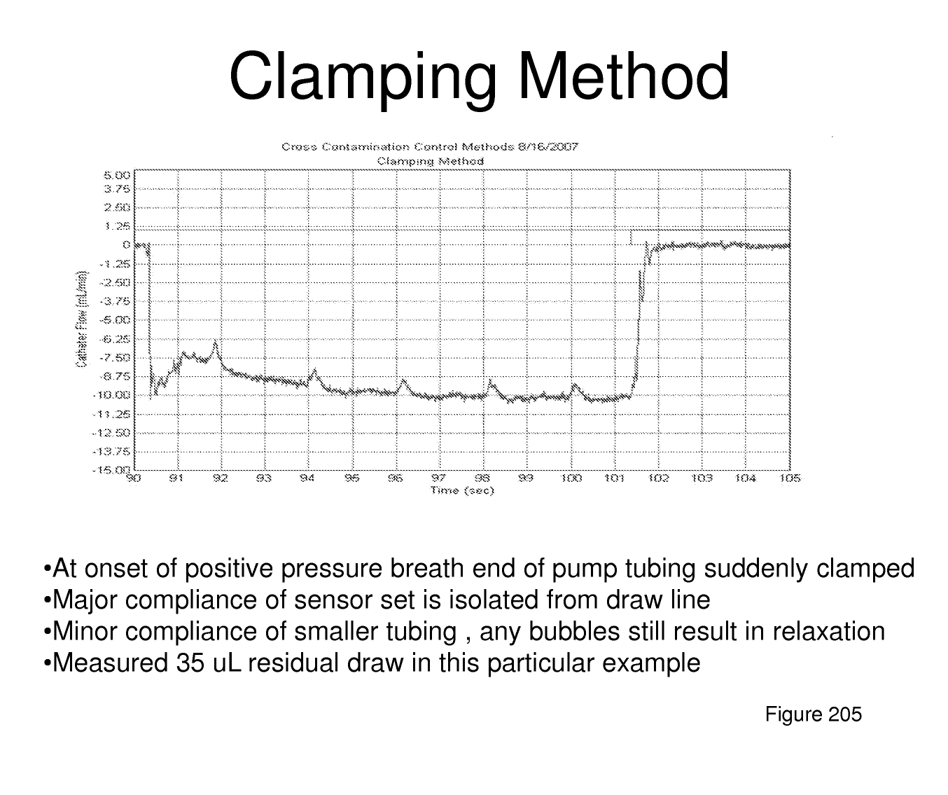

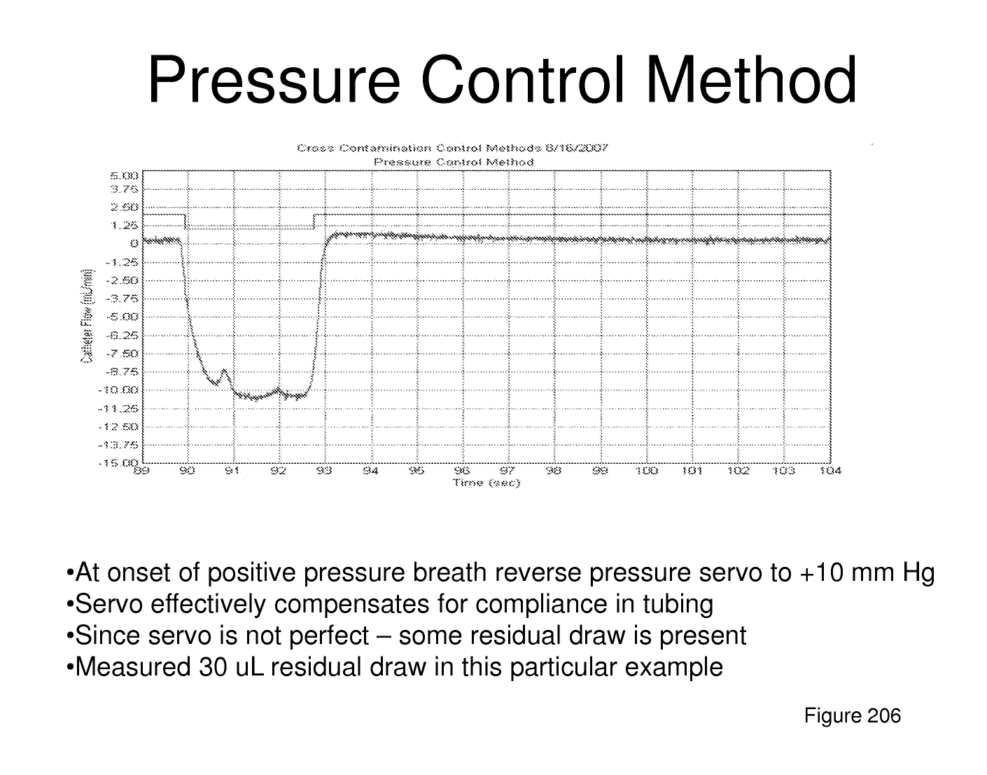

To further confirm the operational principles with respect to controlling flow in a blood access circuit, a simple confirmatory test was conducted in the laboratory. A blood access circuit and pumping mechanism as shown in FIG. 193 was utilized. At the end of the catheter, and ultrasonic flow sensor was placed for the recording of fluid flows. A simulated ventilator signal associated with inspiration was generated such that a stop flow or stop withdrawal signal was generated. The performance characteristics were then documented by the flow measurement system and response times were calculated. This proof of principle investigation sought to demonstrate the performance characteristics of: (1) no control, (2) the compliance isolation method and (3) pressure control method. The no control method was implemented by simply issuing a command to stop pumping via the peristaltic pumps. There is no active control to minimize any residual compliance artifacts in the circuit. In the case of the compliance isolation method the clamping methodology used a controlled hemostat. As can be seen in FIG. 204, the no control methodology can effectively start and stop the circuit but the residual compliance in the circuit results in an undesired continuation of the withdrawal for about 1.5 seconds and an additional unwanted withdrawal volume of approximately 135 uL. FIG. 205 shows the results from the isolation compliance method. The use of a clamp effectively stops flow when used below the compliant pump tubing. The unwanted withdrawal volume is now decreased to only 35 uL. FIG. 206 shows the implementation of the pressure control methodology. In this case the pump control servo mechanism was instructed to operate between −450 mm Hg and +10 mm Hg. As can be seen by the flow tracing this methodology has a very fast response time and results in very little unwanted withdrawal volume. Furthermore for the pressure control method, the set positive pressure during the period of lung inflation can be adjusted so that a small reverse flow is affected to entirely flush back any contaminated sample that might have entered the blood sampling line.

The particular sizes and equipment discussed above are cited merely to illustrate particular embodiments of the invention. It is contemplated that the use of the invention can involve components having different sizes and characteristics.

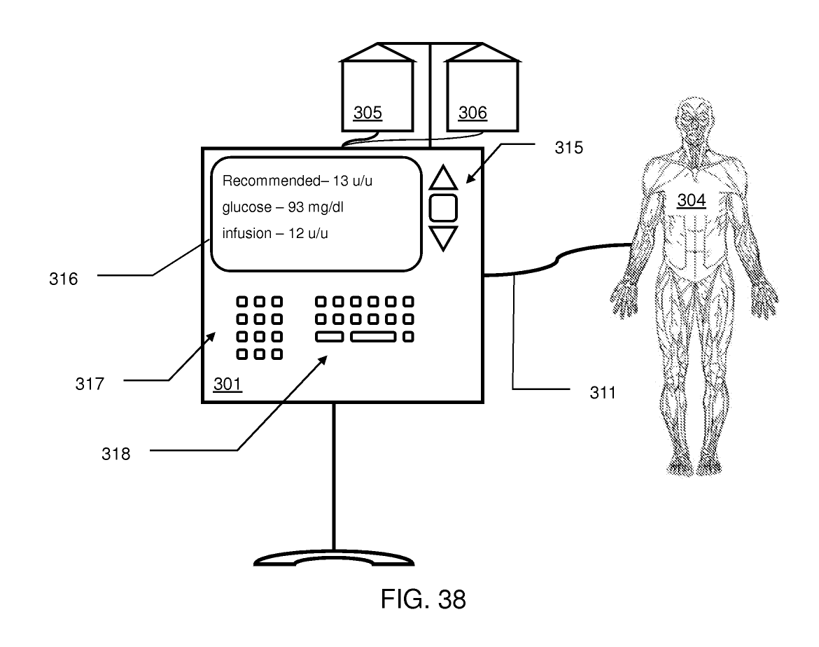

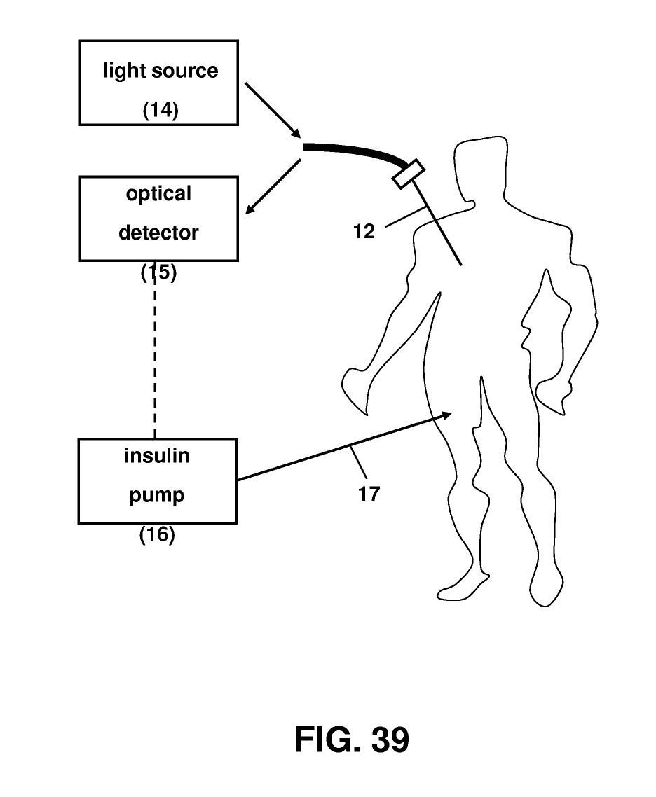

Indwelling Fiber Optic Probe for Blood Glucose Measurements FIG. 39 shows a schematic illustration of a glucose monitoring device comprising an indwelling fiber optic probe according to the present invention. A non-disposable illumination and collection fiber optic 11 can be coupled to a short disposable indwelling fiber optic probe 12 that can be integrated into a catheter that is inserted into a patient 13. The illumination portion of the fiber optic 11 can be connected to an ex vivo light source 14 for delivery of the illumination light to the patient tissue to be analyzed. The light source can be a near-infrared (NIR) light source, such as a thermal source, a tunable laser, or multiple lasers at selected wavelengths. The collection portion of the fiber optic 11 can be connected to an ex-vivo optical detector 15 for the detection of the tissue spectrum in the NIR spectral region. For example, the fiber optic probe 12 can be inserted intravascularly into blood tissue. Glucose in the blood can affect the detected transmitted or reflected tissue spectrum by absorption of light at the overtone and combination band wavelengths. For example, the detector 15 can comprise a Fourier transform infrared (FTIR) spectrometer. The detector 15 can further use signal processing methods, such as multivariate spectral analysis algorithms, to analyze the glucose-specific spectral features of the detected tissue spectrum. The device can further comprise an insulin pump 16 for infusing insulin 17 into the patient 13 in closed-loop response to the blood glucose measurement.

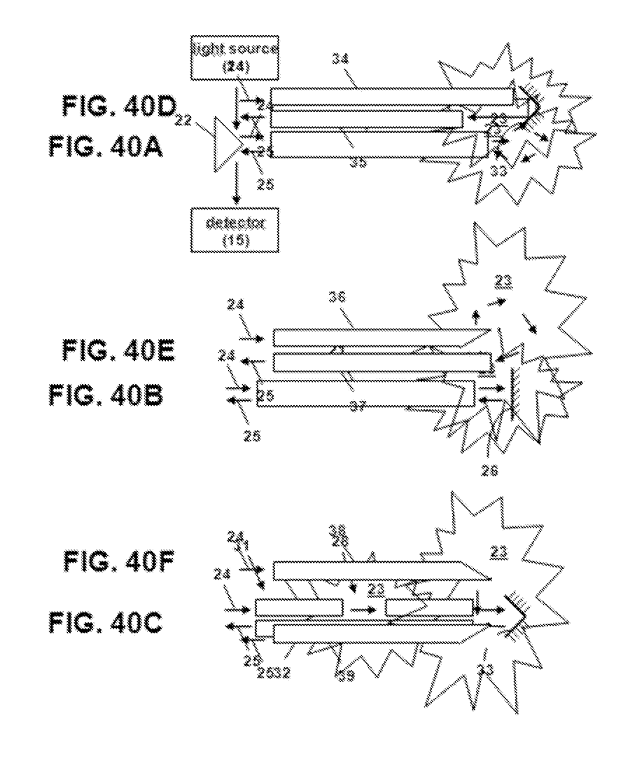

The fiber optic probe can comprise various illumination and collection optical configurations comprising one or more optical fibers having flat faced or shaped ends, and external optical elements, such as micromirrors and microlenses, to optimize the illumination and collection characteristics of the sample volume. Further, the numerical aperture, core and cladding materials, geometry, size, and arrangement and number of optical fibers can be chosen to optimize the delivery of light to and from the blood sample and to enable biocompatibility of the indwelling probe. The optical fibers can be contained in a catheter that can be inserted into a patient's tissue. FIGS. 40A-40F are schematic illustrations of some example optical configurations.

FIG. 40A shows an example configuration comprising a single optical fiber 21 that can be used for both the illumination and the collection of light that is diffusely reflected or scattered by the patient's blood. Near-infrared light 24 is provided by the light source 14 and is coupled into the proximal end of the optical fiber 21 by a reflecting wedge 22. The distal end of the optical fiber 21 can have a flat face for illuminating a blood sample 23 with the light 24 from the light source 14. For example, the fiber 21 can be integrated into a catheter that is inserted into the patient's blood stream and the blood can be sampled through a hole in the catheter. The light 24 can be scattered by the blood sample 23 and the scattered light 25 can be collected through the flat face of the distal end of the fiber 21. The collected light 25 is returned by the optical fiber 21 to the wedge 22 which reflects the collected light to the optical detector 15. Alternatively, lenses or similar optical elements can be used to couple the illumination light and collected light 25 into and out of the fiber. Alternatively, one or more separate illumination fibers can be used to illuminate the blood sample and one or more collection fibers can be used to collect the scattered light and return the collected light to the detector.

FIG. 40B shows an example optical configuration comprising a single optical fiber 21 for both the illumination and the collection of light that is both transmitted through and scattered by the patient's blood. NIR light 24 from a light source enters the proximal end and exits the flat face of the distal end of optical fiber 21 to illuminate the blood sample 23. Both transmitted and scattered light is collected by the fiber 21. Light that is transmitted through the sample is reflected by a flat mirror 26 at the distal end of the probe and is coupled, along with the scattered light, into the distal end of the fiber 21 as collected light 25. The optical path length to and from the end of the fiber to the mirror can be chosen to maximize the glucose signal. The collected light 25 is returned to an optical detector by the optical fiber 21. Alternatively, one or more separate illumination fibers can be used to illuminate the blood sample and one or more collection fibers can be used to collect the transmitted and scattered light and return the collected light to the detector.

FIG. 40C shows an example optical configuration comprising an illumination fiber 31 and a parallel collection fiber 32 that collects the illumination light that is transmitted by the patient's blood. The illumination fiber 31 can have a gap 28 separating a proximal portion 27 and the distal portion 29 of the fiber. NIR light 24 from a light source enters the proximal end of the proximal portion 27 of the fiber. A hole in the side wall of a catheter that contains the fibers can allow blood to flow across the gap 28 in the fiber. The illumination light 24 exits the flat face end of proximal portion 27 of the fiber and is transmitted through the blood sample 23 in the gap 28. The length of the gap 28 can be chosen to provide a suitable glucose signal based upon the penetration depth of the light 24 in the sample 23. The transmitted light enters the flat face entrance of the distal portion 29 of the fiber, exits the flat face end of the distal portion 29, and is reflected by a turning mirror 33 into a collection fiber 32. The collected light 25 is returned to an optical detector by the collection fiber 32. Additionally or alternatively, a gap can be provided in the collection fiber for transmission of the return light through the blood sample.

FIG. 40D shows an example optical configuration comprising an illumination fiber 34 and a parallel collection fiber 35 that collects the illumination light that is transmitted by the patient's blood. The distal end of the illumination fiber 34 is butted up to or in close proximity to the turning mirror 33. The distal end of the collection fiber 35 is retracted from the mirror 33 such that most of the optical pathlength is between the mirror 33 and the distal end of the collection fiber 35. This pathlength can be chosen to provide a suitable glucose signal based upon the penetration depth of the light 24 in the sample 23. The transmitted light enters the flat face distal end of the collection fiber 35 and the collected light 25 is returned to an optical detector by the collection fiber 35. Alternatively, the distal end of the collection fiber can be butted up to the turning mirror and the distal end of the illumination fiber can be retracted from the mirror to provide the desired optical pathlength.

FIGS. 40E and 40F show example optical configurations that use side-looking optical fibers having beveled ends for the collection of both scattered and transmitted light. In FIG. 40E, illumination light 24 from an NIR light source exits the beveled face of the side-looking distal end of an illumination fiber 36 and is scattered by the blood sample 23. Some of the scattered light is collected by the flat-face distal end of a collection fiber 37 and the collected light 25 is returned to an optical detector. In FIG. 40F, illumination light 24 from a side-looking illumination fiber 38 is collected by a side-looking collection fiber 39 and the collected light 25 is returned to an optical detector. This optical configuration preferentially collects light that is transmitted through the blood sample 23.

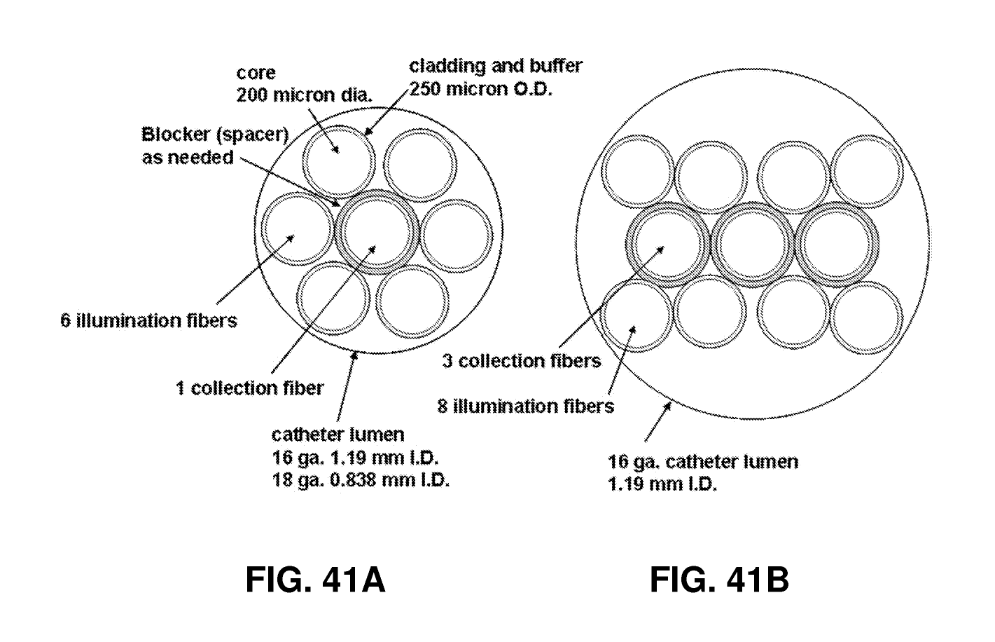

The small dimensions of optical fibers allow multiple illumination and collection fibers to be bundled into a single catheter. FIGS. 41A and 41B show examples of illumination and collection fiber geometries that are compatible with 16 and 18 ga. catheters. The catheter lumen can comprise at least one illumination fiber and at least one collection fiber. The spacing between the illumination and collection fibers, the number of fibers, and the size of the fibers can be optimized to improve the detected signal. The fibers can be step- or gradient-index fibers comprising a high refractive index core and a lower refractive index cladding for efficient guiding of near-infrared light. The core of the fibers can comprise an optical material, such as glass or silica, that is transparent in the near-infrared. The examples shown are for optical fibers with a 200 micron core with a cladding to provide a 250 micron outside diameter fiber.

FIG. 41A shows an example fiber optic probe comprising a catheter containing six parallel illumination fibers surrounding a central collection fiber. The collection fiber can have an opaque blocker or spacer on the outside of the cladding layer to inhibit cross-talk with the illumination fibers. As examples, the catheter lumen can be 16 ga. (1.19 mm inside diameter) or 18 ga. (0.838 mm inside diameter).

FIG. 41B shows an example fiber optic probe comprising a catheter having two planes of four illumination fibers each surrounding a central plane of three collection fibers. As an example, the fibers can be contained in a 16 ga. catheter lumen having a 1.19 mm inside diameter.

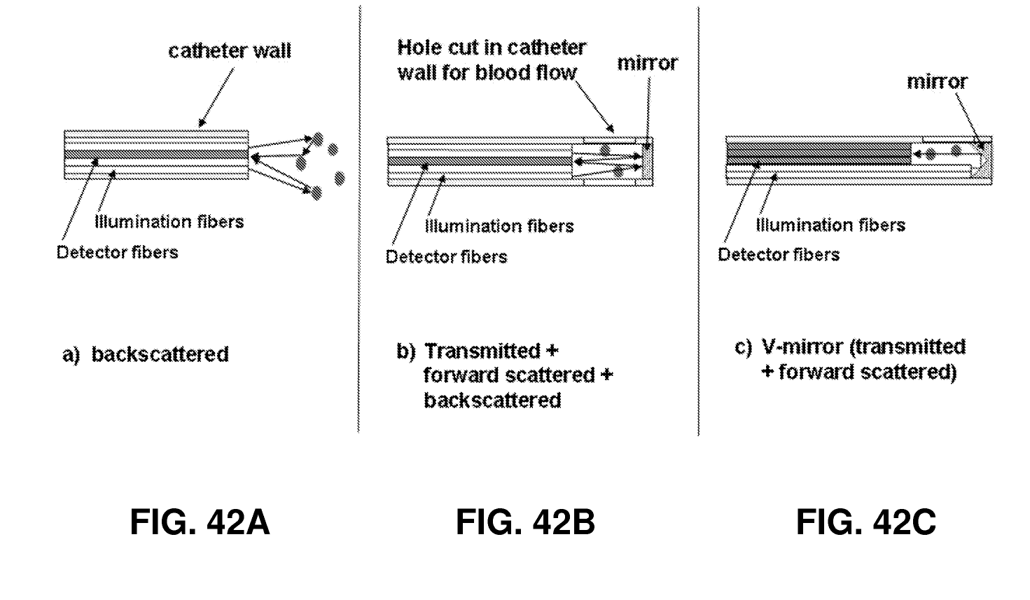

FIGS. 42A-42C show example probe constructions that comprise the optical configurations shown in FIGS. 40A, 40B, and 40D, respectively.

FIG. 42A shows an example probe wherein light from peripheral illumination fibers is backscattered by the blood and the backscattered light is collected at a central collection, or detector, fiber. The fibers can be contained in a catheter having an open distal end exposed to the blood sample. The number of illumination and collection fibers, and their arrangement, controls the pathlength and magnitude of signal detected. The shape of the probe tip and individual fibers can be designed to provide a suitable signal for detection.

FIG. 42B shows an example probe wherein transmitted, forward scattered, and backscattered light is collected by a central collection fiber. Blood flows across the probe through a hole cut in the catheter wall. The distance from the fibers to the mirror allows control of the optical pathlength. Backscattered light (not reflected by the mirror) can also be collected.

FIG. 42C shows an example probe optimized for a transmission measurement which collects transmitted light only. The illumination fibers are butted up to the turning mirror and the collection fibers are retracted from the mirror. In this configuration, the optical pathlength is controlled by the spacing of the collection fibers to the turning mirror. Blood flows across the probe through a hole in the catheter wall.

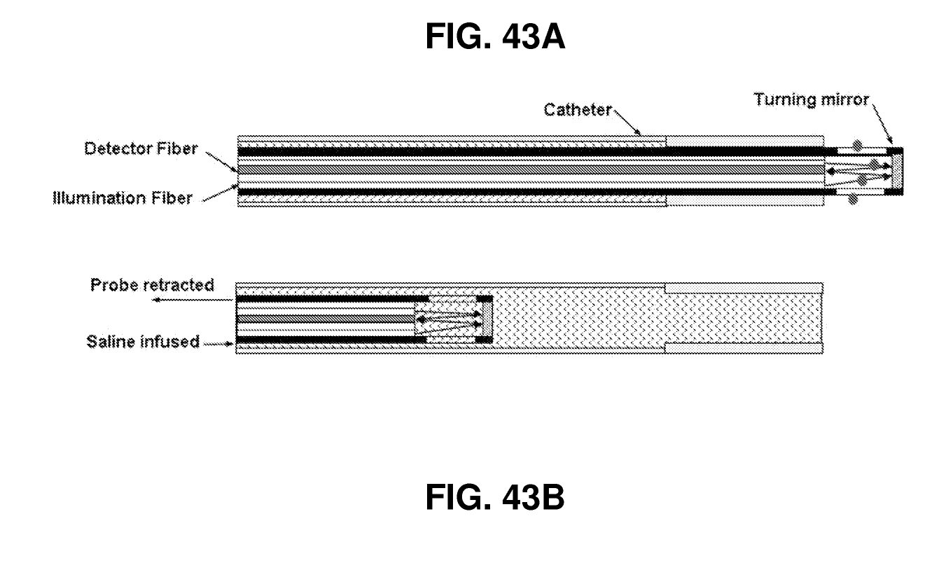

The optical probe can be configured to enable a background reference measurement. FIGS. 43A and 43B show example probes for collecting a reference saline background measurement. FIG. 43A shows the probe in a sample-measuring configuration, similar to the optical configurations shown in FIGS. 40B and 42B. In FIG. 43B, the fiber probe is shown retracted within the catheter lumen. The catheter is sealed and saline is infused into the catheter housing. The infused saline can flow around the probe, enabling a reference saline background measurement.

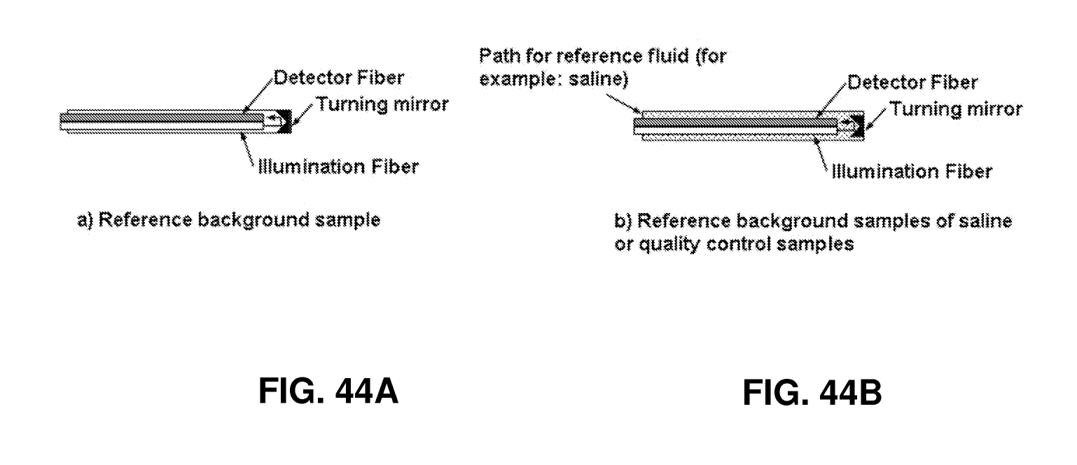

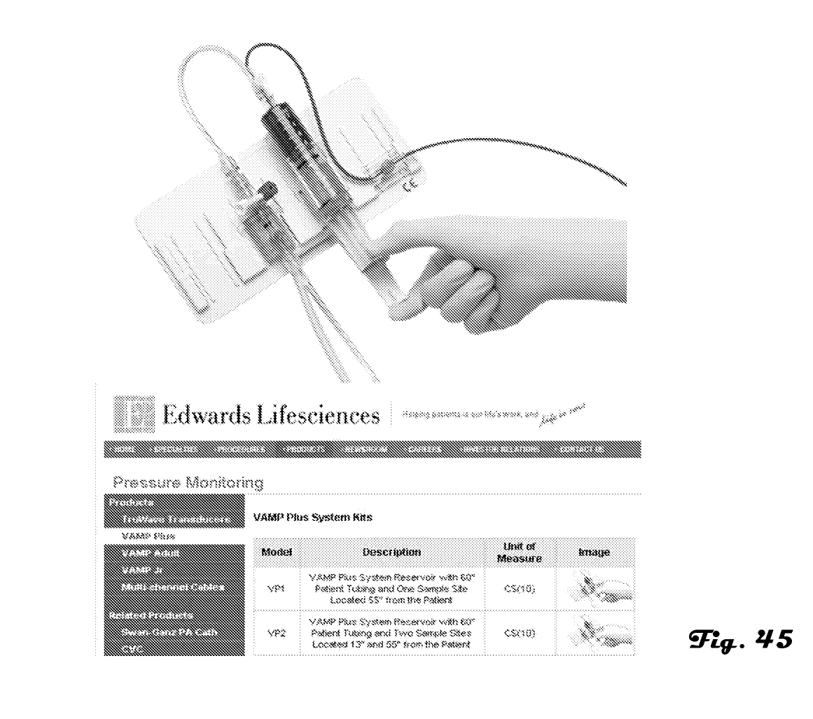

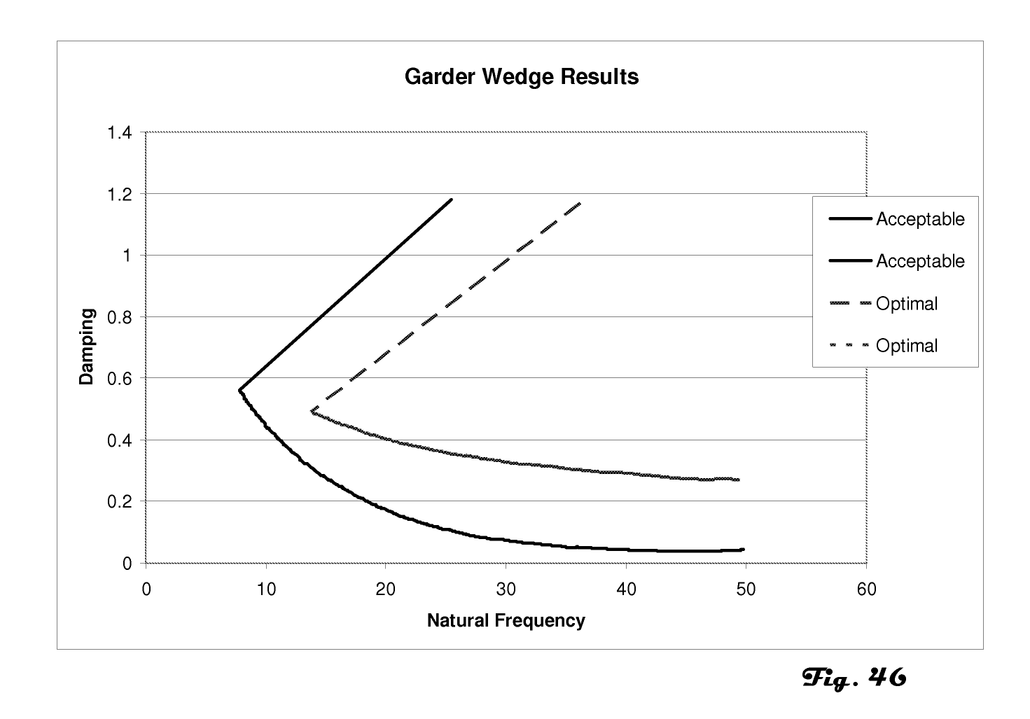

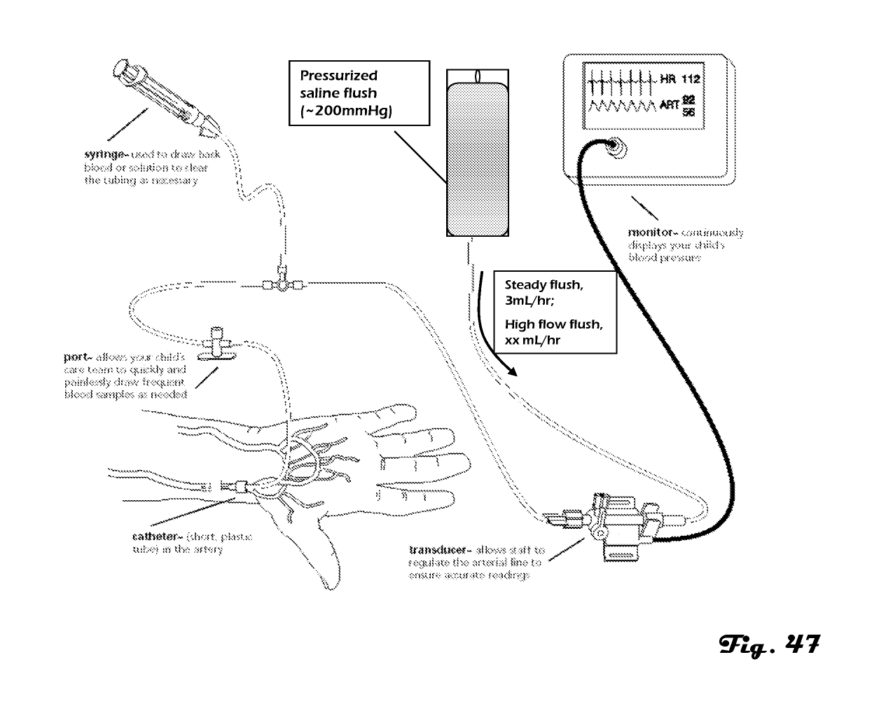

FIGS. 44A and 44B show example probe configurations for an auxiliary fiber optic measurement. FIG. 44A shows an example reference background probe that uses illumination and detection fibers and a turning mirror, but without the sample. The extra channel of information from the reference background probe can be used to compensate for spectra effects resulting from bending of multimode fiber optics. The background probe can run along side the sample probe fibers that are used for the blood measurement, but would not be indwelling. The probe can also provide a reference background measurement to compensate for the stability of the sample probe or to simply monitor the health of the sample probe. FIG. 44B shows an example auxiliary fiber probe that incorporates a fluid measurement, such as saline, within the housing of the probe. This probe can be used as another method of background correction for the sample measurement probe.

An apparatus for hemodvnamic monitoring and analyte measurement The sharing of a single arterial access site for both hemodynamic monitoring as well as blood sample procurement requires attention to a variety of implementation details. In simple terms the automated measurement system should not: (1) change or influence the dynamic response of the hemodynamic monitoring system; (2) create pressure gradients that result in inaccurate measurements; or (3) introduce bubbles. Any of the above may create a situation where the hemodynamic values are inaccurate.

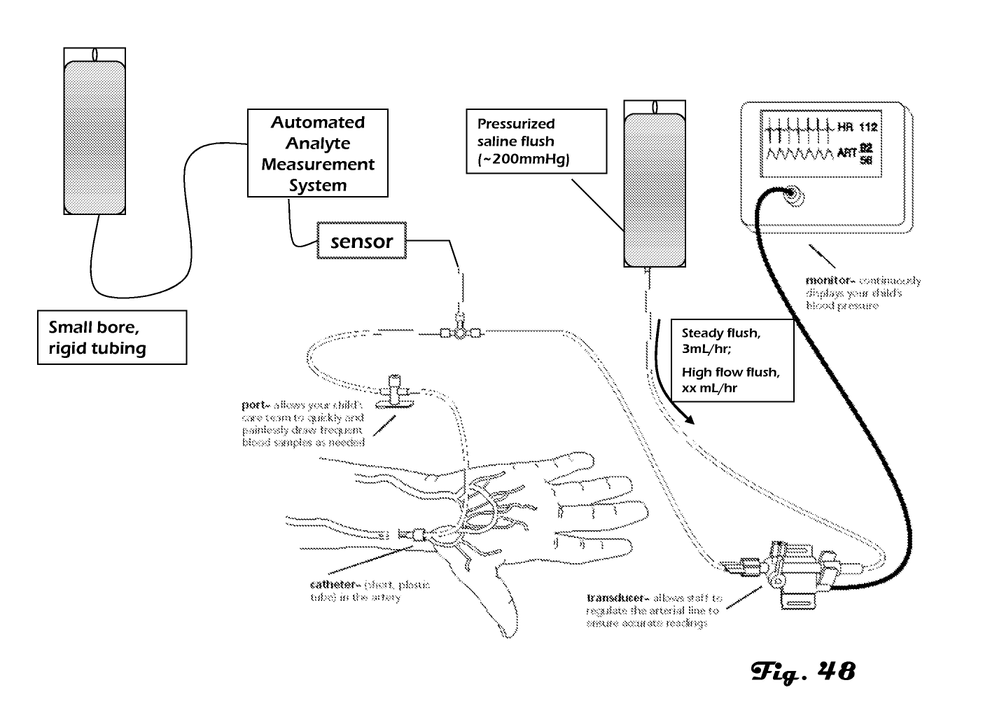

As shown in FIG. 48 an automated sample acquisition and measurement system can be attached in a similar manner. If a stopcock creating a T-junction (typically referred to as a 4-way stopcock) is used then the effects on the hemodynamic trace can be significant. The attachment of the automated blood measurement system can alter the overall response characteristics of the system such that accurate pressure measurements cannot be obtained. Most hemodynamic alarm systems have a minimum pulse pressure as well as a minimum diastolic pressure. The influence of the automated blood measurement system can be mitigated by closing the stopcock before each measurement. However this creates another problem as each measurement is not sufficiently automated due to the need for manual intervention with each sample.

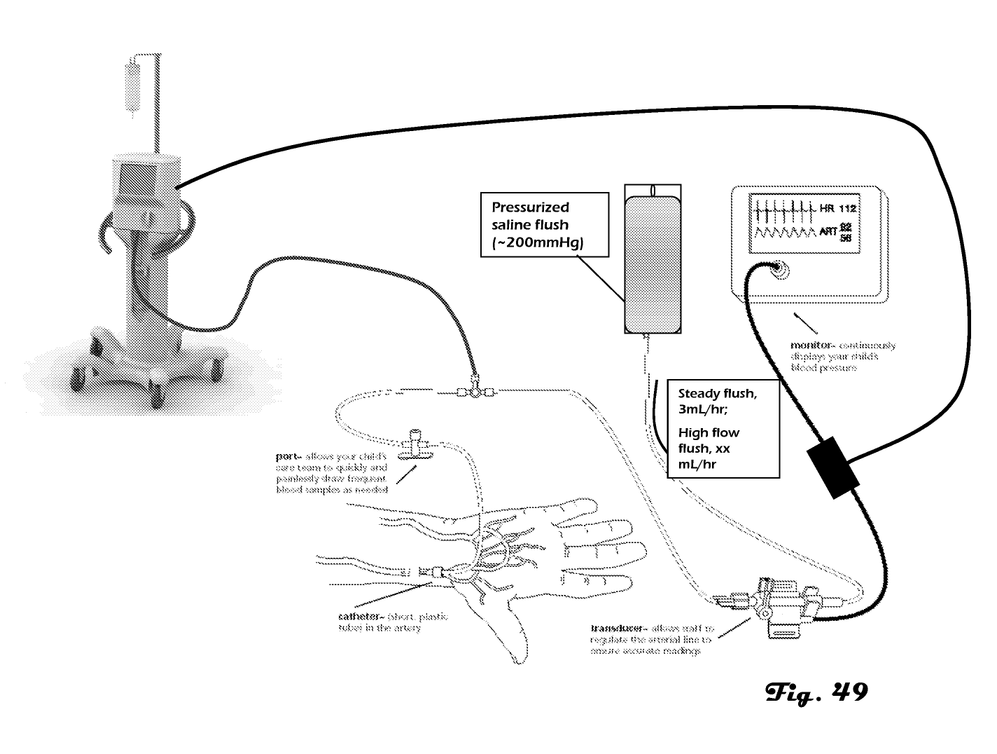

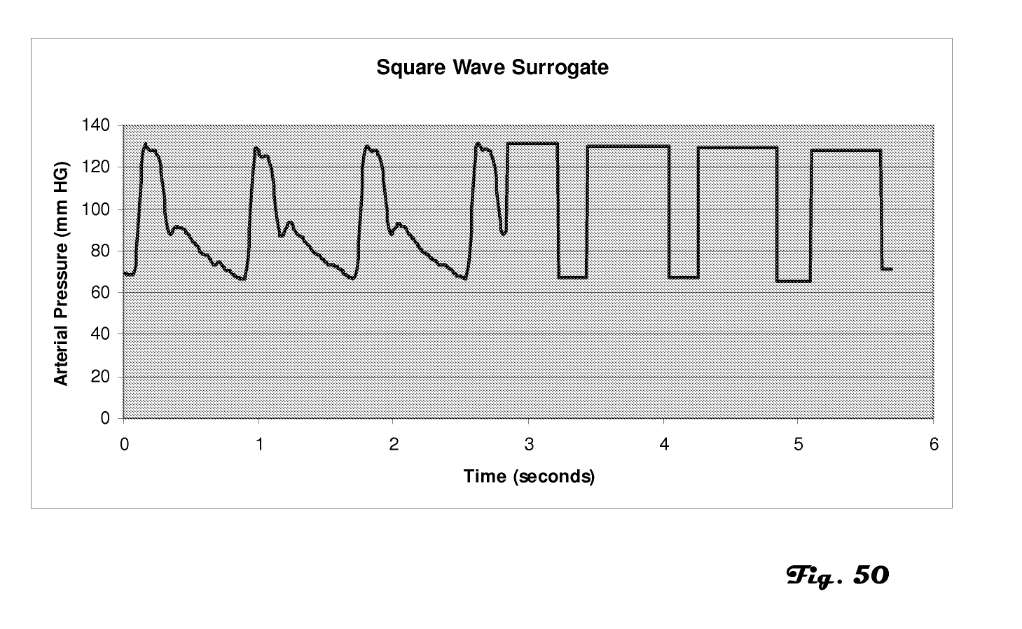

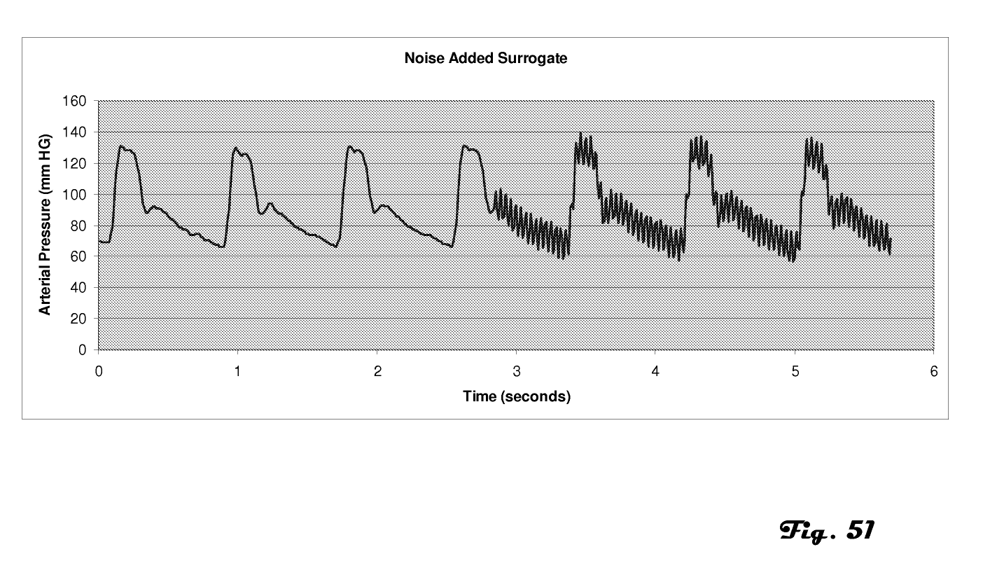

FIG. 49 is a schematic illustration of an example embodiment that addresses the monitoring problems discussed above. In the example embodiment shown, an automated blood glucose monitoring system has the ability to alter, replace or override the signal being delivered from the pressure transducer to the hemodynamic display. The resulting signal will be referred to as a surrogate signal. In FIG. 49, this is shown as a physical connection to the cable between the hemodynamic monitoring system and the pressure transducer. The communication or transfer of information between these two systems can be provided by many embodiments including, as examples, wireless communication or other communication means. An alternative embodiment has a cable from the transducer going to the automated blood measurement system and then a separate cable going from the automated blood measurement system directly to the hemodynamic display. During the period of time that the automated blood analyte measurement causes a disruption of the hemodynamic trace, the signal display on the hemodynamic monitor can be replaced by a surrogate signal. The surrogate signal can be similar to the prior hemodynamic trace but altered in a way that the clinician can readily determine that it is a surrogate or artificial trace. An example of such a surrogate signal is a square wave where the top of a square wave matches the systolic pressure, the bottom of the square wave matches the diastolic pressure and the frequency is the same as the prior arterial waveform. Most arterial pressure monitoring systems do not have the diagnostic capabilities to recognize such a surrogate signal and would therefore not alarm during its use. As further examples, the display of either the automated blood analyte monitor or the hemodynamic monitor can be altered by an alteration in color or background of the display, display of error messages, or by a variety of other means.

FIG. 50 shows an example of a surrogate square wave signal trace. The left-hand portion of the graph shows a true signal (reflective of the actual pressures in the artery) while the right hand portion of the graph shows a square wave with similar measurement values and frequency.

FIG. 51 shows an example of an example surrogate signal trace. The left-hand portion of the graph shows a true signal while the right hand portion of the graph shows a replication of the true signal with a noise artifact added on.

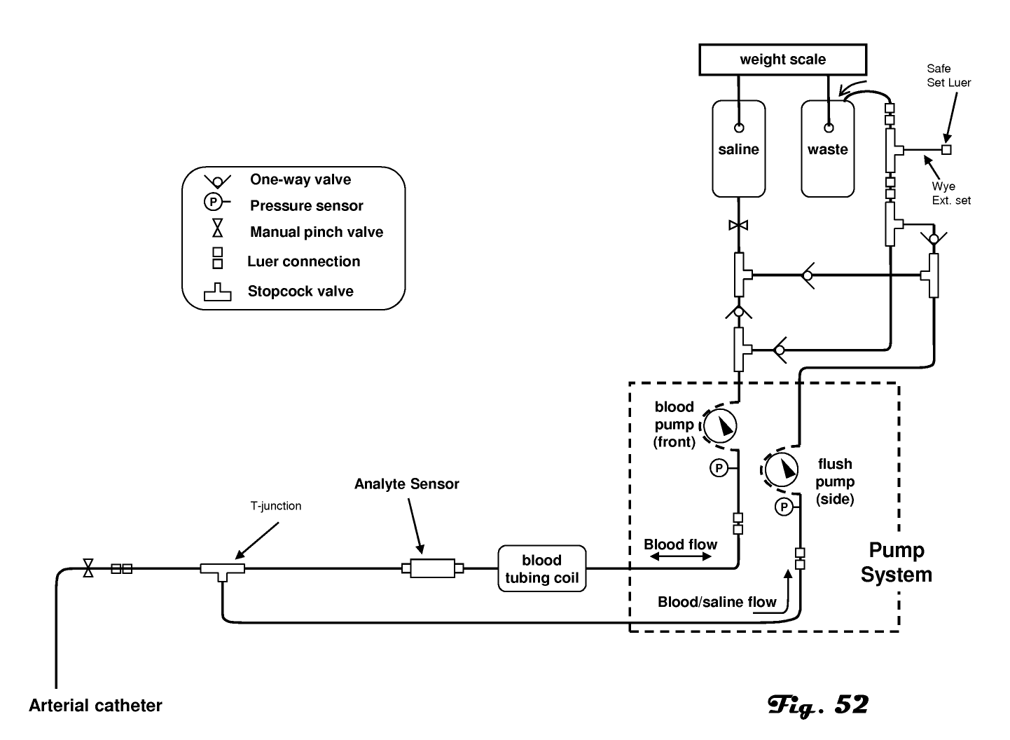

FIG. 52 is an example of an automated blood analyte measurement system. This system differs from the one illustrated in FIG. 48 in that the example system in FIG. 52 has a second tubing loop and pressure transducer that enables more effective cleaning. The blood access system shown in FIG. 52 contains two pressure transducers. During the withdrawal of blood up to the analyte sensor the pressure transducer associated with the blood pump is able to provide real-time pressure measurements associated with the blood withdrawal. During the withdrawal sequence the pressure transducer associated with the flush pump is able to effectively sense the pressure at the T-junction. The information content provided by both pressure transducers as well as the state of each blood pump can provide the basis for pressure measurement during the withdrawal sequence.

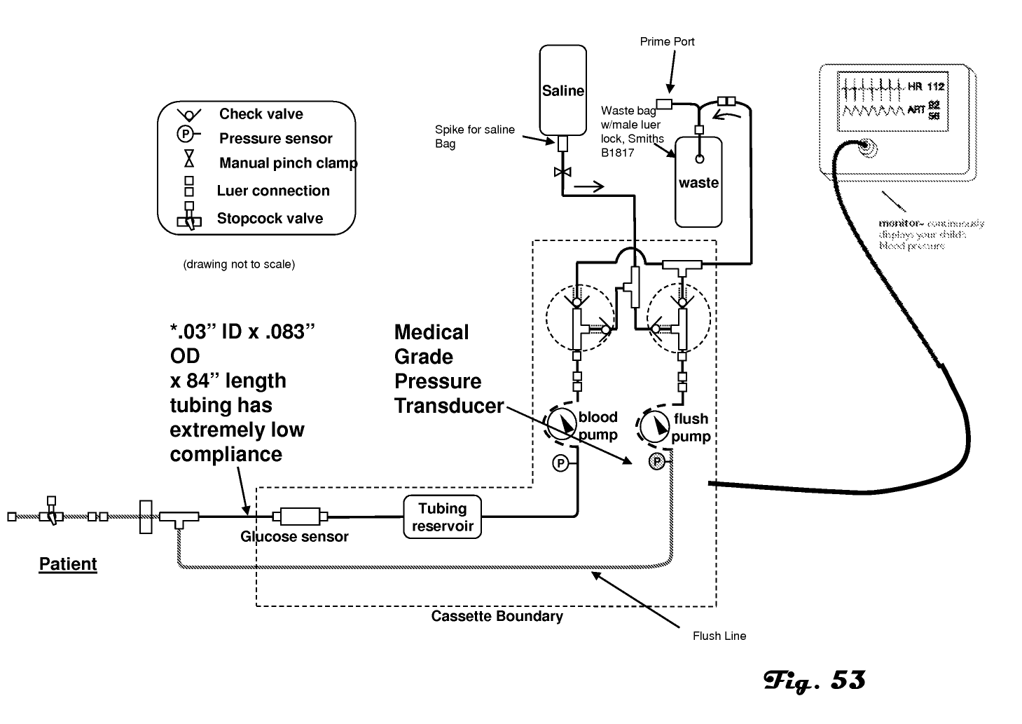

FIG. 53 is an illustration of an example embodiment of an automated blood measurement system that provides concurrent hemodynamic monitoring during the blood analyte measurement process. The automated blood withdrawal system provides a pressure signal for display on a hemodynamic monitor. In operation the blood access system is attached to the arterial catheter (not shown) and saline infused to keep the access site open is provided by the blood access system via the associated pressurized saline bag. At the time an automated blood analyte measurement is initiated, the system can stop the saline infusion into the arterial catheter and initiate a blood withdrawal process. The stoppage of flow typically present to maintain arterial access patency is desired as it enables an undiluted sample to be obtained. As the infusion rates for maintenance of catheter patency may vary by hospital, IV tubing set-up, the pressure of the bag, etc, the ability to procure an undiluted sample is an advantage of the combined system. Through the use of both pressure transducers as well as knowledge regarding the state of both pumps, the system has knowledge of the pressure artifact being created by the automated blood measurement system. These artifacts can be due to the blood withdrawal process, calibration, cleaning, infusion or fluid movement associated with the measurement cycle. Due to knowledge of the artifact created (duration, type and magnitude) the system can create a surrogate signal as described above during the period when the artifact exceeds an acceptable clinical threshold.

Instead of providing a surrogate signal, the system also has the ability to compensate for the pressure artifact being introduced by the automated blood measurement system. Through the use of both pressure transducers as well as knowledge regarding the state of both pumps, the pressure artifact can be determined enabling the determination of the true pressure at the arterial catheter. This process enables the procurement of an undiluted blood sample to the measurement system while concurrently affording real-time hemodynamic monitoring. The ability to determine the pressure gradients being produced by the automated blood measurement system enables hemodynamic monitoring to continue during a greater portion if not all of the measurement cycle. The provision of an accurate pressure trace during the entire automated analyte measurement sequence means that the patient's hemodynamic status and associated alarm methodologies remain fully operational and active during the automated blood analyte measurement.

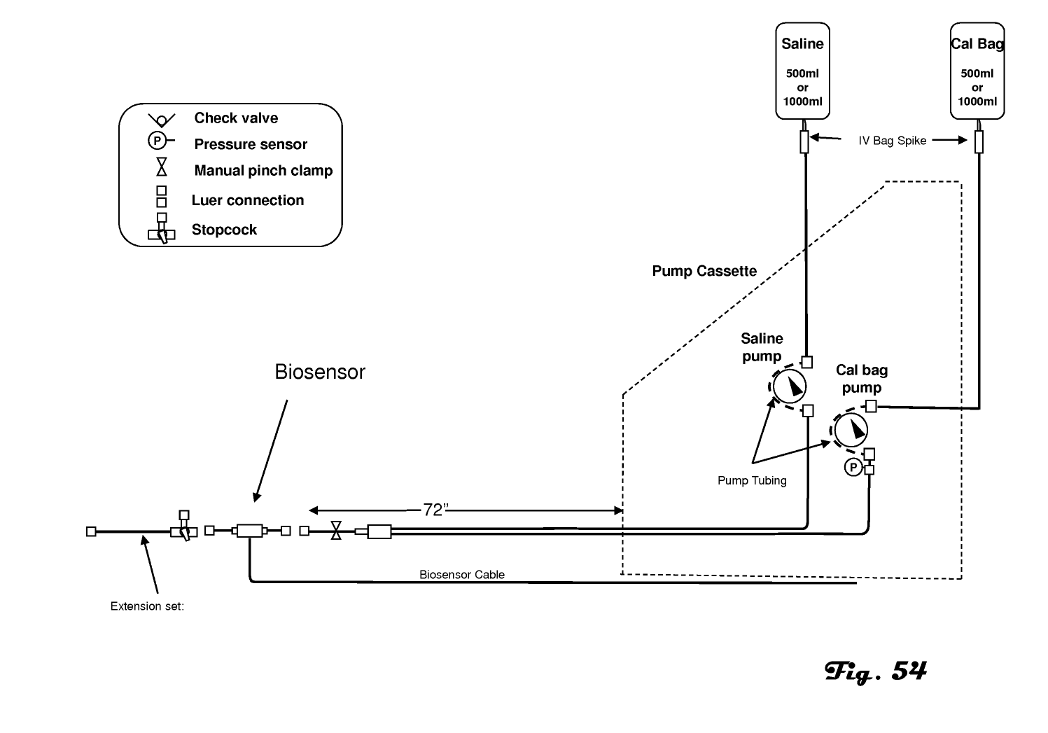

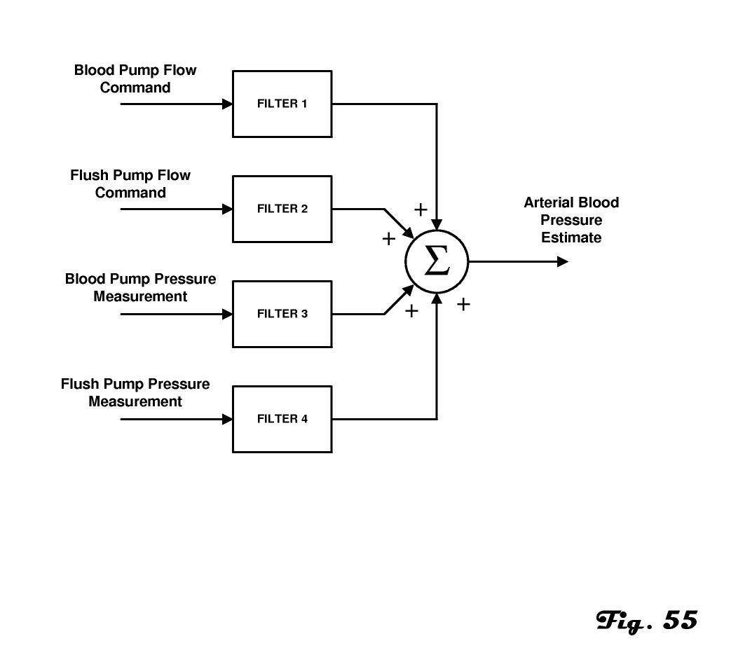

FIG. 54 shows another example embodiment of a blood access system where the sensor is located close to the patient. As shown the blood access system has only one pressure transducer but others can be added. This system with the blood sensor located more proximal to the patient also has the ability to generate surrogate signals as well as to provide direct artifact compensation. FIG. 55 shows an example of an estimator structure suitable for use with embodiments of the present invention such as that in FIG. 53. The disclosed structure enables estimation of the arterial pressure wave during the measurement process. As shown in the example estimator, the inputs to the estimator function are the blood pump flow commands, the flush pump flow commands, the blood pump pressure measurements and the flush pump pressure measurements. These commands can be utilized by a model based estimation function to provide continuous arterial blood pressure waveforms.

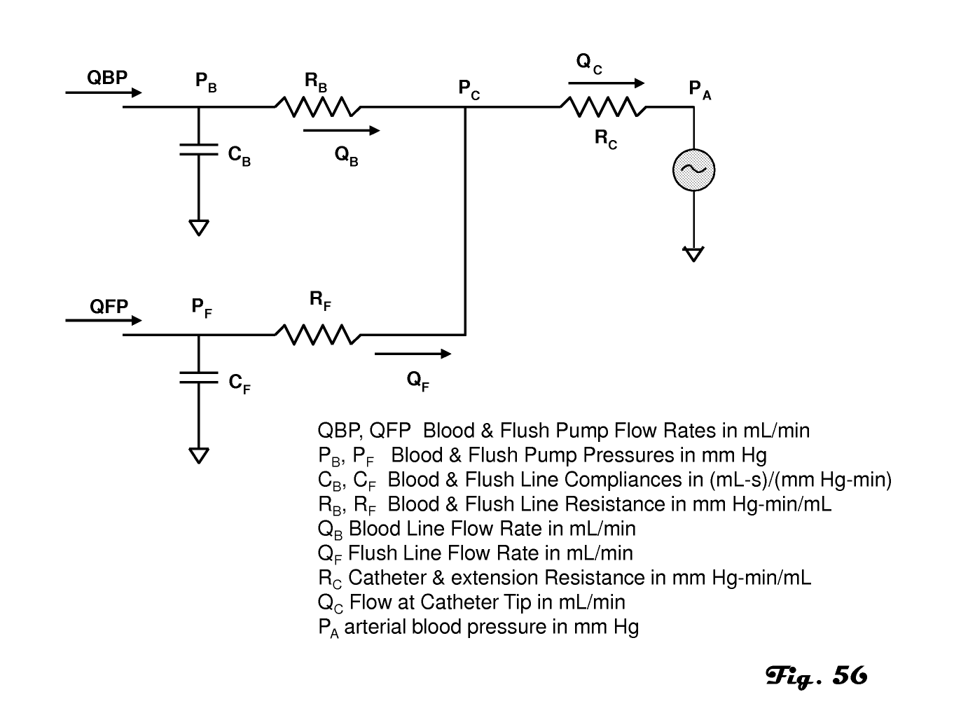

FIG. 56 shows an example method for modeling the performance of the blood access system. This model provides the basis for creating a lumped parameter linear dynamic model. The use of a linear electrical circuit analogy with multiple inputs and multiple outputs provides a basis for determining the arterial pressure during the measurement sequence. The compliances and resistances of the circuit can be accounted for in the model. The flow commands to the pump as well as the pressure measurements made can be utilized as inputs into this model to enable an estimation of the arterial pressure output. The result is a filtered linear combination of measurements and input commands for the effective estimation of the arterial pressure under any set of operational conditions.

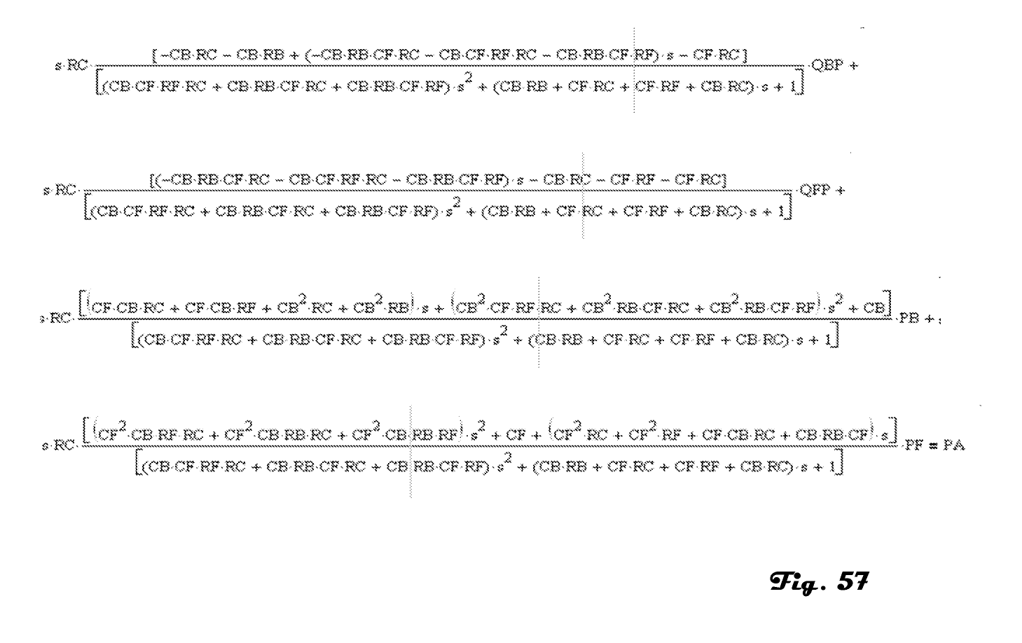

FIG. 57 is an illustration of equations that can be used to estimate the arterial pressure. As an example implementation, these equations can be programmed into the automated analyte measurement system.

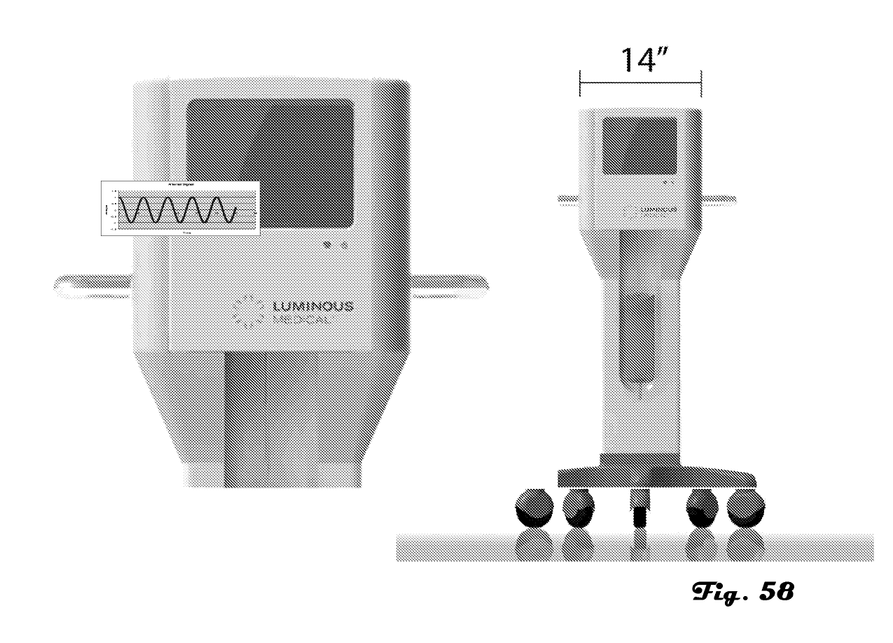

FIG. 58 is an illustration of an alternative embodiment where the arterial pressure trace or hemodynamic monitoring information is displayed on the automated blood analyte system console. In this case the automated analyte system provides analyte measurement results as well as arterial pressure measurements. The console displayed is one from Luminous Medical (a trademark of Luminous Medical, Inc.).

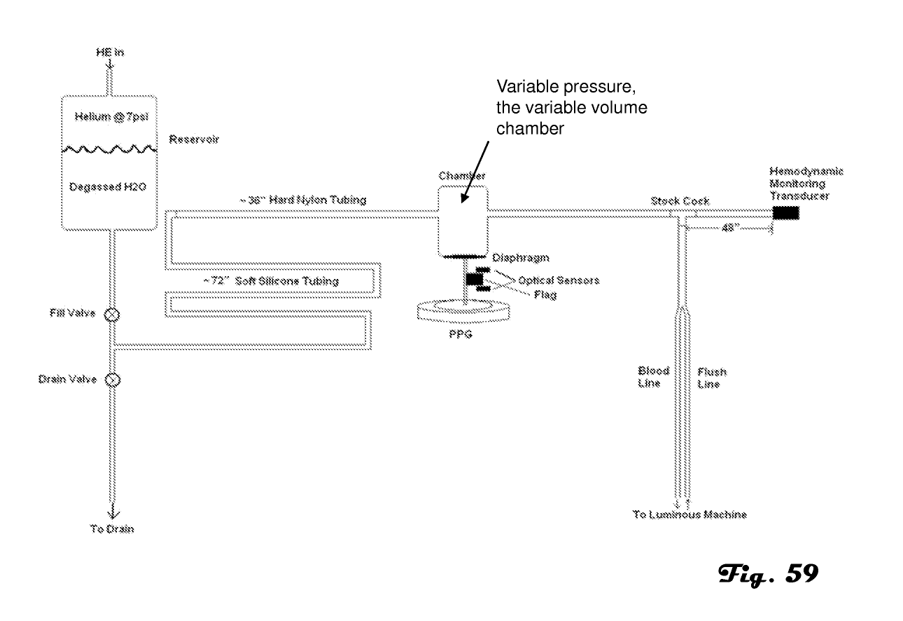

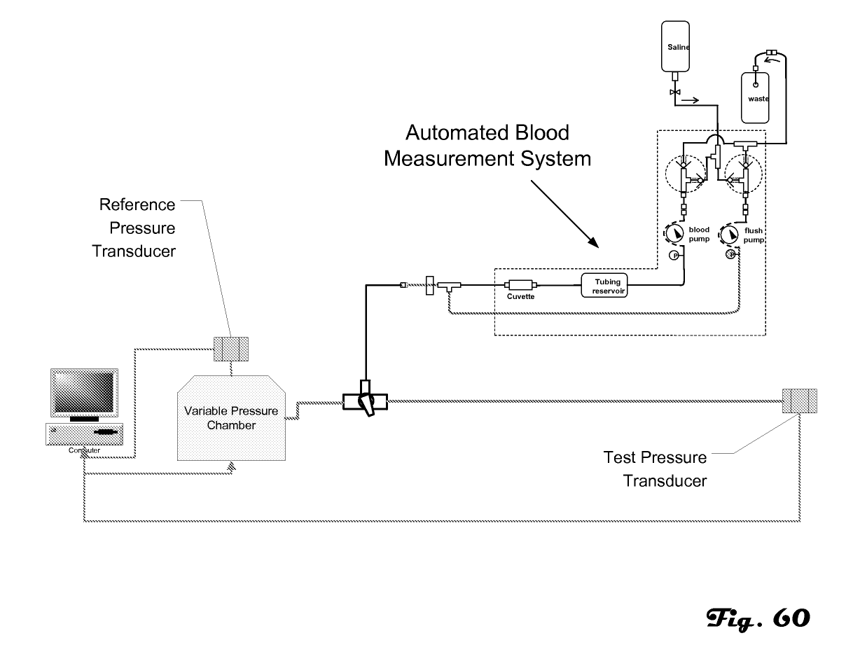

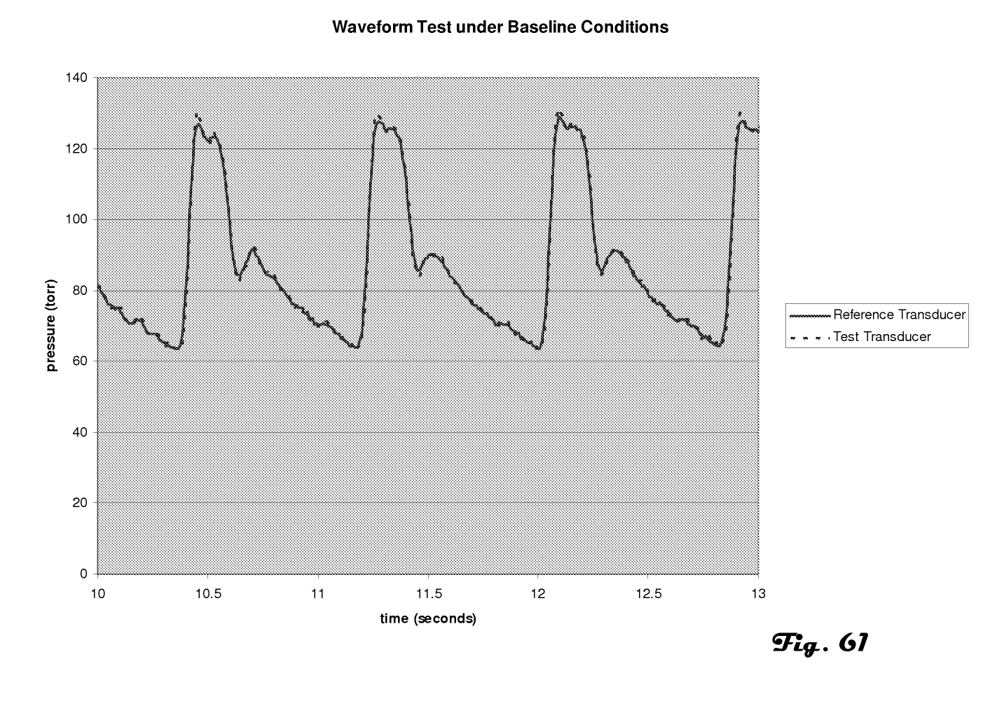

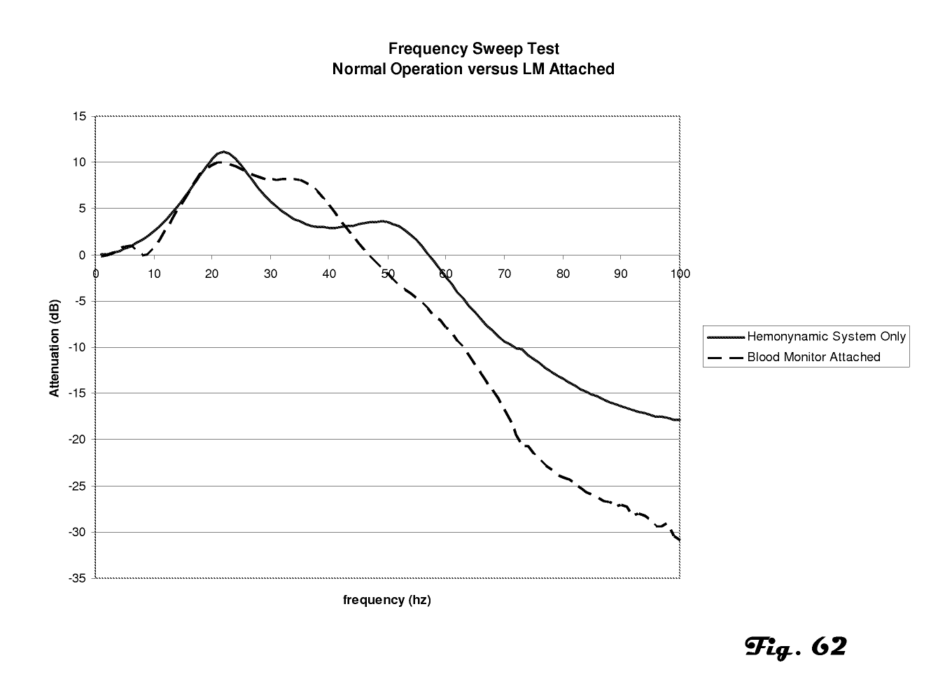

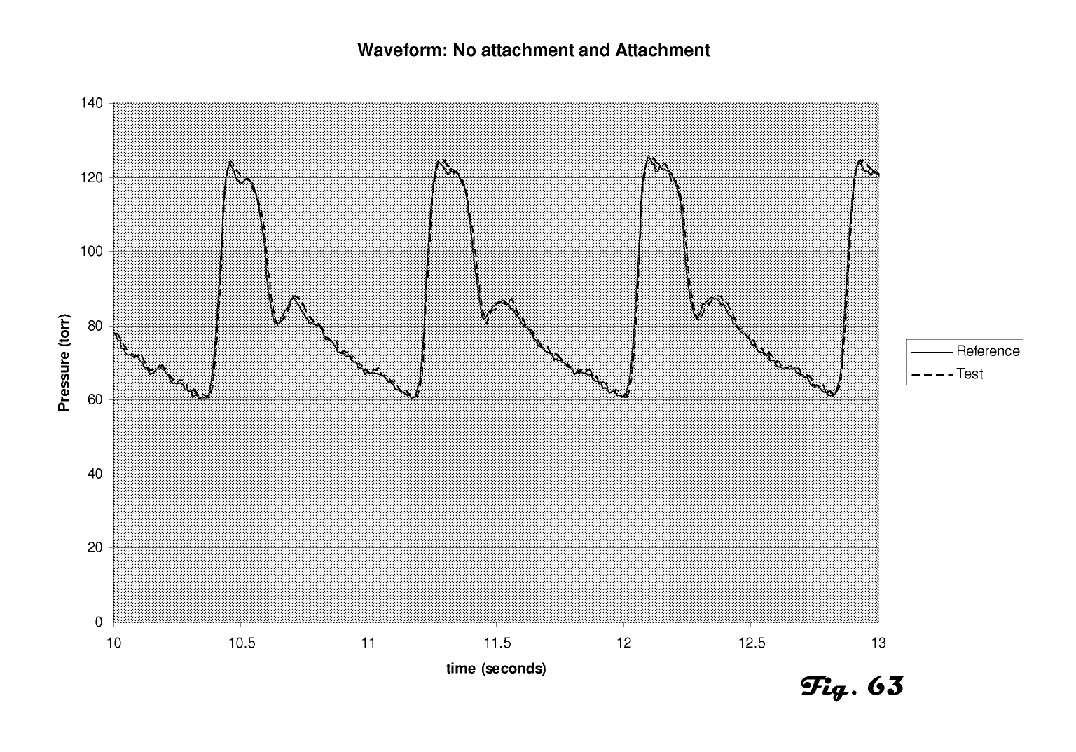

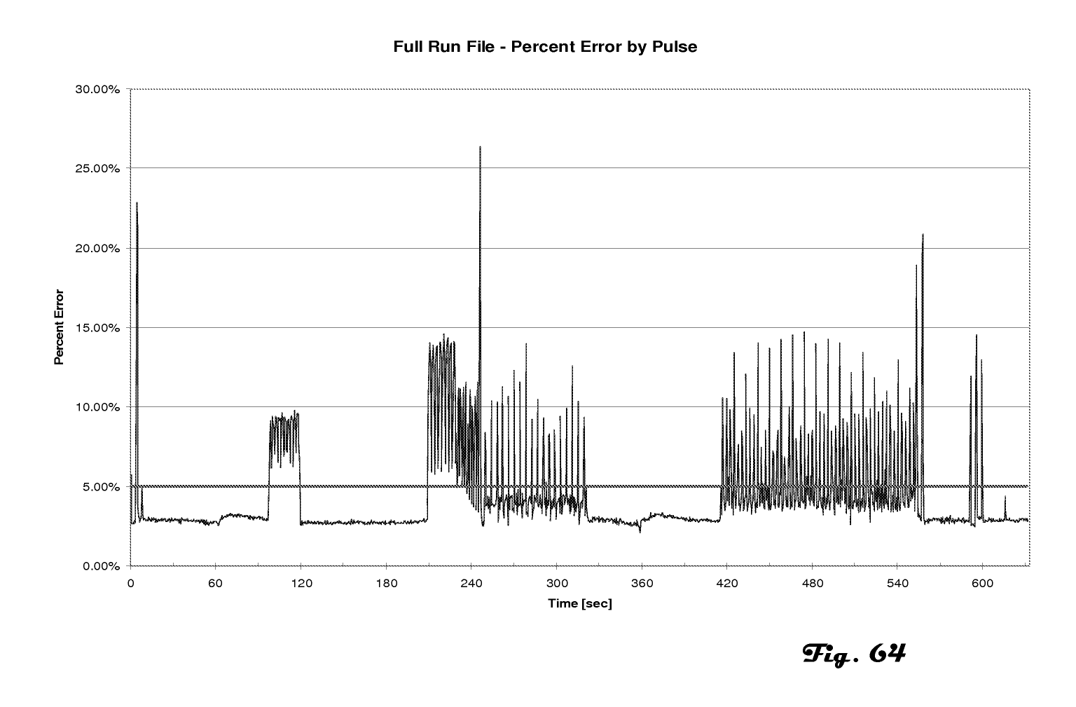

A volume control mechanism maintained the volume of the chamber so that the voice coil operated within its normal/linear range. FIG. 59 shows the overall system configuration. FIG. 60 shows the relationship between the pressure transducers under test and their relationship to the variable pressure chamber. A reference pressure transducer records the pressure generated at the artificial patient while a second test transducer records the pressures in a configuration that mirrors a conventional hemodynamic monitoring setup. Comparison between the reference and test readings enables determination of measurement errors. FIG. 61 shows an illustrative arterial pressure tracing.

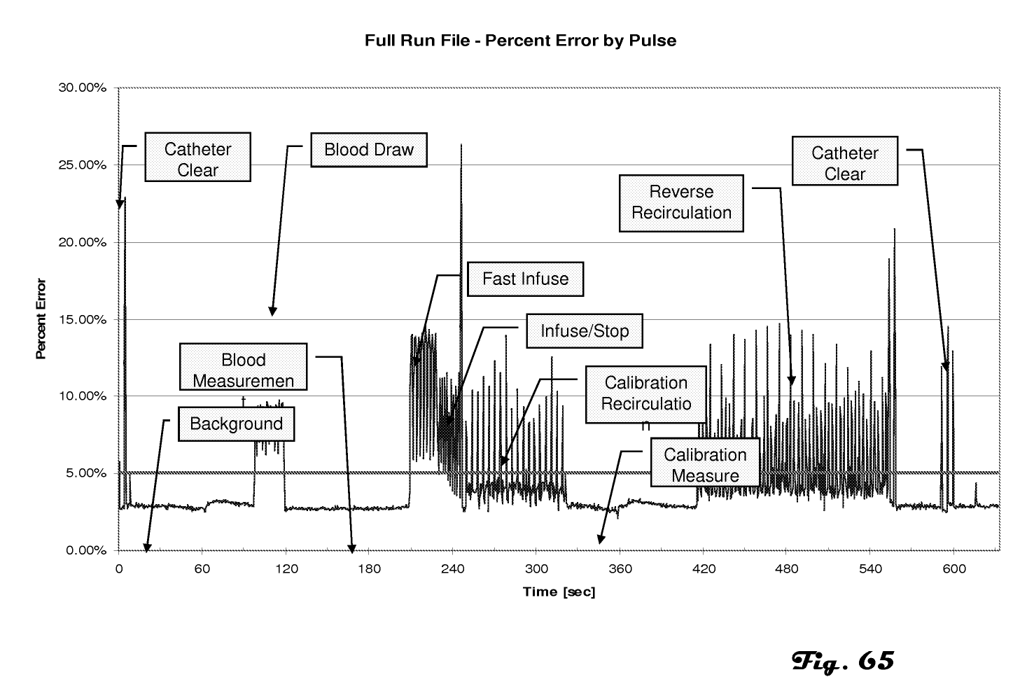

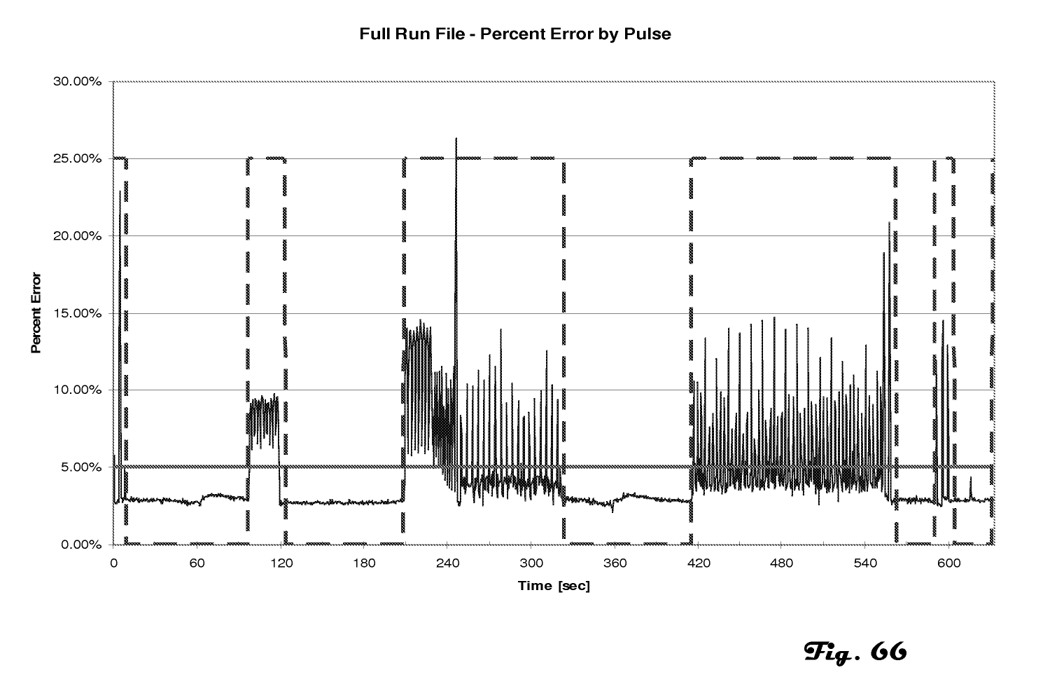

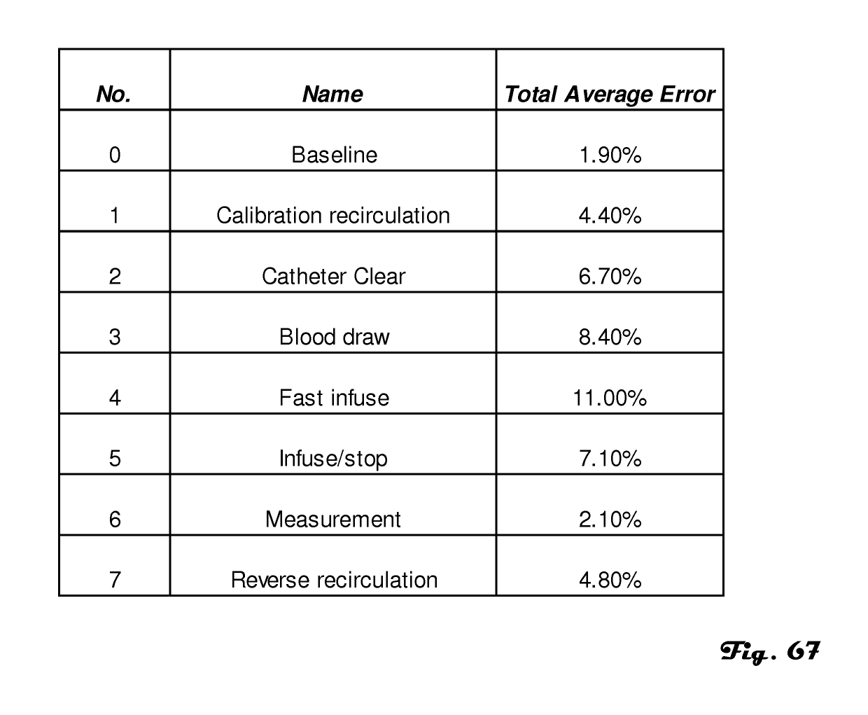

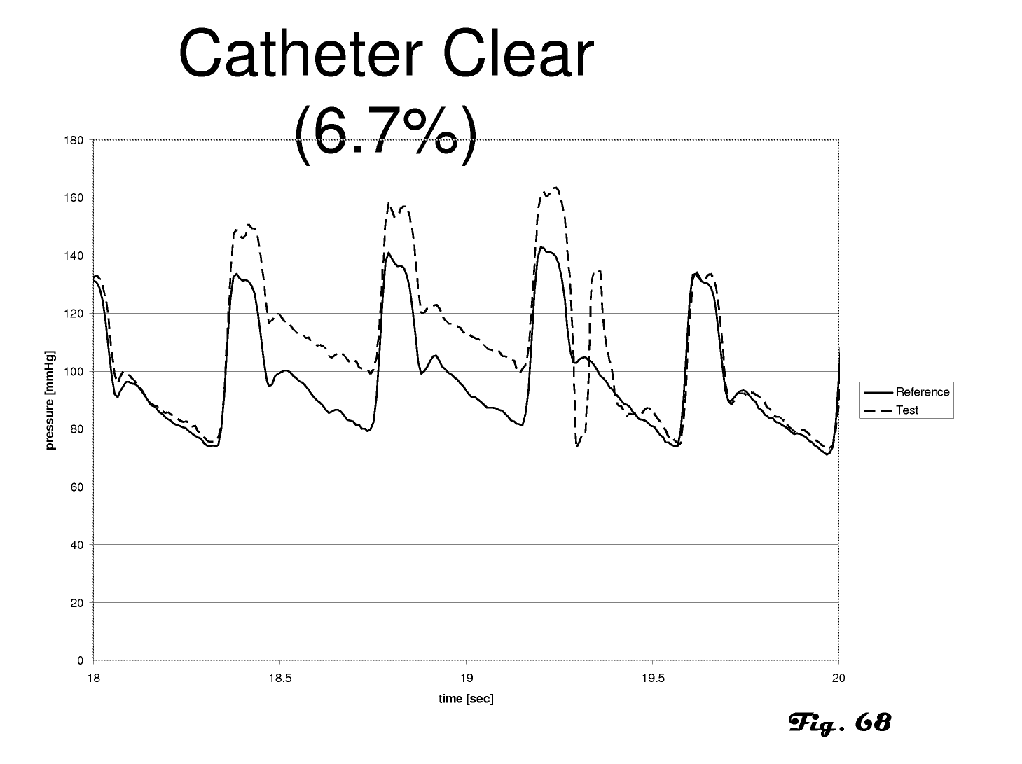

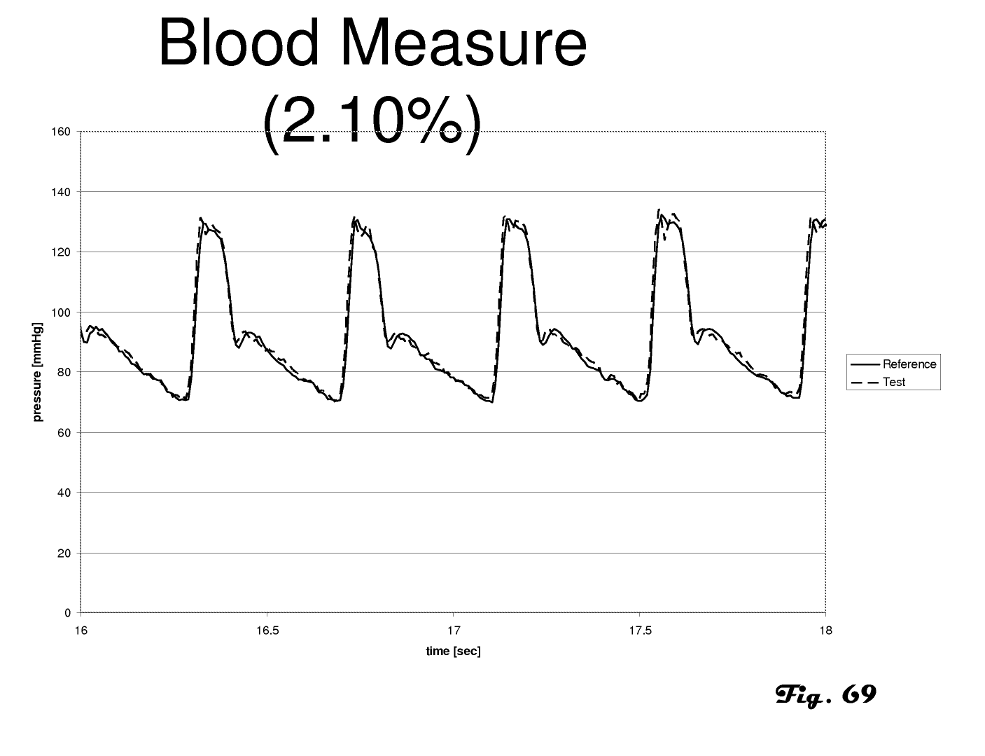

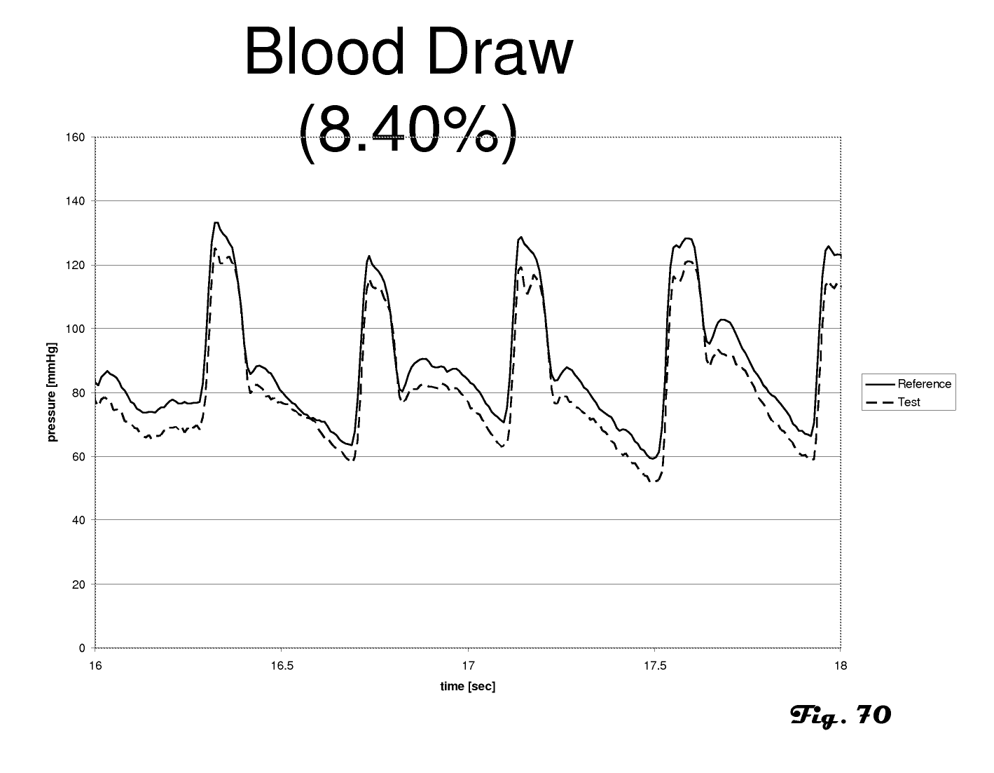

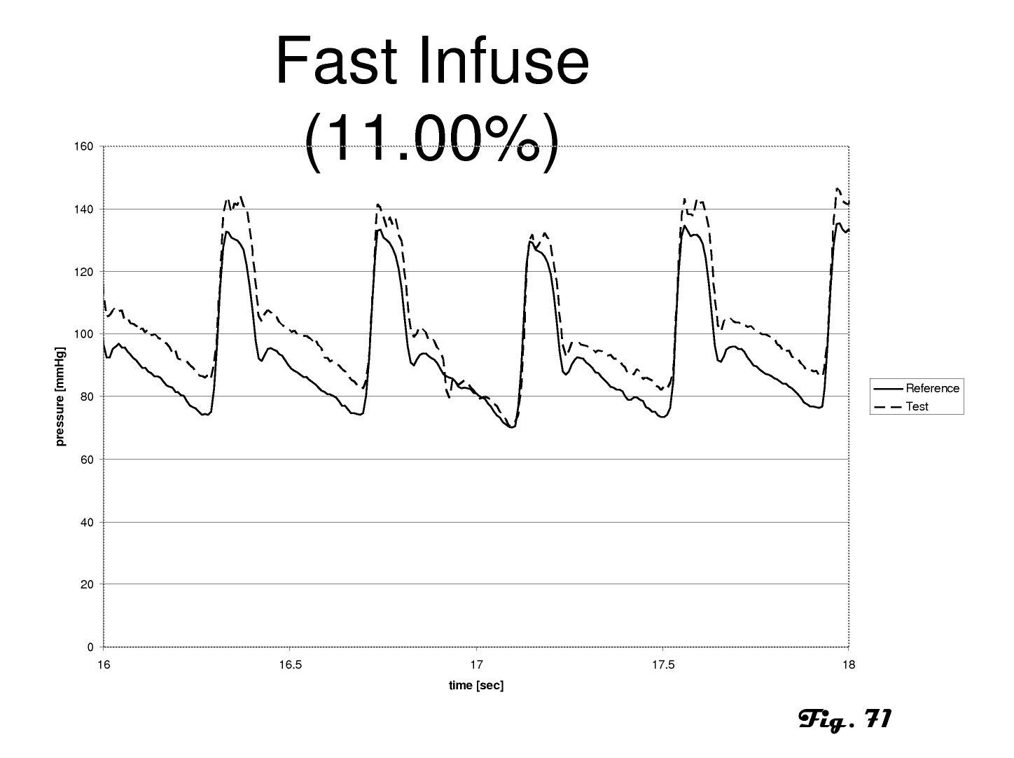

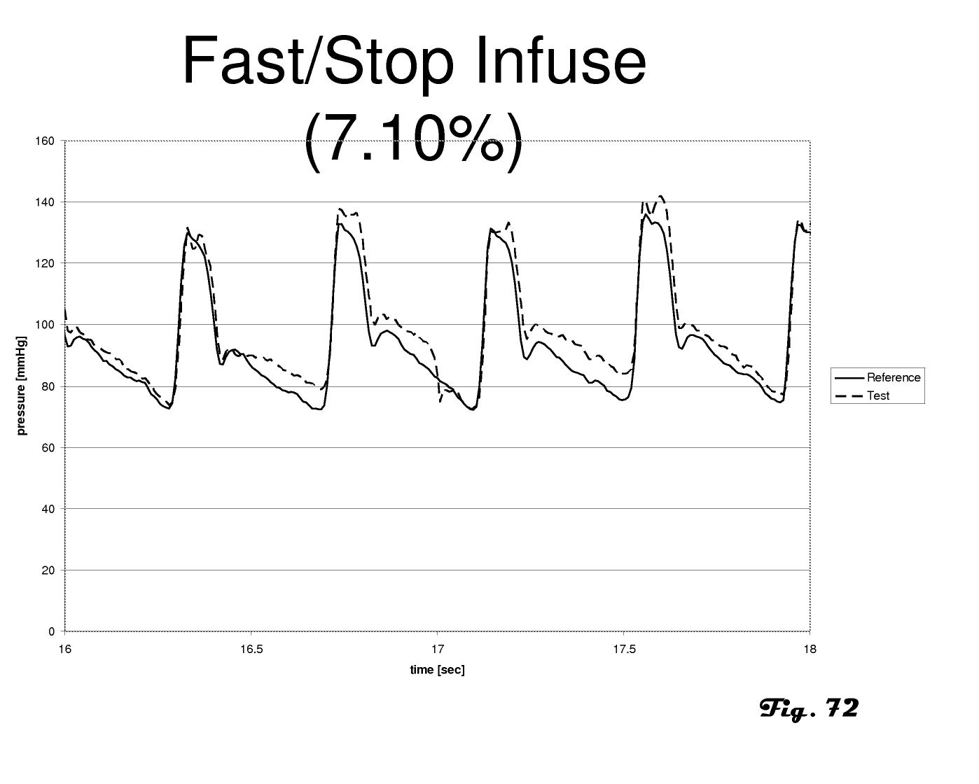

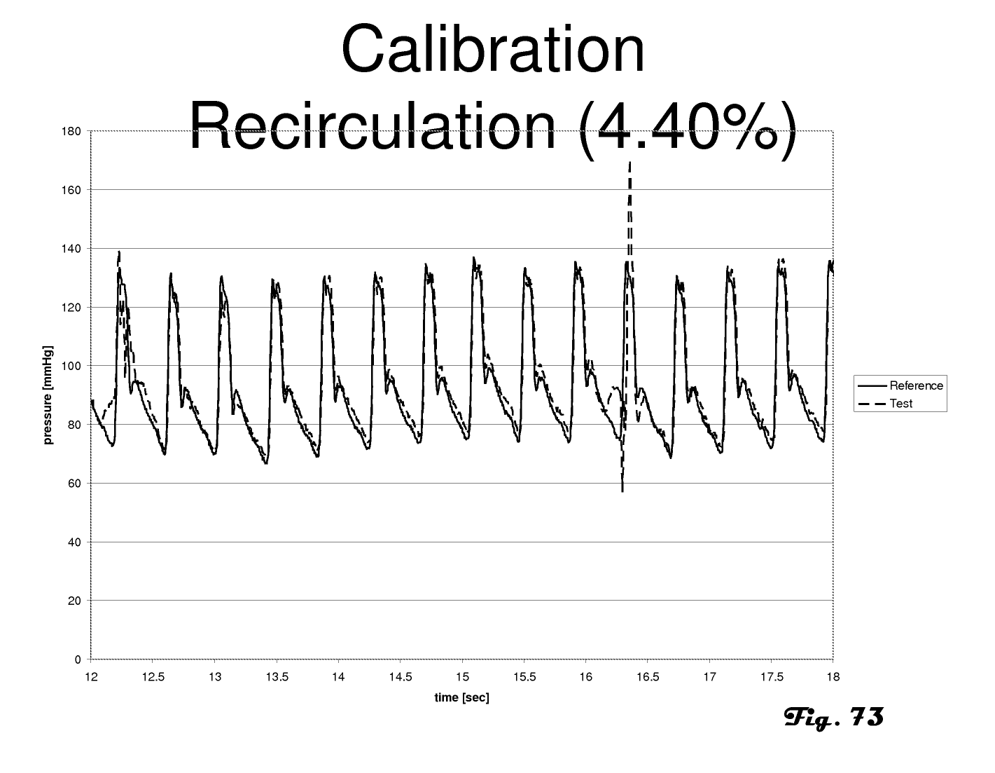

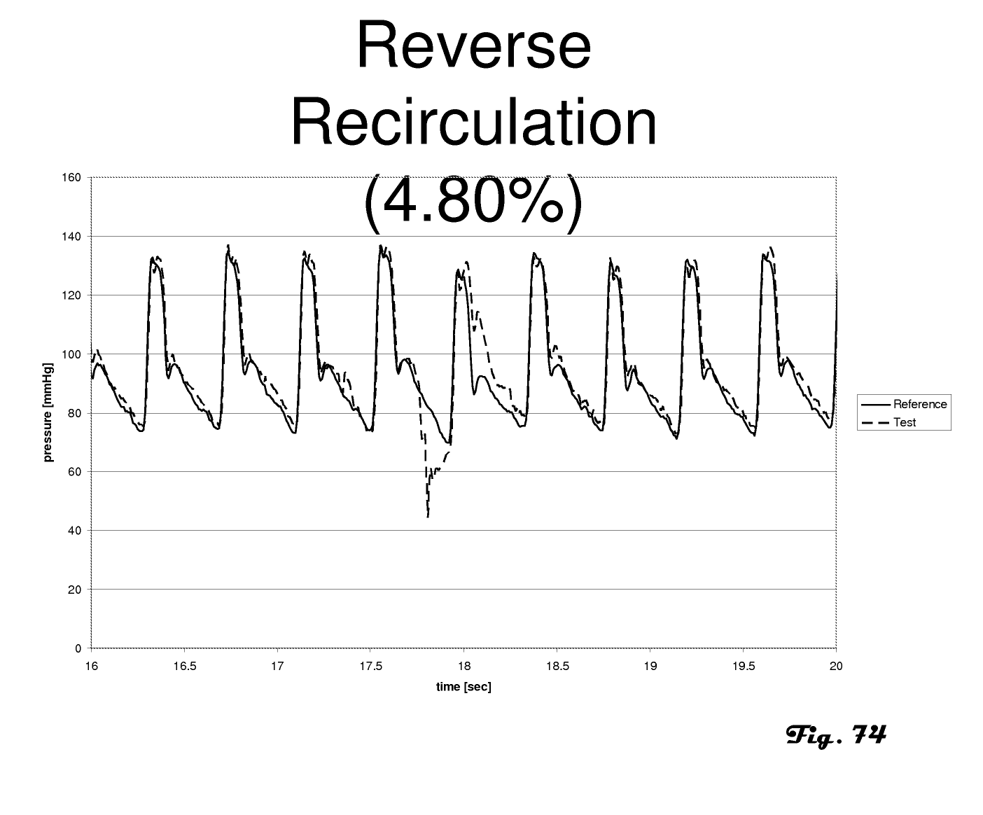

The impact of a measurement cycle on hemodynamic monitoring performance was determined. The variable pressure, variable volume system (aka the artificial patient) was attached as shown in FIG. 60. A standard blood measurement cycle was initiated and reference pressure transducer and test pressure transducer measurements recorded. The comparison of these measurements was done on a pulse by pulse basis. FIG. 64 shows the percent absolute error on a pulse by pulse basis for the entire measurement cycle. The solid line at 5% error enables easy visualization of the measurement cycle stages that create appreciable pressure measurement errors. FIG. 65 has each significant stage of the measurement cycle identified by name. The stages and their corresponding purpose are as follows:

a. Catheter Clear: an infusion pulse to clear catheter before draw
b. Background: a first calibration point at one glucose concentration
c. Blood draw: pulls blood in to the circuit
d. Blood measurement: the period over which a measurement is made
e. Fast infuse: a stage that infuses the blood into the patient
f. Infuse/stop: a stage that infuses blood into the patient but does so by infusing and stopping, a process that improves overall cleaning
g. Calibration recirculation: a combination phase involving cleaning of the circuit in the movement of a second calibration solution to the sensor.
h. Calibration measurement: a second calibration point at a second glucose concentration.
i. Reverse recirculation: a stage to remove the second calibration solution from the sensor.

Hemodynamic monitoring disruption can be mitigated by the use of an access mechanism that provided independent or semi-independent access through a single access location. For example a dual lumen catheter could be used. For example the Arrow International TWINCATH® 20/22 multiple-lumen peripheral catheter could be used in such a situation. The catheter contains two separate non-communicating lumens.

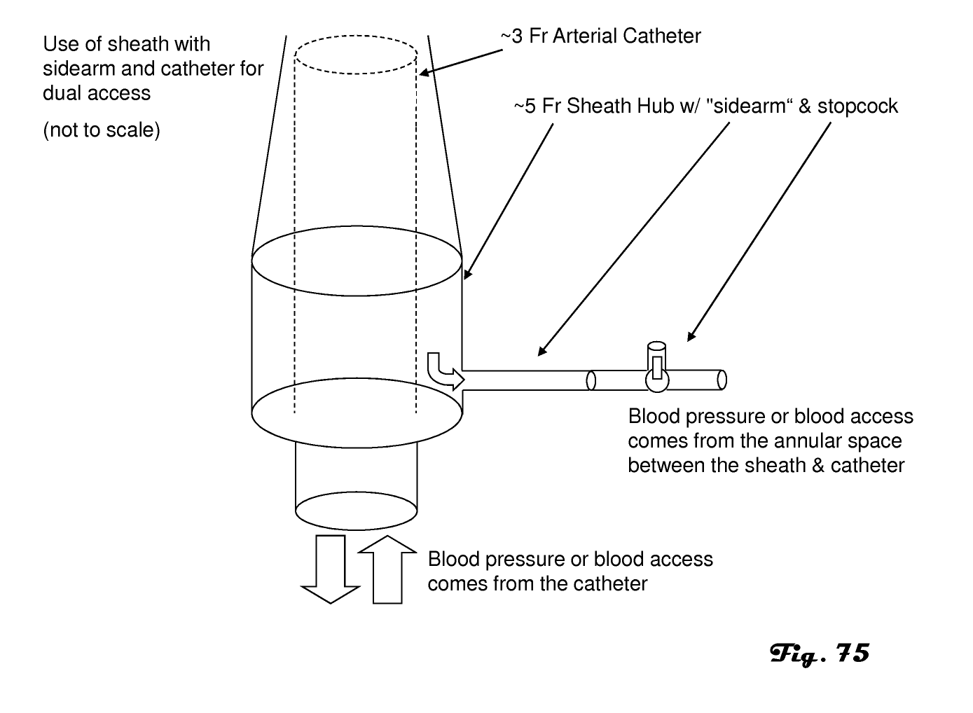

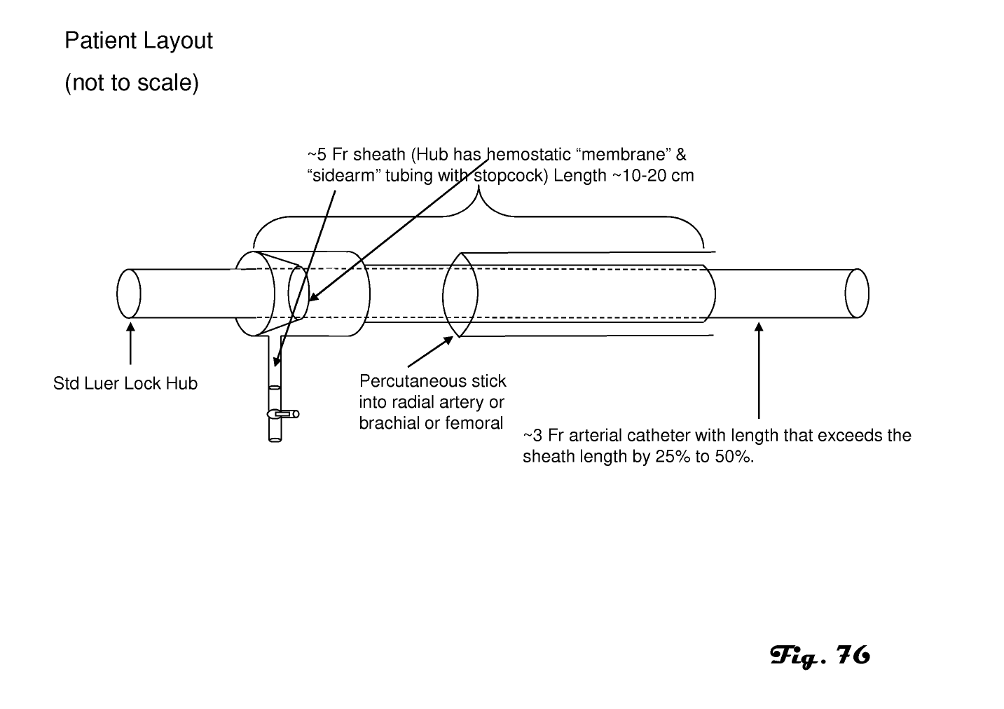

Another mechanism that provides access via two different pathways is the use of a arterial sheath with side arm and catheter. FIGS. 75-76 show an example embodiment of such a system. It is composed of a standard, off-the-sheath used in a variety of arterial-based interventional (radiology, cardiology, neuroradiology) procedures. The sidearm (with stopcock) of the sheath is integrated into the hub of the sheath. The hub typically contains a hemostasis membrane to minimize blood loss during the procedure. A smaller diameter arterial catheter is inserted thru the sheath into the artery. In use maintaining an ˜2 French difference between the sheath and catheter may be optimal for a good annular space. This annular space between the sheath and catheter can be used for blood draw by the automated blood measurement system or connected to the arterial pressure transducer. Correspondingly, the catheter can be used for attachment to the automated blood measurement system or connected to the arterial pressure transducer.

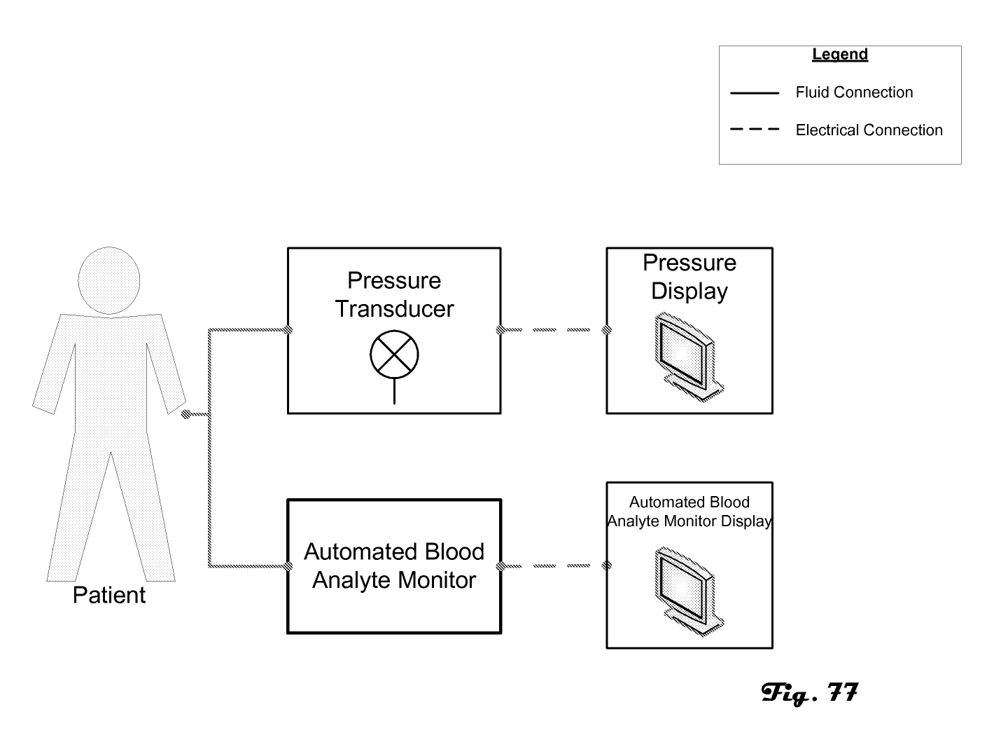

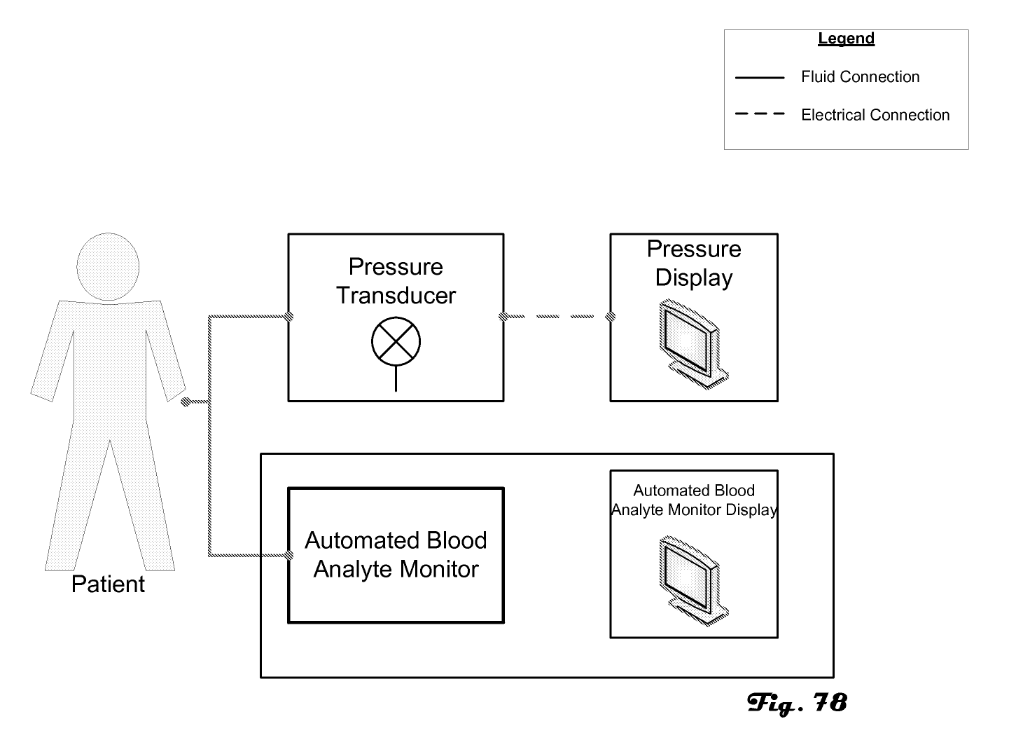

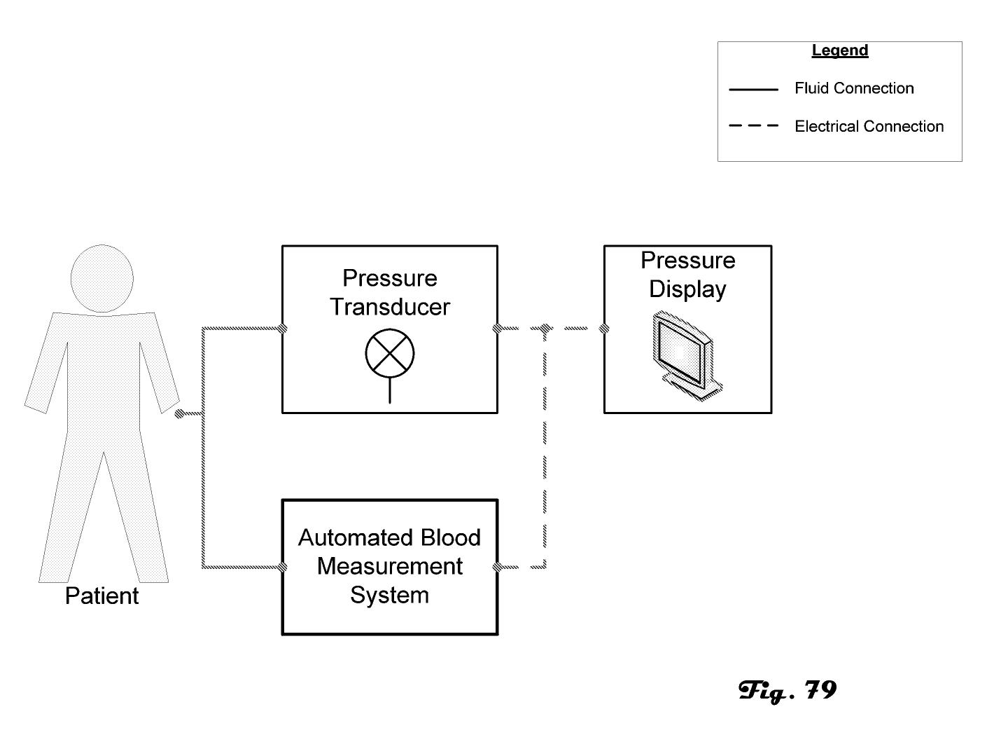

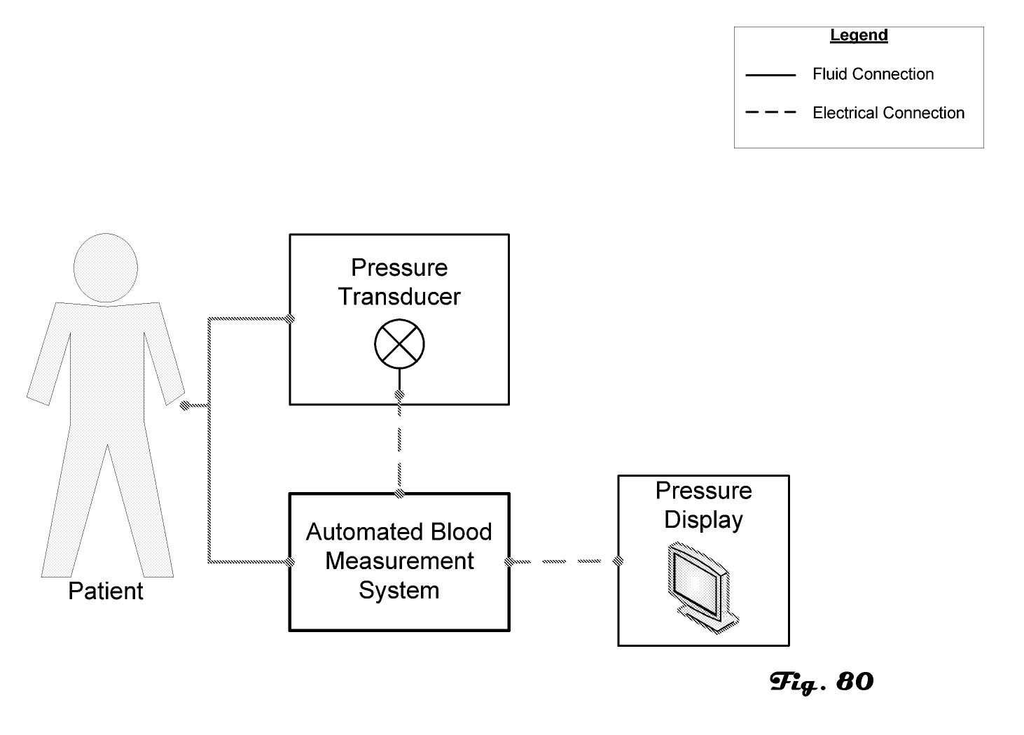

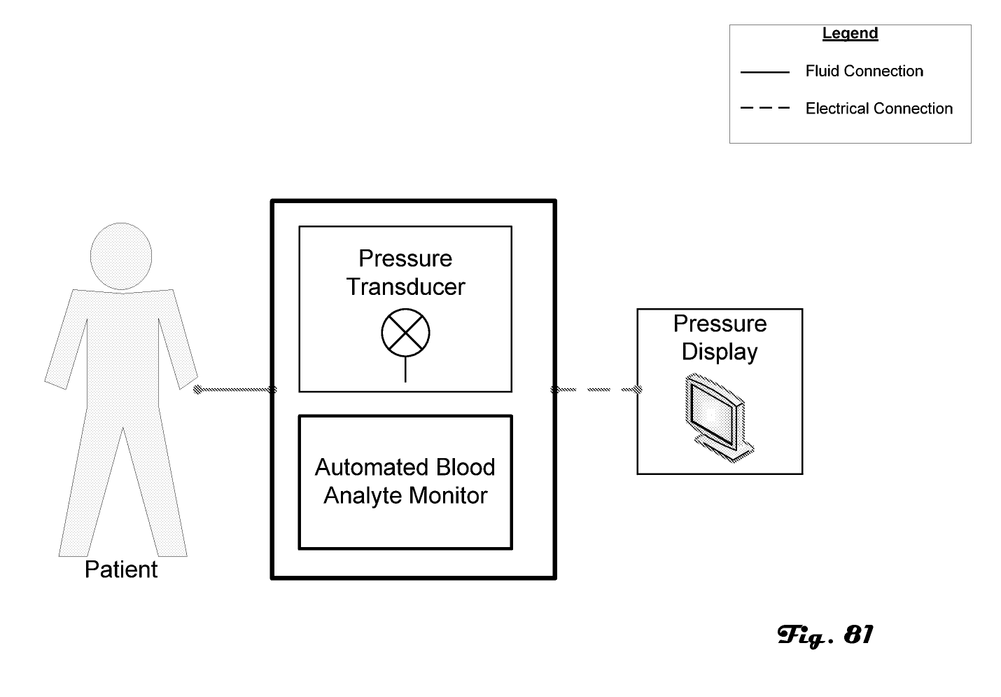

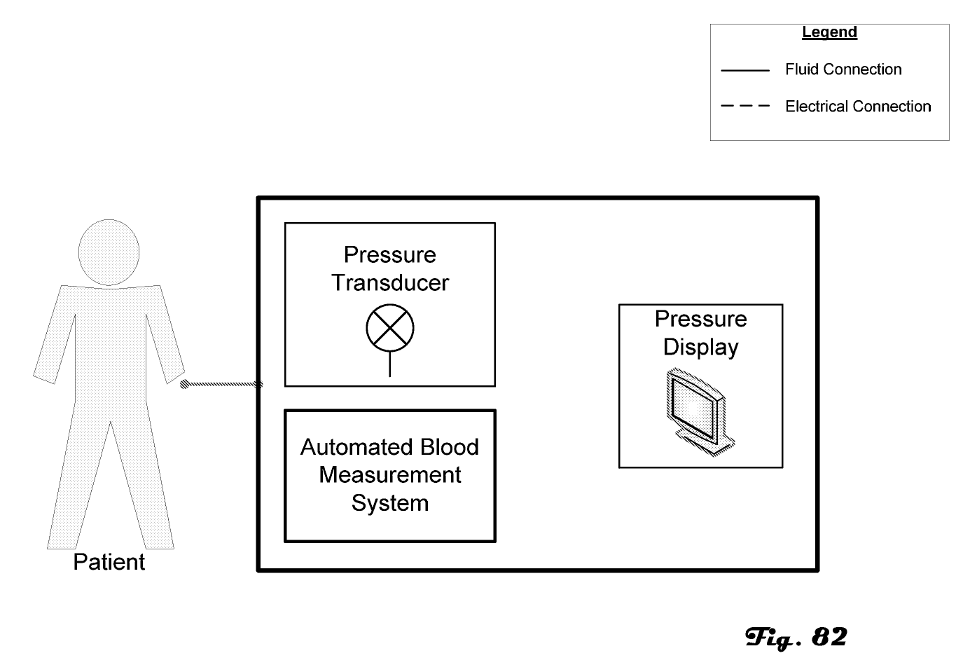

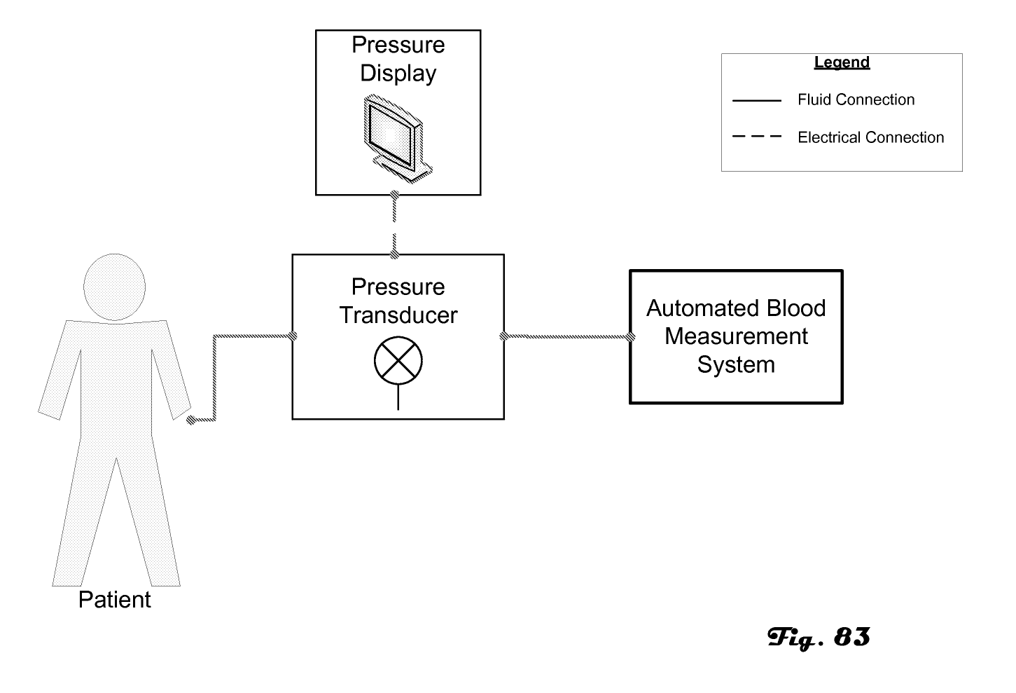

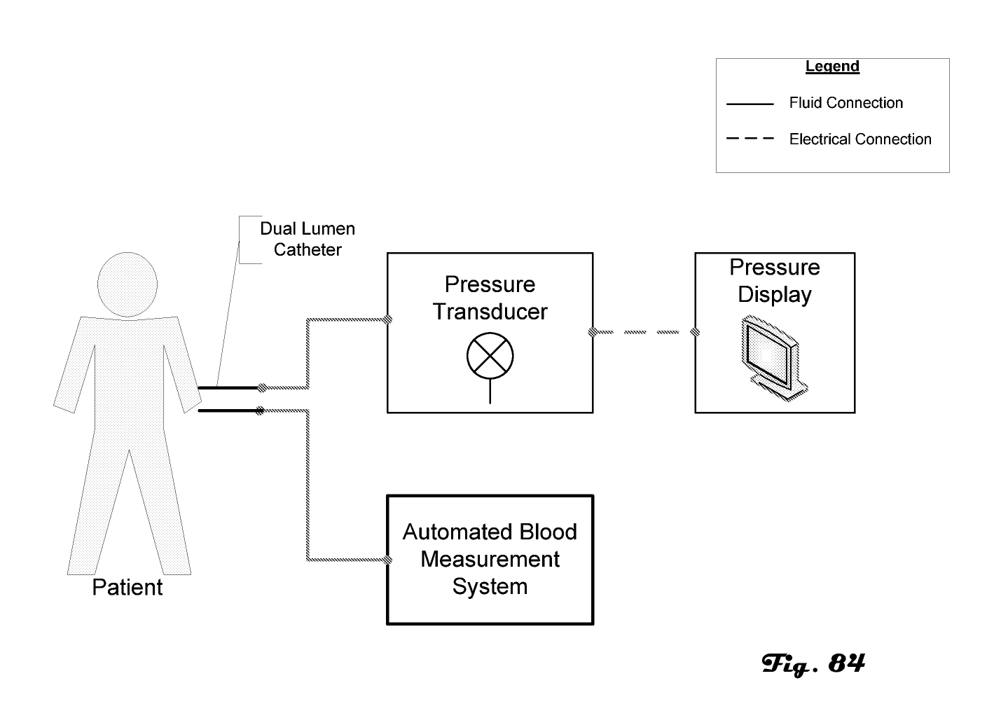

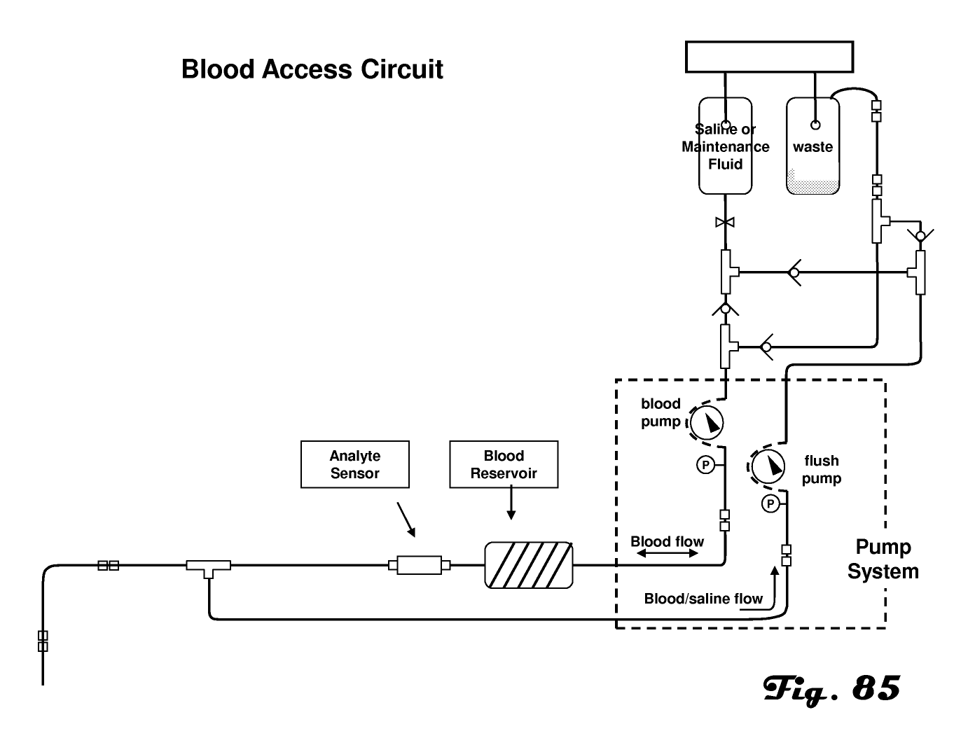

FIGS. 77 to 83 show a variety of configurations that satisfy the general objective of providing both hemodynamic monitoring as well as blood analyte measurements from a single access location. FIG. 77 illustrates a situation where the pressure transducer and the automated blood analyte system share a singular access site. No electrical connectivity is established between the pressure transducer and automated blood measurement system. Electrical connectivity exists between the automated blood analyte system and the automated blood analyte display. If hemodynamic monitoring disruption occurs then the automated blood analyte monitor display notifies the clinician via visual or audible alarms. FIG. 78 illustrates a situation where the pressure transducer and the automated blood analyte system share a singular access site. No electrical connectivity is established between the pressure transducer and automated blood measurement system. FIG. 79 illustrates a situation where the pressure transducer and the automated blood analyte system share a singular access site. Electrical connectivity is established between the pressure transducer, pressure display and automated blood measurement system. FIG. 80 illustrates a situation where the pressure transducer and automated blood measurement system share a single access site. Electrical connectivity exists between the pressure transducer and the automated blood measurement system. Electrical connectivity exists between the automated blood measurement system and the pressure display. FIG. 81 illustrates a situation where the pressure transducer and be automated blood measurement system exist within a single system. Electrical connectivity exists between the combined system and the pressure display. FIG. 82 illustrates a situation where the pressure transducer, automated blood measurement system, and pressure display exist within a single system. FIG. 83 illustrates a system with fluid connectivity between the patient and the pressure transducer. The automated blood measurement system is then in fluid connectivity with the pressure transducer. Electrical connectivity exists between the pressure transducer and the pressure display. FIG. 84 illustrates the use of a duel lumen catheter at a singular arterial access site. The pressure transducer and the automated blood measurement system are in direct fluid contact with the patient. The pressure transducer is electrically connected to the pressure display. Electrical connection between the automated blood measurement system and the pressure display is not shown but one of ordinary skill in the art would appreciate that this can occur.

An example apparatus according to the present invention comprises an arterial catheter, configured to be placed in fluid communication with an artery of a patient; a blood pressure monitoring subsystem mounted with the arterial catheter such that the blood pressure monitoring subsystem can determine the pressure of blood in the artery; and an analyte measuring subsystem mounted with the arterial catheter such that the analyte measuring subsystem can determine the presence, concentration, or both of one or more analytes in blood withdrawn from the artery.

An example apparatus according to the present invention comprises an arterial catheter, configured to be placed in fluid communication with an artery of a patient; a blood pressure monitoring subsystem mounted with the arterial catheter such that the blood pressure monitoring subsystem can determine the pressure of blood in the artery; and an analyte measuring subsystem mounted with the arterial catheter such that the analyte measuring subsystem can determine the presence, concentration, or both of one or more analytes in blood withdrawn from the artery. In such an example apparatus the arterial catheter can have first and second lumens, and the blood pressure measuring subsystem can be mounted in fluid communication with first lumen, and the analyte measuring subsystem can be mounted in fluid communication with the second lumen.

An example apparatus according to the present invention comprises an arterial catheter, configured to be placed in fluid communication with an artery of a patient; a blood pressure monitoring subsystem mounted with the arterial catheter such that the blood pressure monitoring subsystem can determine the pressure of blood in the artery; and an analyte measuring subsystem mounted with the arterial catheter such that the analyte measuring subsystem can determine the presence, concentration, or both of one or more analytes in blood withdrawn from the artery. In such an example apparatus, the arterial catheter can comprise (i) a hub defining an internal volume characterized by an internal diameter and having a fluid port in fluid communication with the internal volume; and (ii) a catheter having an external diameter less than the hub internal diameter and mounted within the internal volume; and the pressure monitoring subsystem can be mounted in fluid communication with either the fluid port of the hub or the catheter, and the analyte measuring subsystem can be mounted in fluid communication with the other of the fluid port of the hub or the catheter.

An example apparatus according to the present invention comprises an arterial catheter, configured to be placed in fluid communication with an artery of a patient; a blood pressure monitoring subsystem mounted with the arterial catheter such that the blood pressure monitoring subsystem can determine the pressure of blood in the artery; and an analyte measuring subsystem mounted with the arterial catheter such that the analyte measuring subsystem can determine the presence, concentration, or both of one or more analytes in blood withdrawn from the artery. In such an example apparatus, the analyte measuring subsystem can transport blood from the catheter; and the apparatus can further comprise an alarm and display subsystem, responsive to the blood pressure monitoring device and the analyte measuring subsystem, configured such that an alarm is indicated when both (i) the pressure monitoring subsystem indicates pressure outside a range of acceptable values and (ii) the analyte measuring subsystem indicates that the pressure monitoring subsystem indication is not invalidated by the analyte measuring subsystem.

In an example apparatus as in the preceding paragraph, the alarm and display subsystem can be further configured to display (i) an indication of pressure responsive to the pressure monitoring subsystem when the analyte measuring subsystem does not indicate interference with the pressure monitoring subsystem, and (ii) an indication that analyte measurement subsystem is interfering with the pressure monitoring subsystem when the analyte measuring subsystem does indicate interference with the pressure monitoring subsystem.

In an example apparatus as in the preceding paragraph, the indication that the analyte measurement subsystem is interfering with the pressure monitoring subsystem can comprise one or more of a text message, a change in color of the display, a change in size of a displayed waveform, or a waveform with a shape recognizably distinct from normal patient pressure waveforms.

An example apparatus according to the present invention comprises an arterial catheter, configured to be placed in fluid communication with an artery of a patient; a blood pressure monitoring subsystem mounted with the arterial catheter such that the blood pressure monitoring subsystem can determine the pressure of blood in the artery; and an analyte measuring subsystem mounted with the arterial catheter such that the analyte measuring subsystem can determine the presence, concentration, or both of one or more analytes in blood withdrawn from the artery. In such an example apparatus, the analyte measuring subsystem can transport blood from the catheter; and the apparatus can further comprise a display subsystem, responsive to the blood pressure monitoring device and the analyte measuring subsystem, configured to display a pressure indicated by the pressure monitoring subsystem when the analyte measuring subsystem is not interfering with the pressure measurement subsystem, and to determine and display a compensated pressure measurement during times when the analyte measurement subsystem is interfering with the pressure measurement subsystem.

In an example apparatus as in the preceding paragraph, the display subsystem can determine a compensated pressure measurement according to the output of the pressure sensor and information provided by the analyte measurement subsystem.

An example apparatus according to the present invention comprises an arterial catheter, configured to be placed in fluid communication with an artery of a patient; a blood pressure monitoring subsystem mounted with the arterial catheter such that the blood pressure monitoring subsystem can determine the pressure of blood in the artery; and an analyte measuring subsystem mounted with the arterial catheter such that the analyte measuring subsystem can determine the presence, concentration, or both of one or more analytes in blood withdrawn from the artery. In such an example apparatus, the mechanical compliance of the combination of the pressure monitoring subsystem and the analyte measuring subsystem satisfies the Gardner wedge criteria.

A method of calibrating any of the example apparatuses described herein can comprise operating the analyte measurement system such that fluid movement during calibration does not introduce errors of more than 5% in the output of the pressure monitoring subsystem.

An example apparatus according to the present invention comprises an arterial catheter, configured to be placed in fluid communication with an artery of a patient; a blood access subsystem, comprising: an analyte measurement device; a pressure sensor; a fluid path from the arterial catheter to the analyte measurement device and to the pressure sensor; at least one pump configured to move fluid in the fluid pathways; and a control system operatively connected to the pump to control operation of the pump; and a pressure determination system responsive to the pressure sensor and to the control system, configured to determine a signal corresponding to pressure in the artery from the pressure sensor and from the characteristics of the pump as indicated by the control system.

In an example apparatus as in the preceding paragraph, the pressure determination system can determine a signal corresponding to pressure in the artery by a lumped parameter model.

An example analyte measurement apparatus according to the present invention comprises a blood access subsystem, configured to transport fluid from a fluid access port connected to an arterial catheter during defined fluid transport times; an analyte measurement subsystem, configured to determine an analyte property of said withdrawn blood; and a pressure signal communication subsystem, configured to accept an input pressure signal from a pressure measurement system in fluid communication with the fluid access port, and to output a signal determined by (i) the input pressure signal except during fluid transport times, and (ii) a determined signal during fluid transport times.

In an example apparatus as in the preceding paragraph, the determined signal can correspond to a compensated pressure signal. In an example apparatus as in the preceding paragraph, the determined signal can comprise a signal having a high value, a low value, and a frequency similar to that of the input pressure signal during times that are not fluid communication times, but that has a waveform shape that is observably different from that of the input pressure signal during times that are not fluid transport times.

In an example apparatus as in the preceding paragraph, the waveform shape can comprise a square wave, a triangle wave, a simulated pressure wave with noise added, or a combination of any of two or more of the preceding.

Calibrating an automated analyte measurement system The present invention is described herein in the context of example blood access and measurement systems, for convenience of description. The present invention can also be used in combination with other blood access systems, such as those described in the applications incorporated by reference above.

FIG. 85 is an illustration of an example embodiment of a blood access and measurement system suitable for use with the present invention. The example automated blood analyte measurement system contains a sterile fluid solution and a waste bag. The saline or maintenance fluid can contain either zero glucose concentration or a known glucose concentration. Such a system provides the glucose sensor with a known calibration point. In use the sensor can be exposed to this known concentration on a periodic basis.

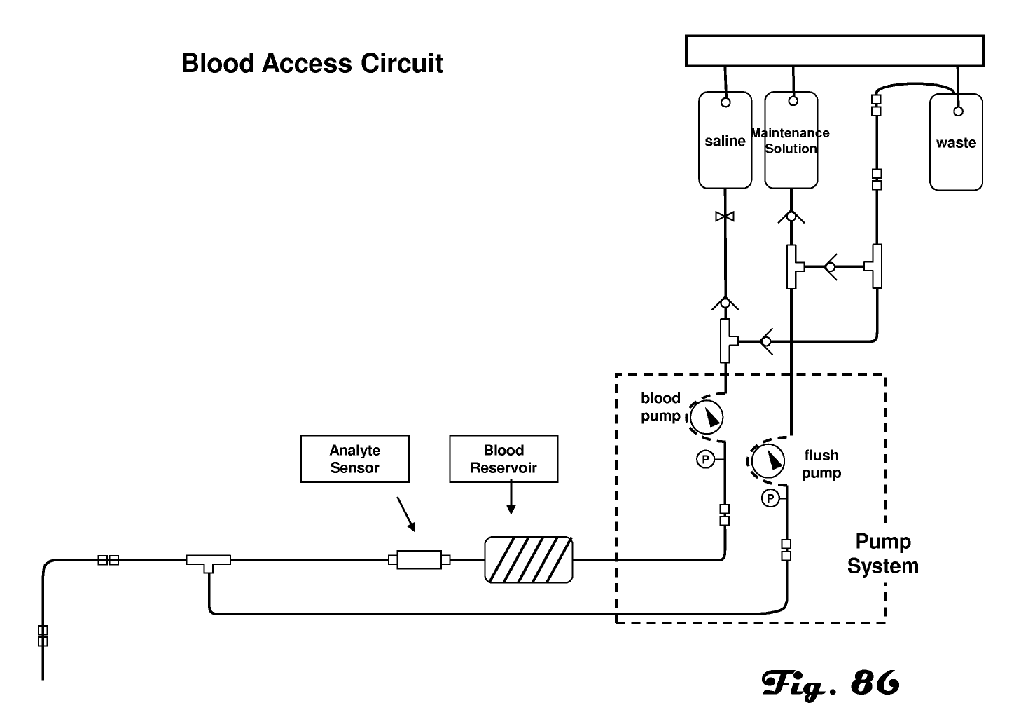

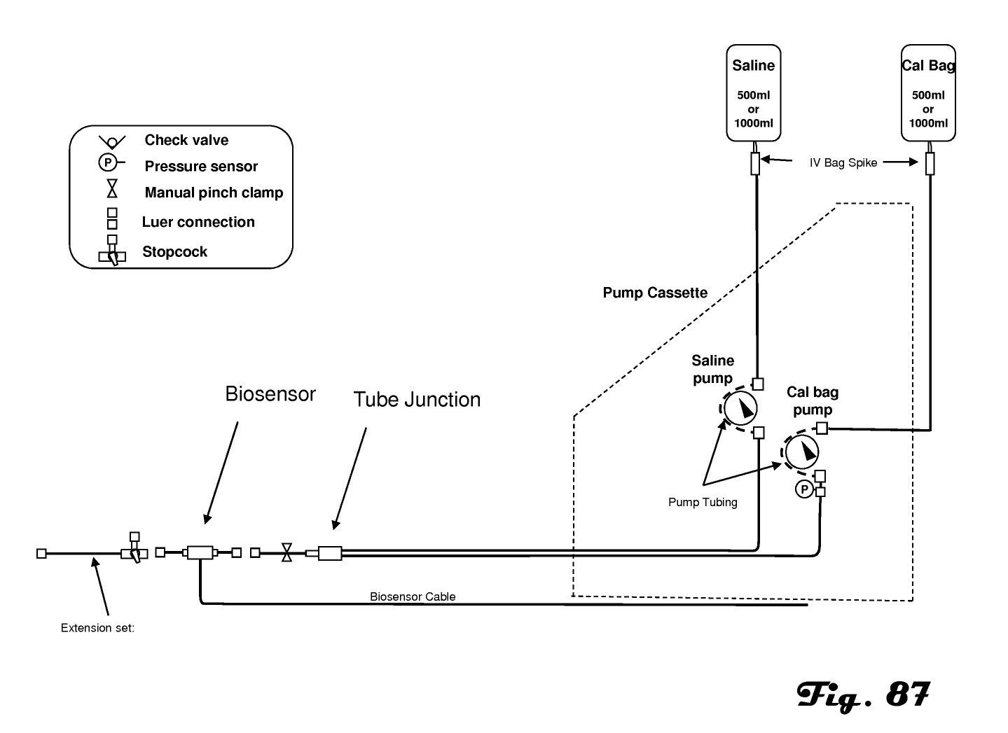

FIG. 86 is an illustration of an example embodiment of a blood access and measurement system suitable for use with the present invention. The example automated blood analyte measurement system contains two fluid bags providing for at least two different calibration points. In use, the analyte sensor can be exposed to a zero or predetermined glucose concentration via fluid from the saline bag. A second glucose concentration can be provided via fluid from the maintenance solution bag. The example system in FIG. 86 provides the opportunity for calibration of the device with a known maintenance fluid while concurrently minimizing the infusion of the maintenance fluid into the patient. In the example system, the maintenance fluid solution can be pumped through the circuit and directly to waste without infusion into the patient. For example, the flush pump can be operated in a manner towards the patient and the blood pump can operate at a similar rate away from the patient. In this manner the analyte sensor is exposed to the maintenance fluid solution but little or no fluid is infused into the patient. Following sensor calibration, fluid from the saline bag can be used to wash the circuit in a similar manner. Such a process can enables the effective calibration of the glucose sensor. Such a system also provides the opportunity to clean or maintain circuit performance with additives where infusion into the subject is not desired.

FIG. 87 is an illustration of an example embodiment where the sensor is located near the patient. The example automated blood analyte measurement system contains two fluid bags providing for at least two different calibration points, labeled as saline and cal bag. In use, the analyte sensor can be exposed to a zero or predetermined glucose concentration via fluid from the saline bag. A second glucose concentration can be provided via fluid from the calibration solution bag. The example system in FIG. 87 provides the opportunity for calibration of the device with a known maintenance fluid while concurrently minimizing the infusion of the maintenance fluid into the patient. In the example system, the calibration solution can be pumped through the circuit so that both tubes going to the sensor are filled with undiluted calibration solution. For example, the cal pump can be operated in a manner towards the patient and the saline pump can operate at a similar rate away from the patient. The fluid can go to a waste outlet (not shown) as needed. Alternately, the tubing can serve as sufficient reservoir for fluid that is undesirable to infuse into the patient, for example when the time of application of the apparatus is not overly long. When the tube junction contains an appropriate calibration solution, the pumps can be activated so as to push the calibration solution to the sensor. The sensor can be calibrated. To re-fill the circuit with a second calibration solution or a saline without glucose the saline pump can be operated in a manner towards the patient and the cal pump can operate at a similar rate away from the patient. This will result in a second solution near the tube junction. Again the solution can be moved to the sensor by operating both pumps toward the sensor or patient. The total amount of saline infused into the subject is dramatically reduced by the use of this “loop” circuit. Such a process can enable the effective calibration of the glucose sensor. Such a system also provides the opportunity to clean or maintain circuit performance with additives where minimizing the amount of infusion into the subject is desired.

The systems shown FIGS. 86 and 87 Fig. are also compatible with use of citrate as an anticoagulant. One example embodiment places citrate in the saline bag, since that is the fluid that makes the most contact with the blood. Contact with citrate effectively anticoagulates the blood during operation of the circuit. If there are concerns regarding binding of calcium at a high level, calcium can be added to the maintenance bag and infused into the patient during those periods between measurements.

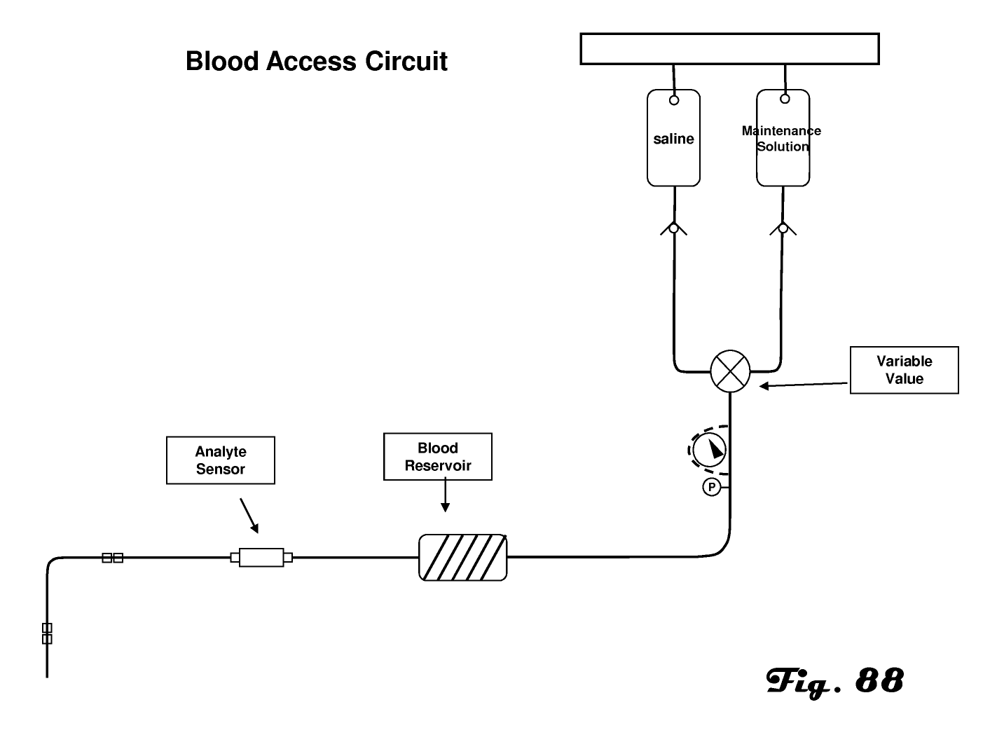

FIG. 88 shows a different implementation of a two level sensor calibration system. The example system in FIG. 88 enables the analyte sensor to be exposed to at least two known glucose concentrations. The variable valve can be a simple stopcock where the solution provided to the analyte sensor is 100% maintenance solution or 100% saline solution. In other embodiments a variable valve can provide for controlled mixing of these two fluid solutions to create multiple glucose concentrations.

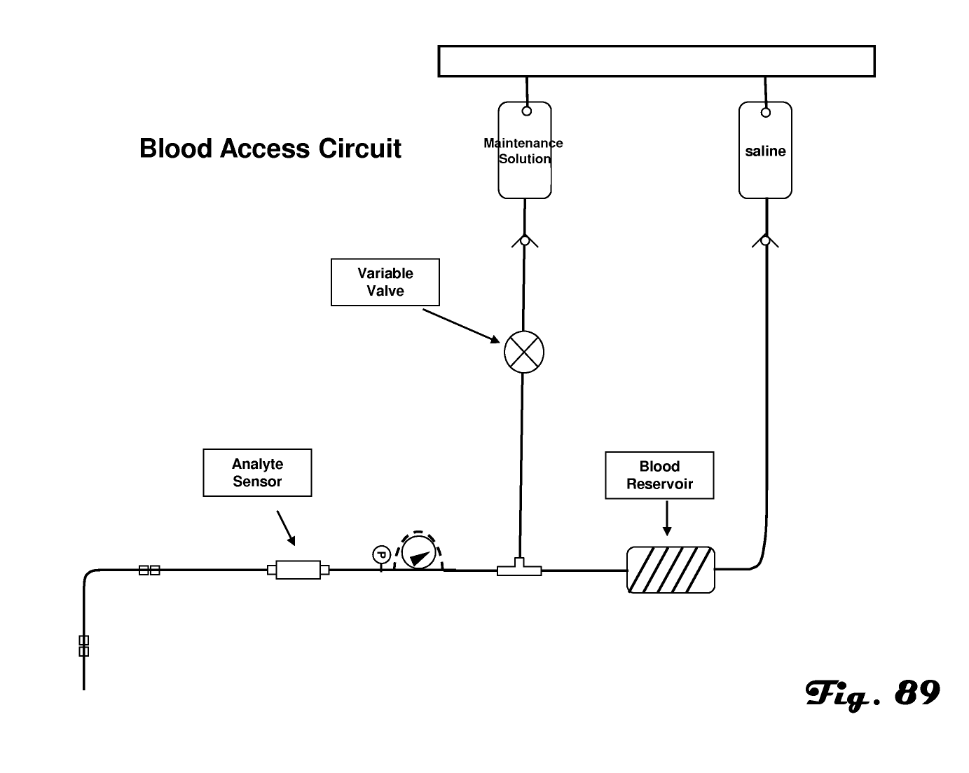

FIG. 89 is an illustration of an example embodiment which enables mixing of glucose into blood obtained from the patient. This example embodiment enables calibration of the analyte sensor at two known glucose concentrations, defined by the maintenance solution and the saline solution. In addition to providing the glucose sensor with non-blood based calibration solutions this system can also enable the calibration of the device using blood. In operation the blood sample can be withdrawn from the patient and exposed to the analyte sensor. Following this baseline measurement a predetermined amount of glucose can be added to the blood as it is pushed back towards the patient. This additive amount enables recalibration of the sensor with a blood based sample with a known additional amount of glucose. It is recognized that the system has the ability to create multiple glucose levels in both saline based calibration standards as well as defined different blood based calibration standards. The ability to manage the amount of mixing occurring at the T-junction and the corresponding glucose concentration at the analyte sensor can be controlled by the variable valve and pump. A blood reservoir is shown in the figure; in practice, such a reservoir can be any structure that allows blood be drawn past the point at which calibration fluid may be mixed with the blood, for example a length of tubing, a bag, fluid space within a pump, and a coil of tubing can all be suitable.

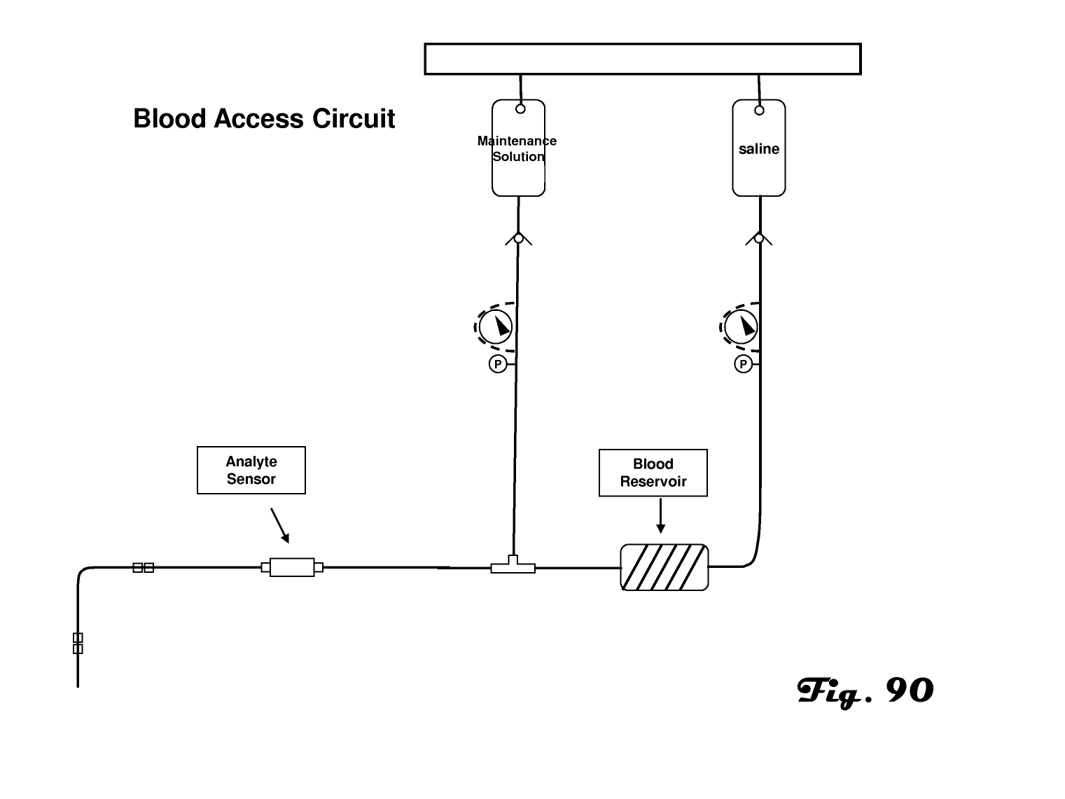

FIG. 90 is an illustration of an example embodiment with similar characteristics as those described in FIG. 89. The example embodiment in FIG. 90 contains two pumps. As shown in FIG. 90, these pumps are peristaltic pumps. Peristaltic pumps enable bidirectional flow as well as support stopped flow conditions. The example embodiment in FIG. 90 has the ability to perform a two point saline based calibration as well as defined glucose additions to the blood sample. The two pumps and reservoir provide the opportunity for assuring good mixing of the glucose throughout the sample. The example shows the use of peristaltic pumps but other pump mechanisms can be used, for example gradient flow, pressurized bags and other pump devices.

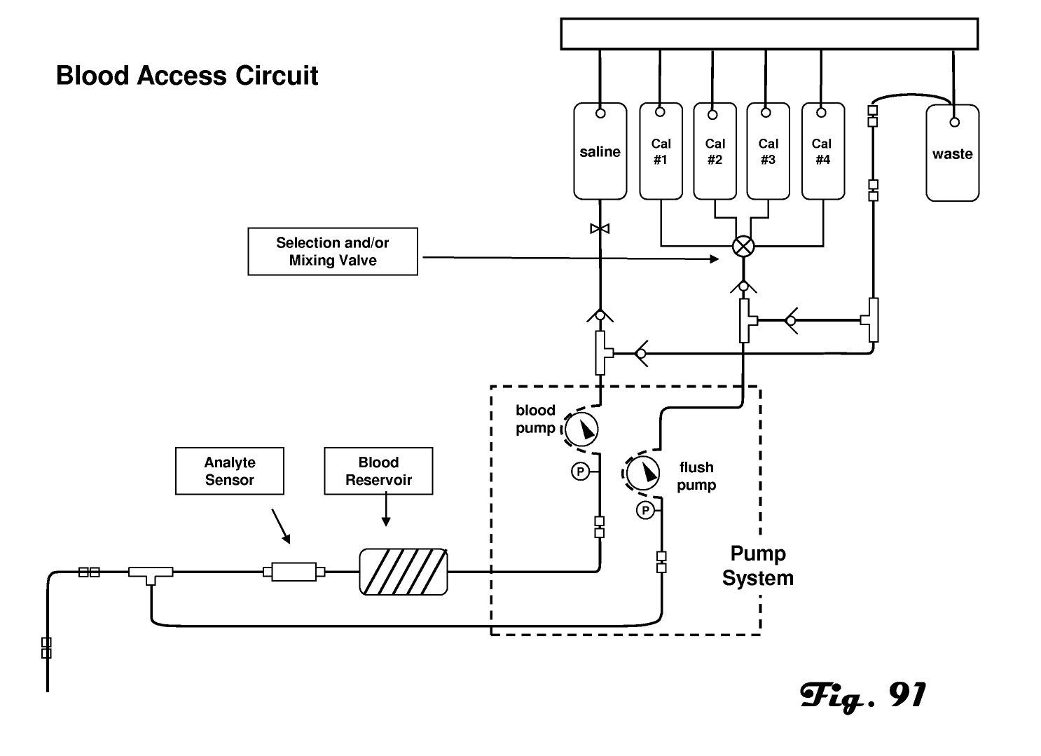

FIG. 91 is an illustration of an example embodiment of a blood access and measurement system suitable for use with the present invention. The example automated blood analyte measurement system contains a saline bag and a plurality of calibration bags. A selectable valve enables selection of the correct calibration solution or the mixing of several calibration solutions in a predetermined manner. In use, the analyte sensor can be exposed to a zero or predetermined glucose concentration via fluid from the saline bag and the calibration solutions. One or more additional glucose concentrations can be provided via fluid from the calibration solutions. The example system in FIG. 91 provides the opportunity for calibration of the device with one or more calibration solutions while concurrently minimizing the infusion of the calibration solutions into the patient. In the example system, the calibration solution can be pumped through the circuit and directly to waste without infusion into the patient. For example, the flush pump can be operated in a manner towards the patient and the blood pump can operate at a similar rate away from the patient. In this manner the analyte sensor is exposed to one or more calibration solutions but no fluid is infused into the patient. Following sensor calibration, fluid from the saline bag can be used to wash the circuit in a similar manner. Such a process can enable the effective calibration of a glucose or other analyte sensor. Such a system also provides the opportunity to clean or maintain circuit performance with additives where infusion into the subject is not desired. Following calibration, sensor performance can be validated by measuring an unused calibration solution or a unique mix of calibration solutions. The system also affords the ability to use one or more validation samples.

The system shown in FIGS. 91, 92, 93, and 94 are compatible with use of citrate as an anticoagulant. One example embodiment places citrate in the saline bag or in one of the calibration solutions, since that the fluid that makes the most contact with the blood. Contact with citrate effectively anticoagulates the blood during operation of the circuit. If there are concerns regarding binding of calcium at a high level, calcium can be added to the maintenance bag and infused into the patient during those periods between measurements.

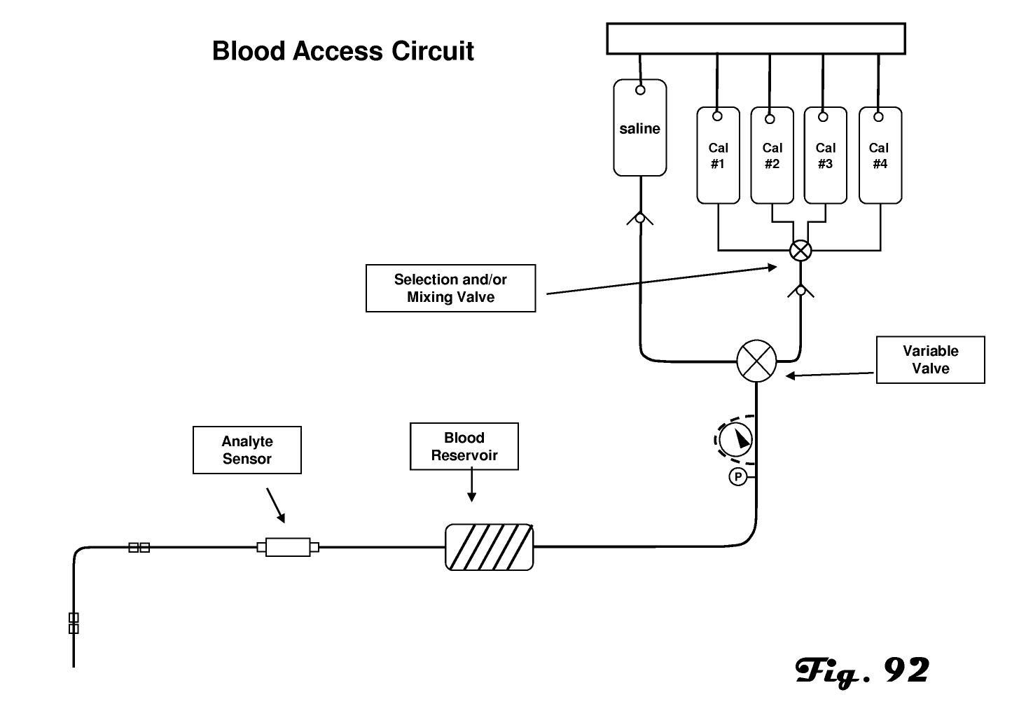

FIG. 92 is an illustration of an example implementation of a multi-level sensor calibration system. The example system in FIG. 92 enables the analyte sensor to be exposed to one or more calibration solutions. The variable valve can be a simple stopcock where the solution provided to the analyte sensory is 100% maintenance solution or 100% saline solution. A selection or mixing valve enables the selection of a particular calibration solution to be used or the creation of a determined mixture of calibration solutions. A variable valve can provide for controlled mixing of the fluid solutions to create multiple analyte concentrations.

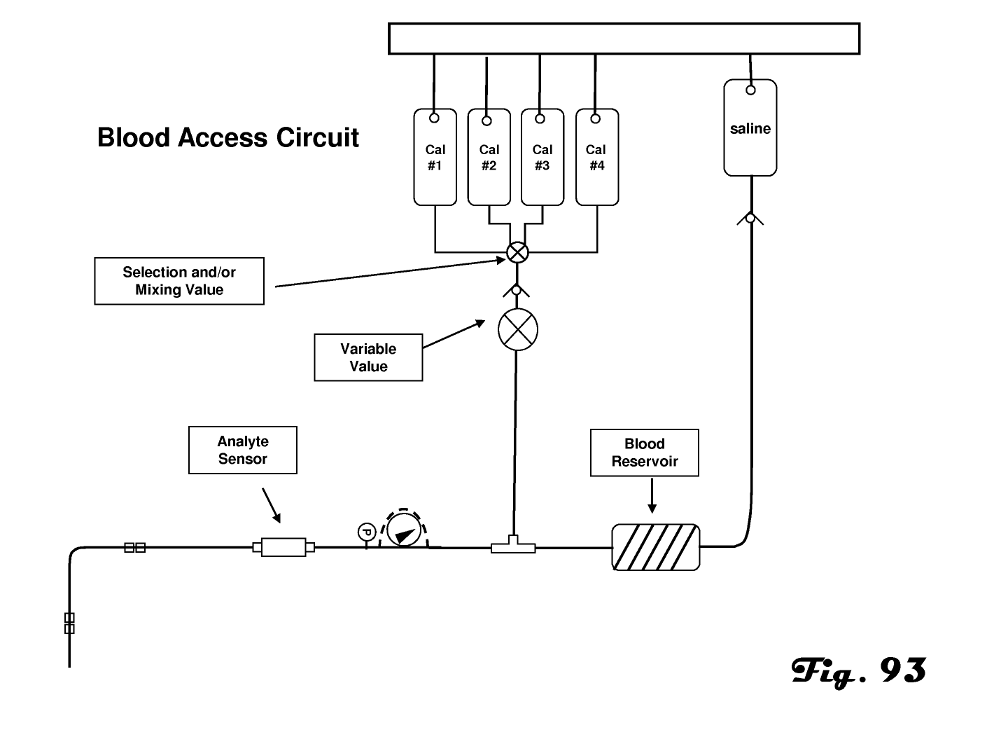

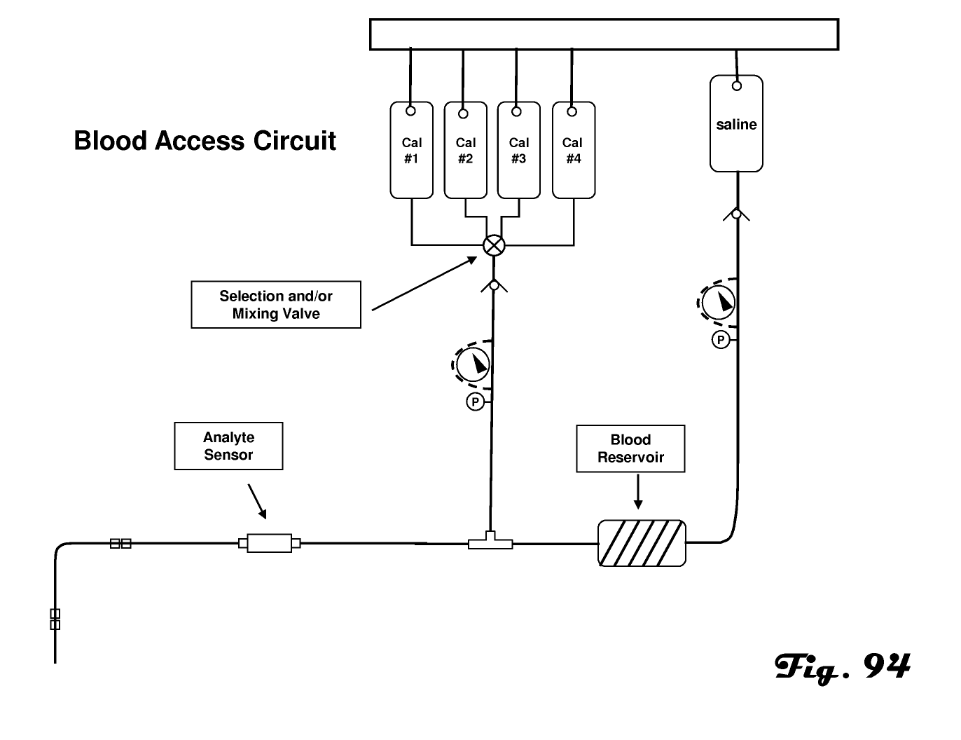

FIG. 93 is an illustration of an example embodiment which enables mixing of glucose into blood obtained from the patient. This example embodiment enables calibration of the analyte sensor at one or more known analyte concentrations, defined by the maintenance solution and the calibration solutions. The set of calibration solutions can allow calibration at a plurality of different analyte concentrations. In addition to providing the glucose sensor with non-blood based calibration solutions this system can also enable the calibration of the device using blood. In operation the blood sample can be withdrawn from the patient and exposed to the analyte sensor. Following this baseline measurement a predetermined amount of glucose can be added to the blood as it is pushed back towards the patient. The embodiment also provides the ability to add a plurality of calibration solutions to the blood sample. This ability to add calibration solutions to the blood sample enables recalibration of the sensor. It is recognized that the system has the ability to create multiple glucose levels in both saline based calibration standards as well as defined different blood based calibration standards. The ability to manage the amount of mixing occurring at the T-junction and the corresponding glucose concentration at the analyte sensor can be controlled by the variable valve and pump. The embodiment also provides the ability to create multiple validation levels both in saline-based solutions and in blood-based solutions.

FIG. 94 is an illustration of an example embodiment with similar characteristics as those described in FIG. 93. The example embodiment in FIG. 94 contains two pumps and a selection and/or mixing valve associated with the calibration solutions. The selection and/or mixing valve can comprise a variety of embodiments, including a simple selection valve and a multipath system that enables mixing in a controlled manner. As shown in FIG. 94, these pumps are peristaltic pumps. Peristaltic pumps enable bidirectional flow as well as support stopped flow conditions. The example embodiment in FIG. 94 has the ability to perform a two point saline based calibration as well as defined glucose additions to the blood sample. The two pumps and reservoir provide the opportunity for assuring good mixing of the glucose throughout the sample.

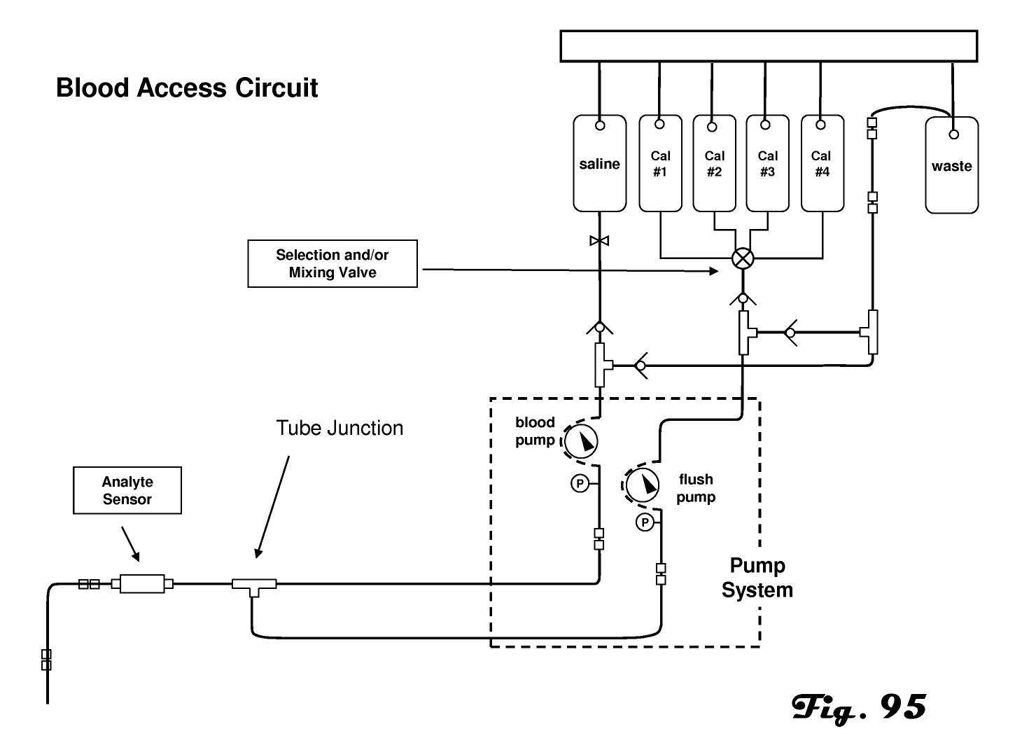

FIG. 95 is an illustration where the sensor is located near the patient and where the tube junction between the blood pump and saline pump is located distal the sensor. The example automated blood analyte measurement system contains a saline bag and a plurality of calibration bags. A selectable valve enables selection of the correct calibration solution or the mixing of several calibration solutions in a predetermined manner. In use, the analyte sensor can be exposed to a zero or predetermined glucose concentration via fluid from the saline bag and the calibration solutions. One or more additional glucose concentrations can be provided via fluid from the calibration solutions. The example system in FIG. 95 provides the opportunity for calibration of the device with one or more calibration solutions while concurrently minimizing the infusion of the calibration solutions into the patient. The overall fluid amount to the patient is minimized by moving the various saline or calibration fluids to the tube junction and only when the appropriate fluid is present near the tube junction is the solution moved to the sensor. For example, the calibration solution #1 can be pumped through the circuit so that the fluid at the tube junction is appropriate for calibration of the sensor. This can be accomplished by having the flush pump operate towards the patient and the blood pump operate at a similar rate away from the patient. The fluid can go to waste via a check value arrangement. When the tube junction contains an appropriate calibration solution, the pumps can be activated so as to push the calibration solution to the sensor, and the sensor calibrated. This fundamental process can be repeated for various calibration solutions and for saline. Thus, the patient only receives a small amount solution, approximately the volume between the tube-junction and the sensor. If no such loop system were employed the subject would receive larger volumes associated with the mixing or transition zone. The mixing or transition zone is the volume where two different solutions mix together. This occurs with or without movement but of a significant volume when solutions are pumped through tubing. Such a process enables the effective calibration of the glucose sensor. Such a system also provides the opportunity to clean or maintain circuit performance with additives where minimizing the amount of infusion into the subject is desired. Following calibration, sensor performance can be validated by measuring an unused calibration solution or a unique mix of calibration solutions. The system also affords the ability to use one or more validation samples. One of skill in the art can appreciate the fact that the number of calibration solutions can be varied from one to many with operation similar that defined above.

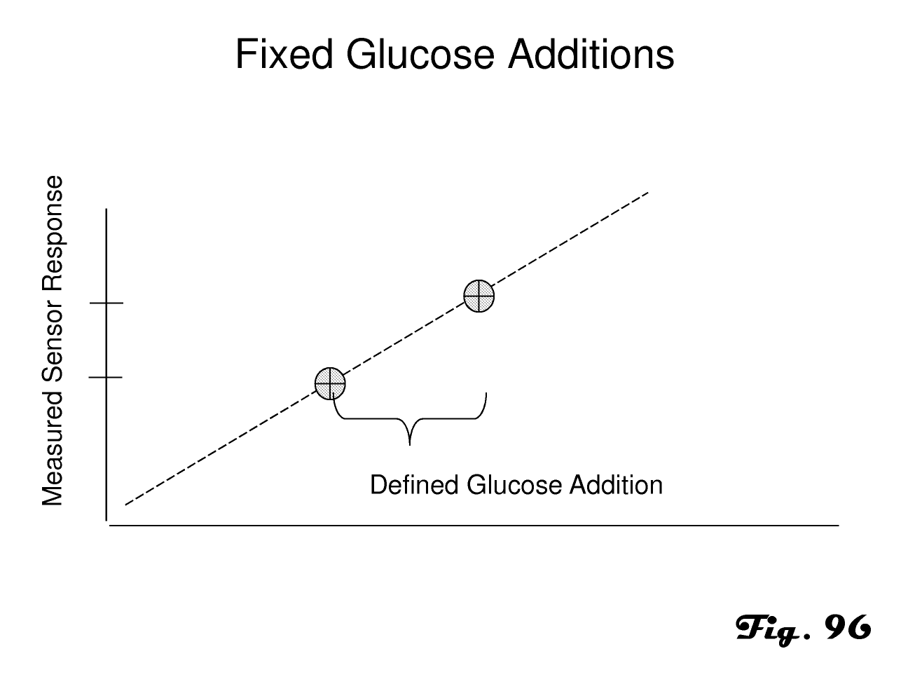

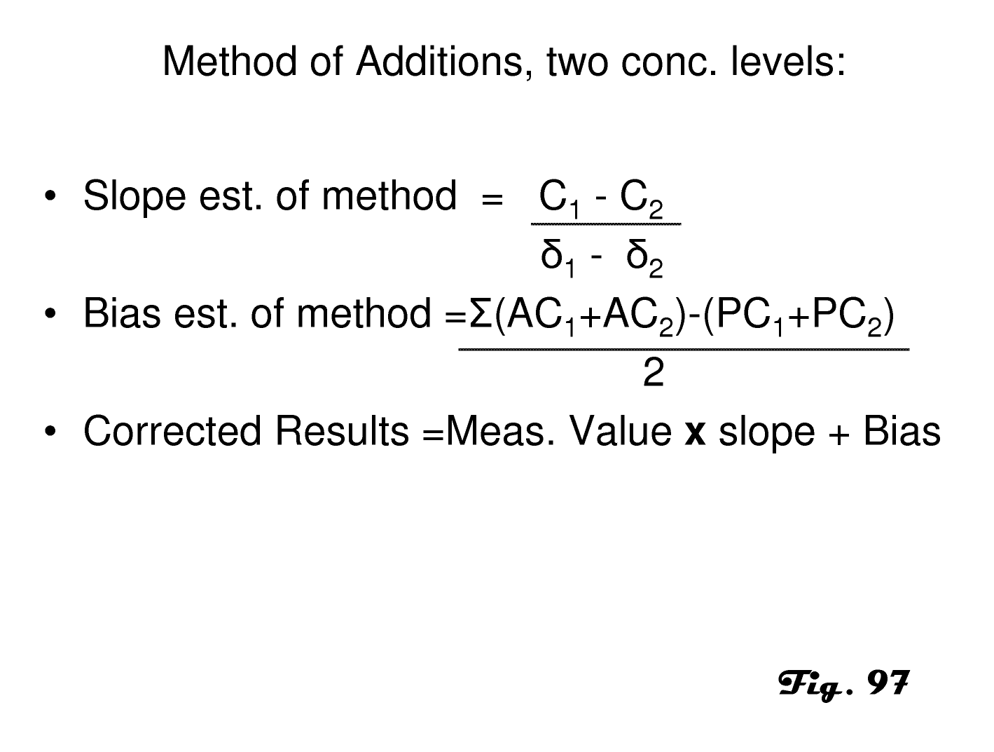

FIG. 96 shows a simplistic example of how a fixed glucose addition to a sample of unknown glucose concentration enables calibration of the device. This concept can be extrapolated to multiple additions or even a response surface mapping with continuous increase or decrease in glucose concentration.

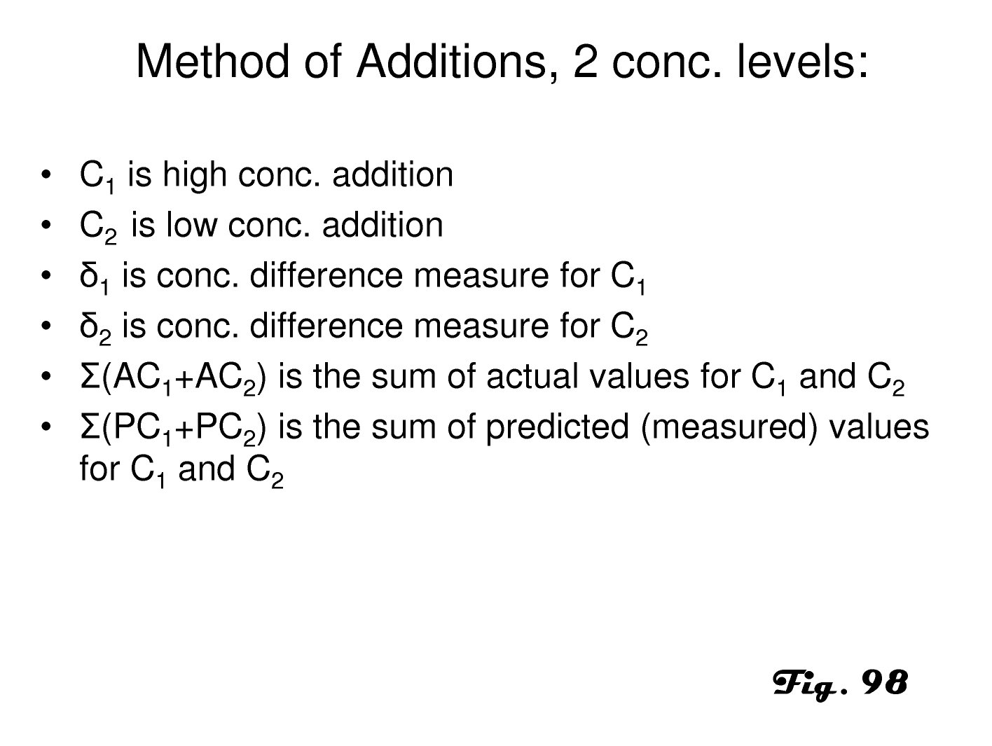

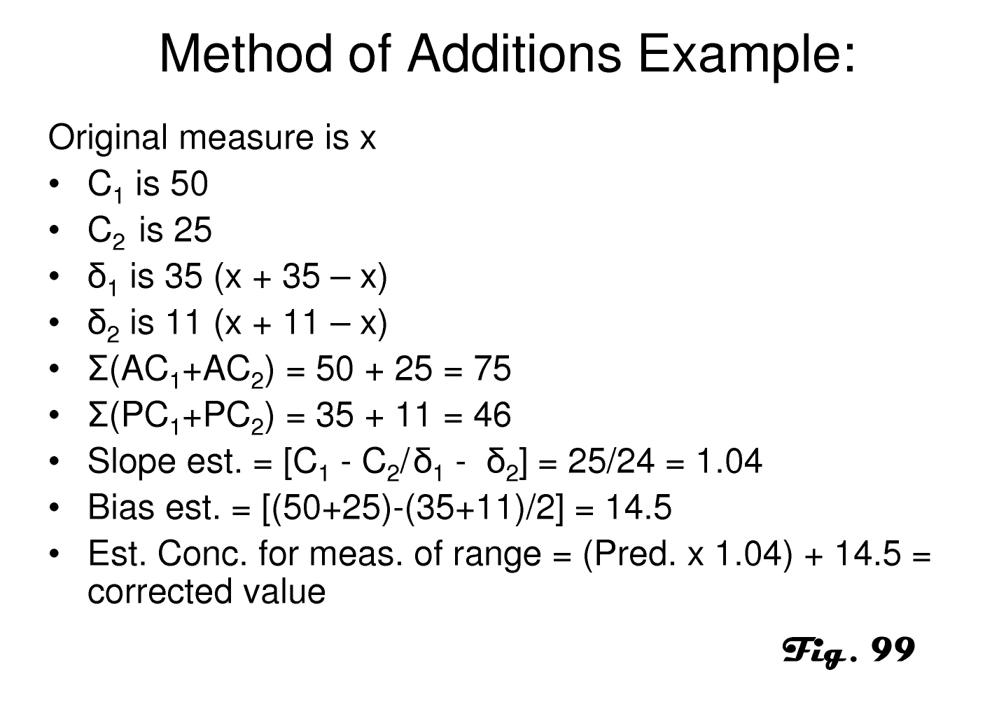

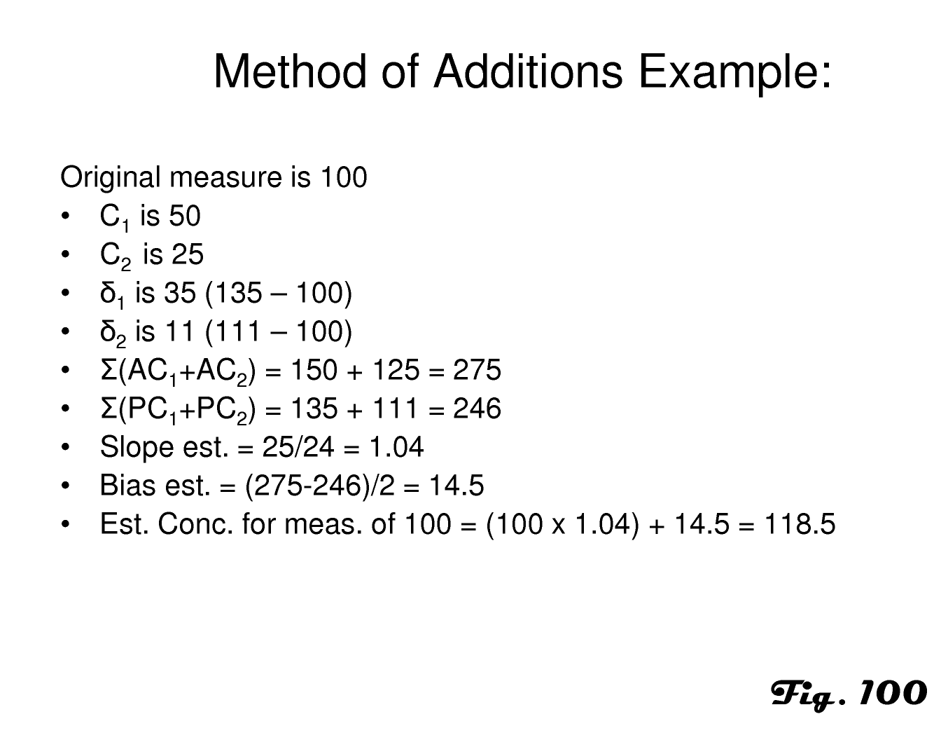

FIGS. 97, 98, 99 and 100 show several examples of how the methods of additions can be used in calibration of the sensor. In FIG. 99, the method is applied where the concentration of the sample is not known but the amount of change to the sample is defined. This process can be used with the current invention to provide for accurate calibration.

In a first example method, the invention provides a method of calibrating an automated analyte measurement system that removes blood from a patient for measurement, comprising passing calibration fluid having at least two different analyte concentrations by an analyte sensor while infusing substantially none of at least one of such calibration fluids into the patient. In such an example, that sensor and calibration fluid can be maintained in a sterile condition.

In a second example method, the present invention provides a method of validating the performance of an automated analyte measurement system, comprising calibrating the system according to the method of claim 1, then determining the sensor response to a calibration fluid having an analyte concentration different from those used in calibration while infusing substantially none of such calibration fluid into the patient.

In a first example apparatus, the present invention provides an apparatus for the measurement of one or more analytes in blood withdrawn from a patient, comprising: a patient connection fluid passage element configured to be placed in fluid communication with the vascular system of a patient; an analyte sensor having first and second ports, the first port in fluid communication with the patient connection fluid passage element; a first fluid source in fluid communication with the second port of the analyte sensor; a second fluid source in fluid communication with the first port of the analyte sensor; a first pump mounted with the apparatus so as to move fluid from the first fluid source towards or away from the analyte sensor; a second pump mounted with the apparatus so as to urge fluid from the second fluid source toward or away from the analyte sensor; and a waste outlet in fluid communication with at least one of the first and second ports of the analyte sensor; wherein at least one of the first fluid source and the second fluid source contains a fluid having a first known analyte concentration suitable for calibration of the analyte sensor.

In an apparatus like the first example apparatus, the first fluid source can contain a fluid having a first known analyte concentration suitable for calibration of the analyte sensor, and wherein the second fluid source contains a fluid having a second known analyte concentration, different from the first known analyte concentration, suitable for calibration of the analyte sensor.

In an apparatus like the first example apparatus, the apparatus can further comprise a third fluid source in fluid communication with at least one of the first port or the second port of the analyte sensor, and containing a fluid having a second known analyte concentration, different from the first known analyte concentration, suitable for calibration of the analyte sensor.

In an apparatus like the first example apparatus, the apparatus can further comprise a selection or mixing valve mounted between either the first fluid source or the second fluid source and the corresponding port of the analyte sensor, and further comprising a third fluid source in fluid communication with the variable mixing valve, and containing a fluid having a second known analyte concentration, different from the first known analyte concentration, suitable for calibration of the analyte sensor.

In a second example apparatus, the present invention provides an apparatus for measurement of one or more analytes in blood withdrawn from a patient, comprising: a patient connection fluid passage element configured to be placed in fluid communication with the vascular system of a patient; an analyte sensor having first and second ports, the first port in fluid communication with the patient connection fluid passage element and separated therefrom by a fluid passage having a first length; a tubing junction comprising first, second, and third ports, the first port in fluid communication with the second port of the analyte sensor and separated therefrom by a fluid passage having a second length; a first fluid source in fluid communication with the second port of the tubing junction and separated therefrom by a fluid passage having a third length, where the sum of the second and third lengths is greater than the first length; a second fluid source in fluid communication with the third port of the tubing junction; a first pump mounted with the apparatus so as to urge fluid from the first fluid source towards or away from the tubing junction; and a second pump mounted with the apparatus so as to urge fluid from the second fluid source toward or away from the tubing junction; wherein the first fluid source contains a fluid having a first known analyte concentration suitable for calibration of the analyte sensor.

In an apparatus like the second example apparatus, the second fluid source can contain a fluid having a second known analyte concentration suitable for calibration of the analyte sensor.

In an apparatus like the second example apparatus, the apparatus can further comprise a selection or mixing valve mounted between the first fluid source and the tubing junction, and further comprising a third fluid source having a fluid having a third known analyte concentration suitable for calibration of the analyte sensor mounted in fluid communication with the selection or mixing valve.

In a third example apparatus, the present invention provides an apparatus for the measurement of one or more analytes in blood withdrawn from a patient, comprising: a patient connection fluid passage element configured to be placed in fluid communication with the vascular system of a patient; an analyte sensor having first and second ports, the first port in fluid communication with the patient connection fluid passage element; a reservoir in fluid communication with the second port of the analyte sensor; a first fluid source in fluid communication with the second port of the analyte sensor, wherein the first fluid source contains a fluid having a first known analyte concentration suitable for calibration of the analyte sensor; a first pump mounted with the apparatus so as to urge fluid from the first fluid source towards or away from the analyte sensor; and a second pump mounted with the apparatus so as to urge fluid from the reservoir toward or away from the analyte sensor.

In an apparatus like the third example apparatus, the apparatus can further comprise a second fluid source in fluid communication with the second port of the analyte sensor, wherein the second fluid source contains a fluid having a second known analyte concentration, different from the first known analyte concentration, suitable for calibration of the analyte sensor.

In third example method, the present invention provides a method of calibrating an apparatus such as the first example apparatus, comprising operating the first and second pumps to flow fluid from the fluid source having a known analyte concentration past the sensor and to the waste outlet, and calibrating the analyte sensor responsive to its response to the fluid having the first known analyte concentration.

In a method like the third example method, wherein the apparatus further comprises a third fluid source in fluid communication with at least one of the first port or the second port of the analyte sensor, and containing a fluid having a second known analyte concentration, different from the first known analyte concentration, suitable for calibration of the analyte sensor, the method can further comprise operating the first and second pumps to flow fluid from the fluid source having a known analyte concentration past the analyte sensor and to the waste outlet, and operating the first and second pumps to flow fluid from the third fluid source past the analyte sensor and to the waste outlet, and calibrating the analyte sensor responsive to its response to the fluid having the first known analyte concentration and its response to the fluid having the second known analyte concentration.

In a method like the third example method, wherein the apparatus further comprises a selection or mixing valve mounted between either the first fluid source or the second fluid source and the corresponding port of the analyte sensor, and further comprising a third fluid source in fluid communication with the selection or mixing valve, and containing a fluid having a second known analyte concentration, different from the first known analyte concentration, suitable for calibration of the analyte sensor, the method can further comprise configuring the selection or mixing valve to pass either of its input fluids or a combination of its input fluids to deliver a first calibration fluid having a first calibration analyte concentration, and operating the first and second pumps to flow the first calibration fluid past the analyte sensor and to the waste outlet, and configuring the selection or mixing valve to pass either of its input fluids or a combination of its input fluids to deliver a second calibration fluid having a second calibration analyte concentration different from the first calibration analyte concentration, and operating the first and second pumps to flow the second calibration fluid past the analyte sensor and to the waste outlet, and calibrating the analyte sensor responsive to its response to the first calibration fluid and its response to the second calibration fluid.

In a method like the third example method, the method can be practiced such that substantially none of the fluid is infused into the patient. In a method like the third example method, the method can be practiced such that an amount of fluid less than the amount that would be likely to cause harm to the patient can be infused into the patient.

In fourth example method, the present invention provides a method of calibrating an apparatus such as in the second example apparatus, comprising operating the first and second pumps to flow fluid from the first fluid source past the analyte sensor while infusing into the patient a volume less than the volume defined by the fluid passage between the tubing junction and the first fluid source, and calibrating the analyte sensor responsive to its response to the fluid.

In a method like the fourth example method, wherein the second fluid source contains a fluid having a second known analyte concentration, different from the first known analyte concentration, suitable for calibration of the analyte sensor, the method can further comprise operating the first and second pumps to flow fluid from the second fluid source past the analyte sensor while infusing into the patient a volume less than the volume defined by the fluid passage between the tubing junction and the second fluid source, and calibrating the analyte sensor responsive to its response to the fluid from the first fluid source and its response to fluid from the second fluid source.

In a method like the fourth example method, wherein the apparatus further comprises a selection or mixing valve mounted between the first fluid source and the tubing junction, and further comprising a third fluid source having a fluid having a third known analyte concentration suitable for calibration of the analyte sensor mounted in fluid communication with the selection or mixing valve, the method can further comprise configuring the selection or mixing valve to deliver a first calibration fluid comprising fluid from the first fluid source, fluid from the third fluid source, or a combination thereof, and operating the pumps to flow the first calibration fluid past the analyte sensor while infusing into the patient a volume less than the volume defined by the fluid passage between the tubing junction and the selection or mixing valve, and configuring the selection or mixing valve to deliver a second calibration fluid comprising fluid from the first fluid source, fluid from the third fluid source, or a combination thereof, and operating the pumps to flow the second calibration fluid past the analyte sensor while infusing into the patient a volume less than the volume defined by the fluid passage between the tubing junction and the selection or mixing valve, and calibrating the analyte sensor responsive to its response to the first and second calibration fluids.

In a fifth example method, the present invention provides a method of calibrating an apparatus such as the third example apparatus, comprising operating the first and second pumps to withdraw blood from the patient past the analyte sensor and into the reservoir, and operating the first and second pumps to draw blood from the reservoir and fluid from the first fluid source to present a mixture of blood from the reservoir and fluid from the first fluid source to the analyte sensor, and calibrating the analyte sensor responsive to its response to the blood and to the mixture of blood and the fluid from the first fluid source.

In a method like the fifth example method, wherein the apparatus further comprises a second fluid source in fluid communication with the second port of the analyte sensor, wherein the second fluid source contains a fluid having a second known analyte concentration, different from the first known analyte concentration, suitable for calibration of the analyte sensor, the method can further comprise operating the first and second pumps to draw blood from the reservoir and fluid from the second fluid source to present a mixture of blood from the reservoir and fluid from the second fluid source to the analyte sensor, and calibrating the analyte sensor responsive to its response to the blood and to the mixture of blood and the fluid from the first fluid source and to the mixture of blood and fluid from the second fluid source.

Method for controlling a level of blood glucose in a patient using an extracorporeal blood circuit] An extracorporeal glucose system and controller has been developed which overcomes many of the limitation of currently proposed glucose control systems by enabling the measurement of the concentration of glucose in blood with little or no delay. This affords a much faster control system while protecting the glucose sensor from contamination by blood and facilitating periodic external calibration.

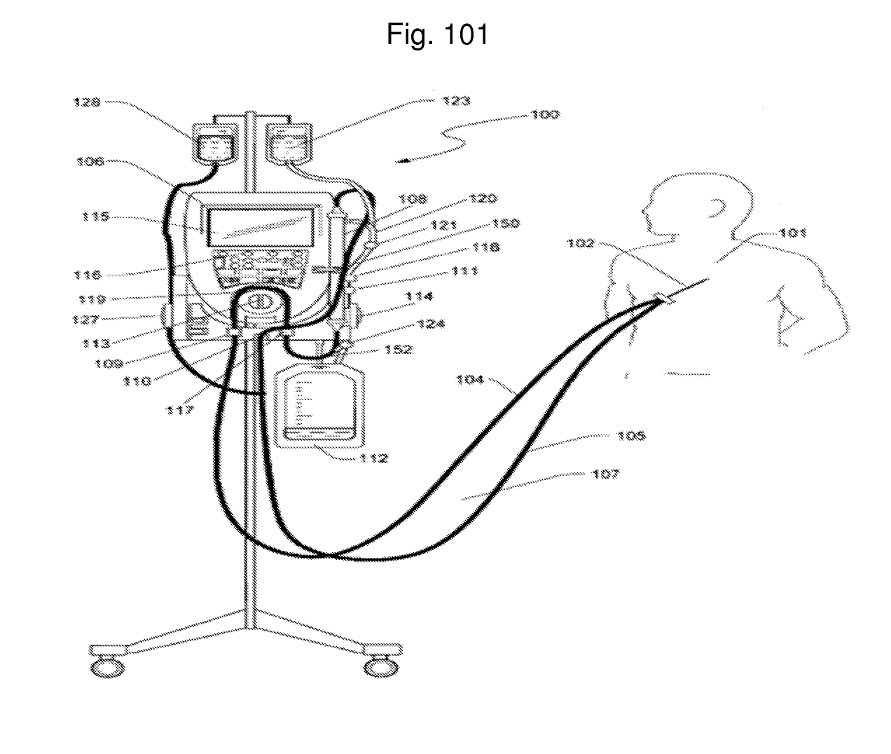

FIG. 101 illustrates the treatment of a patient requiring glucose maintenance with a glucose control apparatus 100. The patient 101, such as a human or other mammal, may be treated while in bed and may be conscious or asleep. The patient need not be confined to an intensive care unit (ICU). To initiate treatment, a standard 7 to 8F, dual or triple lumen CV (central venous) catheter 190 may be used. The catheter is introduced into suitable peripheral or central vein, antecubital, jugular, clavicle or femoral for the withdrawal and return of the blood. The catheter is attached to withdrawal tubing 104 and return tubing 105, respectively. The tubing may be secured to skin with adhesive tape.

The glucose maintenance apparatus includes a blood pump console 106 and a blood circuit 107. The console includes three rotating roller pumps that move blood, ultrafiltrate fluids and insulin through the circuit, and the circuit is mounted on the console. The blood circuit includes a continuous blood passage between the withdrawal line 104 and the return line 105. The blood circuit includes a blood filter 108; pressure sensors 109 (in withdrawal tube), 110 (in return tube) and 111 (in filtrate output tube); an ultrafiltrate collection bag 112 and tubing lines to connect these components and form a continuous blood passage from the withdrawal to the infusion catheters an ultrafiltrate passage from the filter to the ultrafiltrate bag, connections for the attachment of a glucose calibration solution 123 and an insulin infusion bag 128. The ultrafiltrate line 120 is connected to the glucose calibration solution 123 via the tubing 124 by a valve system facilitating the calibration sequence.

The blood passage through the circuit is preferably continuous, smooth and free of stagnate blood pools and air/blood interfaces. These passages with continuous airless blood flow reduce the damping of pressure signals by the system and allows for a higher frequency response pressure controller, which enables the pressure controller to adjust the pump velocity more quickly to changes in pressure, thereby maintaining accurate pressure control without causing instability in control. The components of the circuit may be selected to provide smooth and continuous blood passages, such as a long, slender cylindrical filter chamber, and pressure sensors having cylindrical flow passage with electronic sensors embedded in a wall of the passage. The circuit may come in a sterile package and is intended that each circuit be used for a single treatment.

The circuit mounts on the blood, insulin and ultrafiltrate pumps 113 (for blood passage) 127 for the insulin passage and 114 (for filtrate output of filter). The circuit can be mounted, primed and prepared for operation within minutes by one operator. The operator of the glucose control apparatus 100, e.g., a nurse or medical technician, sets the maximum rate at which fluid is to be removed from the blood of the patient. These settings are entered into the blood pump console 106 using the user interface, which may include a display 115 and control panel 116 with control keys for entering maximum flow rate and other controller settings.

Information to assist the user in priming, setup and operation is displayed on the LCD (liquid crystal display) 115. The operator also sets the target glucose level along with upper and lower control limits whereby the console 100 annunciates an alarm when exceeded.

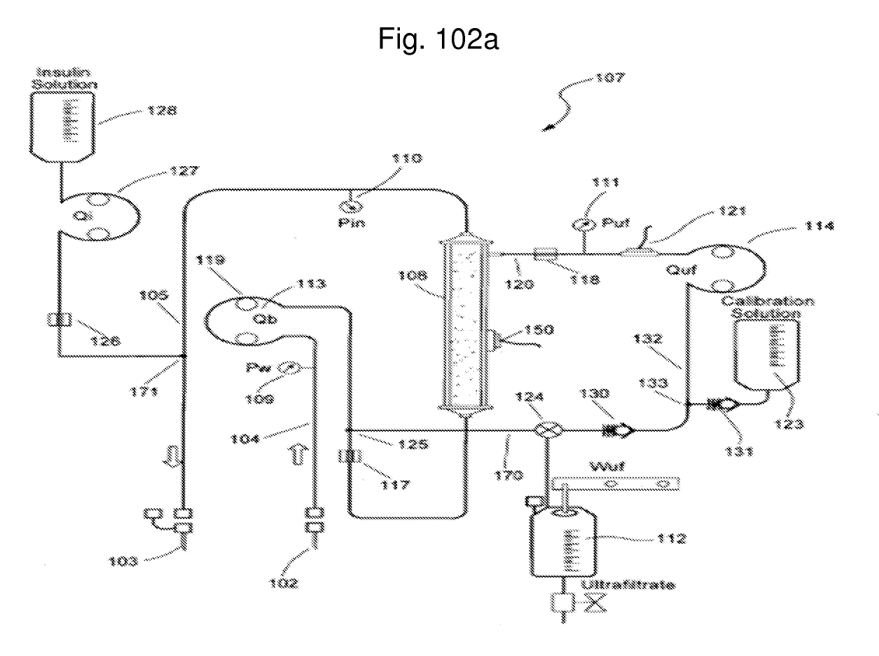

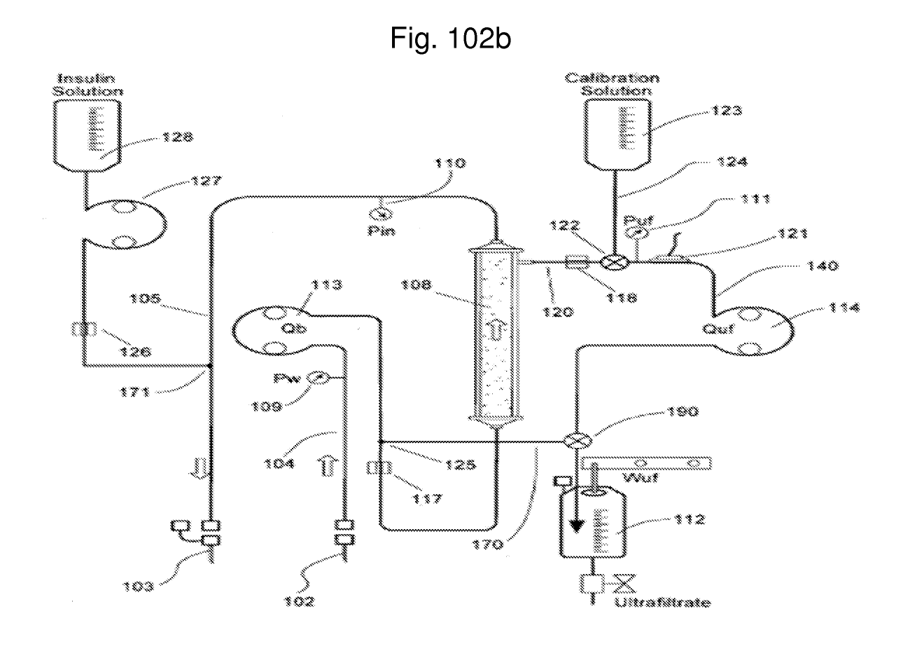

The ultrafiltrate is withdrawn by the ultrafiltrate pump 114 into a graduated collection bag 112 or is returned at the outlet of the blood pump 152 to facilitate predilution of the blood before entering the filter housing 108. The valve 124 may be manually switched by the operator or controlled automatically via a rotary solenoid valve based upon. When the bag is full, ultrafiltration delivery into the bag stops until the bag is emptied. The valve 124 can redirect the ultrafiltrate liquid exiting the ultrafiltrate pump 114 enter the blood line exiting the blood pump and predilute the blood entering the filter 108. The controller may determine when the bag is filled by determining the amount of filtrate entering the bag based on the volume displacement of the ultrafiltrate pump in the filtrate line and filtrate pump speed, or by receiving a signal indicative of the weight of the collection bag. An air detector 117 monitors for the presence of air in the blood circuit, blood is pumped through the circuit. The predilution ultrafiltrate may be returned upstream of the filter and the air detector 117 to ensure that air is not infused into the patient. A blood glucose sensor 150 is connected directly to the filtrate side of the filter with the sensor inserted between the hollow membrane fiber bundles ensuring the fastest signal response possible. A second blood glucose sensor 121 is attached to ultrafiltrate line 120 and can be calibrated with the glucose calibration solution from the bag 123 when the ultrafiltrate pump 114 is reversed via a one way valve 131 (FIG. 102a). A blood leak detector 118 in the ultrafiltrate output line 120 monitors for the presence of a ruptured filter. Signals from the air detector and/or blood leak detector may be transmitted to the controller, which in turn issues an alarm if a blood leak or air is detected in the ultrafiltrate or blood tubing passages of the extracorporeal circuit.

FIG. 102a illustrates the operation and fluid paths of blood, insulin and ultrafiltrate through the blood circuit 107. Blood is withdrawn from the patient through the lumens 102 and 103. The catheter is inserted into a suitable vein defined by current medical practice which can sustain a blood flow of 5 to 40 ml/min. The blood flow from the withdrawal tubing 104 is dependent on the fluid pressure in that tubing which is controlled by a roller pump 113 on the console 106. The algorithms for controlling the withdrawal, infusion and ultrafiltrate pressures are disclosed in U.S. Pat. Nos. 6,796,955; 6,689,083 and 6,706,007 and are incorporated by reference herein.

The pressure sensors may also have a blood passage that is contiguous with the passages through the tubing and the ID of the passage in the sensors may be similar to the ID in the tubing. It is preferable that the entire blood passage through the blood circuit (from the withdrawal catheter to the return catheter) have substantially the same diameter (with the possible exception of the filter) so that the blood flow velocity is substantially uniform and constant through the circuit. A benefit of a blood circuit having a substantially uniform ID and substantially continuous flow passages is that the blood tends to flow uniformly through the circuit, and does not form stagnant pools within the circuit where clotting may occur.

The withdrawal pressure sensor 109 is a flow-through type sensor suitable for blood pressure measurements. It is preferable that the sensor have no bubble traps, separation diaphragms or other features included in the sensor that might cause stagnant blood flow and lead to inaccuracies in the pressure measurement.

The filter 108 is used to:

Ensure that the glucose sensors 150 and 121 are not contaminated and made inoperable by blood components larger than 50,000 daltons.

Ultrafiltrate the blood and decrease the amount of time it takes for the glucose sensor to get an accurate reading of glucose in the blood.

Remove excess fluid from the patient if necessary.

Whole blood enters the filter 108 and passes through a bundle of hollow filter fibers in a filter canister. There may be between 100 to 1000 hollow fibers in the bundle, and each fiber is a filter. In the filter canister, blood flows through an entrance channel to the bundle of fibers and enters the hollow passage of each fiber. Each individual fiber has approximately 0.2 mm internal diameter. The walls of the fibers are made of a porous material. The pores are permeable to water and small solutes, but are impermeable to red blood cells, proteins and other blood components that are larger than 50,000-60,000 Daltons. Blood flows through the fibers tangential to the surface of the fiber filter membrane. The shear rate resulting from the blood velocity is high enough such that the pores in the membrane are protected from fouling by particles, allowing the filtrate to permeate the fiber wall. Filtrate (ultrafiltrate) passes through the pores in the fiber membrane (when the ultrafiltrate pump is rotating), leaves the fiber bundle, and is collected in a filtrate space between the inner wall of the canister and outer walls of the fibers. The volume of the filter that contains the ultrafiltrate has been designed to be as small as possible and still facilitate the manufacturing of the filter. This volume acts to dampen the real time blood glucose measurements by acting as a reservoir for ultrafiltrate. To help reduce this affect, the blood glucose sensor 150 is embedded in the ultrafiltrate compartment of the filter 108 with the sensor measurement site lying within the polysulphone fibers of the filter. The membrane of the filter acts as a restrictor to ultrafiltrate flow. An ultrafiltrate pressure transducer (Puf) 111 is placed in the ultrafiltrate line upstream of the ultrafiltrate roller pump 114. The ultrafiltrate pump 114 is rotated at the prescribed fluid extraction rate which controls the ultrafiltrate flow from the filter. Before entering the ultrafiltrate pump, the ultrafiltrate passes through approximately 10 cm of plastic tubing 120, the blood leak detector 118, the ultrafiltrate pressure transducer (Puf) and the second reference glucose sensor 121. The tubing is made from medical PVC of the kind used for IV lines and has internal diameter (ID) in this case of 3.2 mm. The ultrafiltrate pump 114 is rotated by a brushless DC motor under microprocessor control. The pump tubing segment (compressed by the rollers) has the same ID as the rest of the ultrafiltrate circuit.

In this operational configuration both the control glucose sensor 150 and the reference glucose sensor measure the concentration of glucose in the blood. The reference glucose sensor 121 has an added lag and time delay due to the volume of ultrafiltrate in the filter filtrate cavity and the volume of tubing between the outlet of the filter 120 and the reference glucose sensor 121. To periodically calibrate the reference glucose sensor 121, the ultrafiltrate pump 114 is reversed. When the ultrafiltrate pump 121 is reversed (rotated anticlockwise) the one way valve 130 prevents ultrafiltrate from the ultrafiltrate bag 112 or blood from the output of the blood pump from entering the return ultrafiltrate line 170. At the same time, glucose calibration solution is drawn through a one way valve 131 connected to the ultrafiltrate line 132 at the T-connection 133. The one-way valve 131 opens due to the negative pressure generated by the reversing ultrafiltrate pump 114. The ultrafiltrate pump is only displaced the volume required to flush the ultrafiltrate line 132 and ensure that the reference glucose sensor is reading an uncontaminated reference solution, e.g., the calibration solution 123. The volume of the tubing between the calibration solution 131 and the reference glucose sensor is less than the volume between the reference glucose sensor and the outlet of the ultrafiltrate from the filter 108. This ensures that during reversal the filtrate cavity of the filter 108 is not contaminated with the glucose calibration solution. During the calibration sequence the control glucose sensor 150 relies on diffusion to measure the correct level of glucose in the blood. The sensor 150 provides an uninterrupted signal for control during the calibration sequence.

After the blood passes through the filter 108, it is pumped through a two meter infusion return tube 105 to the infusion needle 103 where it is returned to the patient. The properties of the filter 108 and the infusion needle 103 are selected to assure the desired TMP (Trans Membrane Pressure) of 150 to 250 mm Hg at blood flows of 5 to 40 ml/min where blood has hematocrit of 35 to 48% and a temperature of room temperature (generally 21 to 23.degree. C.) to 37.degree. C.

Insulin is also infused into the return line of 105 of the blood circuit. The measurements taken from the control glucose sensor 150 are used to calculate the rate of infusion of glucose required to keep the patients glucose between 80 and 110 mg/dl. An insulin solution is withdrawn from the insulin solution bag 128 and pumped through an air detector 126 before being infused into the return line 105 via the T-connector 171. This configuration is shown with a peristaltic pump 127 but could be replaced with an infusion syringe pump. The pump 127 controls the rate of insulin injection. The controlled insulin rate is determined based on the measured glucose level.

The blood leak detector 118 detects the presence of a ruptured/leaking filter, or separation between the blood circuit and the ultrafiltrate circuit. In the presence of a leak, the ultrafiltrate fluid will no longer be clear and transparent because the blood cells normally rejected by the membrane will be allowed to pass. The blood leak detector detects a drop in the transmissibility of the ultrafiltrate line to infrared light and declares the presence of a blood leak.

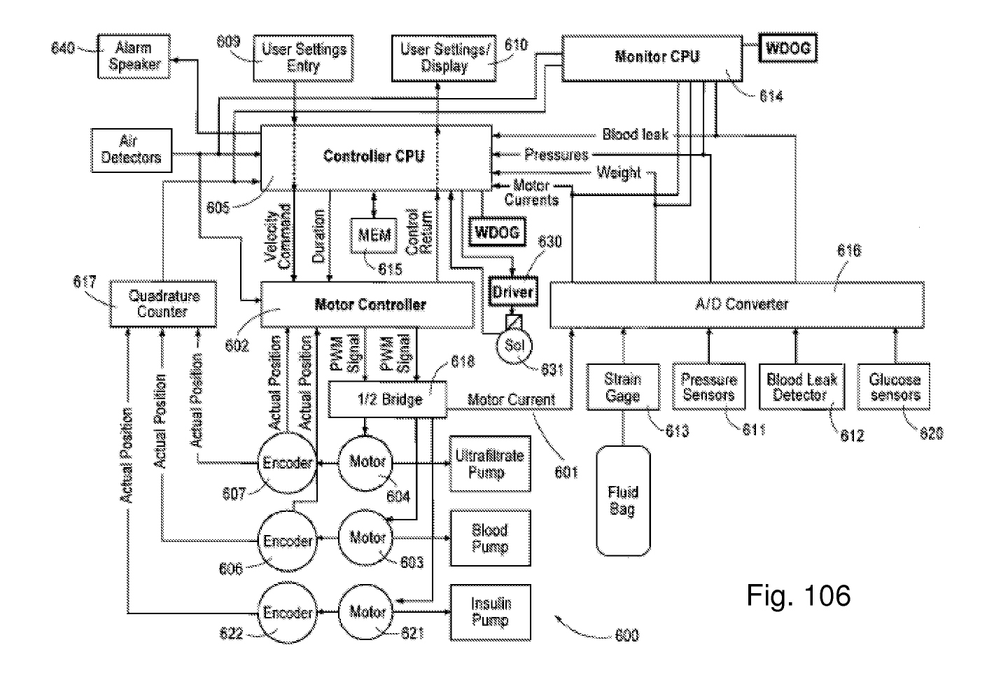

The pressure transducers Pw (withdrawal pressure sensor 109), Pin (infusion pressure sensor 110) and Puf (filtrate pressure sensor 111) produce pressure signals that indicate a relative pressure at each sensor location. Prior to filtration treatment, the sensors are set up by determining appropriate pressure offsets. The offsets are determined with respect to atmospheric pressure when the blood circuit is filled with saline or blood, and the pumps are stopped. The offsets are measures of the static pressure generated by the fluid column in each section, e.g., withdrawal, return line and filtrate tube, of the circuit. Absent these offsets, a false disconnect or occlusion alarm could be issued by the monitor CPU (605 in FIG. 106) because, for example, a static 30 cm column of saline/blood will produce a 22 mm Hg pressure offset.

FIG. 102b illustrates the operation a similar fluid path as that shown in FIG. 102a but in this instance the one way valve system for the infusion of the calibration solution 123 has been replaced with a valve 122 which is capable of switching the flow of fluid to the reference glucose sensor 121 from the output of the ultrafiltrate line 120 to the calibration solution 123. The ultrafiltrate pressure sensor is shown downstream of the valve 122 to ensure maintenance of pressure control limits during calibration. Since the valve and calibration solution lines 124 provide little or no resistance, if the ultrafiltrate pressure is seen to be excessively high when the calibration sequence is in process it is indicative of the calibration solution requiring replenishment or a valve 122 failing to toggle correctly. During calibration, the valve 190 may be toggled to direct the calibration solution to either the ultrafiltrate bag 112 or to the outlet blood line of the blood pump 125. The rest of the fluid path acts in the exact same manner as that outlined in FIG. 102a and is not repeated here.

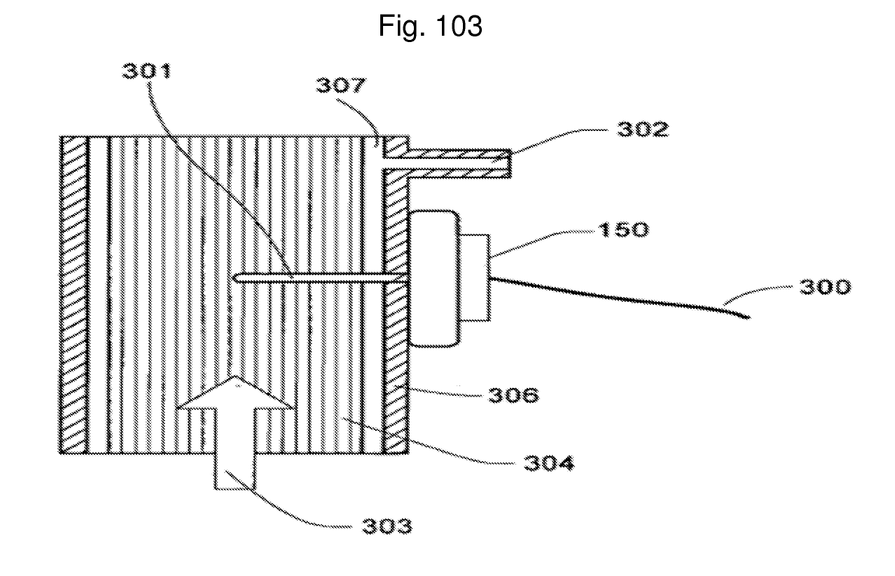

FIG. 103 illustrates the operation and position of the control glucose sensor within the filter fiber bundle. Currently blood glucose sensors are divided into general approaches, electroenzymatic and optical. The electroenzymatic sensors are based upon polarographic principles and utilize the phenomenon of glucose oxidation with a glucose oxidase enzyme. This chemical reaction can be measured electrically by sensing the current output of the sensor. There are two basic optical approaches, infrared absorption spectroscopy and fluorescence based affinity sensors. Any of these sensors can be configured for the approach outlined. As blood 303 passes through the hollow membrane fibers 304 ultrafiltrate is extracted through the permeable wall of the hollow membrane fibers. The sensor 301 is positioned within the fiber bundle to reduce the response time by taking advantage of the diffusion of glucose across the membrane and to minimize the volume of ultrafiltrate that has to be cleared before the control glucose sensor accurately represents the level of glucose in the blood. The control glucose sensor 150 is attached to the wall of the filter canister 306. The ultrafiltrate removed from the blood in the hollow membrane fibers exits the filter canister 306 at the port 302. The filtrate volume represented by 307 in this illustration of the filter canister is minimized to improve signal response time.

Optical sensors which use infra red light of two or more wavelengths either transmissively or reflectively are also well suited for this application. Many of the issues with implanting such devices are now overcome, such as sensor size, variations in tissue and individual calibrations for each patient.

The solenoid controlled valve system shown in FIG. 102b can be implemented with standard stopcocks making the valves disposable and enabling them to be components of the disposable blood circuit.

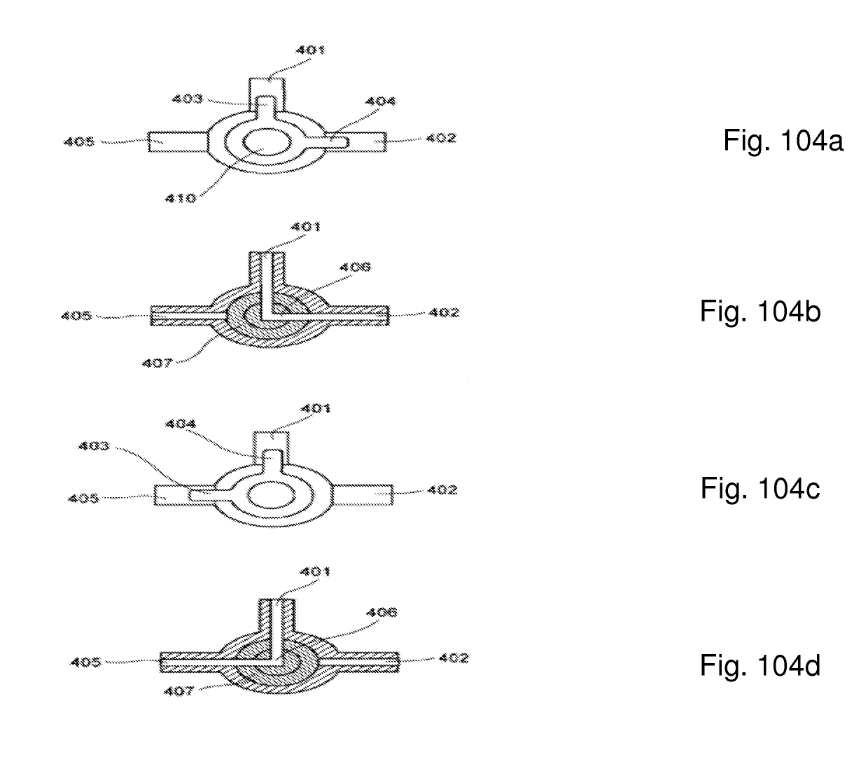

FIG. 104a shows the plan view of a standard three port, two-way stopcock (e.g. Qosina P/N 99743). The stopcock has three ports and can connect two ports together at a time. The lever arm of the stopcock is represented by 410 with arms 403 and 404. The arms point to the ports that are connected 401 and 402. The port 405 is closed in this configuration.

FIG. 104b shows a cross-section of the same valve in the same lever position showing the ports 401 and 402 connected via the conduit 406. The conduit allows fluid to flow from port 401 to 402.

FIG. 104c shows the lever arm 410 rotated 90 degrees anti-clockwise from that displayed in FIG. 104a with the lever arm 404 pointed towards port 401 and lever arm 403 pointed towards port 405. Thus port 401 is the common port and it can be switched from port 402 to port 403 by rotating the lever arm 410 (FIG. 104a)

FIG. 104d shows a cross-section of the valve in the configuration of FIG. 104c with the ports 401 and 405 connected via the conduit 406. The body of the valve 407, swivels as the lever arms are rotated.

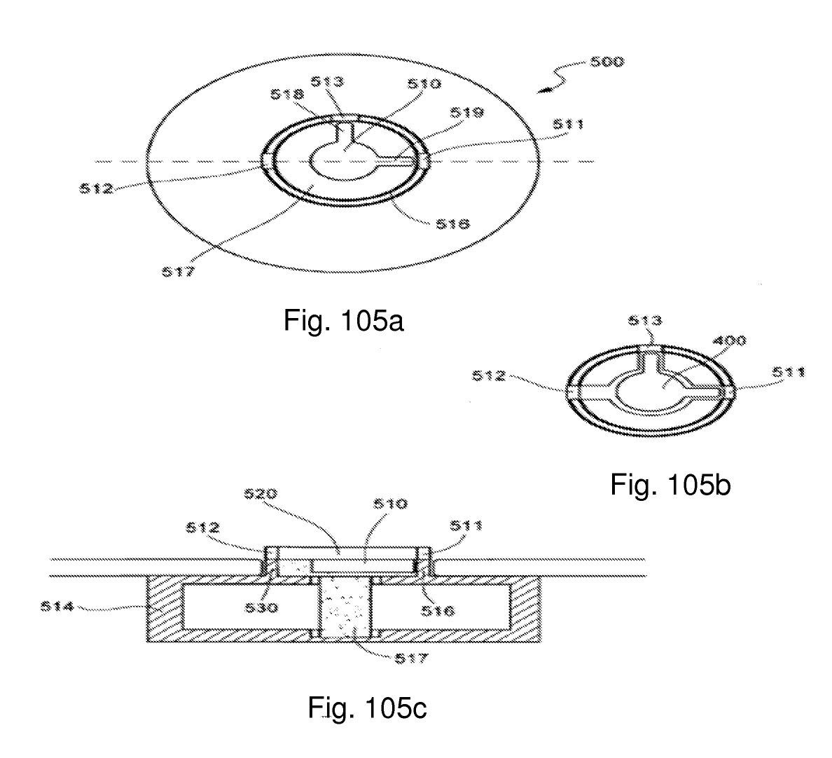

FIGS. 105a, 105b and 105c show a plan and elevation view of a rotary solenoid valve 500 for rotating the stopcock lever arm 410 shown in FIGS. 104a and 104c. The diagram shows how the stopcock 400 (FIG. 104a) fits into a recess in the shaft 520 of the solenoid valve and when rotated redirects flow from ports 401 to 402 to ports 402 to 405 (FIG. 104a). The actuator for rotating the stopcock could also be implemented with a stepper motor or a DC motor.

The one way valves 130 and 131 in FIG. 102a are spring return valves with a cracking pressure of approximately 1 psi. This prevents leaks due to the static head pressure caused by difference in height between the glucose calibration solution and the position of the one way valve 131 and time delays in the closure of the valve if no back pressure exists.

FIG. 106 illustrates the electrical architecture of the glucose control system 600 (100 in FIG. 101), showing the various signal inputs and actuator outputs to the controller. These settings may include the maximum flow rate of blood through the system, maximum time for running the circuit to filter the blood, the maximum ultrafiltrate rate and the maximum ultrafiltrate volume. The settings input by the user are stored in a memory 615 (mem.), and read and displayed by the controller CPU 605 (central processing unit, e.g., microprocessor or micro-controller) on the display 610.

The glucose control systems may also be used solely for the purposes of real time monitoring of blood glucose levels. To select this option the active control of glucose may be disabled via the membrane panel 610 ceasing the infusion of insulin. During this mode the user interface via the LCD displays a message to the user that active control of glucose has ceased. In this mode the device can be used to aid the medical practitioner in determining when it is necessary to titrate insulin manually. The alarm limits can be set to highlight when adjustments to manual titration of insulin are necessary obviating the need for the medical practitioner to continuously or intermittently monitor the patient. The monitoring system will alarm if the patients glucose level exceeds preset set alarm limits.

Glucose control systems mimic the body's natural insulin response to blood glucose levels as closely as possible in implanted glucose control applications, because excursions in the body without regard for how much insulin is delivered can cause excessive weight gain, hypertension and atherosclerosis. The proposed system suffers from very little signal time delay and lag. It is not necessary to wait for the insulin to transport through the interstitial space to the blood volume and back again to interstitial space to reach equilibrium. Insulin is infused directly into the blood and is transported directly to the interstitial space and organs. Control is based upon the measurement of the blood glucose level and the only delays and lag which occur are those of the insulin mixing in the blood volume, the transport of blood from the body to the filter and the transport of the ultrafiltrate to the sensor.

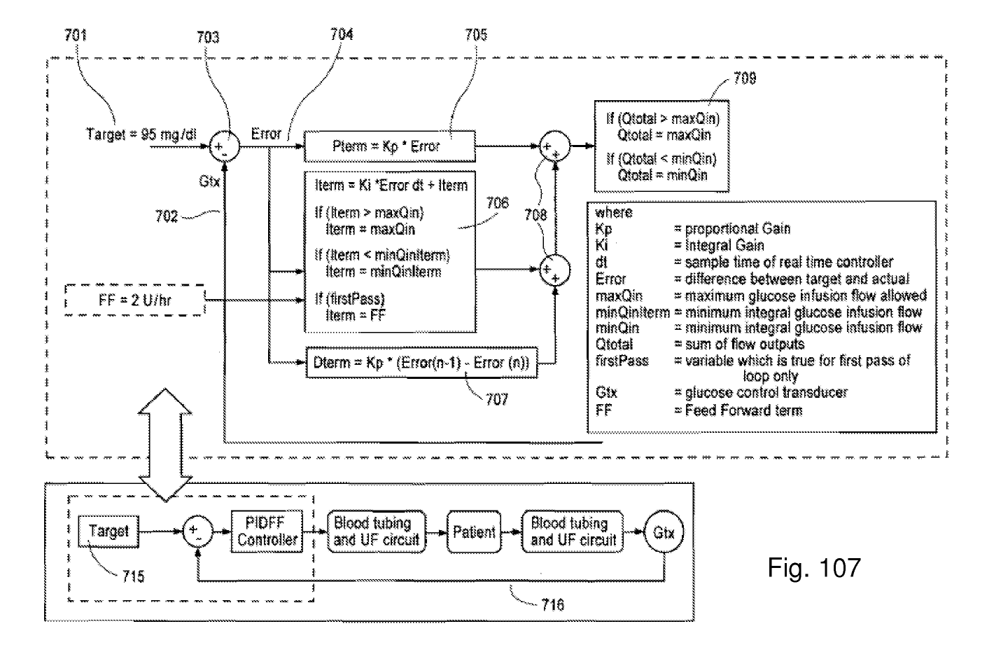

FIG. 107 shows the implementation of a PIDFF (Proportional Integral Derivative Feed Forward) controller whose purpose is to main a target 701 glucose level of the patient of 95 mg/dl. The control glucose sensor is read at a sample rate between 30 seconds and 10 minutes. For the purpose of this explanation it can be assumed that the measurement Gtx 702 is taken every 2 minutes. An error is calculated as Error=Target−Gtx. Based upon this error a proportional 705, integral 706 and determinative term 707 are calculated. The integral term when started for the first time is set to have an output of 2 U/hr of insulin. The integral term is limited in both the positive and negative direction to limit windup. In this case the integral has a separate specific minimum integral term allowed minQinlterm. The outputs of the proportional, integral and derivatives are summed and once again limited. Such a scheme allows for a more stable control system allowing symmetry in the integral controller. Once the insulin infusion rate is calculated a command is sent to the motor controller to implement the infusion rate.

The withdrawal pressure controller is based upon the withdrawal blood flow but the infusion pressure controller is based upon both the blood flow and the insulin infusion. As the blood flow reduces in response to a partial occlusion the ultrafiltrate rate is reduce not to exceed 20% of the blood flow rate. When the blood flow rate is less than 10 ml/min, 25% of the target blood flow rate of for example 40 ml/min ultrafiltration is stopped and the device alarms to inform the user of the condition. If the set blood flow rate was 5 ml/min then ultrafiltration would be stopped when the blood flow dropped below 1.25 mL/min. Glucose infusion rates are well less than 1 ml/min and in reality have little or no affect on the pressure control. During a total occlusion when the system reverses glucose control is terminated for the duration of the reversal.

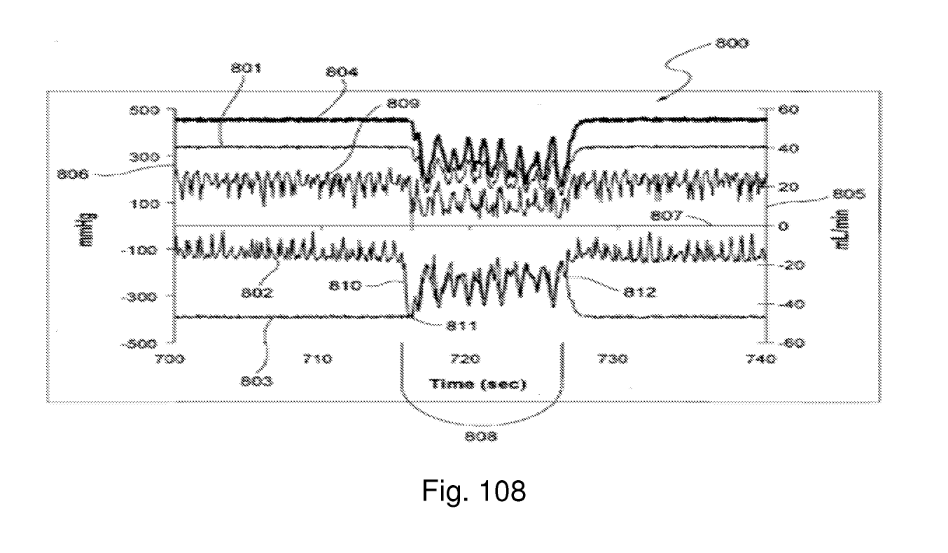

FIG. 108 illustrates the operation of a glucose control device under the conditions of a partial and temporary occlusion of the withdrawal vein. Blood was withdrawn from the left arm and infused into the right arm in different veins of the patient using similar 18 Gage needles. A short segment of data, i.e., 40 seconds long, is plotted in FIG. 108 for the following traces: blood flow in the extracorporeal circuit 804, infusion pressure occlusion limit 801 calculated by CPU 605 (FIG. 106.0), infusion pressure 809, calculated withdrawal pressure limit 803 and measured withdrawal pressure 802. Blood flow 804 is plotted on the secondary Y-axis 805 scaled in mL/min. All pressures and pressure limits are plotted on the primary Y-axis 806 scaled in mmHg. All traces are plotted in real time on the X-axis 807 scaled in seconds.

FIG. 108 illustrates the occlusion of the withdrawal line only. Although the infusion occlusion limit 801 is reduced in proportion to blood flow 804 during the occlusion period 808, the infusion line is never occluded. This can be determined by observing the occlusion pressure 809 always below the occlusion limit 801 by a significant margin, while the withdrawal occlusion limit 803 and the withdrawal pressure 802 intercept and are virtually equal during the period 808 because the PIFF controller is using the withdrawal occlusion limit 803 as a target.

The rapid response of the control algorithm is illustrated by immediate adjustment of flow in response to pressure change in the circuit. This response is possible due to: (a) servo controlled blood pump equipped with a sophisticated local DSP (digital signal processing) controller with high bandwidth, and (b) extremely low compliance of the blood path.

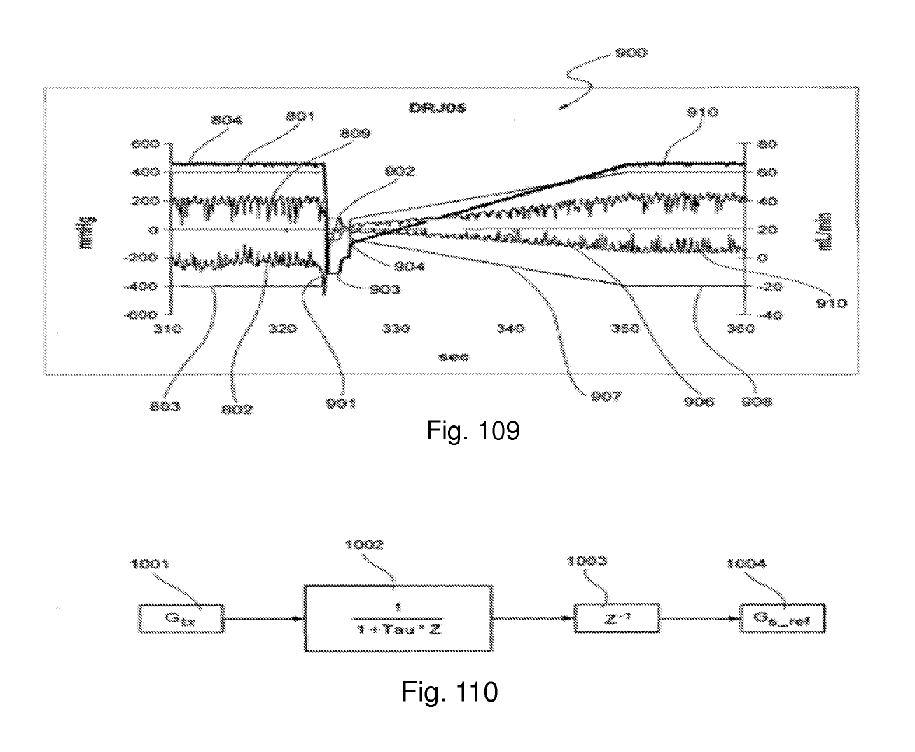

FIG. 109 illustrates a total occlusion of the blood withdrawal vein access in a different patient, but using the same apparatus as used to obtain the data shown in FIG. 108. The blood flow 804 is controlled by the maximum flow algorithm and is equal to 66 mL/min. The withdrawal pressure 802 is at average of −250 mmHg and safely above the occlusion limit 803 at −400 mmHg until the occlusion event 901. Infusion pressure 809 is at average of 190 mmHg and way below the infusion occlusion limit 801 that is equal to 400 mmHg.

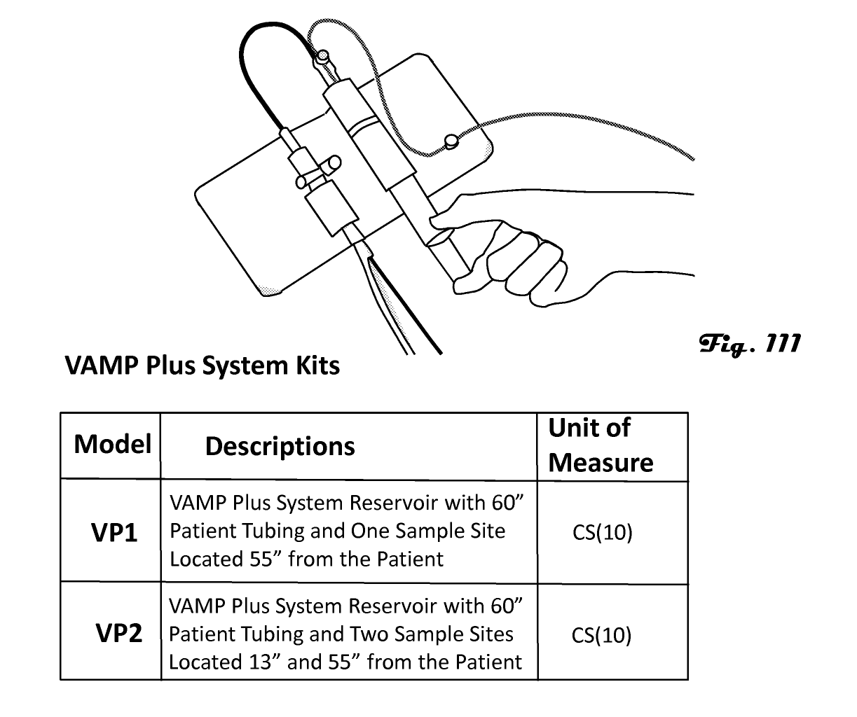

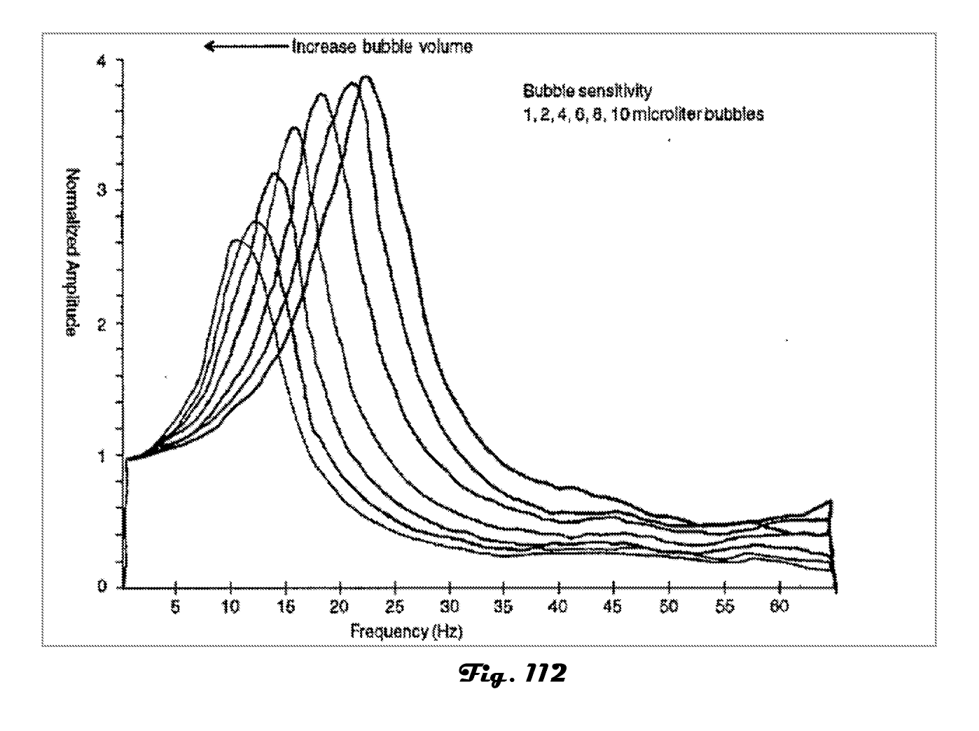

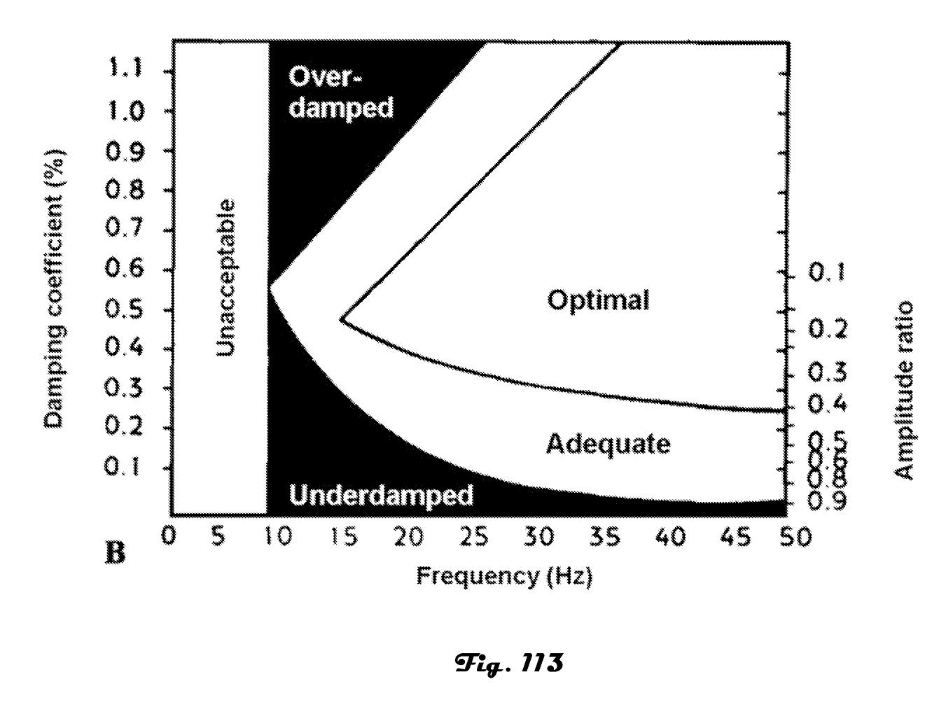

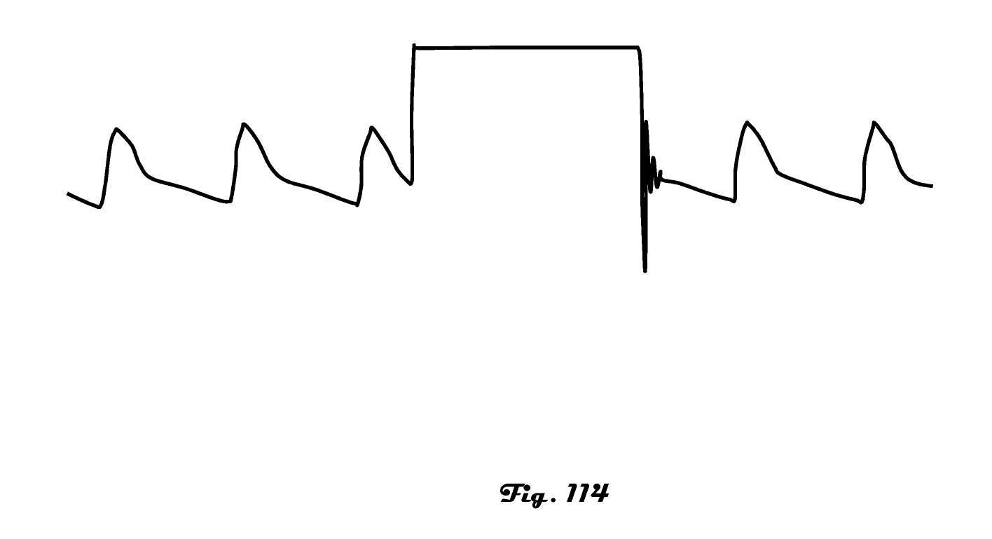

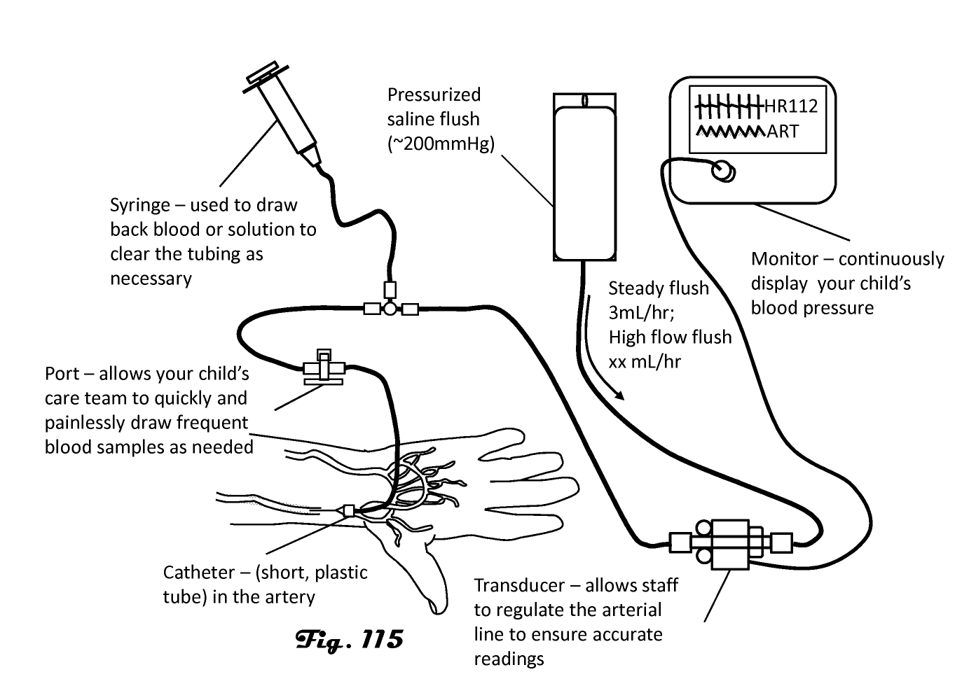

FIG. 110 shows how the reference glucose sensor can be compared directly with the control glucose sensor by modeling the plant between the two sensors. Gtx 101 is first filtered by a low pass filter 1002 that is modeled on the ultrafiltrate volume and ultrafiltrate flow rate. Next the output of the low pass filter 1002 is placed in a delay buffer representing the time delay of the ultrafiltrate to flow from the filter outlet past the reference glucose sensor. This delay is modeled as a function of ultrafiltrate flow and the transit delay between sensors. The output of the buffer Gs_ref 1004 is compared directly to the output of the reference glucose sensor. If the signals differ from each other by more than 5 mg/dl for a 5 minute period a control glucose sensor calibration sequence is initiated. This differs from the reference calibration sequence where the ultrafiltrate pump is reversed and the reference calibration signal is calibrated with the glucose calibration solution. The glucose control sensor calibration sequence consists of adjusting the sensitivity of the control glucose sensor until both sensors match.

Detection of bubbles during hemodvnamic monitoring Example embodiments of the present invention provide methods and apparatuses that enable the detection of bubbles so that hemodynamic performance can be assured following an automated blood analyte measurement. An example apparatus according to the present invention comprises a blood access system, adapted to remove blood from a body and infuse at least a portion of the blood back into the body. The infusion of at least a portion of the blood back in to the body can be done in a manner to assure that no bubbles of clinical significance are injected into the patient. Additionally an example embodiment can assess for the presence of bubbles in the fluid column that can affect hemodynamic monitoring performance. If a condition exists where hemodynamic monitoring performance cannot be assured, an example embodiment can provide appropriate warning or corrective actions.

An example method according to the present invention can comprise a bubble detection system used in conjunction with an automated analyte measurement and a hemodynamic monitoring system. The description herein will refer to an example blood access system for convenience. Other blood access systems and other analyte measurement techniques are also suitable for use with the present invention, as examples including those described in the patents and patent applications incorporated by reference herein.

Some example embodiments of the present invention provide for the detection of bubbles that would adversely impact the performance of the hemodynamic monitoring system. Some example embodiments of the present invention provide for both the detection of bubbles that can adversely impact the performance of the hemodynamic monitoring system and provide for a mechanism to remove these bubbles. Some example embodiments of the present invention can minimize the formation of bubbles during the automated blood measurement process.

An ICU (intensive care unit) pressure monitoring application is illustrated in FIG. 115. A pressure transducer is in direct contact with the arterial blood via a fluid column or stream. In typical operation a pressurized saline bag is used to infuse a small amount of saline into the patient at a constant rate. This saline infusion helps to keep the access site open. During a typical blood withdrawal sequence, the stopcock at the pressure transducer is closed and a sample is procured by a syringe attached to the arterial catheter. During this period of time no hemodynamic monitoring occurs. Following completion of the blood sample procurement, the stopcock is again opened and hemodynamic monitoring is reinitiated. The nurse or clinician will typically examine the arterial waveform for artifacts and inspect the tubing to ensure that no bubbles are present.

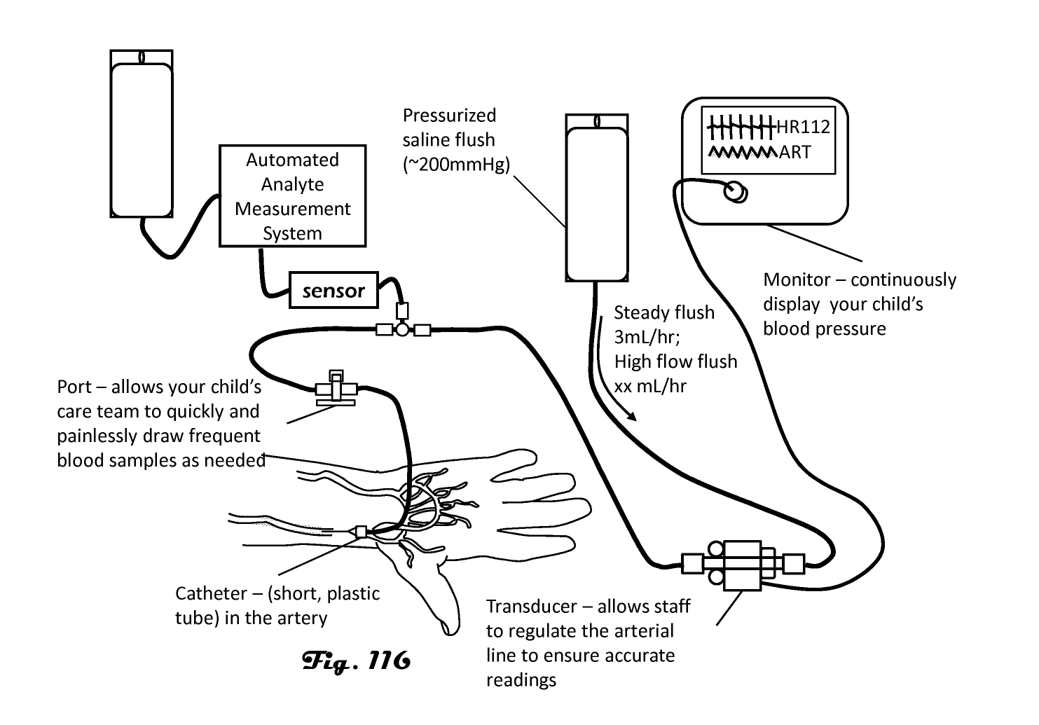

As shown in FIG. 116 an automated sample acquisition and analyte measurement system (e.g., a measurement system that measures one or more analytes in blood, such as glucose, arterial blood gasses, lactate, hemoglobin, and urea) can be attached to a similar system in a manner similar to the syringe blood withdrawal port illustrated in FIG. 115. If the process is to be automated, the patient, the pressure transducer and the analyte measurement system are in fluid connection. By fluid connection, it denotes a condition where fluid can travel between the patient, the analyte measurement system and the pressure transducer without changes to the system or the opening or closing of valves. If during sample procurement by the automated analyte measurement system an air bubble is created it can have some degree of adverse impact on the hemodynamic monitoring system due to the bubble being in fluid connection with the pressure transducer. The impact of the bubble can vary depending upon both size and location in the system. As shown in FIG. 112 even a small bubble can result in inaccurate pressure measurements.

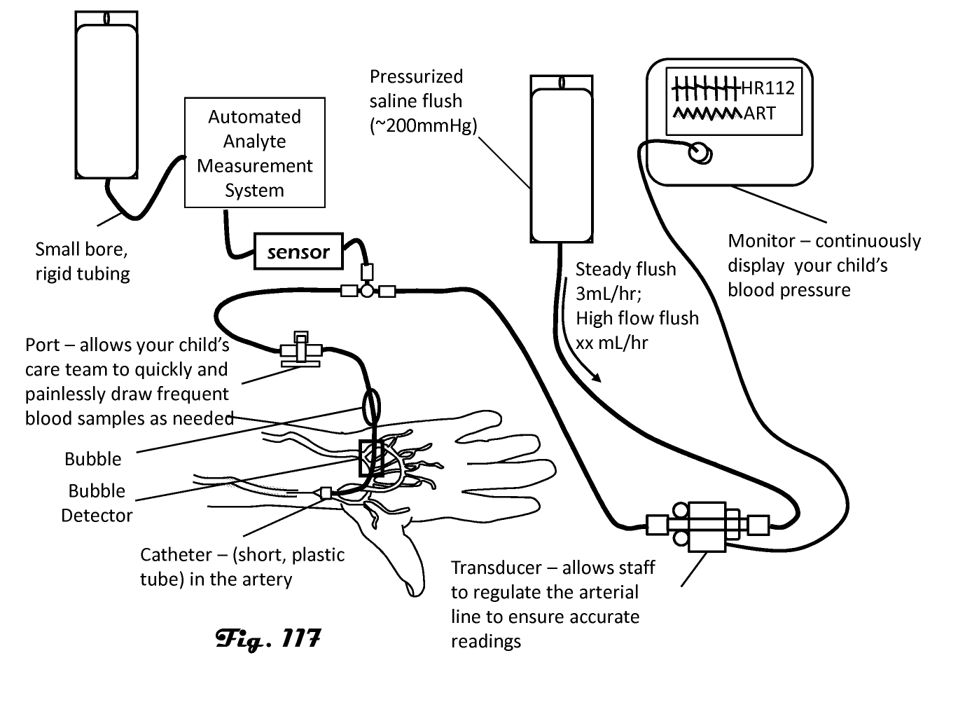

FIG. 117 illustrates a potentially problematic condition where a bubble is present between patient and the pressure transducer but removed from the bubble detector. The detection of such a bubble in this section of tubing is problematic and would historically have required visual inspection of the system or a fast-flush hemodynamic test administered by the clinician.

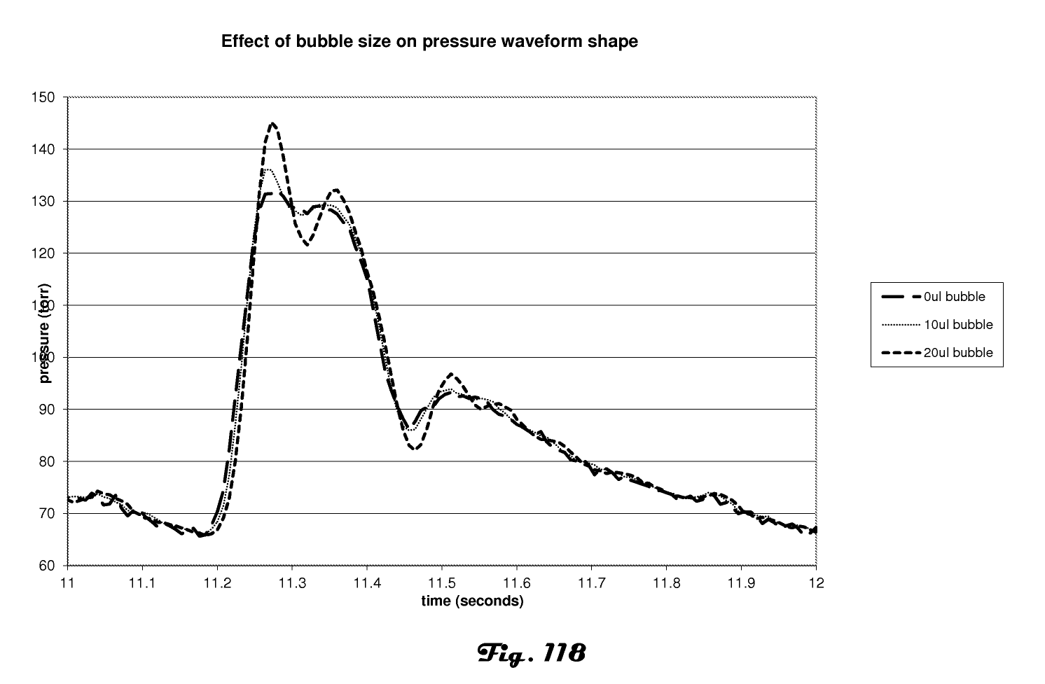

FIG. 118 illustrates the results of a laboratory test that illustrates the impact of bubbles on the resulting recorded waveform. In the laboratory tests, a variable pressure device was programmed to reproduce an arterial waveform. A standard blood pressure transducer in a standard clinical configuration was attached to the variable pressure device and waveform recordings were initiated. An initial test with no air bubbles in the line was recorded. Also recorded was a waveform tracing with a 10 μL bubble present, and a waveform tracing with a 20 μL bubble present. Examination of the corresponding waveforms illustrates that the presence of bubbles in the fluid path causes distortions in the true signal. Examination of the plot shows approximately a 5 mm Hg measurement error for the 10 μL bubble in the systolic pressure readings. The error is approximately 15 mm Hg for the 20 μL bubble. Additionally, the system exhibits signs of being under damped and thus shows some ringing after rapid changes.

A comparison between the pre-measurement waveform and post measurement waveforms can enable the detection of a bubble or bubbles that can affect hemodynamic performance. This comparison can take many forms to include simple subtraction, division, Fourier transform analysis, wavelet analysis, vector comparison, derivative processing, or any other mathematical treatment that enables a comparison between the two waveforms whereby the presence of a bubble can be detected.

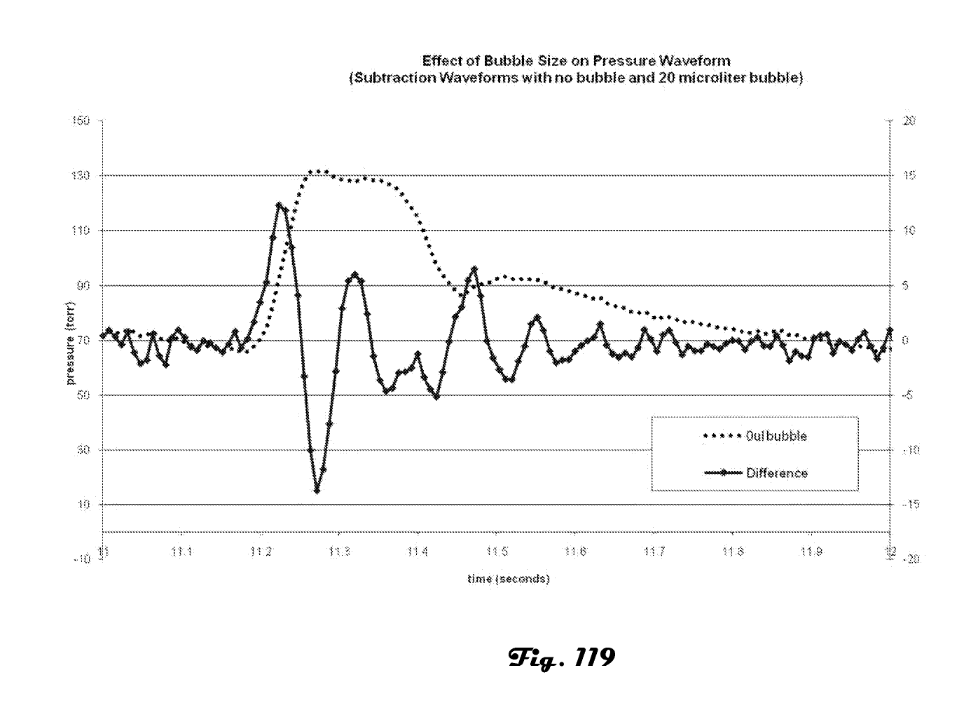

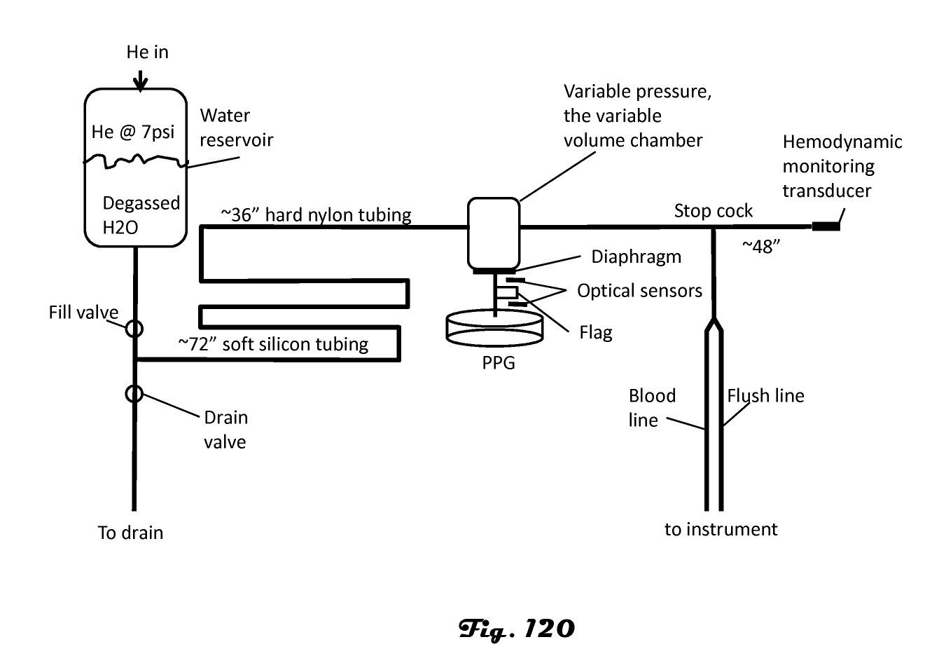

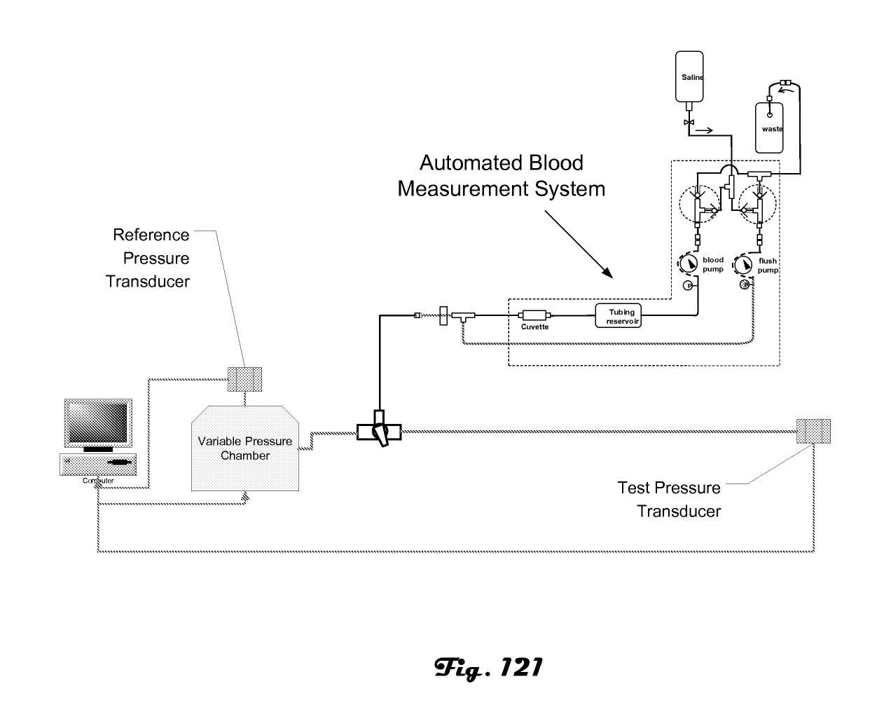

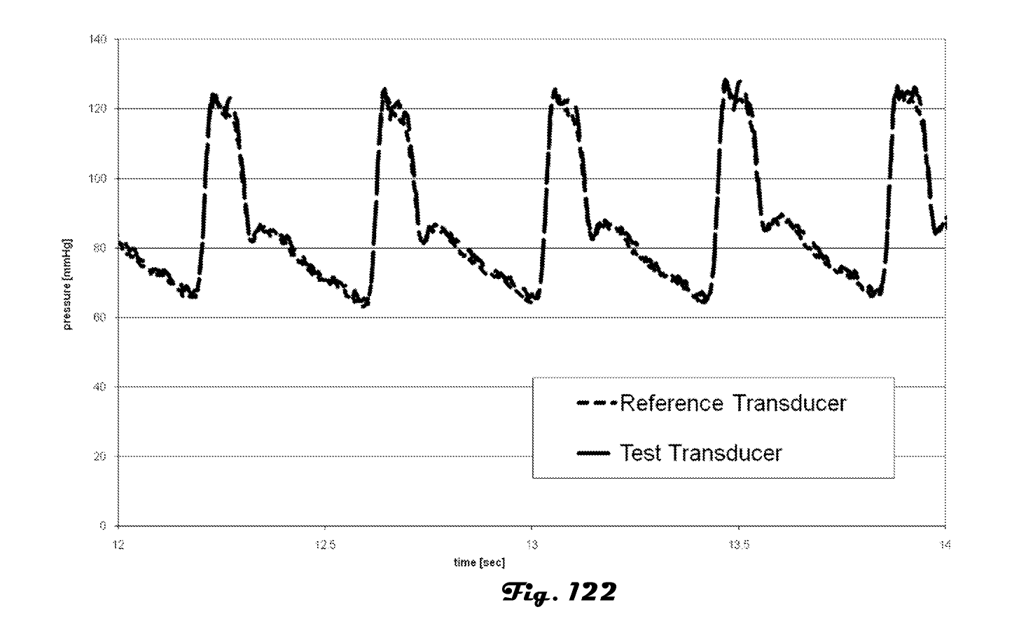

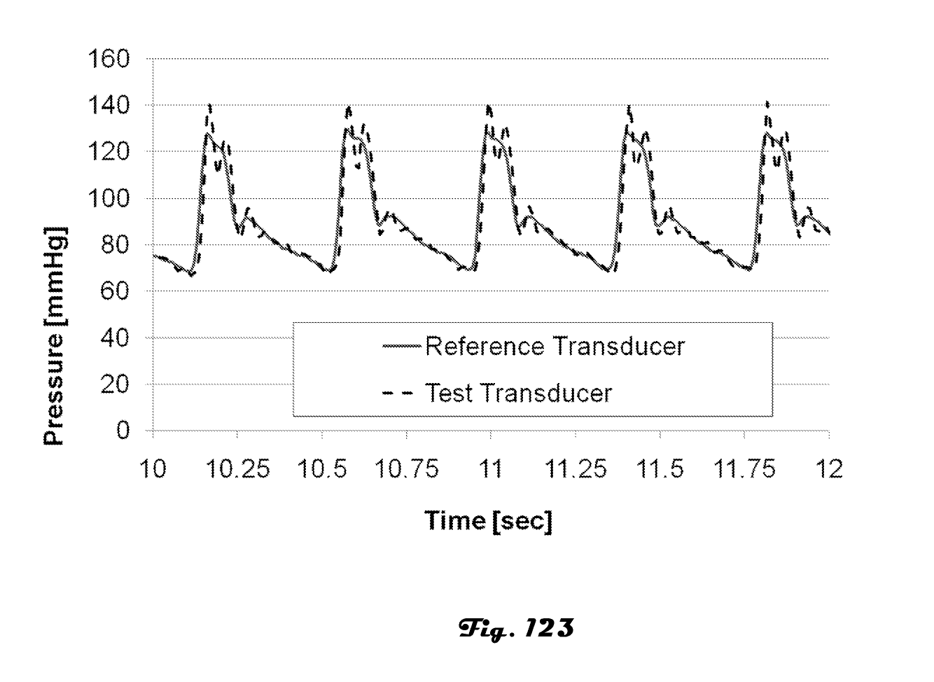

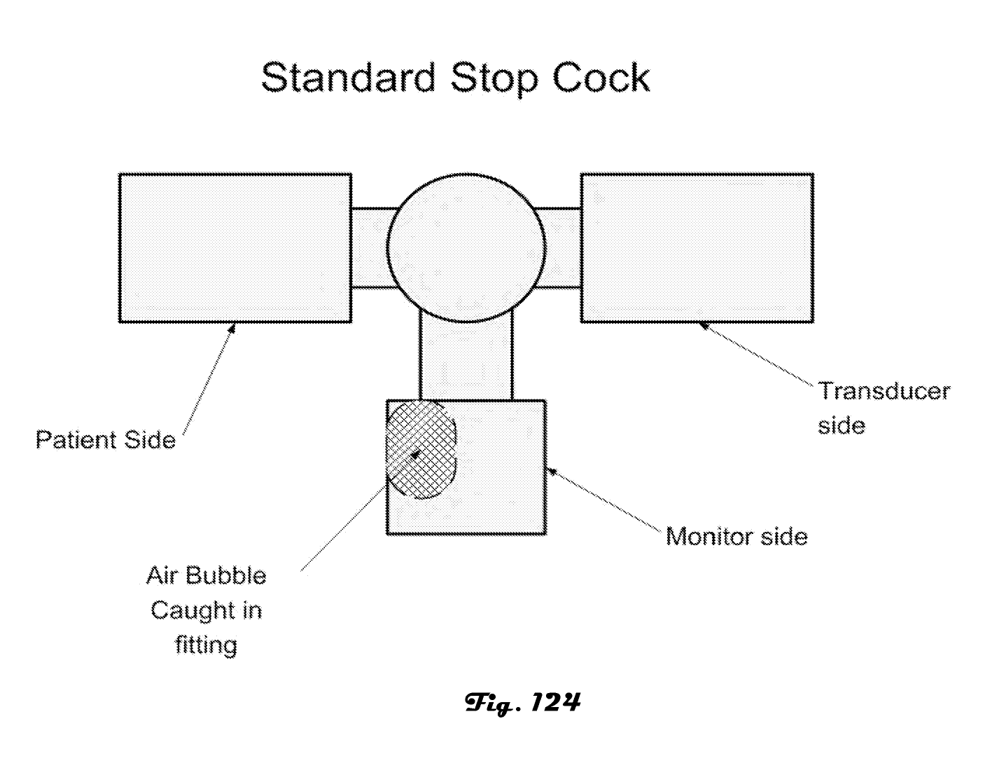

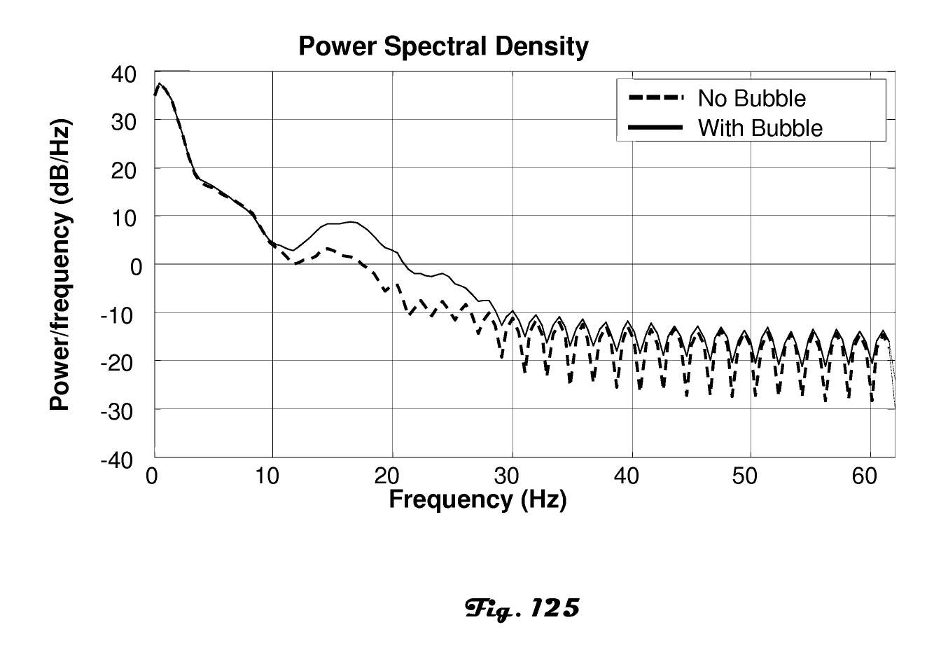

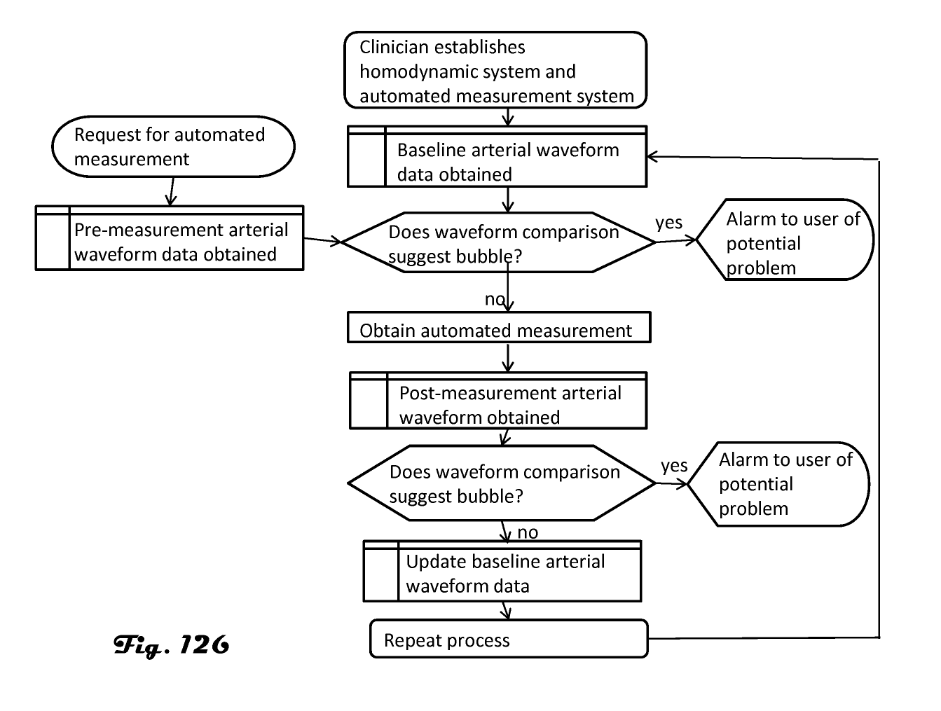

For illustrative purposes FIG. 119 shows a simple subtraction between a waveform with no bubble and a waveform with a 20 μL bubble. The resulting differences are large at the systolic peak and a simple threshold comparison can be used to detect the potential presence of a bubble.

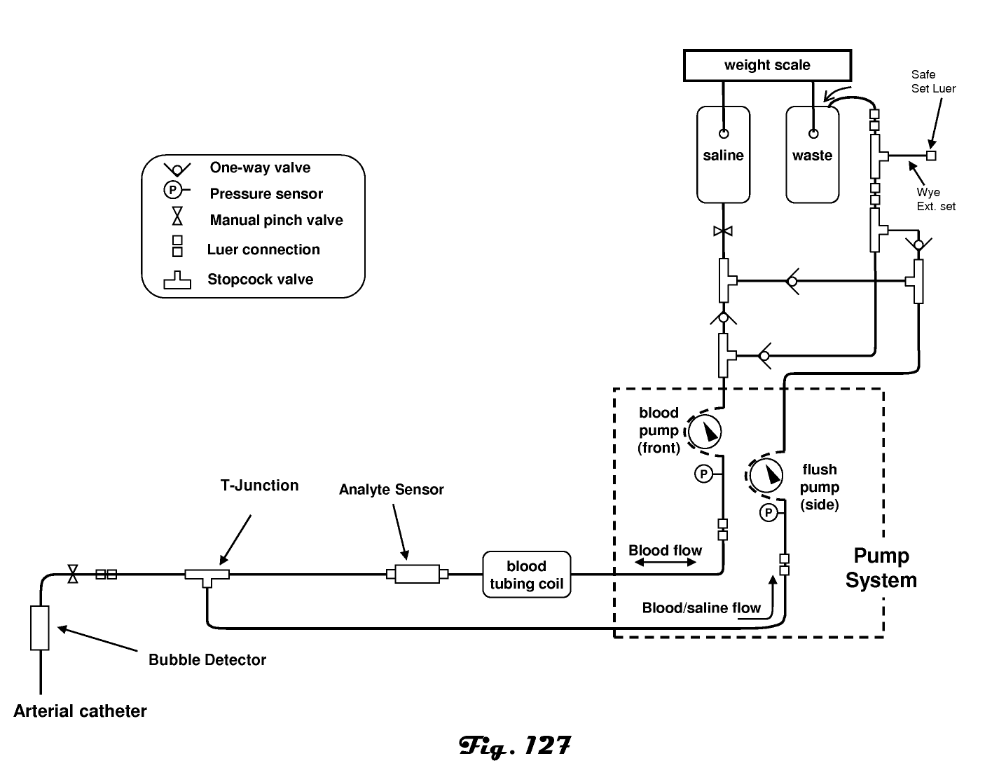

FIG. 127 is an example of an automated blood analyte measurement system. This system has a second tubing loop and pressure transducer that enables the effective removal of bubbles to waste. In practice, the blood for measurement is pulled to the analyte sensor and a measurement made with subsequent re-infusion into the patient. Several steps associated with cleaning the system can be performed after the measurement sequence. If a bubble is detected the system has the ability to move the bubble into the waste bag. An example process such as the following can be used. The blood pump can push fluid toward the patient while the flush pump pulls fluid away from the patient thus moving a bubble located between the pumps and the T-junction to a waste channel such as a waste bag as shown in the figure. By operating the pumps at the same rate but in opposite directions, the bubble can be moved to waste without risk of infusing the bubble into the patient. After an appropriate volume has been pumped the system can conduct a waveform comparison like those described elsewhere herein. If there is still evidence of a bubble then the likely location of the bubble is in the tubing between the bubble detector and the T-junction. To remove this bubble, the system can withdraw fluid toward and past the T-junction such that any bubble originally in the tubing between the T-junction and the patient is now located in the tubing sections between the t-junction and the pumps. Following the withdrawal process, the pumps can be activated in the manner described above so that the bubble is moved to the waste bag. To ensure that the system is now ready to begin hemodynamic monitoring, a final waveform test can be conducted. If such a test continued to indicate evidence of a bubble then the process can be repeated or an alarm initiated such that clinician resolution of the situation was initiated.

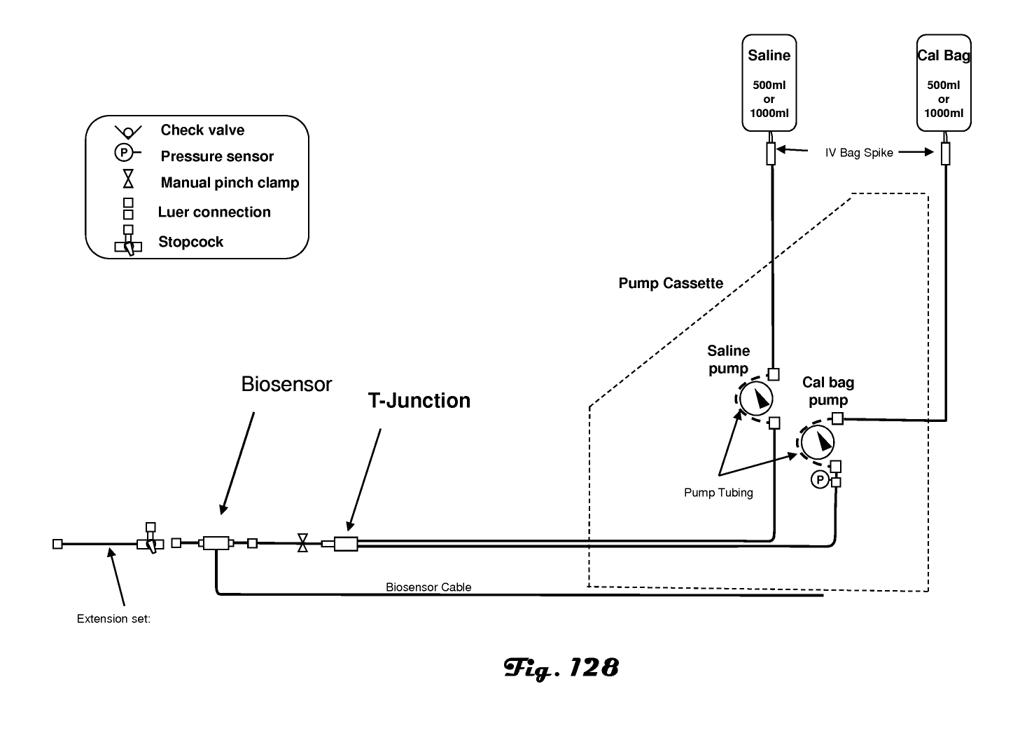

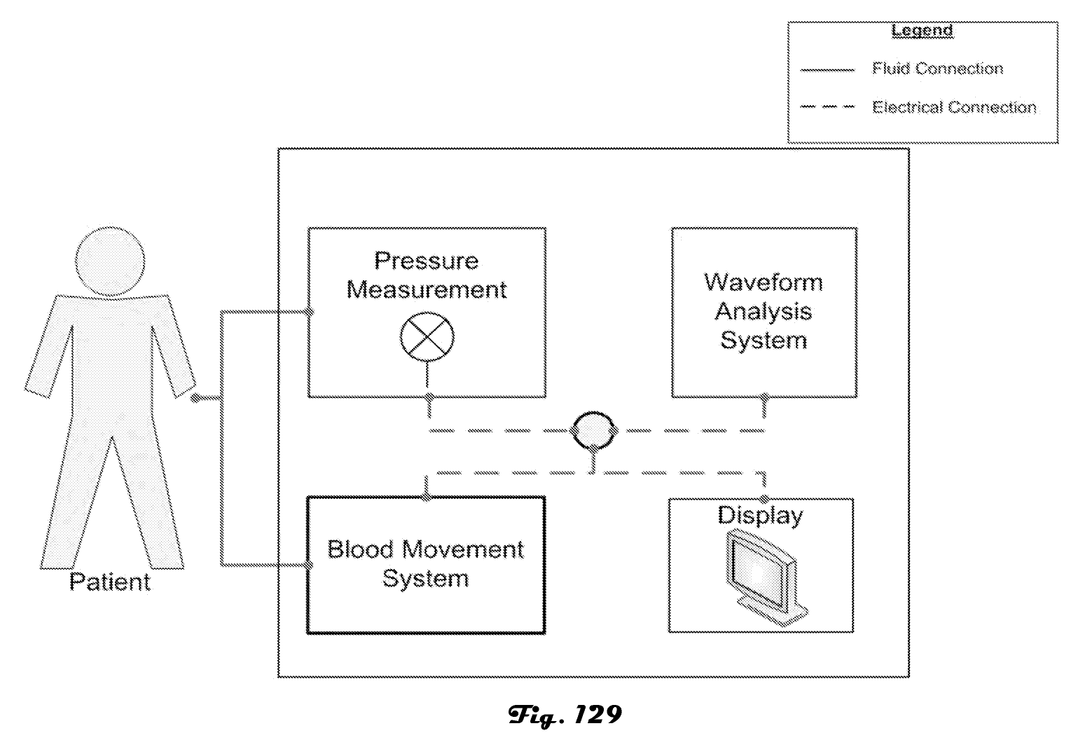

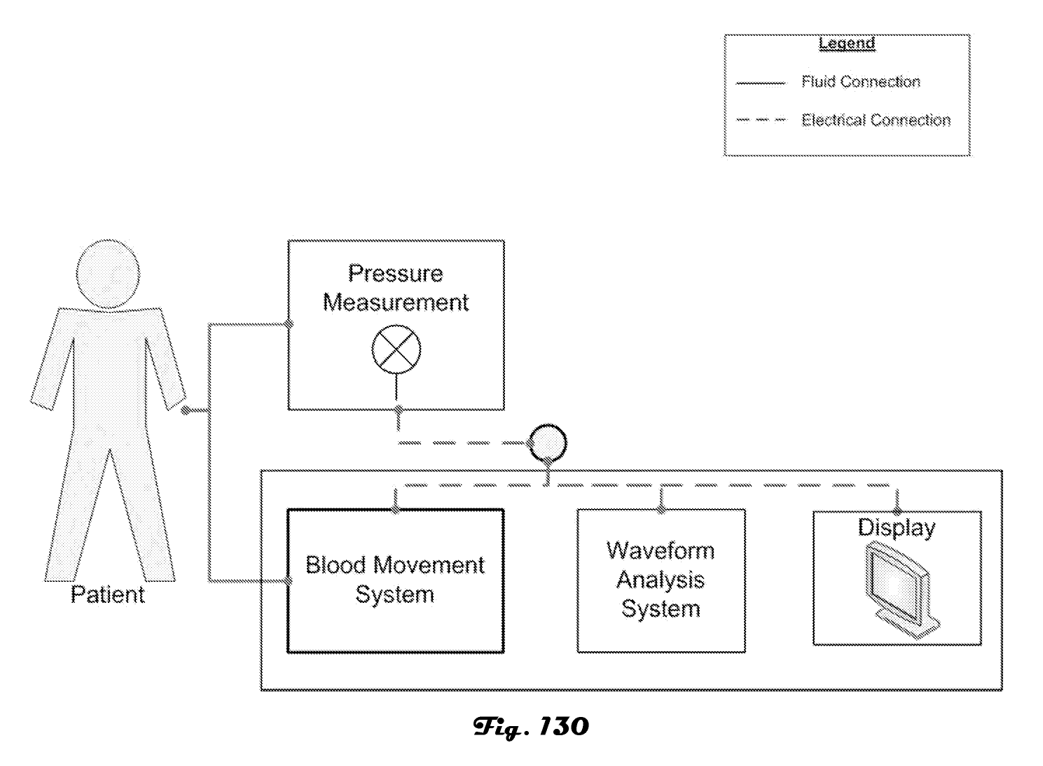

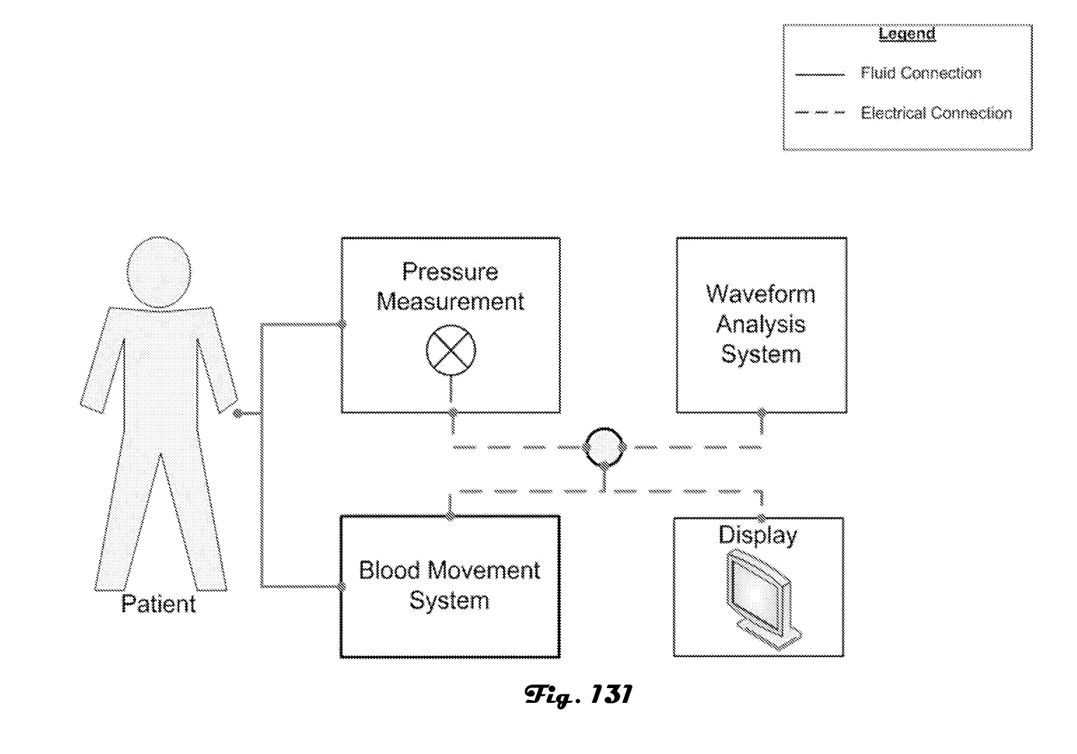

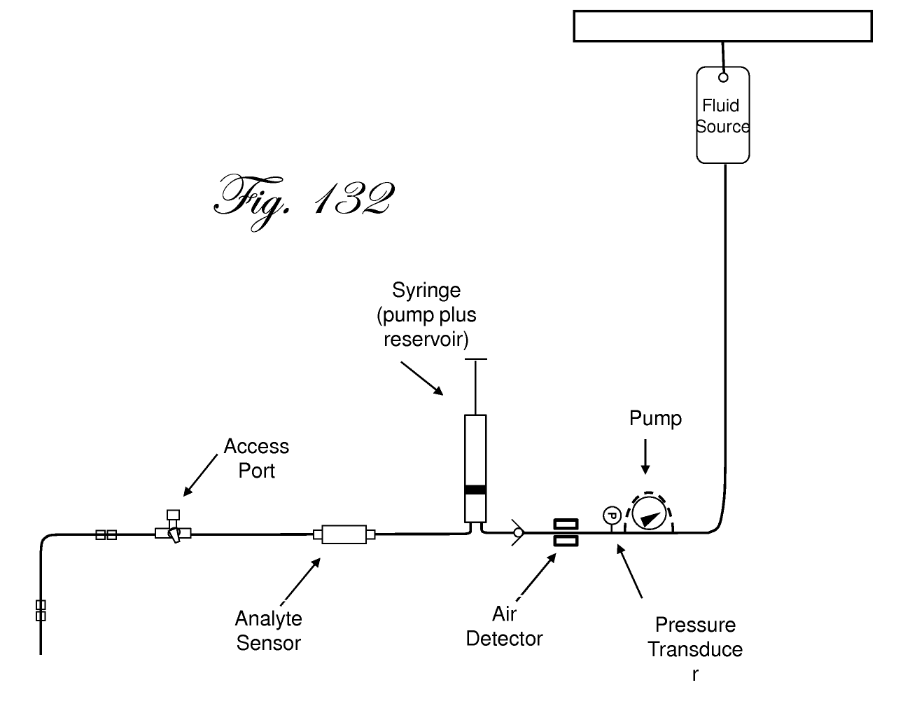

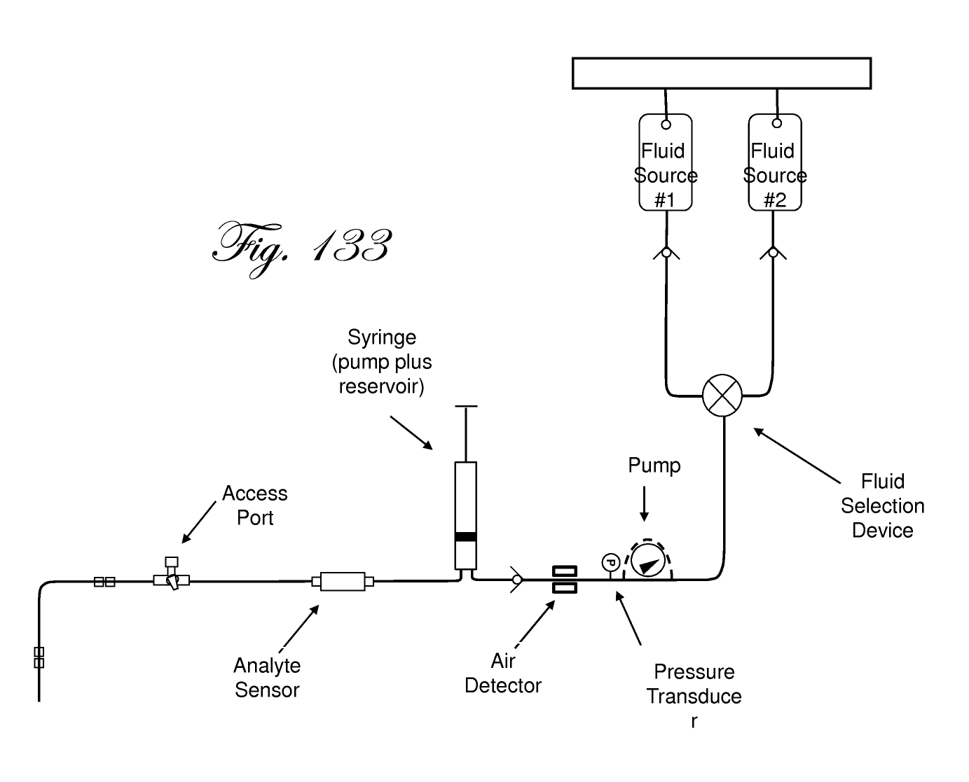

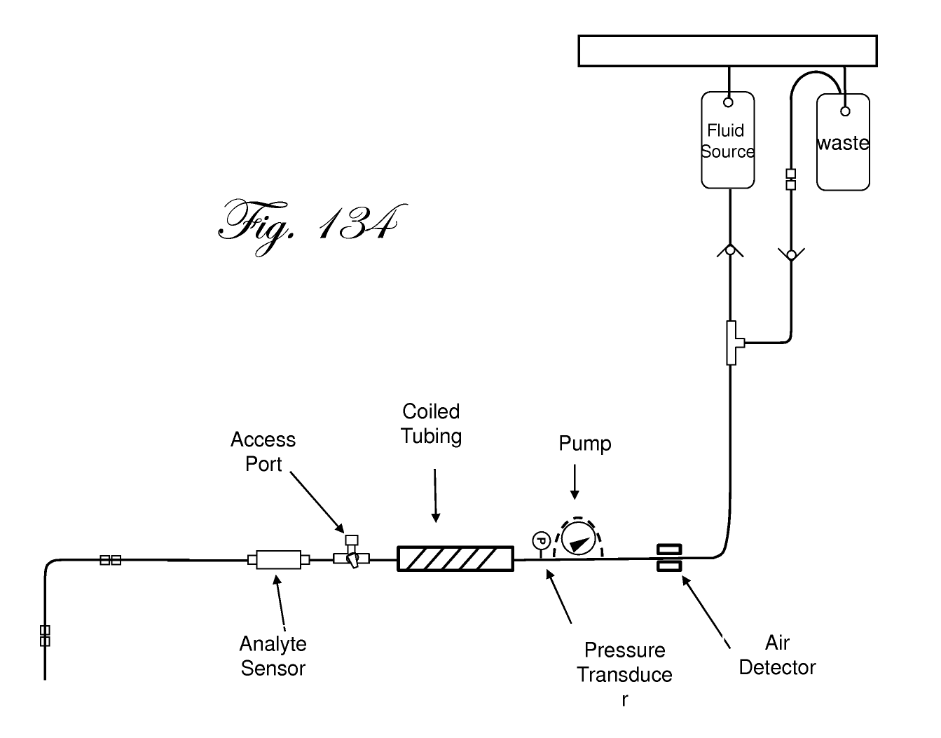

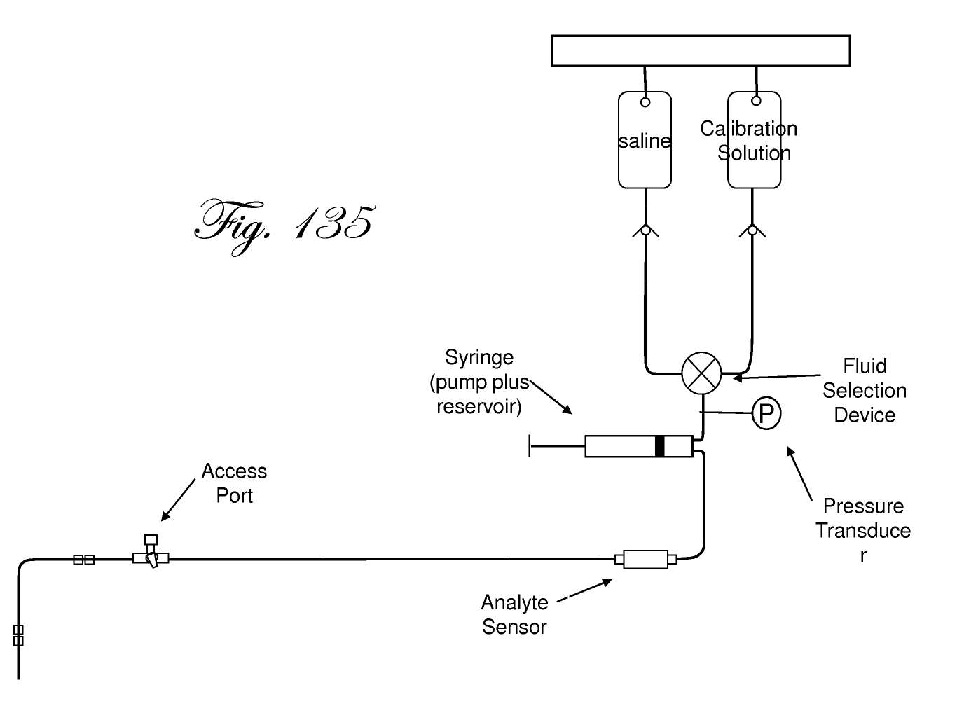

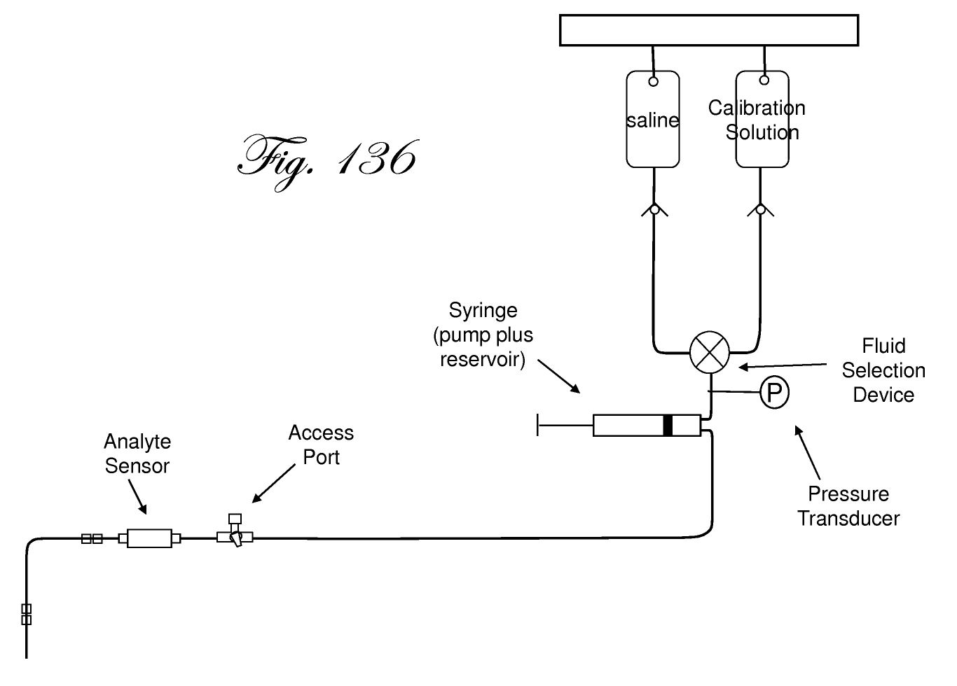

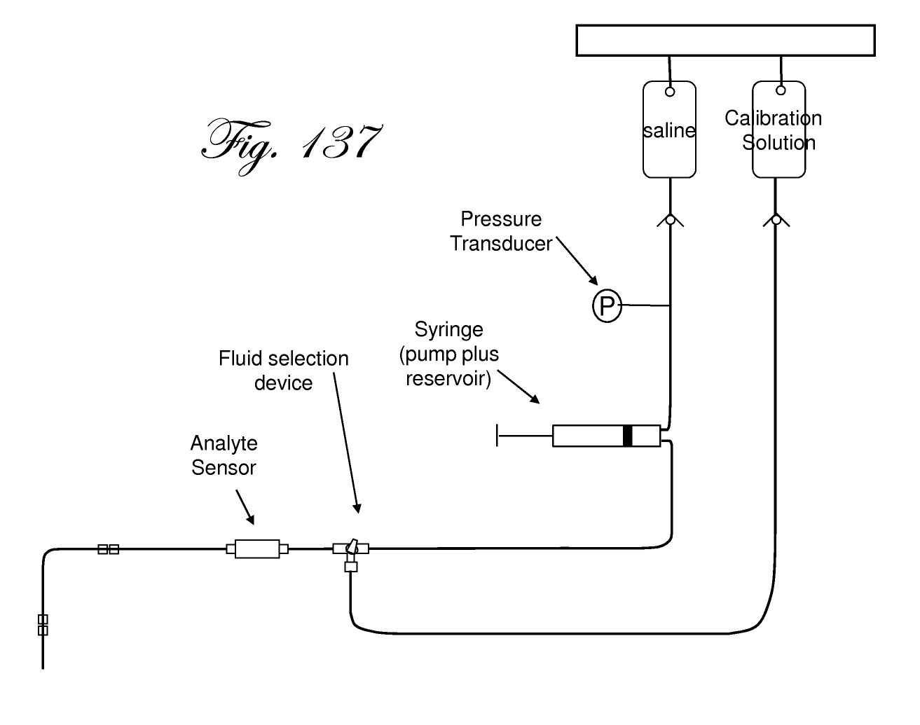

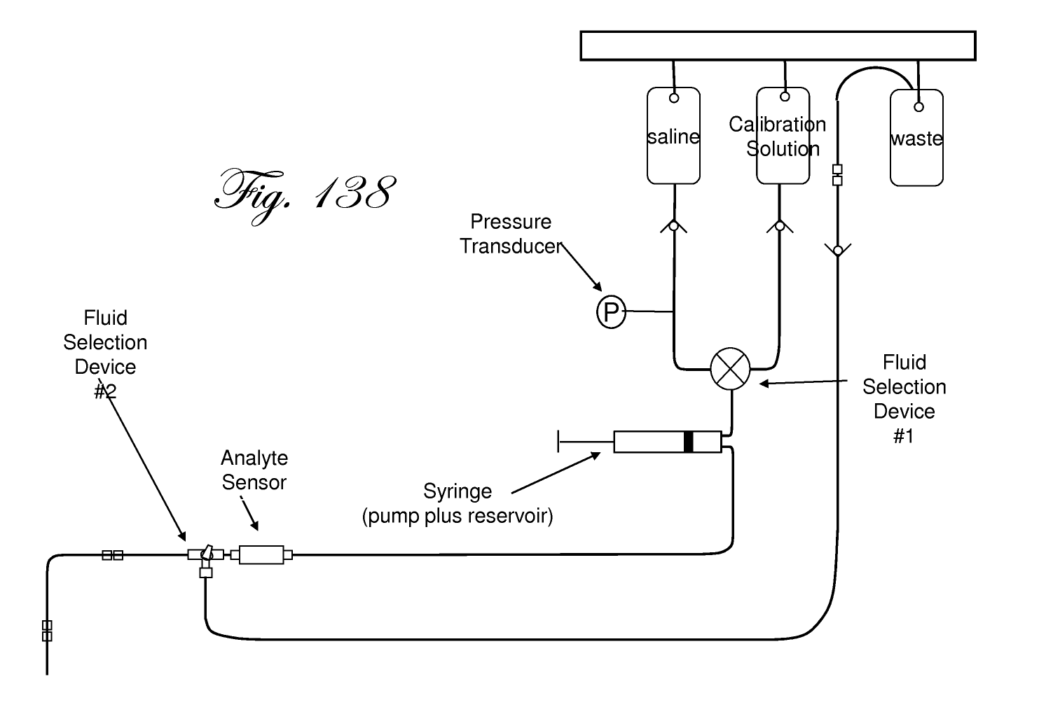

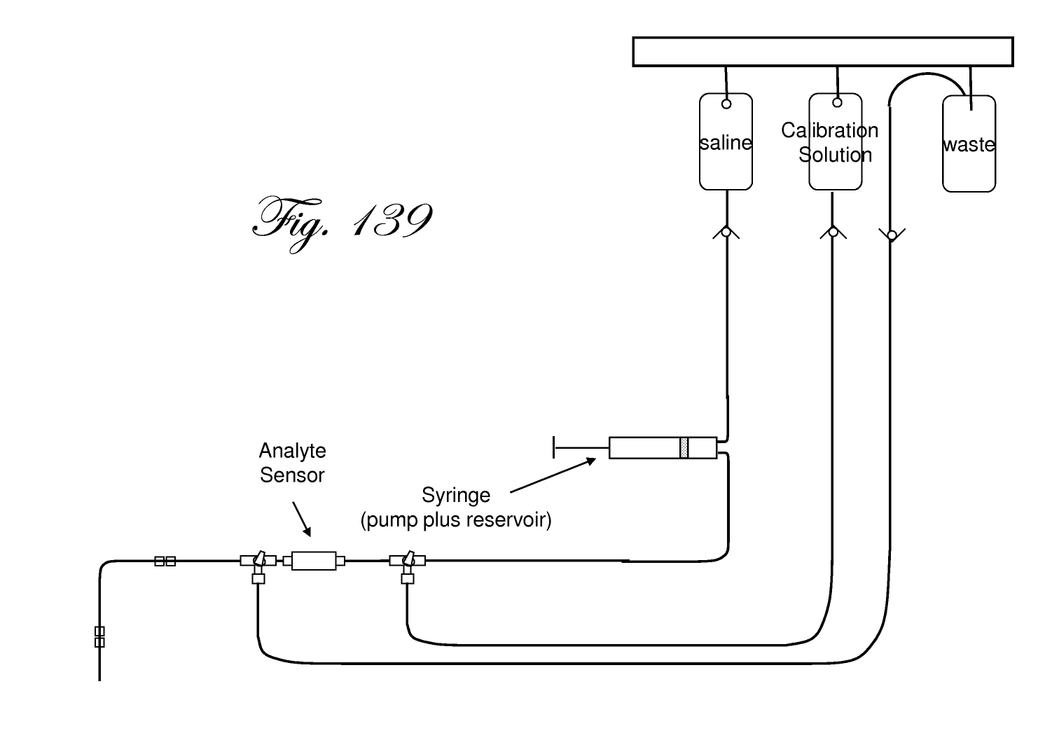

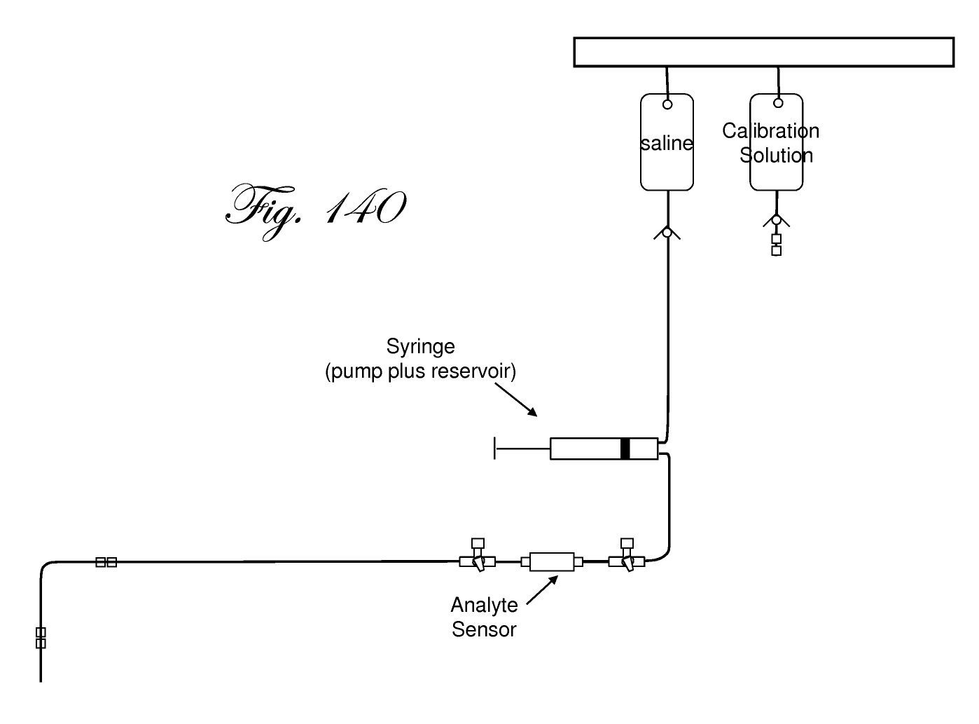

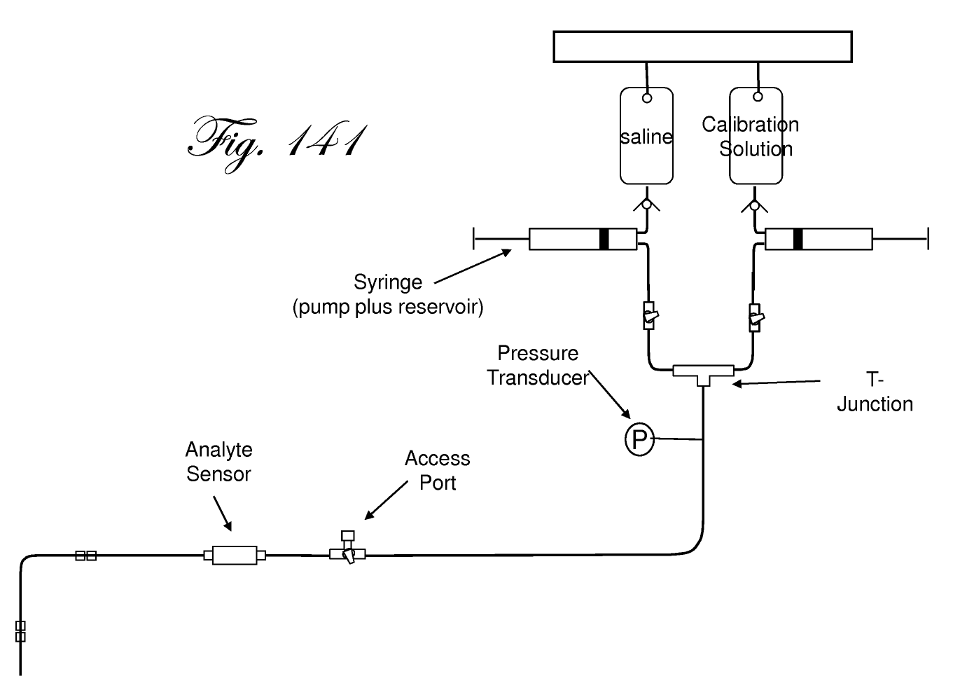

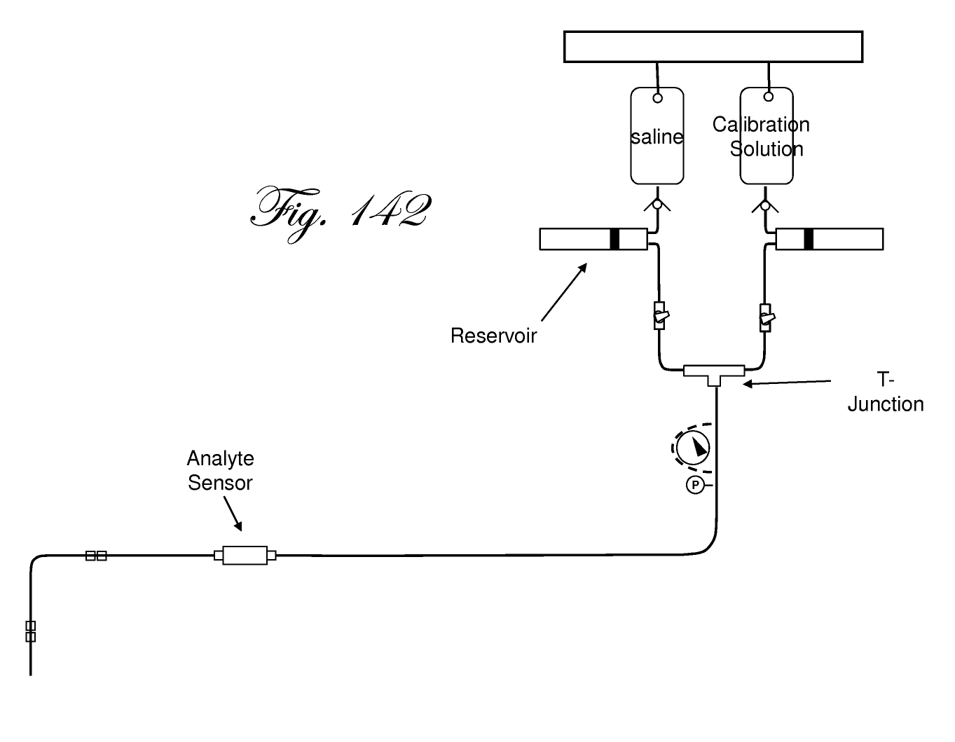

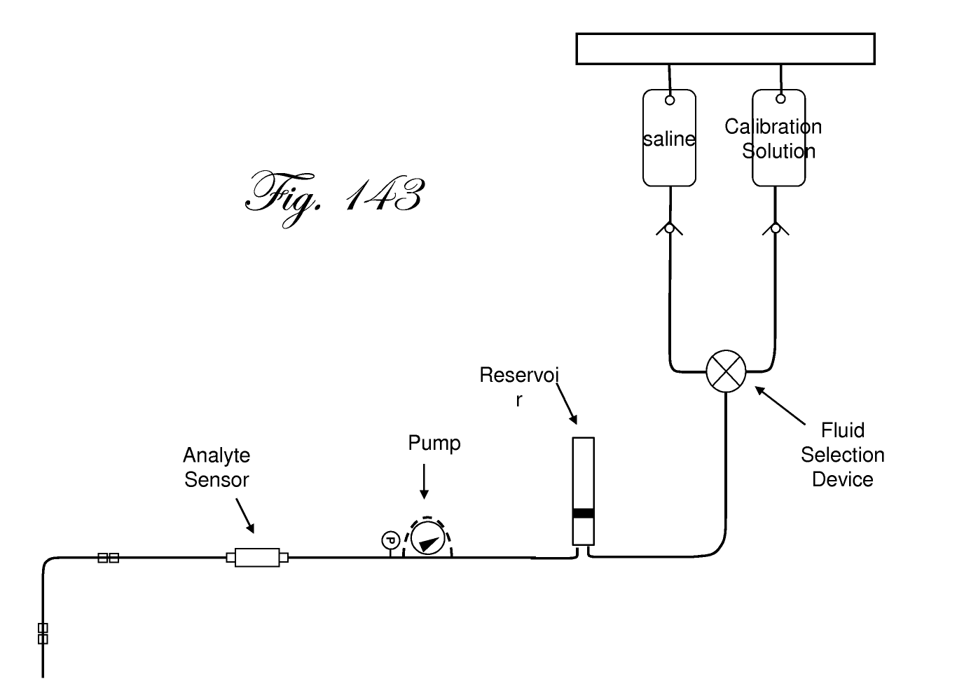

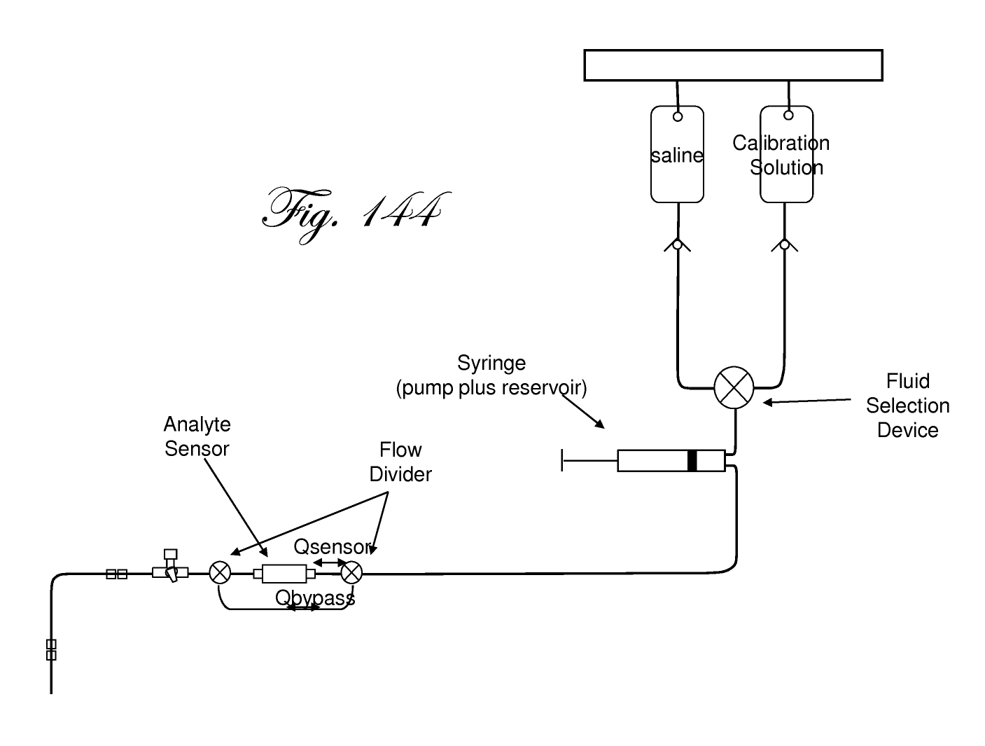

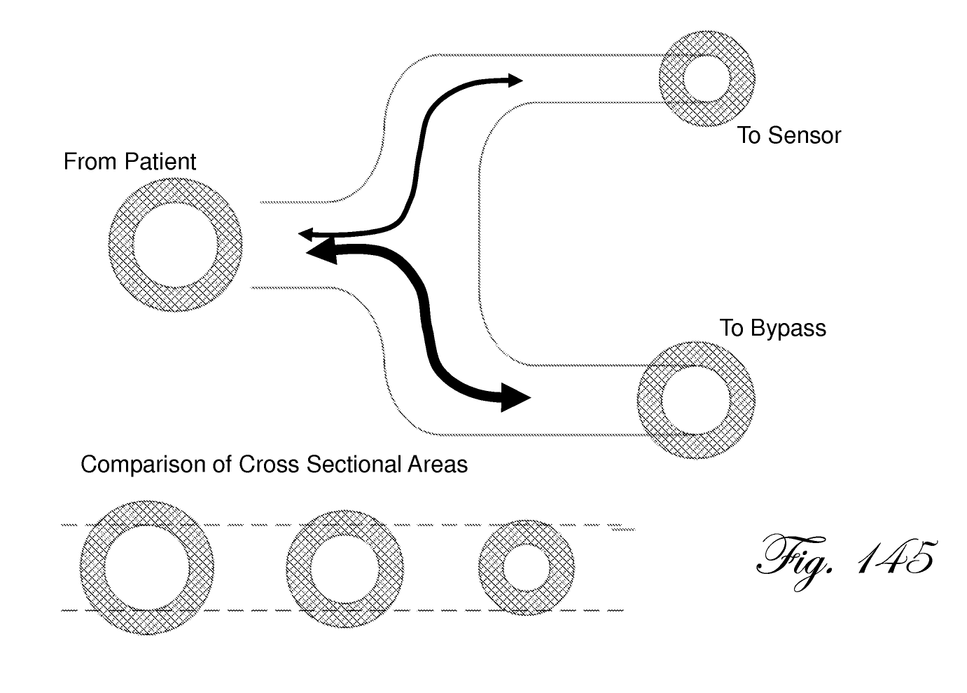

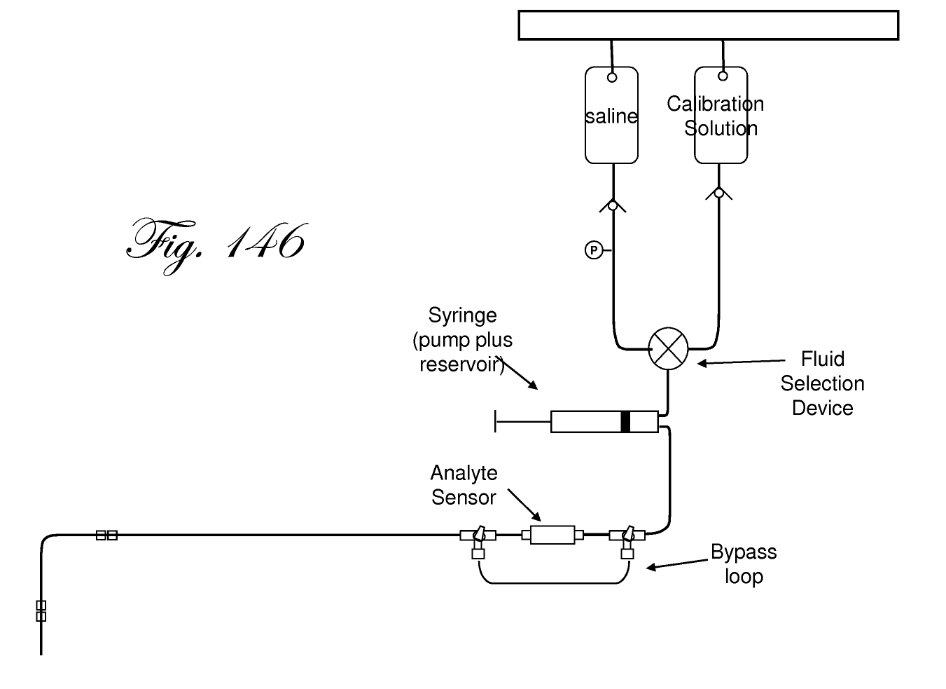

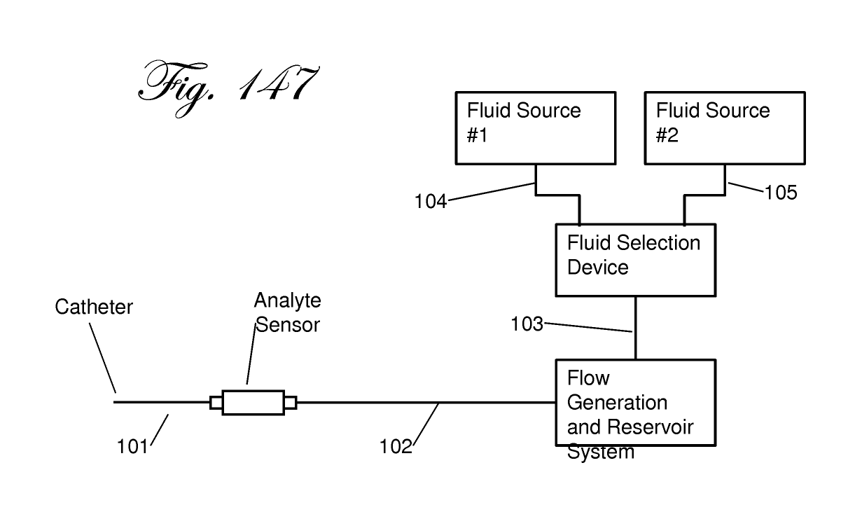

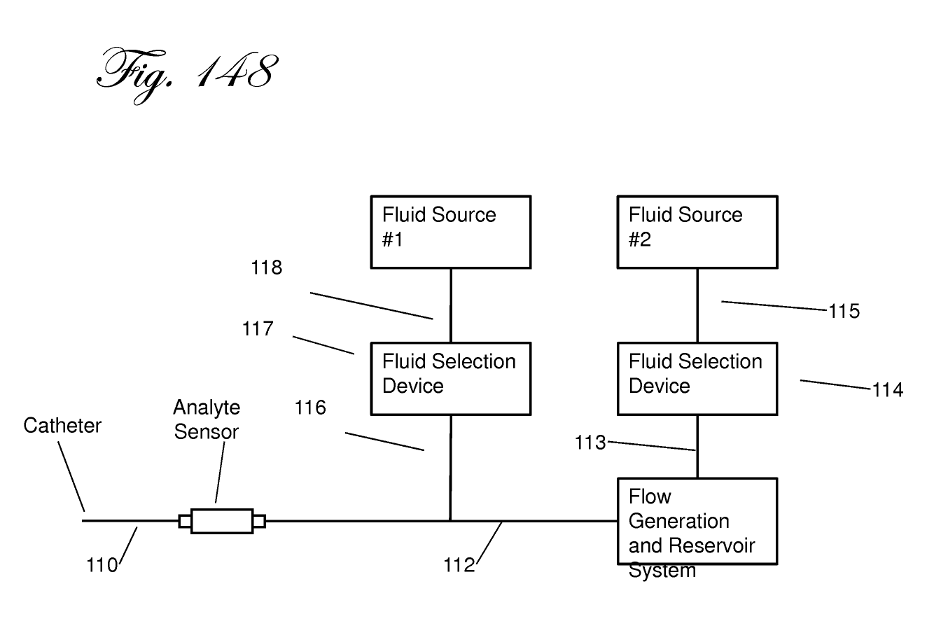

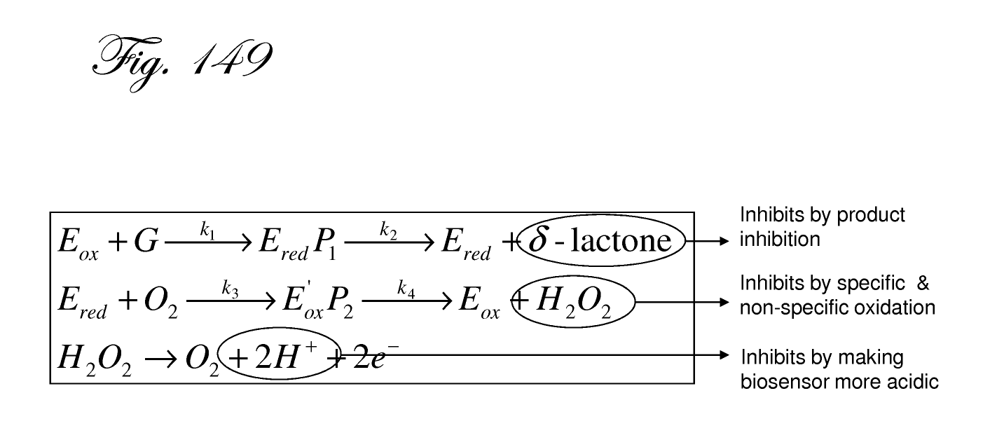

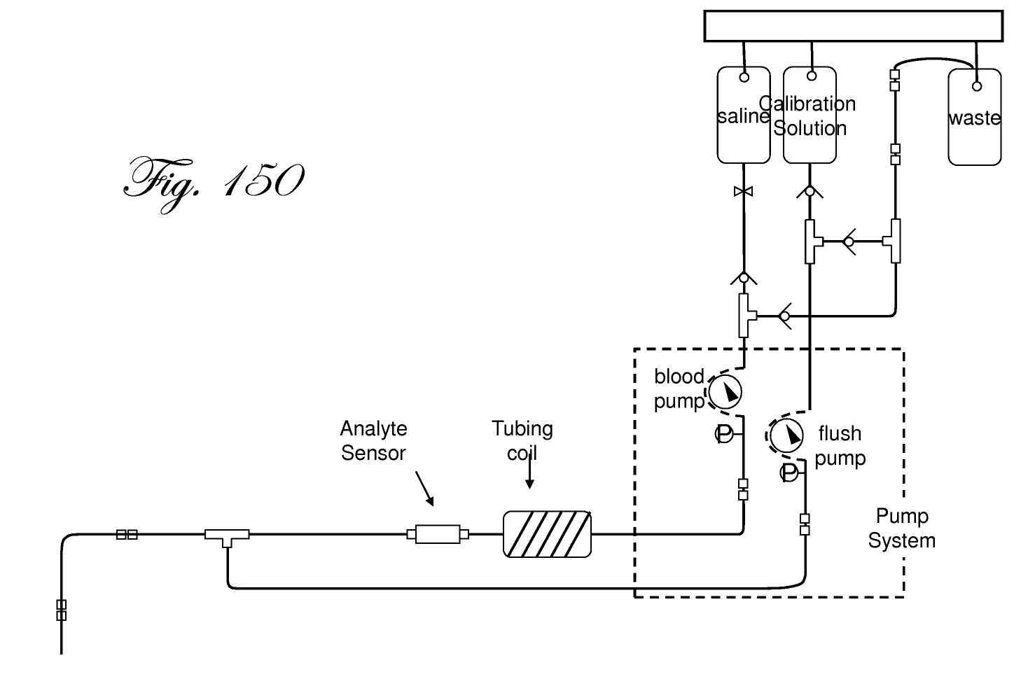

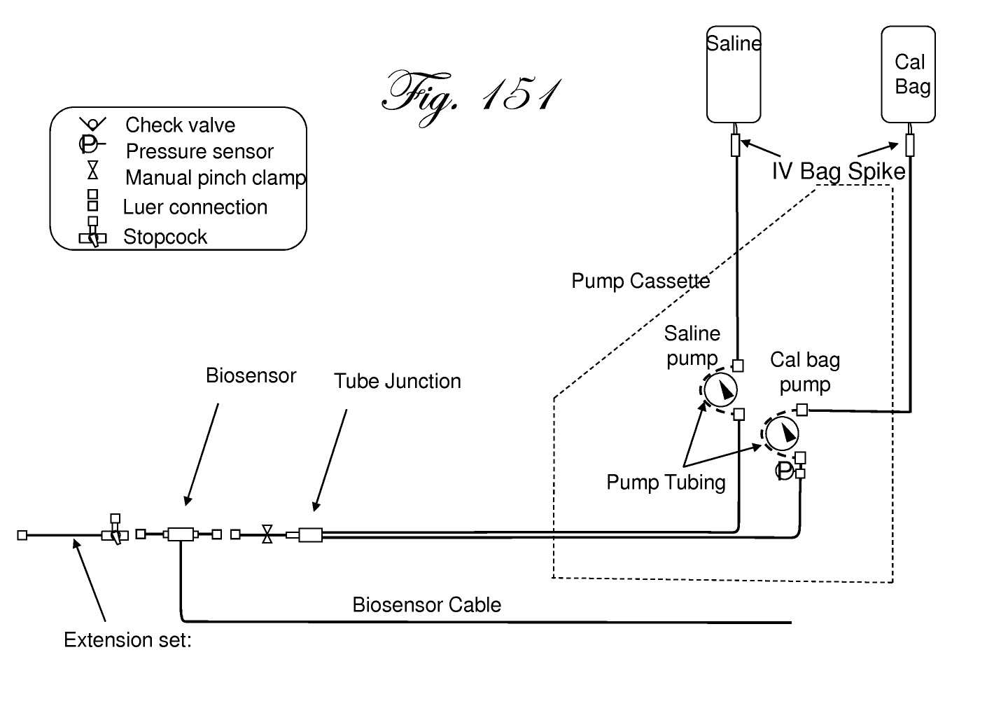

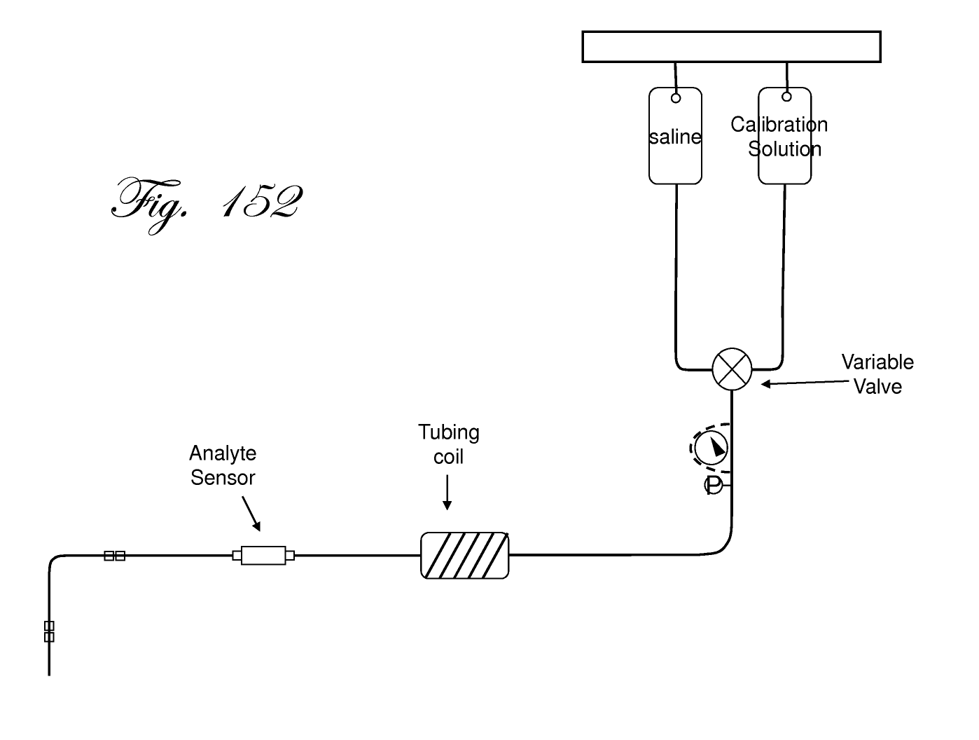

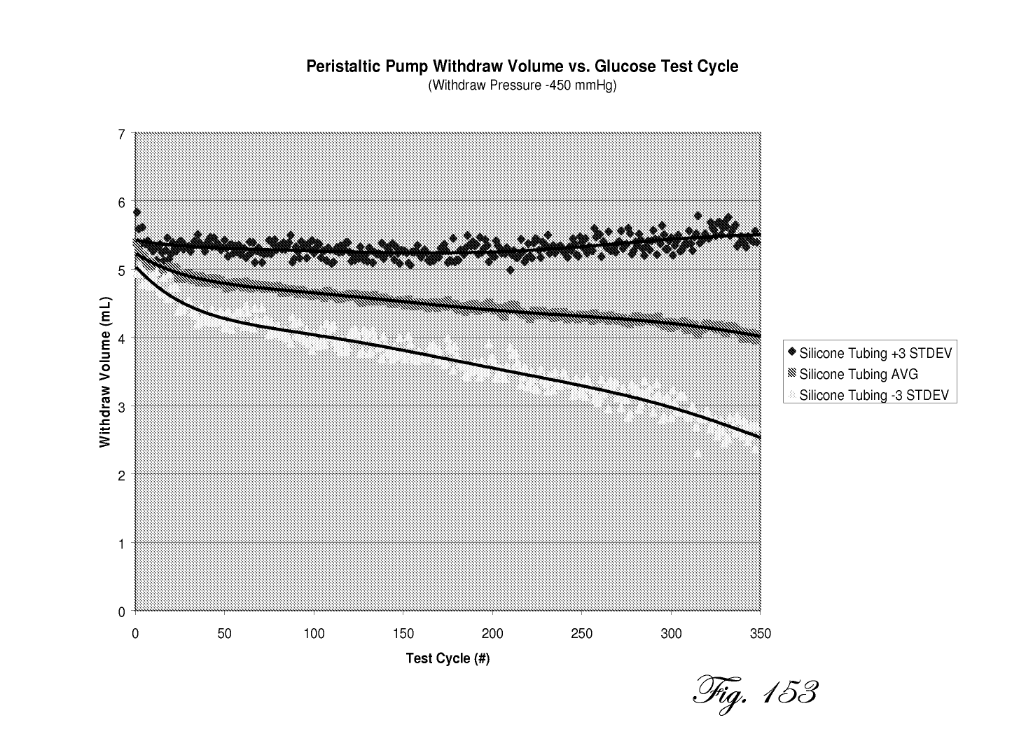

FIG. 128 shows another example embodiment of a blood access system but where the sensor is located close to the patient. As shown the blood access system has only one pressure transducer but others can be added as appropriate for the desired operation. The same general concepts to bubble detection and subsequent management can be applied as described above.

In implementation, the blood access system and the pressure measurement system must be able to exchange information. In general terms the integrated system is composed of four basic parts: (1) Blood movement system (2) pressure measurement system, (3) waveform analysis system and (4) display system. The various systems must be able to exchange information for the effective implementation of the bubble detection methodology. As shown in FIG. 129 these system can be contained in a single box. The communication shown is illustrated as an electrical connection but any form of communication would work to include wireless communication. FIG. 130 shows the pressure measurement system as a separate entity in communication with the other systems. In such a scenario a conventional pressure transducer could provide waveform information to the automated blood analyte measurement system that contains the blood movement system, waveform analysis system and a display. In a final embodiment, FIG. 131, all systems could be physically distinct with only information transfer between the sub-systems.

An apparatus for the measurement of an analyte Embodiments of the present invention can facilitate accurate measurement of blood glucose by the clinician in a sterile manner. Embodiments of the present invention can also enable the calibration of the sensor at one or more calibration points. One desired analyte of measurement is glucose for the effective implementation of glycemic control protocols. Embodiments of the present invention can also be used for the measurement of other analytes such as arterial blood gases, lactate, hemoglobin, potassium and urea. Additionally, embodiments of the present invention can function effectively on a variety of blood access points and specifically enables hemodynamic monitoring. The present invention does not consume a significant amount of blood. Some embodiments of the present invention can re-infuse the blood into the patient, which can facilitate operation of the system in a sterile manner. A blood access system suitable for the applications mentioned above can have any one or combination of several desirable characteristics, described below.

A system according to the present invention can measure the blood by an electrochemical sensor. Such a measurement method need not consume any blood. Embodiments of the present invention provide for movement of blood into and out of the system in a manner that does not damage or activate the blood removed from the patient. One example embodiment uses a syringe although other pressure generating mechanisms can be utilized, including peristaltic pumps.

The blood access system can use fluid sources such as saline as a mechanism for cleaning the system of blood and for pushing the blood back into the patient.

Some example embodiments provide for minimization of mixing use low turbulent draw methods and tubing with low shear forces at the walls. Other considerations include the number of discontinuities included in the system, the number of luer connections and any discontinuity where cells can become trapped via stagnation. In some embodiments, the saline used for the final washing and subsequent cleaning of the circuit can be pumped to waste. The use of a waste or cleaning loop can provide multiple avenues for decreasing the saline infused into the patient.

The blood analyte measurement system must be able to manage or compensate for different vascular pressures. Some embodiments of the present invention enable blood pressure monitoring.

Some embodiments of the present invention enable standard pressure monitoring to occur between measurements. The pressure monitoring device can be located on a fluid pathway that is in fluid communication with the subject. In most embodiments, the pressure transducer is located close to the flow generation device but such a restriction in placement is not required. In fact the pressure monitoring device can be located on any fluid pathway that allows for accurate pressure measurements including waste pathways, calibration pathways, etc.

In some applications, it can be desirable for the blood access system to provide the ability to introduce and subsequently measurement a validation or calibration sample. Such a sample can be placed in the access system or provided in a manner that mimics a sample in the access system. Some embodiments of the present invention provide for a solution to be injected into the blood access system, or injected directly into the sensor.

Another embodiment uses an electronic check-sample to introduce a characteristic voltage or current signal into the instrumentation that verifies the performance of subsequent electronic and computational stages. One embodiment can mimic the detector signal with repeatable voltage waveforms produced by a digital-to-analog converter. These waveforms can mimic known amounts of the glucose signal to verify calibration accuracy.

In practice the vascular point can be kept open by the infusion of about 3 ml/hr of intravenous solution. Some embodiments of the present invention provide a capability to infuse solution at a similar rate to maintain movement of blood or saline across the catheter for the minimization of clot formation. This fluid infusion can be accomplished by gravity flow, a pressurized bag or other means.

It can be desirable for a system to have a cleaning capability, or example to reduce general contamination of the blood tubing and measurement system, the formation of small clots, or for general maintenance of the system. A solution used for cleaning the system can be infused into the patient or can be emptied into a waste bag. A solution used to push blood back into the patient can also accomplish cleaning of the system. Blood can often be a difficult substance to clean from a fluid management system. Accordingly, a cleaning cycle can utilize variable rates of flow, changes in direction of flow, and vibrate modes. A vibrate mode can take many forms; for example, the operator could push on the syringe then stop and push again. Such a push-stop-push technique is commonly used to clean peripherally inserted central catheters.

In some applications, it can be desirable to clean portions of the system with an enhanced cleaner such as one containing a detergent, surfactant, emulsifier, soap or the like. The enhanced cleaner can be used throughout the measurement cycle or introduced into the circuit during the end of an infusion cycle. In some use cases, the infusion cycle can be stopped before a significant portion (e.g., any, or any amount over some threshold) of the enhanced cleaner reaches the patient. A subsequent recirculation or cleaning cycle can cause the enhanced cleaner to flow through the system (but not enter the patient). A non-enhanced cleaner (e.g., saline) can be introduced into the circuit following the enhanced cleaner, such that the enhanced cleaner flows through the system, followed by the non-enhanced cleaner. The volumes of non-enhanced cleaner and enhanced cleaner can be controlled such that enhanced cleaner is not left in a portion of the system where it can be infused into the patient. In some applications, the useful life of the system can be extended by periodic cleaning with an cleaning agent.

The blood access system can contain a method for determining when the system becomes disconnected from the patient. For example, pressure detection, air detection, or the use of sound waves can be used to indicate that the system is not attached to a patient.

The blood access system can detect and prevent the infusion of air bubbles into the vascular system in any of several ways. Air bubbles can be removed prior to infusion into the patient can be by bubble traps or other filter mechanisms. Alternatively, the bubble can be routed to a waste line to clear it from the infusion circuit. In such a waste line embodiment, the system can continue operation without a requirement of pump stoppage.

The detection of vascular occlusion on either a withdrawal or an infusion can be important for patient safety. Some embodiments of the present invention can determine an occlusion by pressure monitoring or by examination of the sensor response. If fluid flow is unexpectedly stopped or slowed, the sensor response can change for multiple reasons such as heating.

A blood access system according to the present invention can be more effectively used for blood gas measurement by providing a means for compensation for such effects. Mechanisms for providing an accurate blood gas measurement can include the use of very short tubing lengths, allowing for equilibration of the blood with the tubing, minimizing the amount of out gassing by the tubing, compensation algorithms to account for changes, or a combination thereof. In the case of a loop system embodiment, the tubing can become equilibrated with the blood. In a second example embodiment, the amount of blood withdrawn can be large enough that the sample measured at the end of the draw has undergone minimal change. Another example embodiment measures the blood gases over the entire sample draw with a projection to an equilibrated point. Different blood draw mechanisms or operating parameters can be used for glucose measurements than are used for blood gas measurements. For example, equilibration concerns can indicate that a larger volume of blood be drawn for blood gas measurements than is required for glucose measurements.

In some applications of the present invention, it can be important to minimize the total amount of blood removed from the body and present in the circuit. For example, the clotting system can become activated when placed in contact with foreign materials. In such applications, a sample can be isolated at a location close to the patient. Any blood beyond that required for the sample can be quickly re-infused to minimize blood residence time. This isolated sample can then be measured without requiring a larger volume of blood to be present in the blood measurement system.

In some applications, the volume of venous blood accessible by the system can be supplemented by use of a standard pressure cuff proximal to the sampling site (e.g., for sampling through access at the lower arm, the cuff might be best positioned at the upper arm). The pressure cuff can be inflated at a preset time period before commencing blood withdrawal, forcing the venous pressure to the cuff pressure, increasing vascular volume, and increasing the available blood flow. As an example, the cuff can be inflated to 40 mmHg or a pressure less than arterial pressure if desired. The cuff can be deflated before commencing infusion, minimizing the back pressure experienced by the system during infusion. A pressure sensor within the circuit can be used as a trigger for the initiating the withdrawal of blood.

As some ICU patients have automatic blood pressure cuffs in place, the system can leverage the increased venous pressure and volume that occurs during the measurement process for the procurement of a blood sample. The operator or the system itself could sense the initiation of an automatic blood pressure measurement by changes in pressure, activation sounds or signals directly from the physiological monitor. For example the GE Dash™ 3000 Patient monitor has an analog blood pressure output that could be utilized for to trigger blood procurement. The blood access system would then utilize the increased venous pressure and associated blood volume due to cuff pressure and procure a blood sample. Such supplementation of the venous blood volume available can help facilitate the procurement of blood samples on a repeatable basis.

The present invention enables a multitude of options in both calibration and validation to ensure effective operation of the system. A basis for calibration is the use of fluid sources that can be used for calibration. These fluid sources can contain known analyte concentrations and can also contain additional additives that improve the overall performance of the system. Specific additives that can be contained in the fluids include additives that reduce bubble formation, facilitate cleaning of the circuit, reduce protein buildup on the sensing element, reduce cellular aggregation or platelet adhesion to the circuit. As examples, heparin and citrate can be used as additives that reduce the possibility of cellular aggregation. As used in this application; fluid sources, saline fluids, calibration fluids, or maintenance fluids are not intended to be restricted to only normal saline but further include any fluid it that can be administered to patients in environments such as the intensive care unit. Such fluids include but are not limited to normal saline, ¼ normal saline, ¼ normal saline, parenteral nutrition, and lactated ringers. Additionally, the fluid source can contain drugs or medications.

An important advantage of some embodiments of a blood analyte measurement system according to the present invention is the ability to perform sensor recalibration in a completely sterile manner. Infection risks within intensive care unit patients are extremely high. Some embodiments of the present invention can provide a calibration procedure that does not require “opening” of the system to potential bacteria.

The following figures illustrate a number of example embodiments of the present invention. Each example embodiment generally provides one or more of the desired attributes of the blood analyte measurement system as described above. For purposes of this disclosure, a fluid selection device will encompass any device that allows the user to select a designated fluid source or to stop fluid flow. Such a device can also have the ability to control flow rate from a fluid source. Some fluid selection devices enable selection of a fluid path that enables the removal or addition of fluid, for example by a syringe. A variety of flow selection devices can be used with the preferred embodiments, including but not limited to stop cocks (two way, three way, four way, etc.), pinch valves, butterfly valves, ball valves, rotating pinch valves and linear pinch valves, cams and the like. In some embodiments, a flow selection device selects the fluid source to be used and controls the flow rate from the fluid source.

As used in the disclosure a flow generation device controls the flow of fluids within the system by creating pressure gradients or allowing existing gradients to be transmitted such that fluid flow occurs. In some example embodiments, a flow generation device is configured to regulate the exposure of the sensor to the fluid sources including calibration fluids and blood from the host. In some example embodiments, the flow generation device is depicted as a syringe, but can include valves, cams, pumps, and the like. In one example embodiment, the flow generation device is a peristaltic pump. Other suitable pumps include volumetric infusion pumps, peristaltic pumps, and piston pumps. Flow generation devices also include any mechanism that creates a needed pressure gradient for operation. Such a pressure gradient can be generated by varying the pressure at the fluid source by raising/lowering the fluid source. Additionally pressure gradients can be created by placement of pressure cuff around a fluid source (typically an IV bag) or through the use of any mechanism that creates a pressurized bag.

As used in the following embodiments, a fluid source is any source of fluid used in the operation of the blood analyte measurement system. These fluid sources can be used for calibration, cleaning, verification and maintenance of the system. The fluid sources can contain known analyte concentrations and can also contain additional additives that improve the overall performance of the system. Specific additives that can be contained in the maintenance fluid include additives that reduce bubble formation, facilitate cleaning of the circuit, reduce protein buildup on the sensing element, reduce cellular aggregation or platelet adhesion to the circuit. As examples, heparin and citrate are known anticoagulants that reduce cellular aggregation. As used in this description fluid sources can include saline fluids or maintenance fluids can include any fluid it that is commonly administered to patients in environments such as the intensive care unit. Such fluids can include but are not limited to normal saline, ½ normal saline, and lactated ringers. In general terms, the saline fluid is the fluid used to maintain the patency of the access site. The calibration fluid is typically considered as a secondary fluid designed specifically to facilitate calibration or the overall operation of the device. These general terms are not intended to be restrictive but to provide a better context for the following descriptions.

Some of the example embodiments use a reservoir for fluid storage. A reservoir as used in this description includes any device that allows for the storage of a variable volume of fluid. Examples include but are not limited to a bag, a flexible pillow, a syringe, a bellows device, a device that can be expanded through pressure, an expandable fluid column, etc.

As shown in some of the example embodiments the flow generation device and reservoir can be combined into a single system, referred to as the flow generation and reservoir system. An example of such a system is a syringe which has both flow generation and reservoir capabilities. A syringe or syringe pump is defined broadly as a simple piston pump consisting of a plunger that fits tightly in a tube or container. The plunger can be pulled and pushed along inside a cylindrical tube (the barrel) or container, allowing the syringe to take in and expel a liquid. Such syringe systems for procurement of blood are used in clinical practice. Known syringe systems include Deltran Plus Needleless Arterial Blood Sampling System, VAMP Venous Arterial blood Management Protection, Portex Line Draw Plus, Becton Dickinson Safedraw, Smiths Saf-T Closed Blood Collection System, and Hospira SafeSet Closed Blood Sampling system (the foregoing are claimed as trademarks by their respective owners). Another example is a standard peristaltic pump coupled with a reservoir to provide both flow generation and reservoir capabilities.

As shown in some example embodiments, there is a waste channel such as a fluid pathway to a waste bag. During the blood withdrawal process, the fluid volume withdrawn can be transferred into a reservoir, returned to one of the fluid sources, or transferred to waste. For infection control purposes and to minimize contamination, it is typically undesirable to return the fluid volume to any of the fluid sources. Such a process can dilute a calibration at a fixed analyte concentration or add glucose or other analytes to a solution containing no analytes. Additionally, the potential introduction of red blood cells or other cellular matter results in contamination of the fluid source. If no reservoir is used and the fluid is not returned to a fluid source, then the fluid displaced by the withdrawal process can be transferred to a waste channel. One way valves can be used to ensure one way flow into the waste bag and out of the fluid source(s). Such unidirectional flows ensure that contamination does not occur

### Example Embodiment

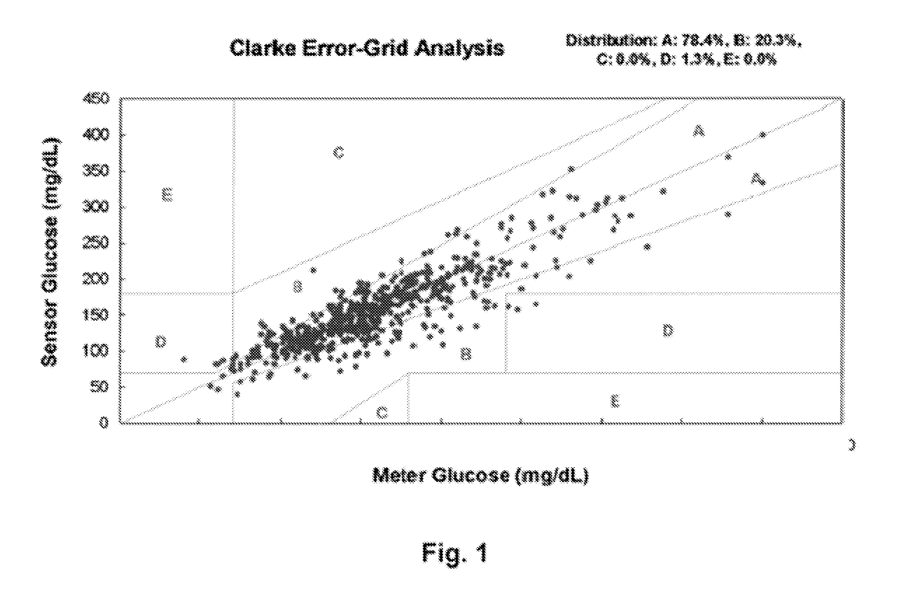

Push-pull system using syringe and peristaltic pump. FIG. 1 is a schematic depiction of an example embodiment of the present invention having a syringe push-pull operation. A syringe is used as a flow generation device. The syringe creates a pressure gradient to withdraw blood from the patient to the sensor. Additionally, the syringe serves as a reservoir since the initial blood present will be mixed with saline. Following completion of the measurement, the syringe can be pushed to remove all fluid from the cylinder. Additional washing of the system can be provided by the peristaltic blood pump shown. The example embodiment comprises: a blood access point, a measurement sensor, a needle-less access port, a syringe, a pressure measurement device, a peristaltic pump, and a saline or calibration bag. The operation of the example embodiment is described below.

Blood Sample and Measurement Process:

1. The syringe is used to initiate the draw by moving the plunger away from the home position. The draw continues until an undiluted sample is present at the measurement sensor.
2. The blood interacts with measurement sensor and an analyte measurement is made.
3. Following completion of the measurement, the syringe is pushed towards the home position so that the blood is returned to the patient.
4. Following the return of the syringe to the home position, the pump is activated so as to move saline or calibration fluid through the system to the patient. This process helps clean the circuit and remove any remaining blood in the circuit.
5. Following cleaning of the circuit, the blood pump may remain active to maintain a “keep vein open” fluid infusion towards the patient.
6. The measurement results and any historical information are communicated to a user, e.g., shown on a display (not shown).

The example embodiment of FIG. 1 can provide several important characteristics:

1. Analyte measurements can be made on a very frequent basis.
2. The system operates with no blood loss.
3. The system operates with very little saline infusion and only during cleaning.
4. The system can work on multiple access locations, including arterial.
5. The system contains a pressure monitor that can provide arterial, central venous, or pulmonary artery catheter pressure measurements after compensation for the pull and push of the blood access system.
6. The system can compensate for different size catheters through the volume pulled via the syringe.
7. The system provides for a one point calibration via the saline or calibration bag.
8. The system provides for access to the blood sample via a port in the circuit.

### Example Embodiment

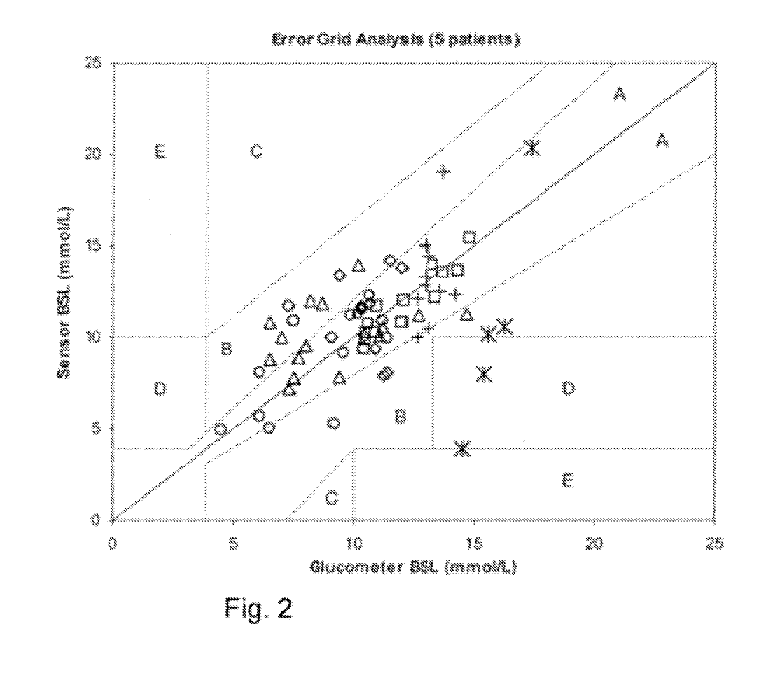

Push Pull System Based upon Syringe and Peristaltic Pump with Two Point Calibration. FIG. 2 is a schematic depiction of an example embodiment of the present invention having a syringe push-pull operation. In the example embodiment, the flow generation device shown is a syringe. The syringe creates a pressure gradient to withdraw blood from the patient to the sensor. Additionally, the syringe serves as a reservoir since the initial blood present will be mixed with saline. Following completion of the measurement, the syringe is pushed to remove all fluid from the cylinder. The system has the ability to perform a two point calibration via selection of the fluid source by the flow selection device. Additional washing of the system is provided by the peristaltic blood pump shown. The system comprises: a patient interface device such as catheter or other blood access point to the patient, a measurement sensor in fluid communication with the patient interface device, a needle-less access port in fluid communication with the sensor, a syringe in fluid communication with the needle-less access port, a pressure measurement device in fluid communication with the syringe, a peristaltic pump in fluid communication with the syringe, a fluid selection valve in fluid communication with the peristaltic pump and, through individual one-way valves, with two fluid bags that can contain two separate calibration fluids. The operation of the example embodiment is described below.

Blood Sample and Measurement Process:

1. The syringe initiates the draw by moving the plunger away from the home position. The draw continues until an undiluted sample is present at the measurement sensor.
2. The blood interacts with measurement sensor and an analyte measurement is made.
3. Following completion of the measurement, the syringe is pushed towards the home position so that the blood is returned to the patient.
4. Following the return of the syringe to the home position, the pump is activated so as to move saline or calibration fluid through the system to the patient. This process helps clean the circuit and removed any remaining blood in the circuit.
5. Following cleaning of the circuit, blood pump may remain active to maintain a “keep vein open” fluid infusion towards the patient.
6. The measurement results and any historical information are communicated to a user, e.g., shown on a display (not shown).

Calibration process. The system has two fluid sources that can be used to facilitate calibration of the sensor. The fluid sources have different glucose levels. The fluid selection device can be used to select the fluid of choice. The peristaltic pump can then move the fluid so that the sensor is exposed to the designated calibration fluid. The pump may remain active during this period and flow calibration fluid over the sensor pump may stop and allow the calibration fluid to simply remain in contact with the sensor.

The example embodiment of FIG. 2 can provide several important characteristics:

1. The system can provide a two point calibration of sensor.
2. Analyte measurements can be made on a very frequent basis.
3. The system operates with no blood loss.
4. The system requires very little saline infusion and only during cleaning.
5. The system can work on multiple access locations including but not limited to arterial.
6. The system contains a pressure monitor that can provide arterial, central venous, or pulmonary artery catheter pressure measurements after compensation for the pull and push of the blood access system.
7. The system can compensate for different size catheters through the volume pulled via the syringe.
8. The system provides for a one point calibration via the saline or calibration bag.
9. The system provides for access to the blood sample via a port in the circuit.

### Example Embodiment

Push-Pull System Based upon Tubing Reservoir and Peristaltic Pump. FIG. 3 is a schematic depiction of an example embodiment of the present invention having a push-pull operation with a fluid pathway to divert fluid to waste. The system prevents possible red blood cell lysis by ensuring that no blood enters the peristaltic pump. The system provides for storage of the blood-saline junction in a tubing coil. The system prevents any contamination of the saline bag by diverting the withdrawal fluid into a waste bag. The system has appropriate occlusion detection via pressure monitoring, blood access via an access port, provides flow control during the measurement process, and the use of the peristaltic pump permits pulsed or variable wash sequences. The system comprises: a blood access point to the patient, a measurement sensor, a needle-less access port, tubing coil, a pressure measurement device, a peristaltic pump, a t-junction, a fluid bag for calibration with a one-way valve allowing fluid flow from the fluid bag to the t-junction, and a waste bag with a one-way valve allowing fluid flow from the t-junction to the waste bag. As one of skill on the art would appreciate, a second calibration fluid or multiple calibration fluids can be added in a manner similar to that described in FIG. 2. The operation of the example embodiment is described below.

Blood Sample and Measurement Process:

1. Peristaltic pump initiates the draw by moving blood toward the sensor. The draw continues until an undiluted sample is present at the measurement sensor.
2. The blood interacts with measurement sensor and an analyte measurement is made.
3. Following completion of the measurement, the peristaltic pump infused the blood back into the patient.
4. Following the return of the blood to the patient, the pump is activated so as to move saline or calibration fluid through the system to the patient for additional cleaning. This process helps clean the circuit and removed any remaining blood in the circuit.
5. Following cleaning of the circuit, blood pump may remain active to maintain a “keep vein open” fluid infusion towards the patient.
6. The measurement results and any historical information are communicated to a user, e.g., shown on a display (not shown).

The example embodiment of FIG. 3 can provide several important characteristics:

1. The system is fully automatic system and does not require nurse intervention.
2. Analyte measurements can be made on a very frequent basis.
3. The system operates with no blood loss.
4. The system requires very little saline infusion and only during cleaning.
5. The system can work on multiple access locations including arterial.
6. The system contains a pressure monitor that can provide arterial, central venous, or pulmonary artery catheter pressure measurements after compensation for the pull and push of the blood access system.
7. The system can compensate for different size catheters through the volume pulled via the syringe.
8. The system provides for a one point calibration via the saline or calibration bag.
9. The system provides for access to the blood sample via a port in the circuit.

### Example Embodiment

Push Pull System Based upon Syringe. FIG. 4 is a schematic depiction of an example embodiment of the present invention with a sensor close to a reservoir. The example embodiment can be described as a push pull system where the flow generation device is a syringe. The syringe creates a pressure gradient to withdraw blood from the patient to the sensor. The system as shown is manually operated. The syringe serves as a reservoir as the initial blood present will be mixed with saline. The use of a reservoir as shown eliminates the need for a separate waste bag. The system has the capability of doing a two point calibration. The stopcock shown allows for procurement of a blood sample or the introduction of additional calibration, validation or check samples. The pressure measurement device allows for pressure monitoring. If attached to an arterial line the fluid bags would be pressurized to create a pressure gradient to create positive flow to the patient. The system operates in an entirely sterile manner. Following completion of the measurement, the syringe is pushed so as to remove all fluid from the cylinder. Additional washing of the system is provided by allowing flow from the fluid sources towards the patient. In the case of venous attachment, this flow can be by gravity. The system comprises: a catheter providing access patient, a stopcock or other access port, a measurement sensor, a syringe, a pressure measurement device, a fluid selection device allowing selection of the fluid sources and fluid sources for maintenance and calibration of the system. One-way valves can be mounted with the bags to allow fluid flow from the bags to the fluid selection device. The operation of the example embodiment is described below.

Blood Sample and Measurement Process:

1. The system is calibrated as described below. Following calibration the operator initiates a blood draw by moving the syringe plunger away from the home position. The draw continues until an undiluted sample is present at the measurement sensor. The determination of an undiluted sample can be by volume drawn, visual inspection or the sensor sample state methods described above.
2. The blood interacts with measurement sensor and an analyte measurement is made. The blood can be flowing or not flowing across the sensor during the measurement.
3. Following completion of the measurement, the syringe is pushed towards the home position so that the blood is returned to the patient. At this juncture the majority of all blood has been returned to the patient.
4. If additional cleaning of the circuit is desired, fluid from either fluid source can be used to clean the circuit further. The fluid can simply be flowed through the system or drawn into the syringe. If drawn into the syringe, the operator can use a push-stop-push flow pattern to facilitate cleaning. The cleaning process helps to maintain the circuit for future use and prevent clotting of the circuit.
5. Following cleaning of the circuit, fluid for may continue to flow toward the patient to create a “keep vein open” fluid infusion towards the patient.
6. The measurement results and any historical information are communicated to a user, e.g., shown on a display (not shown).

Calibration process. The system has two fluid sources that can be used to facilitate calibration of the sensor. The fluid sources have different analyte levels. The fluid selection device can be used to select one of the two fluids. Gravity feed or pressure moves the fluid so that the sensor is exposed to the designated calibration fluid. During the calibration process, calibration fluid can be flowed over the sensor or fluid may simply remain in contact with the sensor. As described elsewhere in this specification it can be advantageous to maintain the sensor in a low analyte containing solution prior to measurement.

The example embodiment of FIG. 4 can provide several important characteristics:

1. Analyte measurements can be made on a very frequent basis.
2. The system operates with no blood loss.
3. The system can work on multiple access locations including arterial.
4. The system contains a pressure monitor that can provide arterial, central venous, or pulmonary artery catheter pressure measurements.
5. The system can compensate for different size catheters through the volume pulled via the syringe.
6. The system provides for a two point calibration via the two fluid sources.
7. The system provides for access to the blood sample via a port or stopcock in the circuit.
8. Additional samples can be inserted into the system via the access port.
9. The system provides completely sterile operation.

### Example Embodiment

Push Pull system based upon Syringe with Sensor Near Patient. FIG. 5 is a schematic depiction of a push pull system based upon a syringe and is very similar to FIG. 4. A difference between the two example embodiments is the location of the sensor. In FIG. 5 the sensor is located very close to the patient. The location of the sensor close to the patient reduces the blood draw volume needed to get an undiluted sample to the sensor. The syringe creates a pressure gradient to withdraw blood from the patient to the sensor. The operational characteristics of the example embodiment of FIG. 5 are very similar to FIG. 4.

FIG. 5 is a push pull system using a syringe as a flow generation device. Prior to initiation of a measurement, the system allows for maintenance of the sensor in a low glucose concentration fluid. To initiate a measurement, the syringe creates a pressure gradient to withdraw blood from the patient to the sensor. The system as shown is manually operated. The syringe serves as a reservoir since the initial blood present will be mixed with saline. The use of a reservoir as shown eliminates the need for a separate waste bag. The system has the capability of doing a two point calibration as described below. The access port shown allows for procurement of a blood sample or the introduction of additional calibration, validation or check samples. The pressure measurement device allows for pressure monitoring. If attached to an arterial line the fluid bags can be pressurized to create a pressure gradient to create positive flow to the patient. The system operates in an entirely sterile manner. Following completion of the measurement, the syringe can be pushed to remove all fluid from the syringe cylinder. Additional washing of the system can be provided by allowing flow from the fluid sources towards the patient. In the case of venous attachment, this flow can be by gravity.

For calibration, the system can use two fluid sources with different glucose concentrations. The fluid selection device can be used to select the fluid of choice, or a controlled combination of fluids. Gravity feed or pressure moves the fluid so that the sensor is exposed to the designated calibration fluid. During the calibration process, calibration fluid can be flowed over the sensor or calibration fluid can simply remain in contact with the sensor. Following calibration the sensor can be exposed to a low glucose containing solution prior to measurement.

FIG. 5 is a schematic illustration of a blood access system using a single access line. The system comprises: a catheter providing access patient, a stopcock or other access port, a measurement sensor, a syringe, a pressure measurement device, a fluid selection device allowing selection of the fluid sources for maintenance and calibration of the system.

### Example Embodiment

Push Pull system based upon Syringe with Calibration Fluid Pathway. FIG. 6 is a schematic illustration of an example embodiment comprising a push pull system based upon a syringe. The syringe creates a pressure gradient to withdraw blood from the patient to the sensor. The system as shown is manually operated. The syringe serves as a reservoir as the initial blood present will be mixed with saline. The use of a reservoir as shown eliminates the need for a separate waste bag. The system has the capability of doing a two point calibration. The system contains a separate fluid pathway with a connection near the sensor. This separate fluid path helps to minimize the amount of calibration solution that is infused into the patient. To effectively expose the sensor to a calibration fluid, the stopcock needs to be opened the sensor exposed to the calibration fluid. The short length of tubing reduces mixing and the total volume of fluid needed. An additional port on the existing stopcock or an additional stopcock or port (not shown) allows for procurement of a blood sample or the introduction of additional calibration, validation or check samples. The pressure measurement device allows for pressure monitoring. The pressure measurement system can be attached to the either fluid pathway and in operation must be exposed to the pressure changes of the patient for effective pressure measurement. If attached to an arterial line the fluid bags would be pressurized to create a pressure gradient to create positive flow to the patient. The system is closed to the environment and operates in an entirely sterile manner. Following completion of the measurement, the syringe is pushed so as to remove all fluid from the cylinder. Additional washing of the system is provided by allowing flow from the fluid sources towards the patient. In the case of venous attachment, this flow is by gravity. The system comprises: a catheter providing access patient, a stopcock or other access port, a measurement sensor, a fluid connection to the calibration fluid, a syringe, a pressure measurement device, a stopcock allowing selection of the fluid sources and fluid sources for maintenance and calibration of the system. One-way valves can be mounted with the system to allow fluid flow from the bags to the system. The operation of the example embodiment is described below.

Blood Sample and Measurement Process.

1. The system is calibrated as described below. Following calibration the operator initiates a blood draw by moving the syringe plunger away from the home position. The draw continues until an undiluted sample is present at the measurement sensor. The determination of an undiluted sample can be by volume drawn, visual inspection or the sensor sample state methods described above.
2. The blood interacts with measurement sensor and an analyte measurement is made. The blood may be flowing or not flowing across the sensor during the measurement.
3. Following completion of the measurement, the syringe is pushed towards the home position so that the blood is returned to the patient. At this juncture the majority of all blood has been returned to the patient.
4. If additional cleaning of the circuit is desired, fluid from either fluid source can be used to clean the circuit further. The fluid can simple by flowed through the system or drawn into the syringe. If drawn into the syringe, the operator can use a push-stop-push flow pattern to facilitate cleaning. The cleaning process helps to maintain the circuit for future use and prevent clotting of the circuit.
5. Following cleaning of the circuit, fluid can continue to flow toward the patient to create a “keep vein open” fluid infusion towards the patient.
6. The measurement results and any historical information are communicated to a user, e.g., shown on a display (not shown).

Calibration Process. The system has two fluid sources that can be used to facilitate calibration of the sensor. The fluid sources have different analyte levels. The fluid selection device can be used to select the fluid of choice. Several different methods can be used to move the fluid over the sensor. As an example, gravity feed can move the fluid so that the sensor is exposed to the designated calibration fluid. As another example, the fluid sources can be pressurized to move the fluid. As another example, additional flow generation devices can be added to create flow. As shown in FIG. 6, the syringe in combination with the flow selection device can be used to pull fluid from the fluid sources with subsequent flow occurring over the sensor. The calibration solution is delivered via the bypass circuit to the sensor. During the calibration process calibration fluid can be flowed over the sensor or fluid can simply remain in contact with the sensor. Following calibration of the sensor with the calibration fluid, the fluid selection device is configured to select the saline fluid. As described elsewhere in this specification it can be advantageous to maintain the sensor in a low analyte containing solution prior to measurement. Based upon these advantages and the general desire not to infuse the patient with high analyte concentration fluid, the higher analyte containing solution would be the calibration solution. The saline solution can be simply saline, other IV fluids, an IV fluid with anticoagulant, or a calibration solution with a lower analyte value.

The example embodiment of FIG. 6 can provide several important characteristics:

1. Analyte measurements can be made on a very frequent basis.
2. The system operates with no blood loss.
3. The system can work on multiple access locations including arterial.
4. The system can contain a pressure monitor that can provide arterial, central venous, or pulmonary artery catheter pressure measurements.
5. The system can compensate for different size catheters through the volume pulled via the syringe.
6. The system provides for a two point calibration via the two fluid sources.
7. The system provides for access to the blood sample via a port or stopcock in the circuit.
8. Additional samples can be inserted into the system via the access port (not shown).
9. The system provides completely sterile operation.
10. The use of the calibration bypass circuit helps to limit the amount of calibration solution infused into the patient.

### Example Embodiment

Push Pull system based upon Syringe with Waste Fluid Pathway. FIG. 7 is a schematic illustration of an example embodiment comprising a push pull system based upon a syringe. The syringe creates the pressure gradient needed to withdraw blood from the patient to the sensor. The system is shown is manually operated. The syringe serves as a reservoir as the initial blood present will be mixed with saline. The use of a reservoir as shown eliminates the need for a separate waste bag. The system has the capability of doing a two point calibration. The system contains a separate fluid pathway to the waste bag. This separate fluid path helps to minimize the amount of solution that is infused into the patient. For example, all fluid used for calibration and or cleaning can be directed to waste bag. Fluid selection device number one is used to define the fluid flowing to the sensor. If the operator desires to have the fluid directed to waste, fluid selection device number #2 can position such that fluid flow is to waste bag. The use of fluid selection device #2 coupled with the waste bypass pathway provides the operator with the opportunity of moving all calibrate and/or waste fluids to the waste bag. An additional port on the existing stopcock or an additional stopcock or port (not shown) allows for procurement of a blood sample or the introduction of additional calibration, validation or check samples. The pressure measurement device allows for pressure monitoring. The pressure monitoring system can be attached to any of the fluid pathways shown provided that in operation the pressure measurement system has appropriate exposure to the pressure variations from the patient. If attached to an arterial line the fluid bags can be pressurized to create a pressure gradient to create positive flow to the patient. The system operates in an entirely sterile manner. Following completion of the measurement, syringe is pushed so as to remove all fluid from the cylinder. Additional washing of the system is provided by allowing flow from the fluid sources towards the patient. This additional washing fluid can be infused into the patient or directed to the waste bag. In the case of venous attachment, this flow can be by gravity. The system comprises: a catheter providing access patient, a stopcock or other access port, a measurement sensor, a fluid connection to the waste bag, a syringe, a pressure measurement device, a stopcock allowing selection of the fluid sources and fluid sources for maintenance and calibration of the system. One-way valves can be mounted with the system to allow fluid flow from the fluid bags to the system, and to allow fluid to flow from the system to the waste bag. The operation of the example embodiment is described below.

Blood Sample and Measurement Process.

1. The system is calibrated as described below. Following calibration the operator initiates a blood draw by moving the syringe plunger away from the home position. The draw continues until an undiluted sample is present at the measurement sensor. The determination of an undiluted sample can be by volume drawn, visual inspection or the sensor sample state methods described above.
2. The blood interacts with measurement sensor and an analyte measurement is made. The blood may be stagnant during the measurement process or flowing across the sensor.
3. Following completion of the measurement, the syringe is pushed towards the home position so that the blood is returned to the patient At this juncture the majority of all blood has been returned to the patient. At any point during the infusion process, the operator may elect to direct the fluid to waste.
4. If additional cleaning of the circuit is desired, fluid from either fluid source can be used to clean the circuit further. The fluid used for cleaning can be directed to waste by fluid selection device #2. The fluid can flow through the system or be drawn into the syringe. If drawn into the syringe, the operator can use a push-stop-push flow pattern to facilitate cleaning. The cleaning process helps to maintain the circuit for future use and prevent clotting of the circuit.
5. Following cleaning of the circuit, fluid can continue to flow toward the patient to create a “keep vein open” fluid infusion towards the patient.
6. The measurement results and any historical information are communicated to a user, e.g., shown on a display (not shown).

Calibration Process. The system has two fluid sources that can be used to facilitate calibration of the sensor. The fluid sources have different glucose levels. The fluid selection device can be used to select the fluid of choice. Gravity feed moves the fluid so that the sensor is exposed to the designated calibration fluid or alternatively, the fluid sources can be pressurized to move the fluid. The calibration solution is delivered to the sensor and either be infused into the patient or directed to the waste bag. During the calibration process, calibration fluid may be flowed over the sensor or fluid may simply remain in contact with the sensor. Following calibration of the sensor with the calibration fluid, the fluid selection device #1 is configured to select the saline fluid. As described elsewhere in this specification it can be advantageous to maintain the sensor in a low analyte containing solution prior to measurement. Based upon these advantages and the general desire not to infuse the patient with high analyte concentration fluid, the higher analyte containing solution would be the calibration solution. The saline solution can be simply saline, other IV fluids, an IV fluid with anticoagulant, or a calibration solution with a lower analyte value.

The example embodiment of FIG. 7 can provide several important characteristics:

1. Analyte measurements can be made on a very frequent basis.
2. The system operates with no blood loss.
3. The system can work on multiple access locations including arterial.
4. The system can contain a pressure monitor that can provide arterial, central venous, or pulmonary artery catheter pressure measurements.
5. The system can compensate for different size catheters through the volume pulled via the syringe.
6. The system provides for a two point calibration via the two fluid sources.
7. The system provides for access to the blood sample via a port or stopcock in the circuit.
8. Additional samples can be inserted into the system via the access port (not shown).
9. The system provides completely sterile operation.
10. The use of the waste bypass pathway helps to limit the amount of solution infused into the patient.

### Example Embodiment

Push Pull system based upon Syringe with Calibration and Waste Fluid Bypass Circuits. FIG. 8 is a schematic illustration of an example embodiment that combines characteristics of the example embodiments illustrated in FIGS. 6 and 7. The system is push pull based via the use of a syringe. The syringe creates the pressure gradient needed to withdraw blood from the patient to the sensor. The system is shown is manually operated. The syringe serves as a reservoir as the initial blood present will be mixed with saline. The use of a reservoir as shown eliminates the need for a separate waste bag. The system has the capability of doing a two point calibration. The system contains two separate fluid pathways. The first is between the calibration solution and a fluid selection device in fluid connectivity with the sensor. The second pathway is between the waste bag and a second fluid selection device in fluid connectivity with a sensor. These separate fluid paths can be used to minimize the amount of solution that is infused into the patient. An additional port on the existing stopcock or an additional stopcock or port (not shown) allows for procurement of a blood sample or the introduction of additional calibration, validation or check samples. The pressure measurement device allows for pressure monitoring. If attached to an arterial line the fluid bags would be pressurized to create a pressure gradient to create positive flow to the patient. The system operates in an entirely sterile manner. Following completion of the measurement, syringe is pushed so as to remove all fluid from the cylinder. Additional washing of the system is provided by allowing flow from the fluid sources towards the patient. This additional washing fluid can be infused into the patient or directed to the waste bag. In the case of venous attachment, this flow is by gravity. The system comprises: a catheter providing access patient, a stopcock or other access port, a measurement sensor, a fluid connection to the calibration bag, a fluid connection to the waste bag, a syringe, a pressure measurement device, a stopcock allowing selection of the fluid sources and fluid sources for maintenance and calibration of the system. One-way valves can be mounted with the system to allow fluid flow from the fluid bags to the system, and from the system to the waste bag.

### Example Embodiment

Push Pull System Based upon Syringe with Sensor Access. FIG. 9 is a schematic illustration of an example embodiment comprising a push pull system based upon a syringe. The syringe creates the pressure gradient needed to withdraw blood from the patient to the sensor. The system as shown is manually operated. The syringe serves as a reservoir as the initial blood present will be mixed with saline. The use of a reservoir as shown eliminates the need for a separate waste bag. The system has the capability of doing a one, two or multi-point calibration. The system contains two fluid selection devices located on either side of the sensor. These fluid selection devices provide fluid access sites that can be used to calibrate the sensor, procure blood samples, and run additional validation samples separate. As an example, two syringes can be attached to the two fluid selection devices shown. Fluid can be transferred from one syringe to the other such that flow occurs over the sensor. Such a manual process can have advantages in quality control and the amount of fluid infused into the patient. The existing ports or an additional stopcock or port (not shown) allows for procurement of a blood sample or the introduction of additional calibration, validation or check samples. The pressure measurement device allows for pressure monitoring. If attached to an arterial line the fluid bags would be pressurized to create a pressure gradient to create positive flow to the patient. The system operates in an entirely sterile manner. Following completion of the measurement, syringe is pushed so as to remove all fluid from the cylinder. Additional washing of the system is provided by allowing flow from the fluid sources towards the patient. This additional washing fluid can be infused into the patient or directed to the waste bag. In the case of venous attachment, this flow is by gravity. The system comprises: a catheter providing access patient, two fluid selection devices, a measurement sensor, a syringe, a pressure measurement device, and fluid sources for maintenance and calibration of the system. One-way valves can be mounted with the system to allow fluid flow from the fluid bags to the system.

### Example Embodiment

Two Syringe Push Pull System. FIG. 10 is a push pull system based upon two syringes. The syringes create the pressure gradient needed to withdraw saline or blood away from the patient to the sensor. The system is shown is manually operated. The syringe serves as a reservoir as the initial blood present will be mixed with saline. The use of a reservoir as shown eliminates the need for a separate waste bag. The two syringes provide flexibility in operation. For example, only saline could be pulled into a first syringe while mostly blood is pulled into a second syringe. Such a division of blood and saline might limit the amount of anticoagulant needed to prevent clotting. The system has the capability of doing a two point calibration. The stopcock shown allows for procurement of a blood sample or the introduction of additional calibration, validation or check samples. The pressure measurement device allows for pressure monitoring. If attached to an arterial line the fluid bags would be pressurized to create a pressure gradient to create positive flow to the patient. The system operates in an entirely sterile manner. Following completion of the measurement, the syringe is pushed so as to remove all fluid from the cylinder. Additional washing of the system is provided by allowing flow from the fluid sources towards the patient. In the case of venous attachment, this flow is by gravity. The two syringes can be used individually or in combination to facilitate cleaning of the system. The system comprises: a catheter providing access patient, a stopcock or other access port, a measurement sensor, a T-junction, a pressure measurement device, two syringes, and appropriate check and fluid sources for maintenance and calibration of the system. One-way valves can be mounted with the system to allow fluid flow from the fluid bags to the system.

### Example Embodiment

Two Reservoir Push Pull System with Peristaltic Pump. FIG. 11 is a schematic illustration of an example embodiment comprising an automated system using two reservoirs and a pumping mechanism. The pump creates the pressure gradient needed to withdraw saline or blood away from the patient to the sensor. The fluid withdrawn can be directed into one or two available reservoirs. The use of a reservoir(s) as shown eliminates the need for a separate waste bag. If two reservoirs are utilized, they provide flexibility in operation. For example, only saline could be pulled into one reservoir while mostly blood is pulled into the other reservoir. Such configuration might limit the amount of anticoagulant needed to prevent clotting. The system has the capability of doing a two point calibration. The valves shown allow the operator to select the associated fluid pathway. The pressure measurement device allows for pressure monitoring. If attached to an arterial line the fluid bags would be pressurized to create a pressure gradient to create positive flow to the patient. The system operates in an entirely sterile manner. Following completion of the measurement, syringe is pushed so as to remove all fluid from the cylinder. Additional washing of the system is provided by allowing flow from the fluid sources towards the patient. In the case of venous attachment, this flow is by gravity. The pump can be operated to facilitate cleaning of the system. The system comprises: a catheter providing access to a patient, a stopcock or other access port, a measurement sensor, a pump, a pressure measurement device, a T-junction, two reservoirs, two valves, appropriate check (one-way) valves and fluid sources for maintenance and calibration of the system.

### Example Embodiment

Push Pull System based upon Peristaltic Pump. FIG. 12 shows a push pull system based upon a peristaltic pump. The system configuration is similar to FIG. 4 except that the pressure gradient for flow is provided by a pump. The pump creates a pressure gradient to withdraw blood from the patient to the sensor. The blood reservoir serves as a reservoir as the initial blood present will be mixed with saline. The use of a reservoir as shown eliminates the need for a separate waste bag. The system has the capability of doing a two point calibration. The pressure measurement device allows for pressure monitoring. If attached to an arterial line the pump can create the appropriate pressure gradient needed to enable fluid infusion. The system operates in an entirely sterile manner. Following completion of the measurement, the pump is activated to push the blood towards the patient. Additional washing of the system can be provided by the pump, specifically the pump can provide a stop-push or back and forth cleaning action.

FIG. 12 is a schematic illustration of a blood access system using a single access line. The system comprises: a catheter providing access patient, a pump, a measurement sensor, a reservoir, a pressure measurement device, a fluid selection device allowing selection of the fluid sources and fluid sources for maintenance and calibration of the system. One-way valves can be mounted with the system to allow fluid flow from the fluid bags to the system.

### Example Embodiment

Push Pull System Based upon Syringe with Flow Divider Bypass. FIG. 13 is a schematic illustration of an example embodiment comprising a push pull system where the flow generation device is a syringe. The syringe creates the pressure gradient needed to withdraw blood from the patient to the sensor. The system is shown is manually operated. The syringe serves as a reservoir as the initial blood present will be mixed with saline. The system also contains a bypass configuration intended to limit the flow rate through sensor during the filling and reinfusion phases. The slower flows through the sensor limit the shear caused by flow through the small diameter of the sensor. The flow divider is designed to divide the flow between the two channels in a manner that allows for a good measurement and cleaning of the sensor while concurrently limiting the shear stress on the blood and sensor. One possible embodiment uses different cross sectional areas to provide the appropriate flow resistance to achieve the above goals. See FIG. 14 for an example flow divider. The use of a reservoir as shown eliminates the need for a separate waste bag. The system has the capability of doing a two point calibration. The stopcock shown allows for procurement of a blood sample or the introduction of additional calibration, validation or check samples. The pressure measurement device allows for pressure monitoring. If attached to an arterial line the fluid bags would be pressurized to create a pressure gradient to create positive flow to the patient. The system operates in an entirely sterile manner. Following completion of the measurement, syringe is pushed so as to remove all fluid from the cylinder. Additional washing of the system is provided by allowing flow from the fluid sources towards the patient. In the case of venous attachment, this flow is by gravity. The system comprises: a catheter providing access patient, a stopcock or other access port, a flow divider, a measurement sensor, a syringe, a pressure measurement device, a fluid selection device allowing selection of the fluid sources and fluid sources for maintenance and calibration of the system. One-way valves can be mounted with the system to allow fluid flow from the fluid bags to the system.

FIG. 14 is a schematic illustration of a flow divider. The cross section areas of the three tubes are sized so that appropriate flow and associated sheer is achieved through the sensor. The lower part of FIG. 14 shows the different cross sectional areas.

### Example Embodiment

Push Pull System Based upon Syringe with Flow Divider Bypass. FIG. 15 is a schematic illustration of an example embodiment comprising a push pull system where the flow generation device is a syringe. The syringe creates the pressure gradient needed to withdraw blood from the patient to the sensor. The system is shown is manually operated. The syringe serves as a reservoir as the initial blood present will be mixed with saline. The system also contains a bypass configuration which allows flow to be diverted around the sensor. For the reduction of shear within the sensor, it may be desirable to bypass during periods of maximum flow periods. Additionally, the bypass is configured with stopcocks on either side of the sensor to allow user to put the sensor in-line for measurement phase, then isolate the sensor from the circuit to prevent sensor-related disruption of the blood pressure signal. The use of a reservoir as shown eliminates the need for a separate waste bag. The system has the capability of doing a two point calibration. The stopcock shown allows for procurement of a blood sample or the introduction of additional calibration, validation or check samples. The pressure measurement device allows for pressure monitoring. If attached to an arterial line the fluid bags would be pressurized to create a pressure gradient to create positive flow to the patient. The system operates in an entirely sterile manner. Following completion of the measurement, syringe is pushed so as to remove all fluid from the cylinder. Additional washing of the system is provided by allowing flow from the fluid sources towards the patient. In the case of venous attachment, this flow is by gravity. The system comprises: a catheter providing access patient, a stopcock or other access port, a flow divider, a measurement sensor, a syringe, a pressure measurement device, a fluid selection device allowing selection of the fluid sources and fluid sources for maintenance and calibration of the system. One-way valves can be mounted with the system to allow fluid flow from the fluid bags to the system.

The example embodiment of FIG. 15 can provide several important characteristics:

1. Analyte measurements can be made on a very frequent basis.
2. The system operates with no blood loss.
3. The system can work on multiple access locations including arterial.
4. The system contains a pressure monitor that can provide arterial, central venous, or pulmonary artery catheter pressure measurements.
5. The system can compensate for different size catheters through the volume pulled via the syringe.
6. The system provides for a two point calibration via the two fluid sources.
7. The system provides for access to the blood sample via a port or stopcock in the circuit.
8. Additional samples can be inserted into the system via the access port (not shown).
9. The system provides completely sterile operation.
10. If the sensor has a small cross sectional area or significant compliance, then the bypass circuit enables pressure monitoring without corruption of the signal during non-measurement periods.
11. If the sensor has a small cross sectional area or can be damaged by flow, then the bypass circuit can be used. In practice, an undiluted sample could be drawn to the sensor location via the bypass loop. At this point in the measurement cycle, the fluid selection devices changes to flow through the sensor occurs. The additional blood needed to fill the sensor is small in comparison the amount needed to get an undiluted sample to the sensor.

### Example Embodiment

System configuration. FIG. 16 is a block diagram of an example embodiment. The system comprises a catheter (or similar blood access device) suitable to be placed in fluid communication with the vascular system of a patient, and in fluid communication with an analyte sensor via a first fluid transport apparatus 101. A second fluid transport apparatus 102 connects the analyte sensor with the flow generation and reservoir system. A third fluid transport apparatus 103 connects the flow generation device with a fluid selection device. The fluid selection device is connected to one of more fluid sources via fourth 104 and fifth 105 fluid transport apparatuses. The flow generation and reservoir system can be a single device such as a syringe or can include separate devices such as a pump and bag. In operation, the flow generation device uses the first fluid transport apparatus to draw blood from the patient to the analyte sensor. Fluid exits the sensor into the second fluid transport apparatus. The fluid is moved by the flow generation device and stored in the fluid reservoir. The operator can use the flow generation device to flow blood over the sensor during the measurement, or measurements can be made with the fluid in a stagnant state. Following completion of the measurement the flow generation device infuses the withdrawn fluid into the patient. Additional cleaning can be conducted as needed. The example embodiment has the ability to conduct a two point calibration by using the fluid selection device. The fluid selection device can be used to select the desired fluid source to enable calibration of the sensor. Multiple methods and fluid sequences can be used for calibration within the context of the example embodiment. As examples of such calibration, see U.S. patent application Ser. No. 12/576,303 “Method for Using Multiple Calibration Solutions with an Analyte Sensor with Use in an Automated Blood Access System” filed Oct. 9, 2009, incorporated herein by reference. When the system is not making a measurement or being calibrated, the flow generation device in combination with the flow selection device can be used to flow a fluid source through first and second fluid transport apparatuses toward the patient to maintain open access to the circulatory system of the patient.

### Example Embodiment

System configuration. FIG. 17 is a block diagram of an example embodiment. The system comprises a catheter (or similar blood access device) suitable to be placed in fluid communication with the vascular system of a patient, and in fluid communication with an analyte sensor via a first fluid transport apparatus 110. A second fluid transport apparatus 112 connects the analyte sensor with the flow generation and reservoir system. A third fluid transport apparatus 113 connects the flow generation and reservoir system with a fluid selection device 114. The fluid selection device is connected to a fluid source #2 via a fourth fluid transport apparatus 115. A fifth fluid transport apparatus 116 connects fluid selection device 117 to fluid transport apparatus 112. A sixth fluid transport apparatus 118 connects the fluid selection device 117 to a fluid source #1. The flow generation and reservoir system can be a single system such as a syringe or can include separate devices such as a pump and a bag. In operation, the flow generation device uses the first fluid transport apparatus to draw blood from the patient to the analyte sensor. Fluid exits the sensor into the second fluid transport apparatus. The fluid is moved by the flow generation device and stored in the fluid reservoir. The operator can use the flow generation device to flow blood over the sensor during the measurement, or measurements can be made with the fluid not flowing. Following completion of the measurement the flow generation device infuses the withdrawn fluid into the patient. Additional cleaning can be conducted as needed. The example embodiment has the ability to conduct a two point calibration by using the fluid selection devices 117 and 114. Fluid selection device 117 can be configured (e.g., opened to fluid flow) so the analyte sensor is exposed to fluid source #1. Fluid selection device 114 can be configured (e.g., opened to fluid flow) to provide the sensor access to fluid source #2. The fluid selection devices can be used to select the desired fluid source to enable calibration of the sensor. Multiple methods and fluid sequences can be used for calibration within the context of the example embodiment. As examples of such calibration, see U.S. patent application Ser. No. 12/576,303 “Method for Using Multiple Calibration Solutions with an Analyte Sensor with Use in an Automated Blood Access System” filed Oct. 9, 2009, incorporated herein by reference. When the system is not making a measurement or being calibrated, the flow generation device in combination with the flow selection device can be used to flow a fluid source through first and second fluid transport apparatuses toward the patient to maintain open access to the circulatory system of the patient.

Calibration and Maintenance. The present invention can also provide improved methods for maintaining and calibrating an analyte sensor such as a glucose sensor for improved performance and safety. Via recognition of enzyme kinetics, the improved methods facilitate a faster measurement response which limits the potential for blood coagulation. The improved methods also reduce enzyme suppression which can lead to inaccurate results. The improved methods, via the use of a low glucose concentration maintenance fluid, create a safer system by limiting the potential for erroneously high readings.

FIG. 19 is an illustration of an example embodiment of a blood access and measurement system suitable for use with the present invention. The example automated blood analyte measurement system contains two fluid bags providing for at least two different calibration points. In use, the analyte sensor can be exposed to a zero or predetermined low glucose concentration via fluid from the saline bag. A second glucose concentration can be provided via fluid from the calibration solution bag. The example system in FIG. 19 provides the opportunity for calibration of the device with a known calibration fluid while concurrently minimizing the infusion of the calibration fluid into the patient. In the example system, the calibration fluid solution can be pumped through the circuit and directly to waste without infusion into the patient. For example, the flush pump can be operated in a manner towards the patient and the blood pump can operate at a similar rate away from the patient. In this manner the analyte sensor is exposed to the calibration fluid solution but no fluid is infused into the patient. Following sensor calibration, fluid from the other fluid bag can be used to wash the circuit in a similar manner. Such a process can enable the effective calibration of the glucose sensor at a second glucose concentration. The system also enables the sensor to be maintained in a solution with low glucose concentration. The system then enables the effective calibration of the system as well as the maintenance of the sensor in a solution that facilitates rapid and accurate results.

FIG. 20 is an illustration of an example embodiment where the sensor is located near the patient. The sensor can be located in the IV catheter, immediately adjacent to the catheter or generally near the patient. The example automated blood analyte measurement system contains two fluid bags providing for at least two different calibration points, labeled in the figure as saline and cal bag. In use, the analyte sensor can be exposed to a zero or predetermined glucose concentration via fluid from the saline bag. A second glucose concentration can be provided via fluid from the calibration solution bag. The example system in FIG. 20 provides the opportunity for calibration of the device with a known calibration fluid while concurrently minimizing the infusion of the calibration fluid into the patient. In the example system, the calibration solution can be pumped through the circuit so that both tubes going to the sensor are filled with undiluted calibration solution. For example, the cal pump can be operated in a manner towards the patient and the saline pump can operate at a similar rate away from the patient. The fluid would go to waste as needed, (not shown). When the tube junction contains an appropriate calibration solution, the pumps can be activated so as to push the calibration solution to the sensor. The sensor can then be calibrated. To re-fill the circuit with a second calibration solution or a saline without glucose the saline pump can be operated in a manner towards the patient and the cal pump can operate at a similar rate away from the patient. This would result in a second solution near the tube junction. Again the solution can be moved to the sensor by operating both pumps toward the sensor or patient. The total amount of saline infused into the subject is very small when using this “loop” circuit. Such a process enables the effective calibration of the glucose sensor and enables the sensor to be maintained in a low glucose concentration prior to measurement. The location of the sensor near the patient, combined with a method to facilitate fast response from the enzyme sensor, creates a circuit design that can limit the amount of time the blood needs to be out of the body.

FIG. 21 shows an example implementation of a two level sensor calibration system. The example system in FIG. 21 enables the analyte sensor to be exposed to at least two known glucose concentrations. The variable valve can be a simple stopcock where the solution provided to the analyte sensor is either 100% calibration solution or 100% saline solution. In other embodiments a variable valve can provide for controlled mixing of these two fluid solutions to create multiple glucose concentrations. In any of the envisioned configurations, the system allows for calibration of the sensor and maintenance of the sensor in a low glucose concentration.

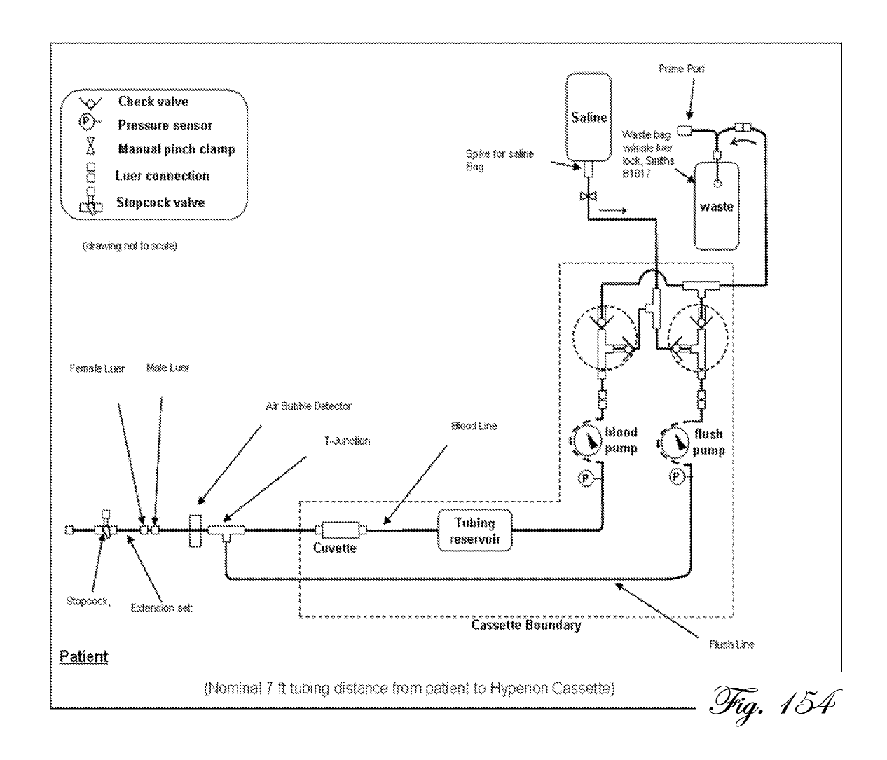

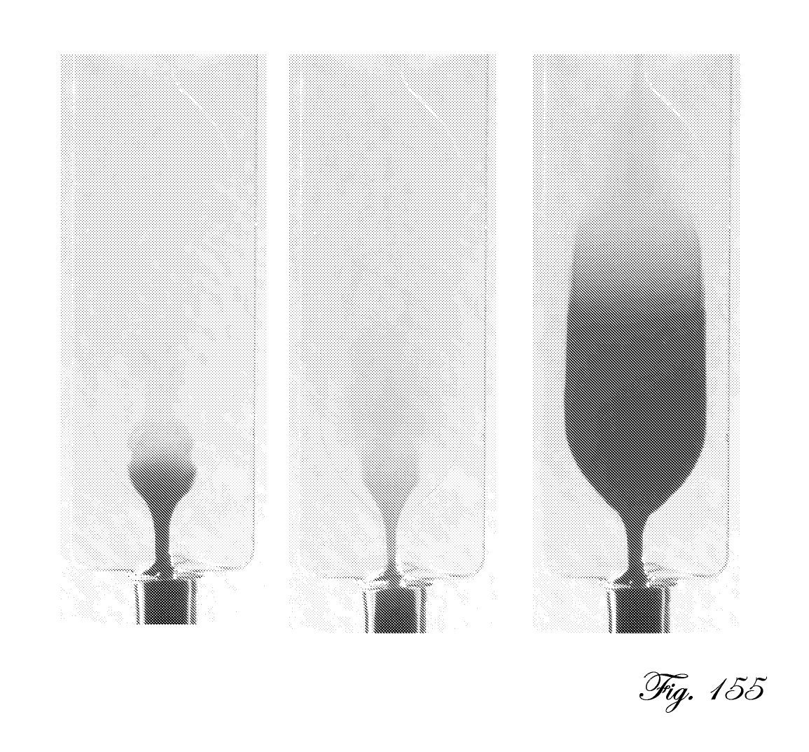

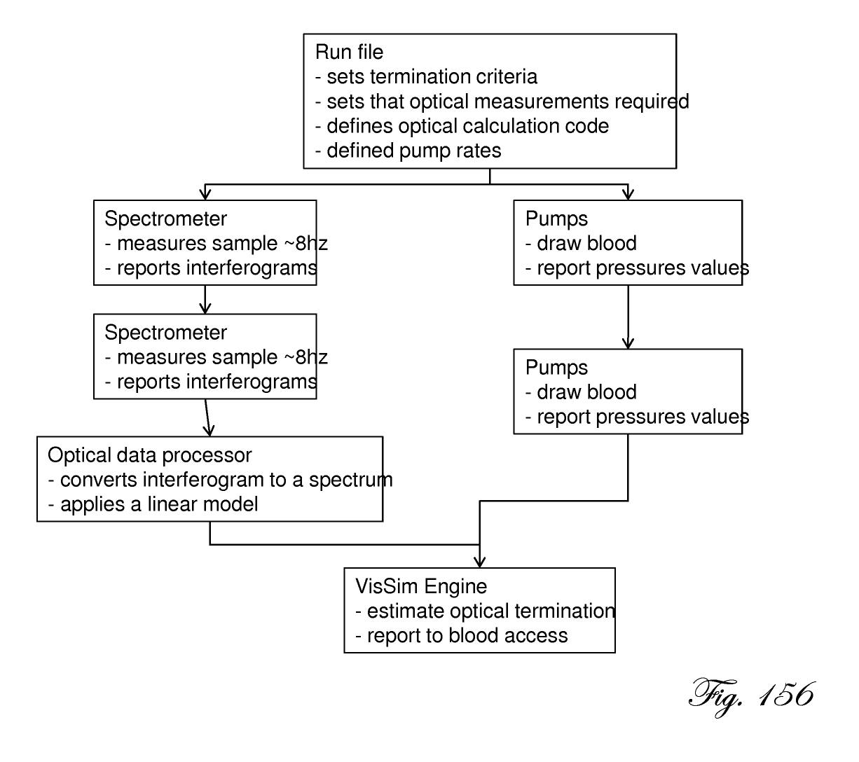

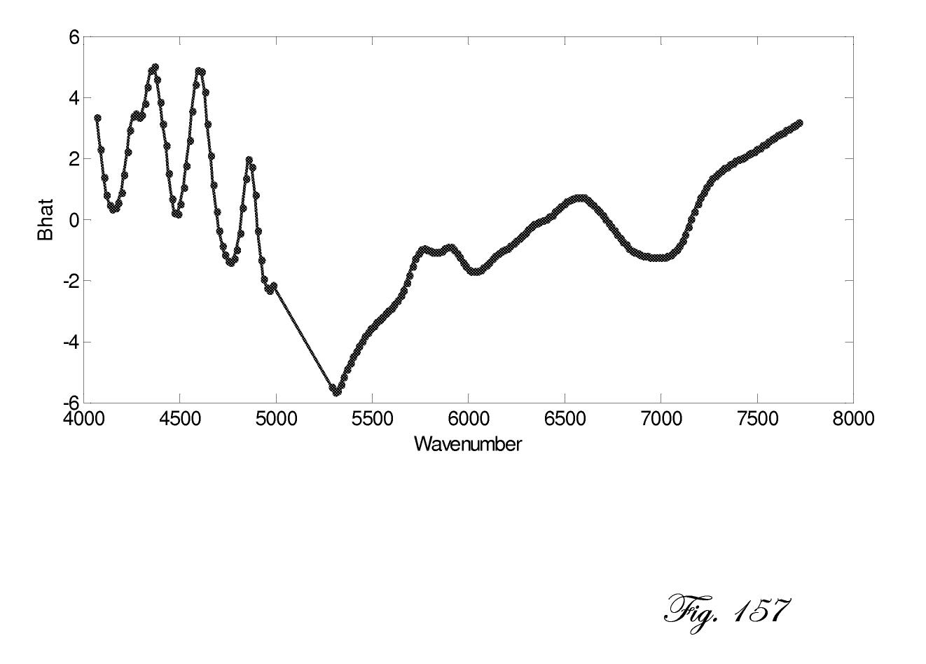

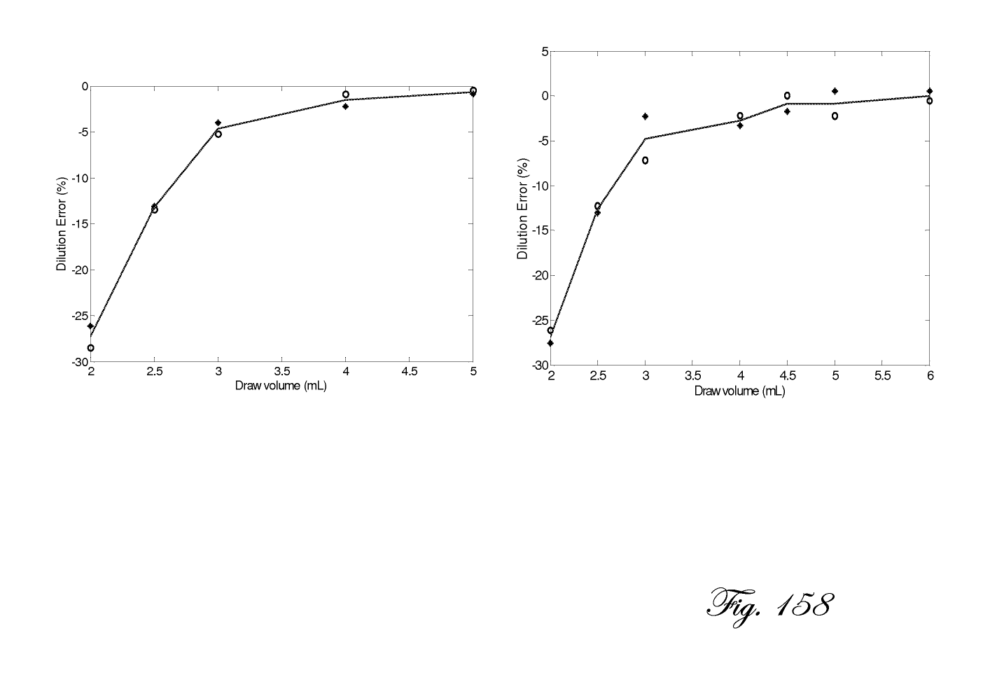

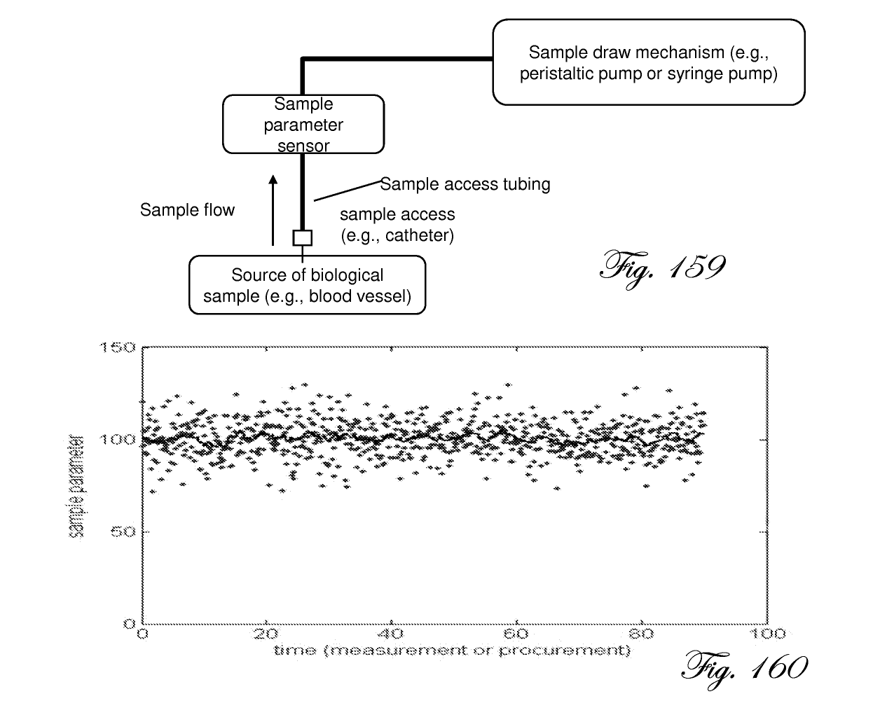

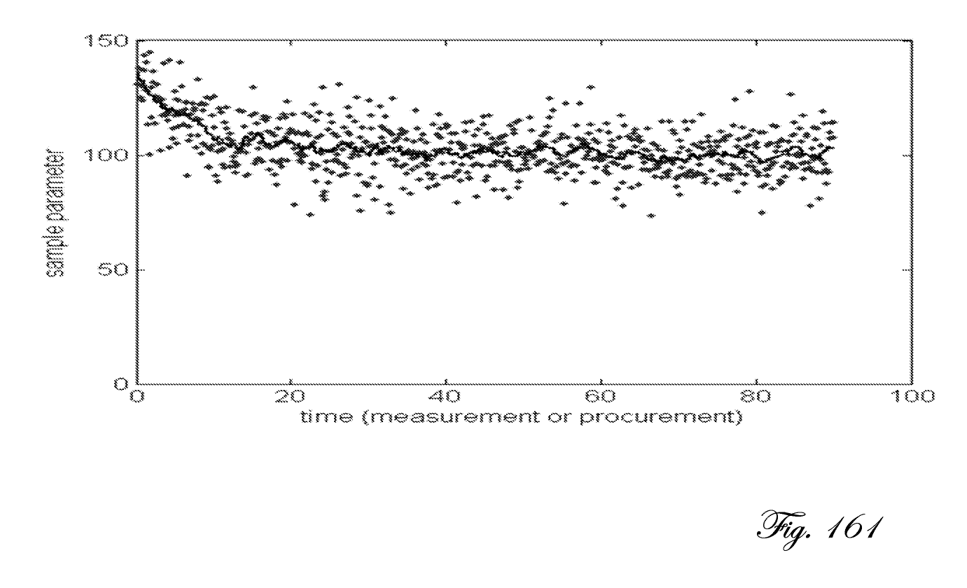

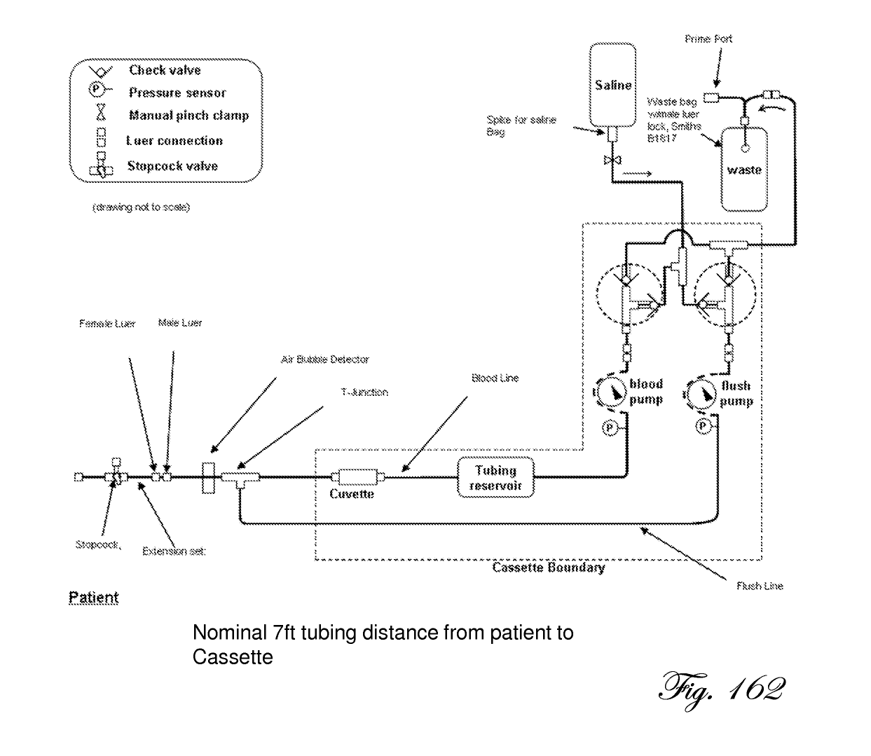

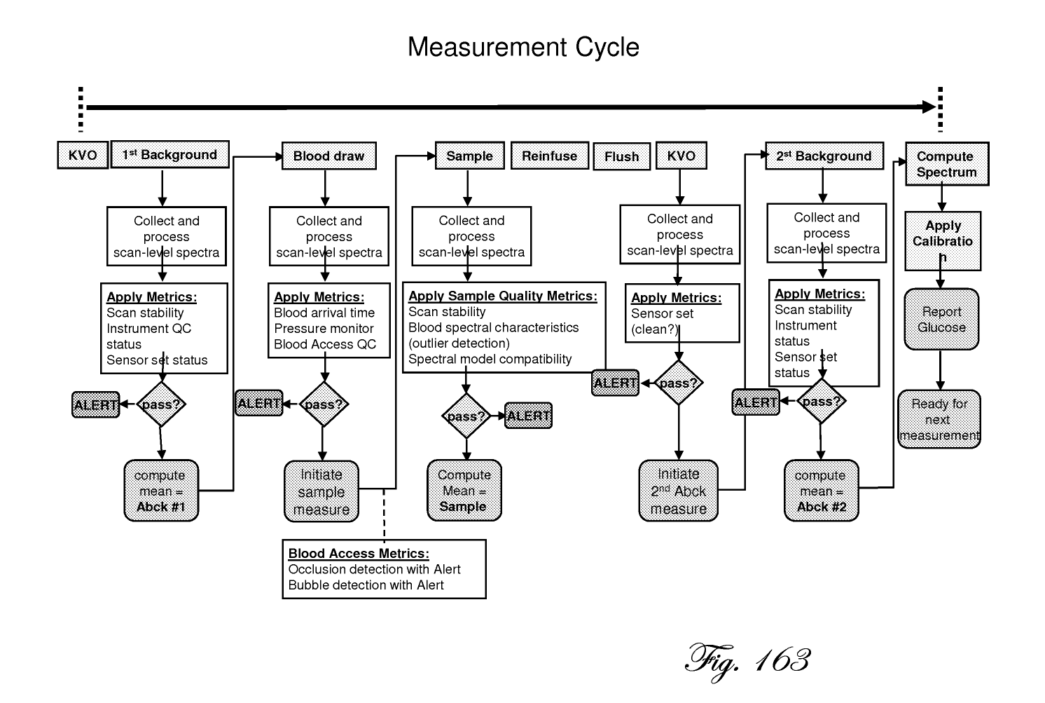

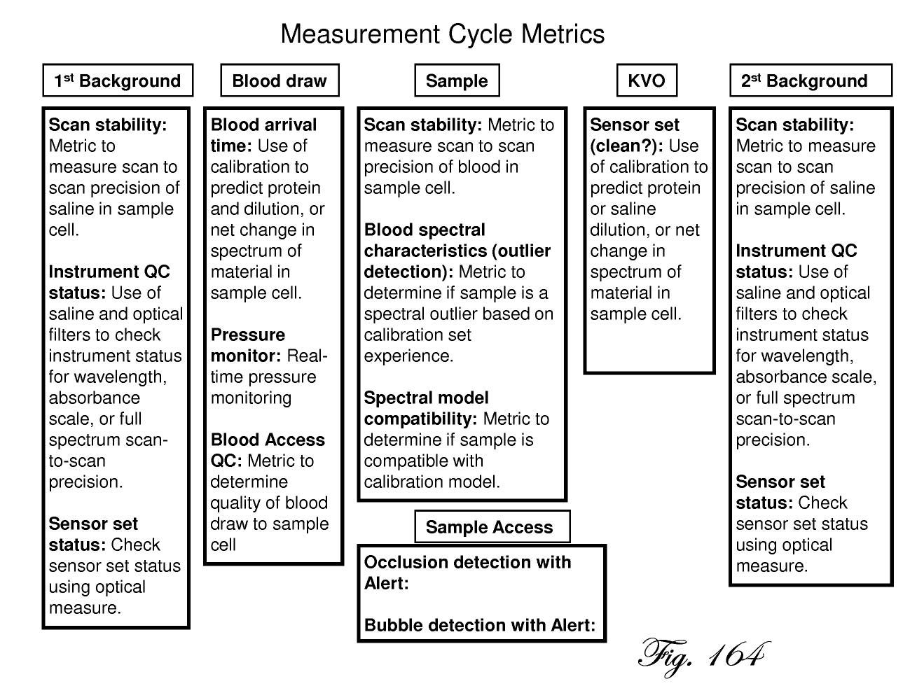

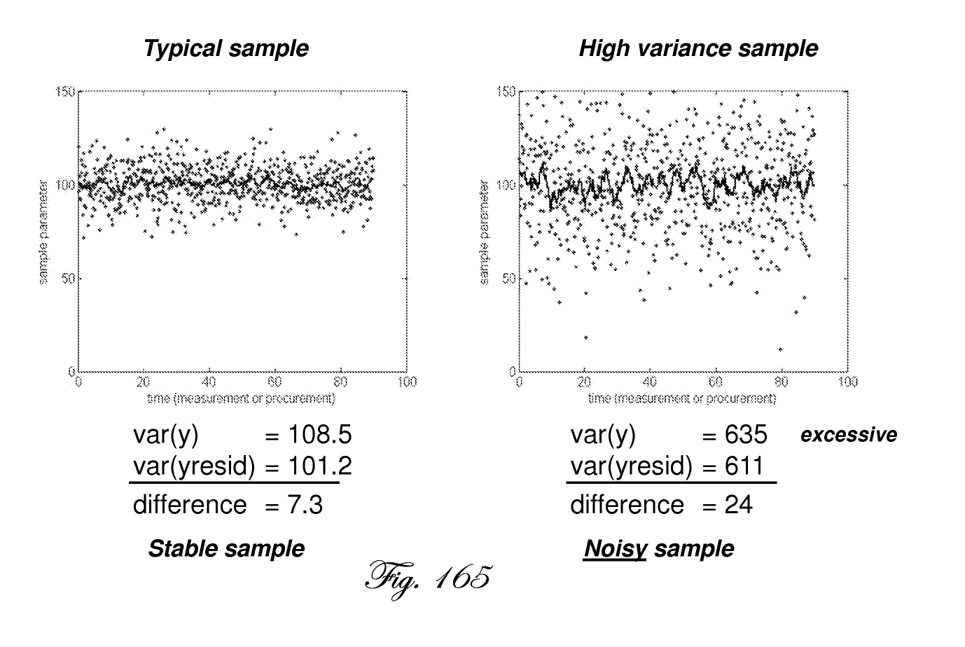

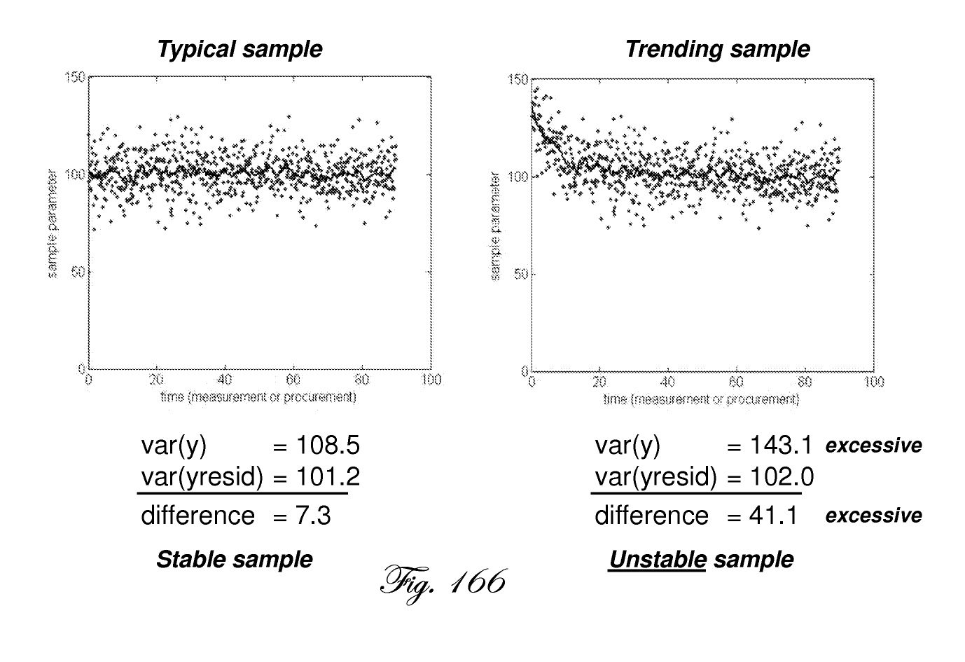

Method for determining the quality of a biological sample procured for ex vivo analysis The example blood access system is shown in FIG. 154, and can be described by considering three main component groups: 1) pump and measurement console, 2) a disposable sensor set, and 3) fluid bags that attach to the circuit.

The console can be attached to the patient through a sterile disposable sensor set designed for use with the console. As an example, the sensor set can be intended for use on a single patient for up to 72 hours. The sensor set, which can be attached to the patient using a dedicated peripheral venous catheter or other access location, provides convenient vascular access that enables automated withdrawal of a whole blood sample into an in-line optical cuvette for glucose measurement by means of optical transmission spectroscopy. When a glucose measurement is made, the system withdraws blood into the sensor set under controlled flow and pressure conditions. The system maintains flow of the blood during the glucose measurement, and reinfuses at least a portion of the blood to the patient once the glucose measurement is complete. The sensor set is connected to a saline bag which provides a flushing solution that keeps the lines and catheter free of thrombus formation and blood accumulation. In addition, the sensor set has a second path that connects to a waste bag through a T-junction near the patient connection. This path to waste enables thorough flushing and cleaning of the system between measurement cycles without infusing excess fluid to the patient.

The Console comprises:

Pumps—Pumps provide the ability to move blood and saline between the patient and the optical cuvette. There are two peristaltic pumps, the blood pump and the flush pump, that execute a programmed flow control sequence for the procurement of a blood sample for measurement, reinfusion of the blood following measurement and thorough cleaning of the sensor set after reinfusion. The sampling sequence is initiated by a manual request or pre-programmed, for example at a frequency or interval specified by the user.

Control System—Electronic controls and software manage pump speeds and directions and monitor the sensor set pressures during the blood measurement cycle. The blood measurement cycle will 1) maintain patency between blood samples, 2) withdraw a blood sample, 3) return the blood sample, and 4) clean the sensor set. If the Control System detects fault events in the blood access cycle, the control system will either execute automated procedures to clear the faults, or it will alert the user when faults cannot be automatically cleared. The Console also contains the optical measurement system, consisting of a light source and spectrometer for making the NIR glucose measurement. Glucose measurement algorithms can be resident in system nonvolatile memory.

Touch Screen—The Console can incorporate a touch screen computer for entering patient information and setting device operation parameters. The Console also provides visual display of measured glucose values as well as information associated with system operation including visual and audible alerts and alarms.

The Sensor Set includes:

Circuit Tubing—There are two tubes extending to the patient from the Cassette. One tube is used to convey blood and saline between the patient and optical cuvette. A second tube to the patient aids catheter flushing and returns saline used to clean the optical cuvette to the sensor set waste bag. Within the Cassette are a number of one-way valves used to isolate returned waste fluid from the patient.

Extension Set—The extension set connects the patient catheter to the disposable sensor set. The extension set includes a stopcock for lab blood draws and catheter maintenance, provides strain relief for ease of use and patient safety, and facilitates the attachment of the automated glucose measurement system to the patient.

Pump Cassette—The cassette attaches directly to the console and includes all electrical connections, peristaltic pump loops and one-way valves needed for operation. The cassette components comprise:

Pressure Sensors—Measure pressures inside the sensor set in the proximity of the pump tubing. There are two pressure sensors: the blood line pressure sensor and the flush line pressure sensor. Each sensor measures pressures on the patient side of the pump.

Tubing Reservoir—As a blood sample is withdrawn from the patient to the cuvette the first portion of the sample is diluted with saline. The diluted blood is pumped past the cuvette into the Tubing Reservoir. This overdraw enables measurement of an undiluted blood sample in the optical cuvette. The Tubing Reservoir is comprised of a vertical coil of tubing.

Bubble detector—The Blood Access System has a bubble detector that detects the presence of bubbles in the sensor set near the Extension Set. The bubble detector is used to ensure patient safety and to improve overall system functionality.

Cuvette—a glass tube with rectangular cross-section and fixed path length in which the blood measurement is made. The cuvette provides the interface between the sensor set and the spectrometer in the Optical Measurement System.

Two Fluid Bags can be useful for system operation:

Saline bag (user-supplied)—The Blood Access System pumps are able to move blood by pumping a column of sterile saline in advance of the blood sample. The sensor set accordingly requires a connection to a sterile saline bag. The sensor set is designed so that either the blood or flush pumps can pump fluid from the saline bag. One way valves ensure fluids cannot be pumped into the saline bag.

Waste bag—The Blood Access System requires a waste bag for collection and disposal of waste fluid generated during the flush and cleaning cycles. The sensor set is designed so that either the blood pump or flush pump can pump fluid into the waste bag. One way valves ensure fluids cannot be pumped out of the waste bag.

Operation of an Example Embodiment From an operational standpoint, the instrument can be separated into two primary functional subsystems that work in tandem to achieve the automated glucose measurement: 1) Blood Access System and 2) Optical Measurement System. The role of the Blood Access System is to safely and reliably draw a homogeneous blood sample from the patient into the optical cuvette, maintain the sample in a stable condition during the course of the optical measurement, return the blood to the patient and then flush and prepare the system for the next measurement cycle. The role of the optical measurement system is to collect NIR transmission spectra from the blood contained within the sensor set cuvette and to apply the appropriate signal conditioning and spectral data processing to confirm that an undiluted sample is present in the cuvette and to make a glucose determination from that sample.

The Blood Access System (see FIG. 154) can deliver an undiluted blood sample from the patient to the optical measurement system at a distance of approximately 7 feet from the patient. The system initiates a blood draw, pulls the blood from the patient and through the optical cuvette for glucose measurement, then reinfuses the blood to the patient following the measurement cycle. The system addresses the following issues:

Procurement of an undiluted blood sample for optical measurement;

Minimization of blood loss and fluids infusion;

Continued patency of the catheter, tubing and optical cuvette.

Procurement of an undiluted blood sample for optical measurement. The automated blood access system can use a sensor set that is primed with saline for safe and effective blood flow control. As the blood is drawn from the patient through the tubing, the blood/saline interface exhibits a parabolic flow profile and is characterized by a broadened transition zone of blood mixed with saline. The transition zone between undiluted blood and saline increases as the draw continues. Since the glucose measurement system can be sensitive to dilution effects, diluted blood is drawn past the glucose sensor and collected in the tubing reservoir until an undiluted sample is present in the cuvette. The present invention can be used to determine when an appropriate sample is present in the optical cuvette. Upon arrival of an appropriate sample, the system can initiate the measurement process. In the example embodiment, the measurement system is an optical measurement system but other measurement methods can be used. Other suitable methods can include indwelling electrochemical sensors, enzymatic sensors, sensors that work when in contact with blood such as those made by Dexcom and Abbott, standard sensors that work on a sample of blood and other optical sensing methods that use serum, plasma, supernatants or ultrafiltrates.

Minimization of blood loss and fluids infusion. Because of the blood-saline mixing at the interface between the two fluids, reinfusion of blood can involve some saline infusion to the patient. Similarly, when the system diverts fluid to waste during the cleaning process there can be an amount of residual blood in the tubing that goes to waste with the flush solution. There is a tradeoff between the amount of saline infused to the patient with each cycle versus the amount of blood diverted to waste. The automated Blood Access System can provide an optimized balance to minimize blood loss while simultaneously minimizing the saline infused to the patient with each sample. Typical standard maintenance intravenous fluid infusion rates are 125 mL/hr (3.0 liters per day) for a typical sized person. The procurement of automated measurements every 30 minutes would result in 48 paired measurements over the 24 hour period. If each measurement cycle infuses 9 ml of saline to the patient this will represent approximately 15% of a typical fluid maintenance rate. To minimize saline infusion during the measurement cycle and subsequent cleaning requires careful monitoring of infused volume to compensate for blood-saline mixing, and the use of specific fluid flow rates and patterns that optimize cleaning of the tubing during the blood infusion and cleaning. In regular operation, the only blood that is lost is that which is cleared from the walls of the tubing into the waste bag during the flush cycle. The amount of blood lost is less than 1004 per sample or approximately 5 mL/day at a 30 minute sample interval.

Patency of the catheter, tubing and cuvette. Stationary extracorporeal blood, unless treated with anticoagulants, tends to adhere to foreign surfaces and coagulates within a few minutes. To avoid these issues the process of blood withdrawal, measurement, reinfusion and cleaning can be completed effectively within a time frame that prevents blood coagulation and achieves effective cleaning of the circuit so aggregation of blood components within the walls of the tubing, cuvette and catheter of the sensor set does not occur.

The plumbing network (see FIG. 154) contains check valves configured to allow saline to be drawn from the saline bag into either the blood or flush line, and waste fluids to be pumped through either of these lines into the waste bag. The valves prevent the system from drawing fluid from the waste bag or from pumping fluid into the saline bag. Both the blood pump and the flush pump can provide flow in either direction. For example, during infusion saline is pulled from the saline bag and flows toward the patient. During withdrawal, fluid from the blood line is pumped towards the waste bag. The pumps can be operated independently or together at matched, opposite or different flow rates. Independent clockwise rotation from either pump causes blood to be drawn from the patient towards that pump and counterclockwise rotation causes fluid to be infused into the patient from either pump. Since the blood line and flush line are connected to each other and to the patient through a “T” near the patient, if the blood pump is operated in a counterclockwise direction and the flush pump is operated in a clockwise direction at a matched rate, then fluid will flow from the blood line into the flush line, pulling saline from the saline bag and pumping it into the waste bag.

Exception Detection and Management. Exceptions to the normal operation of the automated glucose measurement system occur when occlusions and air bubbles appear during the operation of the Blood Access System. The Blood Access System detects and manages occlusions, restrictions and air bubbles that can occur during any phase of the operational cycle. The system utilizes different recovery methods depending upon the stage of operation. Using measurements from the two pressure transducers near the pumps, the system can identify the location of a problem and will automatically clear the problem or alert the user so that it can be cleared manually. If the exception requires the user to take an action this is called an intervention.

In the operation of a Blood Access System, interventions that can occur include:

Occlusions due to positional occlusions of the catheter;

Air bubbles (typically from saline out gassing) when the system cannot automatically flush them to waste.

The automated glucose measurement system can use the following information for occlusion detection and management:

Pressure thresholds based upon the stage of operation;

Relationship of pressure between the two pressure sensors;

The time history of the pressure relationships between the pressure sensors;

The time history of pressure measurements (trend changes);

Dissipation of pressure within the circuit (the pressure change between the withdrawal and sample stages);

Time to complete a stage or time to complete stages;

Pressure trends between subsequent withdrawals;

Estimated flow rates based on pump rotational speeds and differential pressure readings.

This information can be incorporated into a decision flow chart that determines if an occlusion has occurred and initiates an appropriate recovery process. Generally, the system determines the stage of operation, the presence of blood in the circuit, the location of the occlusion and implements a recovery process to the extent possible. Depending upon the recovery results, an operator such as a nurse can be alerted. For example if an occlusion occurs in withdrawal, the system automatically re-infuses any blood withdrawn and a small amount of additional saline. The system will re-attempt a second blood draw. If occlusion is detected a second time the system again re-infuses any blood removed and automatically returns to a safe condition and alerts the care provider to address the problem.

The example system can also detect air bubbles in the line and prevent them from being infused to the patient. Common causes of air bubbles include out-gassing of the saline as it is subjected to negative pressure, and an increase in ambient temperature compared to the storage temperature of the saline. The automated glucose measurement system detects air bubbles below the T-junction near the Extension Set and stops flow upon detection. The system then determines the stage of operation, and the presence of blood in the circuit. Based upon this information a bubble management protocol is initiated. In most cases the bubble is pulled from the air bubble detector and past the T-junction into the flush line. Once isolated in the flush line, the system can flush the bubble to the waste bag for disposal. The system then resumes normal operation and provides an alert to an operator such as a nurse.

The Blood Access System operation can be described as 6 primary stages:

Draw initialization and clearing the catheter access;

Blood withdrawal;

Optical measurement;

Infusion;

Cleaning (incorporating Scrub, Recirculation, and Catheter Flush sub-stages);

KVO (“keep vein open”).

Draw Initialization Stage; Clearing Catheter Access. Before the blood draw is started, both the blood and flush pumps are controlled to issue a pulse of saline to clean away any residual blood in the catheter tip. This prepares the catheter for the subsequent withdrawal of blood.

Blood Withdrawal Stage. The blood pump is used to withdraw the blood sample and position non-diluted blood in the cuvette. To minimize the total draw time, about 80% of the total required blood volume is first drawn at a rapid flow rate. A constant-pressure-based draw method is used to compensate for the varying mix of saline and blood, and to achieve maximum flow rate constrained by the constant upstream negative pressure that keeps fluid degassing minimized. As blood replaces saline in the blood line, viscosity and resistance to flow increase so that for a constant upstream pressure, flow rate decreases over time. The termination of this stage of the draw is determined by what is referred to as optical termination. Optical termination is the optical detection of when a sample appropriate for measurement has filled the cuvette. After the optical termination of the withdrawal stage, the measurement of the sample can be initiated. An example of a specific optical termination method will be disclosed in detail below. Non-optical methods of detecting the arrival of an undiluted blood sample, such as those described elsewhere herein, can also be used.

Optical Measurement Stage. Following the rapid draw, the pump flow rate is slowed to a constant flow rate of 0.5 mL/min to maintain suspension of the red blood cells in plasma during optical measurement. During the 60 second measurement period an additional 500 μL of blood is withdrawn.

Infusion Stage. After the measurement is completed, reinfusion immediately begins as a progression of stages that are designed to return the blood quickly to the patient and clean the tubing and optical cuvette. The initial stage of infusion uses a constant pressure-based control which results in a variable flow rate that minimizes the time to reinfuse the blood to the patient. This stage reinfuses nearly all of the blood that was withdrawn, leaving a remaining saline-blood mixture at the end of the blood line. The first stage of the reinfusion can be completed within three minutes of the initiation of blood withdrawal.

The 2nd stage of infusion involves a repetitive back and forth motion of the blood pump such that during half of one cycle the pump pushes blood forward at a constant flow rate, and during the second half of the cycle blood is pulled back at about half the rate. The asymmetric cycle helps wash away any cells or other blood products that could potentially adhere to the tubing walls. During this stage of infusion, flow is controlled to limit the pressure.

The 3rd stage of infusion begins with the blood pump executing a repetitive alternating forward-pause motion that provides pulsatile acceleration and washing of blood products from the tubing walls. The flow in this stage is also pressure controlled.

It is possible to use another optical termination type measurement to determine when the majority of blood has been re-infused back into the patient and exited the optical cell. The basic principles are the same but in this application the termination measurement is looking for stability in the saline sample instead of stability in the blood sample. The method can be used to make sure there is no residual blood in the cell.

Cleaning Stages. At this point in the cycle more than 97% of the blood has been returned to the patient; the next stages focus on a more thorough cleaning of the cuvette, tubing and catheter.

Scrub Stage. The first stage of cleaning is known as ‘scrub’. The scrub stage involves rapid, reverse-synchronized back and forth motion of the blood and flush pumps so that fluid movement occurs only within the blood and flush lines with minimum net fluid flow to or from the patient. The flow is not turbulent, but the rapid oscillations create accelerations that help to wash any small amount of residual blood that can collect on the walls of the tubing and cuvette.

Recirculation Stage. Once the remaining blood products have been lifted off the tubing and cuvette walls into the mainstream by the scrub stage, the blood and flush pumps are operated at a constant, nearly synchronized rate, flushing the lines into the waste bag while flushing a small amount of saline to the patient to keep blood from migrating back into the catheter.

It is also possible to use an optical termination type measurement to access when the cell has been adequately cleaned. Even small amounts of protein can be assessed optically. Thus, the optical measurement method can be used to determine when adequate cleaning of the cell has occurred. In use the method can compare the spectral response from a prior measurement to the current measurement. If there is optical evidence of additional protein in the cell then additional cleaning might be indicated.

Catheter Flush Stage. In the final cleaning stage high flow rate controlled volume pulses completely clear the catheter extension line, tubing connectors and the catheter itself.

KVO Stage. The period between measurement cycles is KVO (Keep Vein Open). KVO provides a low, constant flow rate into the patient to prevent blood from migrating into the catheter thus maintaining an open blood access connection between draws.

The Blood Access System operation comprises 6 primary stages:

Draw initialization and clearing the catheter access;

Blood withdrawal;

Optical measurement;

Infusion;

Cleaning (incorporating Scrub, Recirculation, and Catheter Flush sub-stages);

KVO (“keep vein open”).

Draw Initialization Stage; Clearing Catheter Access. Before the blood draw is started, both the blood and flush pumps are controlled to issue a pulse of saline to clean away any residual blood in the catheter tip. This prepares the catheter for the subsequent withdrawal of blood.

Blood Withdrawal Stage. The blood pump is used to withdraw the blood sample and position non-diluted blood in the cuvette. To minimize the total draw time, about 80% of the total required blood volume is first drawn at a rapid flow rate. A constant-pressure-based draw method is used to compensate for the varying mix of saline and blood, and to achieve maximum flow rate constrained by the constant upstream negative pressure that keeps fluid degassing minimized. As blood replaces saline in the blood line, viscosity and resistance to flow increase so that for a constant upstream pressure, flow rate decreases over time. The termination of this stage of the draw is determined by what is referred to as optical termination. Optical termination is the optical detection of when a sample appropriate for measurement has filled the cuvette. After the optical termination of the withdrawal stage, the measurement of the sample can be initiated. An example of a specific optical termination method will be disclosed in detail below. Non-optical methods of detecting the arrival of an undiluted blood sample, such as those described elsewhere herein, can also be used.

Optical Measurement Stage. Following the rapid draw, the pump flow rate is slowed to a constant flow rate of 0.5 mL/min to maintain suspension of the red blood cells in plasma during optical measurement. During the 60 second measurement period an additional 500 μL of blood is withdrawn.

Infusion Stage. After the measurement is completed, reinfusion immediately begins as a progression of stages that are designed to return the blood quickly to the patient and clean the tubing and optical cuvette. The initial stage of infusion uses a constant pressure-based control which results in a variable flow rate that minimizes the time to reinfuse the blood to the patient. This stage reinfuses nearly all of the blood that was withdrawn, leaving a remaining saline-blood mixture at the end of the blood line. The first stage of the reinfusion can be completed within three minutes of the initiation of blood withdrawal.

The 2nd stage of infusion involves a repetitive back and forth motion of the blood pump such that during half of one cycle the pump pushes blood forward at a constant flow rate, and during the second half of the cycle blood is pulled back at about half the rate. The asymmetric cycle helps wash away any cells or other blood products that could potentially adhere to the tubing walls. During this stage of infusion, flow is controlled to limit the pressure.

The 3rd stage of infusion begins with the blood pump executing a repetitive alternating forward-pause motion that provides pulsatile acceleration and washing of blood products from the tubing walls. The flow in this stage is also pressure controlled.

It is possible to use another optical termination type measurement to determine when the majority of blood has been re-infused back into the patient and exited the optical cell. The basic principles are the same but in this application the termination measurement is looking for stability in the saline sample instead of stability in the blood sample. The method can be used to make sure there is no residual blood in the cell.

Cleaning Stages. At this point in the cycle more than 97% of the blood has been returned to the patient; the next stages focus on a more thorough cleaning of the cuvette, tubing and catheter.

Scrub Stage. The first stage of cleaning is known as ‘scrub’. The scrub stage involves rapid, reverse-synchronized back and forth motion of the blood and flush pumps so that fluid movement occurs only within the blood and flush lines with minimum net fluid flow to or from the patient. The flow is not turbulent, but the rapid oscillations create accelerations that help to wash any small amount of residual blood that can collect on the walls of the tubing and cuvette.

Recirculation Stage. Once the remaining blood products have been lifted off the tubing and cuvette walls into the mainstream by the scrub stage, the blood and flush pumps are operated at a constant, nearly synchronized rate, flushing the lines into the waste bag while flushing a small amount of saline to the patient to keep blood from migrating back into the catheter. It is also possible to use an optical termination type measurement to access when the cell has been adequately cleaned. Even small amounts of protein can be assessed optically. Thus, the optical measurement method can be used to determine when adequate cleaning of the cell has occurred. In use the method can compare the spectral response from a prior measurement to the current measurement. If there is optical evidence of additional protein in the cell then additional cleaning might be indicated.

Catheter Flush Stage. In the final cleaning stage high flow rate controlled volume pulses completely clear the catheter extension line, tubing connectors and the catheter itself.

KVO Stage. The period between measurement cycles is KVO (Keep Vein Open). KVO provides a low, constant flow rate into the patient to prevent blood from migrating into the catheter thus maintaining an open blood access connection between draws.

FIG. 163 provides a block diagram of the measurement sequence for an automated blood glucose monitor as described in the preceding section. During each phase of the measurement cycle, various parameters are monitored to determine proper operation and functionality of the system. An overview of the parameters used to monitor the system and the sample are indicated in FIG. 164.

In the “1st Background” phase, measurements can be taken of the fluid present at the measurement site, which fluid should be primarily saline (or other system fluid, and not blood). The measurements can be analyzed for variance and trends as described elsewhere herein. If the variance and trends do not match those expected for this phase of operation, then an error can be indicated.

In the “Blood draw” phase, measurements can be taken of the fluid that is present at the measurement site, which fluid should be transitioning from primarily saline (or other system fluid) to a mix of saline and blood to blood with minimal saline. The measurements can be analyzed for variance and trends as described elsewhere herein. As examples, any parameters that are present differently in blood than in saline (e.g., optical scatter, or some analyte concentrations) should show a time trend from the saline value to the blood value, then become stable after the measurement site is largely filled with blood. If the measurements do not indicate that the fluid is transitioning to substantially pure blood, then an error can be indicated.

In the “Sample” phase, the measurement site should be exposed to substantially pure blood sample. Measurements taken should show variance and stability consistent with such a sample, e.g., generally little or no trends, and variability within the range established by the measurement system itself. If the measurements are not consistent with a substantially pure blood sample, then an error can be indicated.

In the “Reinfuse”, “Flush”, and “KVO” phases, the measurement site should be exposed to varying combinations of blood and saline, ending with substantially pure saline by the KVO phase. Measurements taken during these phases should have trends and variability consistent with a declining proportion of blood present at the measurement site. If they do not, then an error can be indicated.

FIGS. 160, 161, and 167 comprise plots of a sample parameter exhibiting three different overall characteristics. The parameter can be determined in various ways, for example using an optical measurement system, or using an electrochemical measurement sensor, or using an ultrasound sensor. The parameter can comprise a single property of the sample, or a combination of properties. The parameter used for quality assessment can be the same parameter as that desired to be measured, or can be a different parameter that can serve as an indicator of the quality of the desired parameter measurement. The parameter used for quality assessment can be measured using the same sensor as used for the parameter desired to be measured, or can be measured using a different sensor system.

FIG. 160 is a plot of a parameter used to assess quality, where the parameter does not exhibit significant time trends or variability greater than that expected for the parameter and sensor used. For example, the parameter can comprise concentration of an analyte, in which case the plot indicates that the analyte concentration is stable over time and has a value near 100. As another example, the parameter can comprise a measurement of sample temperature or optical scattering, while the parameter of interest is concentration of an analyte. In this case, the plot indicates that the temperature or optical scattering measure is stable over time, indicating that the sample present for analyte concentration measurement is stable and the corresponding analyte measurement is likely to be accurate.

FIG. 161 is a plot of a parameter used to assess quality, where the parameter shows a decreasing value over time (also referred to as a “trend” or “time trend”). For example, the parameter can comprise concentration of an analyte, in which case the plot indicates that the analyte concentration is decreasing over time and approaching a stable value of about 100. This analysis can be used to indicate when an acceptable sample measurement has been made, i.e., when the time trend decreases and leaves a stable value. As another example, the parameter can be a measurement of sample temperature or optical scattering, in which case the plot indicates that the sample is changing over time, for example as the sample presented to the measurement system changes from saline to blood/saline mix to blood. Measurements of the desired blood property can be determined to be inaccurate while the dilution is changing, as indicated by the time trend of the sample quality parameter.

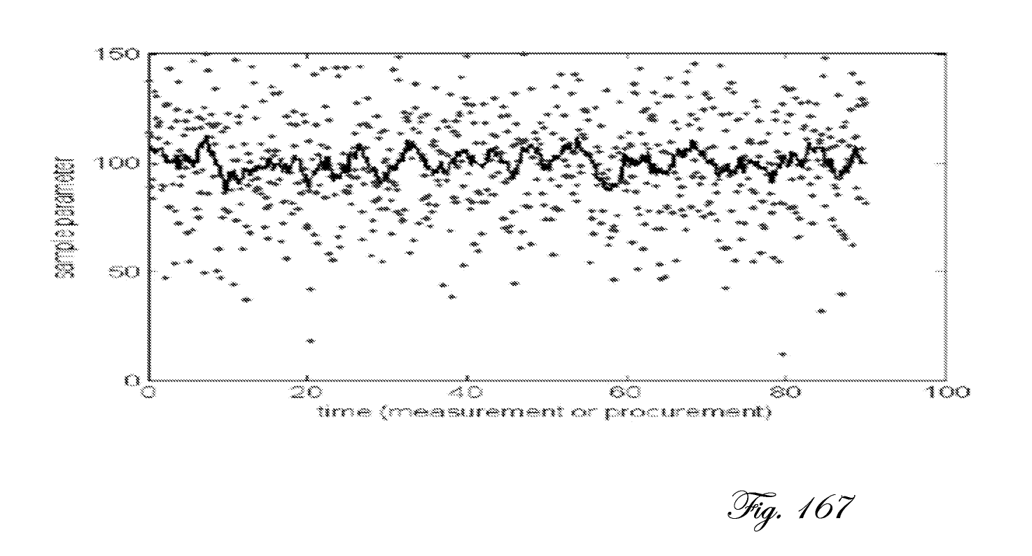

FIG. 167 is a plot of a parameter used to assess quality, where the parameter does not exhibit a significant time trend but does exhibit variability greater than the expected range for the parameter and sensor. As an example, the parameter can be concentration of an analyte in the sample, and the variability can indicate that the sensor system is not operating in acceptable performance limits. As another example, the parameter can be a measurement of sample temperature or optical scattering, in which case the excessive variability can indicate that the system has presented an unacceptable sample to the analyte measurement system, and the accuracy of the analyte measurement can be in question. This can be important if the nature of the excessive variability can lead to inaccurate but stable analyte measurement, so analysis of the analyte measurement itself might not reveal the error.

Having thus described in detail certain embodiments of the present invention, it is to be understood that the invention described herein is not to be limited to particular details set forth in the above description as many apparent variations and equivalents thereof are possible without departing from the spirit or scope of the present invention.

What is claimed is:

1. A method of measuring an analyte in a patient, comprising:
(a) removing a sample of blood from the patient; and
(b) measuring the analyte in the sample.

2. A method as in claim 1, comprising
(a) removing a sample of blood from the patient;
(b) transporting the sample of blood in a sterile manner to an analyte measurement system;
(c) measuring the analyte parameter in the transported sample using the analyte measurement system;
(d) transporting at least a portion of the measured blood to the patient in a sterile manner and infusing the portion into the patient;
(e) transporting a maintenance substance to the analyte measurement system without infusing a substantial amount of the maintenance substance into the patient;
(f) transporting at least a portion of the maintenance substance from the analyte measurement system to a waste channel.

3. A method as in claim 1, comprising:
(a) Measuring the value of the analyte at a plurality of times, with each pair of successive measurements separated by a time interval;
(b) Wherein the time intervals are not all the same duration;
(c) And wherein at least one time interval is determined from at least one patient condition, or at least one environmental condition, or a combination thereof.

4. A method of withdrawing a blood sample from a withdrawal catheter port, wherein an infusate is infused through an infusion catheter port, comprising:
(a) determining patient conditions related to blood flow or pressure that are likely to lead to contamination of the withdrawn blood sample with the infusate, wherein the withdrawal port is distal from the heart relative to the infusion port;
(b) withdrawing a sample from the withdrawal port under withdrawal conditions determined in part from the patient conditions.

5. A method as in claim 1, comprising comparing an indicator characteristic of blood from the patient determined at a first time with the indicator characteristic of the blood determined at a second time, and evaluating the comparison against a metric.

6. A method as in claim 1, further comprising calibrating an automated analyte measurement system by passing calibration fluid having at least two different analyte concentrations by an analyte sensor while infusing substantially none of at least one of such calibration fluids into the patient.

7. A method as in claim 1, further comprising controlling a level of blood glucose in a patient using an extracorporeal blood circuit, and comprising:
(a) withdrawing blood from a vascular system in the patient to the extracorporeal circuit;
(b) removing ultrafiltrate from the withdrawn blood in the circuit and passing the ultrafiltrate through an ultrafiltration passage;
(c) determining a level of glucose present in the blood using a glucose sensor monitoring ultrafiltrate flowing through an ultrafiltration passage;
(d) infusing insulin into the vascular system to control the blood glucose, wherein a rate of insulin infused is based on the determined level of glucose;
(e) introducing a calibration solution into the ultrafiltrate passage; and
(f) calibrating the glucose sensor based on a measurement made by the sensor of the calibration solution flowing through the ultrafiltrate passage.

8. A method of determining the presence of a bubble in a blood access system comprising at least one pressure detector, comprising:
(a) Using the pressure detector to determine a first frequency response of the system at a first time;
(b) Using the pressure detector to determine a second frequency response of the system at a second time;
(c) Determining if a bubble is present in the system by comparing the first and second frequency responses.

9. A method as in claim 1, comprising:
(a) Placing a blood access system in fluid communication with the circulatory patient, wherein the blood access system comprises at least one pressure sensor, at least one analye sensor, and at least one pump;
(b) Using the pressure sensor to determine the frequency response of the blood access system at a first time before step c;
(c) Operating the pump to withdraw blood from the patient to the analyte sensor;
(d) Operating the analyte sensor to determine the presence, concentration, or both of an analyte in the withdrawn blood;
(e) Using the pressure sensor to determine the frequency response of the blood access system at a second time after step c;
(f) Determining if a bubble is present in the blood access system by comparing the frequency response determined at the first time with the frequency response determined at the second time.

10. A method as in claim 1, further comprising determining the quality of a biological sample procured for ex vivo analysis, by:
(a) measuring a parameter of the biological sample at two or more distinct times;
(b) analyzing the measurements to determine a relationship between the two or more measurements;
(c) determining whether the relationship within predetermined limits.

11. An apparatus that measures an analyte in a patient, comprising a subsystem configured to remove a sample of blood or other fluid from the patient, and a subsystem configured to measure the analyte in the sample.

12. An apparatus as in claim 11, comprising:
(a) An analyte measurement system;
(b) A fluidics system, configured to remove blood from a body, transport a portion of the removed blood to the analyte measurement system for measurement, infuse a portion of the blood measured by the analyte measurement system back into the patient, flow a maintenance substance to the analyte measurement system without infusing a substantial amount of the maintenance substance into the patient, and flow at least a portion of the maintenance substance from the analyte measurement system to a waste channel.

13. An apparatus as in claim 11, comprising:
(a) A blood removal element, configured to communicate blood with the circulatory system of a patient;
(b) A first fluid transport apparatus, in fluid communication with the blood removal element;
(c) A second fluid transport apparatus, in fluid communication with the blood removal element and the first fluid transport apparatus;
(d) An analyte sensor, in fluid communication with the first fluid transport apparatus;
(e) A fluid management system, in fluid communication with the first and second fluid transport apparatuses and configured to control fluid flow in the first and second fluid transport apparatuses.

14. An apparatus as in claim 11, comprising:
(a) a blood removal element, configured to communicate blood with the circulatory system of a patient;
(b) a first fluid transport apparatus, in fluid communication with the blood removal element;
(c) a second fluid transport apparatus, in fluid communication with the blood removal element and the first fluid transport apparatus;
(d) an analyte sensor, in bidirectional fluid communication with at least one of the first fluid transport apparatus and second fluid transport apparatus;
(e) a first fluid pump, mounted with the first fluid transport apparatus such that the first fluid pump can draw fluid into and push fluid out of the first fluid transport apparatus;
(f) a second fluid pump, in fluid communication with the second fluid transport apparatus;
(g) a maintenance fluid reservoir, in fluid communication with the first fluid pump and configured to supply a maintenance fluid to the first fluid pump;
(h) a waste system, in fluid communication with the second fluid pump.

15. An apparatus as in claim 1, comprising:
(a) A fluid access system, configured to withdraw a sample of a bodily fluid from a patient;
(b) An analyte measurement system, configured to measure the value of an analyte in a sample withdrawn from the patient by the fluid access system;
(c) A controller, configured to respond to a patient condition, an environment condition, or a combination thereof, and to cause the fluid access system to withdraw a sample for measurement by the analyte measurement system

16. An apparatus as in claim 11, comprising:
(a) A patient interface device, capable of interfacing with the circulatory system of a patient;
(b) An analyte sensor having first and second ports, with the first port in fluid communication with the patient interface device;
(c) A flow generation and reservoir system having first and second ports, with the first port in fluid communication with second port of the analyte sensor; and
(d) A first fluid source, mounted such that it can be placed in fluid communication with the second port of the flow generation and storage system, wherein the first fluid source provides a first fluid having a first predetermined analyte concentration.

17. An apparatus as in claim 11, comprising:
(a) A patient interface device, capable of interfacing with the circulatory system of a patient;
(b) An analyte sensor having first and second ports, with the first port in fluid communication with the patient interface device;
(c) A flow generation and reservoir system having first and second ports, with the first port in fluid communication with second port of the analyte sensor;
(d) A first fluid source, mounted such that it can be placed in fluid communication with the second port of the flow generation and reservoir system, wherein the first fluid source provides a first fluid having a first predetermined analyte concentration; and
(e) A second fluid source, mounted such that it can be placed in fluid communication with the second port of the analyte sensor, wherein the second fluid source provides a second fluid having a second predetermined analyte concentration, where the second predetermined analyte concentration is different than the first predetermined analyte concentration.

18. An apparatus as in claim 11, comprising:
(a) A patient interface device capable of interfacing with the circulatory system of a patient;
(b) An analyte sensor having first and second ports, with the first port in fluid communication with the patient interface device;
(c) A flow generation device having first and second ports, with the first port in fluid communication with second port of the analyte sensor;
(d) A waste channel in fluid communication with the second port of the flow generation device through a first flow control device that allows fluid flow from the flow generation device to the waste channel but substantially prevents fluid from the waste channel to the flow generation device;
(e) A first fluid source, mounted such that it can be placed in fluid communication with the second port of the flow generation device through a second flow control device that allows fluid flow from the first fluid source to the flow generation device but substantially prevents fluid from the flow generation device to the first fluid source, wherein the first fluid source provides a first fluid having a first predetermined analyte concentration.

19. An apparatus as in claim 11, comprising:
(a) An arterial catheter, configured to be placed in fluid communication with an artery of a patient;
(b) A blood pressure monitoring subsystem mounted with the arterial catheter such that the blood pressure monitoring subsystem can determine the pressure of blood in the artery; and
(c) An analyte measuring subsystem mounted with the arterial catheter such that the analyte measuring subsystem can determine the presence, concentration, or both of one or more analytes in blood withdrawn from the artery.

20. An apparatus as in claim 11, comprising:
(a) an analyte measurement system, configured to measure the level of an analyte in a patient's blood, or an indicator thereof;
(b) an infusion recommendation system, configured to recommend medication infusion parameters based on information comprising the measured blood analyte level;
(c) an infusion control system, configured to infuse a medication into the patient;
(d) an authorization system configured to allow a clinician to authorize an infusion of the medication into the patent by the infusion control system based on a recommendation to the of infusion parameters by the infusion recommendation system.

21. An indwelling fiber optic probe, comprising at least one optical fiber having a proximal end and a distal end, wherein illumination light from a near-infrared light source is coupled into the proximal end and directed to the distal end of the fiber and wherein the distal end is inserted into a patient tissue and wherein light from the tissue is collected by the distal end of the at least one optical fiber and returned to the proximal end of the fiber as collected light.
